(19) United States
(12) Patent Application Publication
(10) Pub. No.: US 2015/0050728 A1
(43) Pub. Date: Feb. 19, 2015

(54) Identification Of Novel Cell Surface Markers For Pancreatic Progenitor Cells And Definite Endodermal Cells

(71) Applicants: Nissim Benvenisty, Jerusalem (IL); Joseph Itskovitz-Eldor, Haifa (IL); Bettina Fishman, Haifa (IL); Hanna Segev, Doar-Na Emek Hefer (IL); Danny Kitsberg, Jerusalem (IL)

(72) Inventors: Nissim Benvenisty, Jerusalem (IL); Joseph Itskovitz-Eldor, Haifa (IL); Bettina Fishman, Haifa (IL); Hanna Segev, Doar-Na Emek Hefer (IL); Danny Kitsberg, Jerusalem (IL)

(21) Appl. No.: 14/528,837

(22) Filed: Oct. 30, 2014

Related U.S. Application Data
(63) Continuation of application No. 13/903,815, filed on May 28, 2013.
Application No. 13/903,815 is a continuation of application No. PCT/IB2011/055283, filed on Nov. 24, 2011.
(60) Provisional application No. 61/536,099, filed on Sep. 19, 2011.
(60) Provisional application No. 61/417,364, filed on Nov. 26, 2010.

(51) Int. Cl. C12N 5/071

(52) U.S. Cl. CPC C12N 5/0678
USPC 435/325

(57) ABSTRACT

Methods of identifying, isolating and qualifying pancreatic progenitor cells and definite endodermal cells. An isolated population of pancreatic progenitor cells, including at least 75% of cells having a TROP-2+ and/or TROP-2+/GPR50+ expression pattern and an isolated population of definite endodermal cells, including at least 50% of cells having a SOX17+/SOX7+/GSC+/CER+/FOXA2+/CXCR4+/NANOG expression pattern. Nucleic acid constructs including a reporter protein under the transcriptional regulation of SOX17 regulatory sequence or of PDX1 regulatory sequence, and cells comprising same, and methods and kits using same.

### FIELD OF THE INVENTION

The present invention, in some embodiments thereof, relates to methods of identifying, isolating and qualifying pancreatic progenitor cells and definite endodermal cells, and, more particularly, but not exclusively, to isolated cell populations generated thereby.

### BACKGROUND OF THE INVENTION

Type 1 Diabetes Mellitus is an autoimmune disease affecting the life of millions world wide with enormous financial costs. It is caused by the destruction and loss of function of beta cells in the pancreatic Islets of Langerhans. The lack of insulin production causes deregulation of blood glucose levels, and leads to a large number of symptoms that can eventually be fatal. Daily insulin injections are the most prevalent treatment for type I Diabetes Mellitus and for insulin-dependent type II Diabetes Mellitus. However, insulin injections are expensive, cumbersome and do not enable the patient to attain a real steady state in blood glucose levels, but instead lead to fluctuations above and below the optimal base line, which do not ultimately prevent complications of diabetes. A potential cure for these diseases is transplantation therapy whereby islets are transplanted into the patient. However, the limited number of donor organs presently restricts the use of this procedure.

New sources of beta cells are needed in order to develop cell therapies for patients with insulin-dependent diabetes. An alternative to forced expansion of post-mitotic beta cells is the induction of differentiation of pluripotent stem/progenitor cells (which have a natural self-expansion capacity) of different origins, into insulin-producing cells. Various publications describe protocols for differentiation of human embryonic stem cells (hESCs) (5-9), induced pluripotent stem cells (iPSCs) (10, 11), or cord blood mesenchymal stem cells (Chao C K et al., PLoS ONE. 2008; 3(1): e1451; Santos T M et al., Transplantation Proceedings, 42: 563-565, 2010; Bhandari D R et al., Differentiation. 2011 Jul. 20. [Epub ahead of print]) into pancreatic endocrine cells which might provide a source of insulin producing cells for diabetics.

The in vitro differentiation of cells to pancreatic beta cells, like in vivo embryonic development, is a stepwise process by which the initially pluripotent/multipotent cells, such as human ESCs or iPSCs, progressively commit towards a more specialized cell fate ultimately resulting in insulin producing cells. Though insulin is a classic marker for pancreatic endocrine cells it is only a useful marker for cells that have reached the endpoint of differentiation and are fully functional beta islet cells.

Two significant points along the differentiation process are the endodermal progenitor stage and the pancreatic progenitor stage. These stages are characterized by the expression of stage specific transcription factors. Two examples of these are SRY box 17 (SOX17) and pancreatic and duodenal homeobox 1 (PDX1), which are expressed relatively early in pancreatic differentiation. SOX17 is expressed at the earliest stage of hESC differentiation towards definitive endoderm and PDX1 is expressed at onset of the earliest commitment stages towards pancreas (12).

Lavon et al. (25) transfected hESCs with a reporter construct which included the enhanced green fluorescent protein (EGFP) under the albumin promoter (ALB-eGFP).

Additional background art includes Wang P., et al. Cell Stem Cell, 8: 335-346, 2011; WO 2005116073; Zwaka T P and J A Thomson, 2003 [Homologous recombination in human embryonic stem cells. Nat Biotechnol 21:319-321]; Micallef S J., 2005 (Diabetes 54: 301-305); Nikakan K K., 2010 (Genes Dev. 24:312-326); D'Amour K A, et al., 2005 (Efficient differentiation of human embryonic stem cells to definitive endoderm. Nat Biotechnol 23:1534-1541); Yasunaga M, et al., 2005 (Induction and monitoring of definitive and visceral endoderm differentiation of mouse ES cells. Nat Biotechnol 23:1542-1550); US 2011/0070645 (Chen et al.); WO 2005/116073 A2 (D'Amour K A et al.); Borowiak M, 2009 (Cell Stem Cell 4, 348-358); Jiang W., 2007 (Cell Res 17:333-344); D'amour 2006 (Nat Biotechnol 24:1392-1401); and Kroon 2008 (Nat Biotechnol 26:443-452).

### SUMMARY OF THE INVENTION

According to an aspect of some embodiments of the present invention there is provided a method of identifying pancreatic progenitor cells, comprising determining in a population of cells which comprises pancreatic progenitor cells at least one marker that is: (i) positively associated with pancreatic differentiation, the marker being selected from the group consisting of: TACSTD2 (TROP-2), GPR50, BST2, NTRK2, ITGA4, KDR, PTPRN, LGI1, VIPR2, SLC2A1, MUC15, MUC12, LPHN3, MUC16, VTCN1, MMP16, FZD3, ITGB6, GFRA3, ROBO1, NLGN1, BST2, MUC12, CNTFR, LPHN1, SULF1, ADAM23, SCUBE3, PLAU, CDON, SLIT2, C7orf68, PLXDC2, CD74, MUC15, GPR56, VTCN1, ITGB6, NTRK2, AREG, BOC, ITGA4, KLRK1, PRTG, PTPRZ1, GABRP, SILV, KIAA1772, PLP1, OVOS, HAPLN1, EPHA7, ENO2, PCDHB5, OVOS, SYT1, DCT, GPAM, SLITRK6, DCC, FREM2, SDK2, CGA, ATP1B2, SEMA3D, PCDHB15, CDH1, WNT8B, LPAR4, NPIPL3, FAM171B, PTN, ABCC2, ADAMTS3, RASA4, CPT1C, SLC6A6, PCDHB3, LRRC37B, RNFT2, KCNG3, TRPC1, ALPPL2, OR4F21, CCL2, KIF5A, OLFM2, CACNG7, MPHOSPH9, SLC13A4, MOXD1, C6orf186, SLC4A8, STX16, AMY2A, SPARCL1, MGP, A2M, DCN, ATP8B1, MMRN1, EMP1, PLA2G2A, PDE3A, TLR3, CYP1B1, PTGIS, RFTN2, PLEKHA2, SMOC1, STOM, JAM2, CHL1, SCG5, IGFBP7, NPR3, IFI6, CR1L, OR2A4, OR2A7, KCNG3, CACNG7, GRID2, CDH1, LPAR3, SEMA6A, PTPRZ1, ATP1A3, CAMKV, SCNN1G, SYT6, SLC18A2, PCDHB5, ABCG2, HLA-DRA, CR1L, HTR2C, EDNRB, PCDH11X, SLC17A7, SCNN1A, CD9, CXCL16, FXYD5, GABRQ, GFRA3, CACNA2D2, CLDN4, PTPRN, PLP1, PDPN, MMP24, SDK2, GPR176, GPR64, GPR160, PCDH11Y, NKAIN4, ATP1B2, SCN8A, THBS4, CR2, HLA-DQA1, HLA-DRA, HTR7, SLC2A1, HLA-DRA, KCNS3, SLC7A3, HLA-DPB2, CACNA1B, and GPR143, wherein an upregulation above a predetermined threshold of an expression level of the marker positively associated with the pancreatic differentiation as compared to the expression level of the marker in a reference cell, wherein said reference cell is a definite endodermal cell which expresses SOX17, is indicative of a pancreatic progenitor cell, and alternatively or additionally (ii) negatively associated with the pancreatic differentiation, the marker being selected from the group consisting of: LGR5, CXCR4, FLRT3, TRPA1, SLC40A1, LRIG3, COLEC12, EPSTI1, GPR128, CDH2, SLC5A9, FSHR, SLC30A10, IL13RA1, SLC7A7, PCDH10, SLC1A1, GPR141, LIFR, TMEM27, GPC4, LYPD6B, FOLH1, TRPC4, PCDH7, KEL, KCNJ3, OR2T4, VIPR2, FLRT2, CD34, SLC39A8, CLDN11, CXCR7, ITGA5, ITGAV, CALCR, CLDN18, CCKBR, SLC7A5, TMBIM4, SLCO2A1, CDH10, AMHR2, ASAM, CLDN1, DSCAM, TMEM88, PLXNA2, CD177, TMEM144, GPR37, GJA5, SEMA6D, NIPAL2, GPR151, MCC, TMEM136, KCNG1, LHFPL2, MOSPD1, SLC37A1, LRIT3, EPHA4, GPR177, and IL1R1, wherein a downregulation above a predetermined threshold of an expression level of the marker negatively associated with the pancreatic differentiation as compared to the expression level of the marker in a reference cell, wherein said reference cell is a definite endodermal cell which expresses SOX17, is indicative of a pancreatic progenitor cell, thereby identifying the pancreatic progenitor cells.

According to an aspect of some embodiments of the present invention there is provided a method of identifying pancreatic progenitor cells, comprising determining in a population of cells which comprises pancreatic progenitor cells at least one marker that is: (i) negatively associated with the pancreatic differentiation, the marker being selected from the group consisting of: LGR5, CCKBR, CXCR4, FLRT3, TRPA1, SLC40A1, LRIG3, COLEC12, EPSTI1, GPR128, CDH2, SLC5A9, FSHR, SLC30A10, IL13RA1, SLC7A7, PCDH10, SLC1A1, GPR141, LIFR, TMEM27, GPC4, LYPD6B, FOLH1, TRPC4, PCDH7, KEL, KCNJ3, OR2T4, VIPR2, FLRT2, CD34, SLC39A8, CLDN11, CXCR7, ITGA5, ITGAV, CALCR, CLDN18, SLC7A5, TMBIM4, SLCO2A1, CDH10, AMHR2, ASAM, CLDN1, DSCAM, TMEM88, PLXNA2, CD177, TMEM144, GPR37, GJA5, SEMA6D, NIPAL2, GPR151, MCC, TMEM136, KCNG1, LHFPL2, MOSPD1, SLC37A1, LRIT3, EPHA4, GPR177, IL1R1, CST1, CER1, ANKRD1, TRY6, HAS2, DKK1, PRSS2, HP, APOA2, RHOBTB3, BMP2, ACE2, STC1, PDZK1, HHEX, VIL1, PRDM1, EOMES, DNAJC15, TNIK, IGFBP5, RLBP1L2, ADAMTS9, EPSTI1, C5, ARHGAP24, TRY6, ANGPT2, TTR, MYL7, FST, KITLG, GATA3, ST8SIA4, CCDC141, TSPYL5, EGFLAM, TTN, LEFTY2, FOXA2, FAM184A, STMN2, DIO3, FN1, PRSS1, NPPB, and OTX2, wherein a downregulation above a predetermined threshold of an expression level of the marker negatively associated with the pancreatic differentiation as compared to the expression level of the marker in a reference cell, wherein said reference cell is a definite endodermal cell which expresses SOX17, is indicative of a pancreatic progenitor cell and alternatively or additionally; (ii) positively associated with the pancreatic differentiation, the marker being selected from the group consisting of: TACSTD2 (TROP-2), GPR50, BST2, NTRK2, ITGA4, KDR, PTPRN, LGI1, VIPR2, SLC2A1, MUC15, MUC12, LPHN3, MUC16, VTCN1, MMP16, FZD3, ITGB6, GFRA3, ROBO1, NLGN1, MUC12, CNTFR, LPHN1, SULF1, ADAM23, SCUBE3, PLAU, CDON, SLIT2, C7orf68, PLXDC2, CD74, MUC15, GPR56, VTCN1, ITGB6, AREG, BOC, KLRK1, PRTG, PTPRZ1, GABRP, SILV, KIAA1772, PLP1, OVOS, HAPLN1, EPHA7, ENO2, PCDHB5, OVOS, SYT1, DCT, GPAM, SLITRK6, DCC, FREM2, SDK2, CGA, ATP1B2, SEMA3D, PCDHB15, CDH1, WNT8B, LPAR4, NPIPL3, FAM171B, PTN, ABCC2, ADAMTS3, RASA4, CPT1C, SLC6A6, PCDHB3, LRRC37B, RNFT2, KCNG3, TRPC1, ALPPL2, OR4F21, CCL2, KIF5A, OLFM2, CACNG7, MPHOSPH9, SLC13A4, MOXD1, C6orf186, SLC4A8, STX16, AMY2A, SPARCL1, MGP, A2M, DCN, ATP8B1, MMRN1, EMP1, PLA2G2A, PDE3A, TLR3, CYP1B1, PTGIS, RFTN2, PLEKHA2, SMOC1, STOM, JAM2, CHL1, SCG5, IGFBP7, NPR3, IFI6, CR1L, OR2A4, OR2A7, KCNG3, CACNG7, GRID2, CDH1, LPAR3, SEMA6A, PTPRZ1, ATP1A3, CAMKV, SCNN1G, SYT6, SLC18A2, PCDHB5, ABCG2, HLA-DRA, CR1L, HTR2C, EDNRB, PCDH11X, SLC17A7, SCNN1A, CD9, CXCL16, FXYD5, GABRQ, GFRA3, CACNA2D2, CLDN4, PDPN, MMP24, SDK2, GPR176, GPR64, GPR160, PCDH11Y, NKAIN4, ATP1B2, SCN8A, THBS4, CR2, HLA-DQA1, HLA-DRA, HTR7, SLC2A1, HLA-DRA, KCNS3, SLC7A3, HLA-DPB2, CACNA1B, GPR143, ALPPL2, DPPA5, H19, CRYZ, CXCL12, TYW3, ZYG11A, CRABP1, IDO1, POU5F1, HEY2, HIST1H1A, TFAP2C, DPPA2, ZFP42, LECT1, NECAB1, CKMT1A, SAMHD1, FGF2, PLA2G2A, PRDM14, POU5F1, GLI3, GSTT2, OLFML3, DAZL, GALNT3, SOX2, POU5F1B, ACTN3, CPT1A, DCLK1, EDIL3, NANOG, THUMPD3, VASH2, ATCAY, USP44, HIST1H4F, NANOG, PIM2, DNMT3B, ZNF483, FEZF1, SCARNA9L, and SILV, wherein an upregulation above a predetermined threshold of an expression level of the marker positively associated with the pancreatic differentiation as compared to the expression level of the marker in a reference cell, wherein said reference cell is a definite endodermal cell which expresses SOX17, is indicative of a pancreatic progenitor cell.

According to an aspect of some embodiments of the present invention there is provided a method of identifying pancreatic progenitor cells, comprising determining in a population of cells which comprises pancreatic progenitor cells at least one marker that is: (i) negatively associated with pancreatic differentiation, the marker being selected from the group consisting of: LGR5, CCKBR, CXCR4, FLRT3, TRPA1, SLC40A1, LRIG3, COLEC12, EPSTI1, GPR128, CDH2, SLC5A9, FSHR, SLC30A10, IL13RA1, SLC7A7, PCDH10, SLC1A1, GPR141, LIFR, TMEM27, GPC4, LYPD6B, FOLH1, TRPC4, PCDH7, KEL, KCNJ3, OR2T4, VIPR2, FLRT2, CD34, SLC39A8, CLDN11, CXCR7, ITGA5, ITGAV, CALCR, CLDN18, SLC7A5, TMBIM4, SLCO2A1, CDH10, AMHR2, ASAM, CLDN1, DSCAM, TMEM88, PLXNA2, CD177, TMEM144, GPR37, GJA5, SEMA6D, NIPAL2, GPR151, MCC, TMEM136, KCNG1, LHFPL2, MOSPD1, SLC37A1, LRIT3, EPHA4, GPR177, and IL1R1, wherein downregulation above a predetermined threshold of an expression level of the marker negatively associated with the pancreatic differentiation as compared to the expression level of the marker in a reference cell, wherein said reference cell is a definite endodermal cell which expresses SOX17, is indicative of a pancreatic progenitor cell, and alternatively or additionally; (ii) positively associated with pancreatic differentiation, the marker being selected from the group consisting of: TACSTD2 (TROP-2), GPR50, BST2, NTRK2, ITGA4, KDR, PTPRN, LGI1, VIPR2, SLC2A1, MUC15, MUC12, LPHN3, MUC16, VTCN1, MMP16, FZD3, ITGB6, GFRA3, ROBO1, NLGN1, MUC12, CNTFR, LPHN1, SULF1, ADAM23, SCUBE3, PLAU, CDON, SLIT2, C7orf68, PLXDC2, CD74, MUC15, GPR56, VTCN1, ITGB6, AREG, BOC, KLRK1, PRTG, PTPRZ1, GABRP, SILV, KIAA1772, PLP1, OVOS, HAPLN1, EPHA7, ENO2, PCDHB5, OVOS, SYT1, DCT, GPAM, SLITRK6, DCC, FREM2, SDK2, CGA, ATP1B2, SEMA3D, PCDHB15, CDH1, WNT8B, LPAR4, NPIPL3, FAM171B, PTN, ABCC2, ADAMTS3, RASA4, CPT1C, SLC6A6, PCDHB3, LRRC37B, RNFT2, KCNG3, TRPC1, ALPPL2, OR4F21, CCL2, KIF5A, OLFM2, CACNG7, MPHOSPH9, SLC13A4, MOXD1, C6orf186, SLC4A8, STX16, AMY2A, SPARCL1, MGP, A2M, DCN, ATP8B1, MMRN1, EMP1, PLA2G2A, PDE3A, TLR3, CYP1B1, PTGIS, RFTN2, PLEKHA2, SMOC1, STOM, JAM2, CHL1, SCG5, IGFBP7, NPR3, IFI6, CR1L, OR2A4, OR2A7, KCNG3, CACNG7, GRID2, CDH1, LPAR3, SEMA6A, PTPRZ1, ATP1A3, CAMKV, SCNN1G, SYT6, SLC18A2, PCDHB5, ABCG2, HLA-DRA, CR1L, HTR2C, EDNRB, PCDH11X, SLC17A7, SCNN1A, CD9, CXCL16, FXYD5, GABRQ, GFRA3, CACNA2D2, CLDN4, PTPRN, PLP1, PDPN, MMP24, SDK2, GPR176, GPR64, GPR160, PCDH11Y, NKAIN4, ATP1B2, SCN8A, THBS4, CR2, HLA-DQA1, HLA-DRA, HTR7, SLC2A1, HLA-DRA, KCNS3, SLC7A3, HLA-DPB2, CACNA1B, and GPR143, wherein upregulation above a predetermined threshold of an expression level of the marker positively associated with the pancreatic differentiation as compared to the expression level of the marker in a reference cell, wherein said reference cell is a definite endodermal cell which expresses SOX17, is indicative of a pancreatic progenitor cells, and alternatively or additionally; (iii) negatively associated with pancreatic differentiation, the marker being selected from the group consisting of: TMPRSS11E, LGR5, SLC39A8, TM4SF18, CUZD1, GPC4, SLC22A3, CXCR4, NRN1, TMBIM4, THBS2, SLC7A5, and TMEM47, wherein downregulation above a predetermined threshold of an expression level of the marker negatively associated with pancreatic differentiation as compared to the expression level of the marker in a reference cell, wherein said reference cell is an undifferentiated human embryonic stem cell (hESC), is indicative of a pancreatic progenitor cell, and alternatively or additionally; (iv) positively associated with pancreatic differentiation, the marker being selected from the group consisting of: TACSTD2 (TROP-2), LRP2, LAMA1, GPC3, LGI1, FN1, LPHN3, FGB, KIT, DPP4, TRO, SPA17, ROBO2, DLK1, BST2, PRTG, LRP2, CACNG7, DLK1, CACNA2D2, CR1L, SCNN1G, HTR2C, LPAR3, THBS3, KCNG3, SDK2, HLA-DRA, SLC18A2, CXCL16, TMEM63C, SLC17A7, GFRA3, PRTG, DKK1, APOA2, AFP, APOB, FLRT3, KIAA1772, FBN2, FRZB, KLK6, FREM2, RSPO3, APOA1, APOA1, SEMA3D, KEL, BMP5, HEPH, STMN2, CACNB3, CHST9, TMC7, FAM171B, ATP5G1, FOLR1, ABCC2, EFNA5, CPT1C, MAGED1, RASA4, KDELR3, ST6GALNAC2, EDNRA, LRRC37B, ABCC4, CLCN5, MBOAT2, SLC44A5, NPC2, ELFN1, MMD, SLC5A3, IMPAD1, OSTC, DSC2, SLC31A1, SLC5A12, LRRN3, NPIPL3, GDPD1, NPIPL3, NPIPL3, LINGO2, and LYPD6B, wherein upregulation above a predetermined threshold of an expression level of the marker positively associated with pancreatic differentiation as compared to the expression level of the marker in a reference cell, wherein said reference cell is an undifferentiated human embryonic stem cell (hESC), is indicative of a pancreatic progenitor cell; thereby identifying the pancreatic progenitor cells.

According to an aspect of some embodiments of the present invention there is provided a method of isolating pancreatic progenitor cells, comprising: (a) identifying the pancreatic progenitor cells according to the method of some embodiments of the invention, and (b) isolating the pancreatic progenitor cells identified according to step (a) to thereby obtain isolated pancreatic progenitor cells, thereby isolating the pancreatic progenitor cells.

According to an aspect of some embodiments of the present invention there is provided a method of qualifying a pancreatic progenitor cell population, comprising: determining in a sample of the cell population a percentage of the pancreatic progenitor cells which are identified according to the method of some embodiments of the invention out of the total cells in the sample, thereby qualifying the pancreatic progenitor cell population.

According to an aspect of some embodiments of the present invention there is provided a method of isolating endocrine progenitors or insulin producing cells, comprising culturing the pancreatic progenitor cells isolated by the method of some embodiments of the invention or the pancreatic progenitor cell population qualified according to the method of some embodiments of the invention under conditions suitable for maturation of the pancreatic progenitor cells into endocrine progenitors or beta cells, thereby generating insulin producing cells.

According to an aspect of some embodiments of the present invention there is provided a method of transplanting pancreatic progenitor cells or cells derived therefrom in a subject, comprising (a) qualifying the pancreatic progenitor cells according to the method of some embodiments of the invention, wherein presence of at least a predetermined percentage of the pancreatic progenitor cells in the cell sample indicates the suitability of the pancreatic progenitor cells for transplantation in a subject, to thereby obtain a pancreatic progenitor cell population being suitable for transplantation in a subject, (b) transplanting in the subject the pancreatic progenitor cell population being suitable for transplantation in a subject or cells derived therefrom, thereby transplanting the pancreatic progenitor cells or cells derived therefrom in the subject.

According to an aspect of some embodiments of the present invention there is provided a method of identifying definite endodermal cells, comprising determining in a population of cells which comprises definite endodermal cells at least one marker that is negatively associated with definite endodermal cells, the marker being selected from the group consisting of: KDR, PCDHB5, FAT4, FLT1, NRN1, THBS2, PTPRZ1, SLC6A15, GPR176, SEMA6A, THBS1, CDH11, GRID2, SLC7A11, CDH1, LRFN5, EDNRB, THY1, NETO1, KCND2, TMPRSS11E, CD44, PDPN, SLC7A1, KAL1, KCNG3, GPM6B, FXYD5, PCDH18, ICAM3, MCTP1, TACR3, and TMEM155, wherein downregulation above a predetermined threshold of an expression level of the marker negatively associated with the definite endodermal cells as compared to the expression level of the marker in a reference cell, wherein said reference cell is an undifferentiated human embryonic stem cell (hESC), is indicative of a definite endodermal cell.

According to an aspect of some embodiments of the present invention there is provided a method of identifying definite endodermal cells, comprising determining in a population of cells which comprises definite endodermal cells at least one marker that is positively associated with definite endodermal cells, the marker being selected from the group consisting of: FSHR, COLEC12, ROR2 ITGA5, LRP2, CD177, CCKBR, TRPA1, KEL, FOLR1, FOLH1, APOA1, APOA1, FZD5, FN1, BMP2, ADAMTS9, DPP4, FGA, GPR128, IGFBP5, LIFR, FZD4, PRTG, STC1, TNFSF4, CD177, IHH, LAMA1, GPC3, LGI1, FN1, LPHN3, FGB, KIT, DPP4, TRO, SPA17, ROBO2, DLK1, CST1, DKK1, HAS2, APOA2, CDH2, LGR5, AFP, FLRT3, APOB, DGKK, HP, SYTL5, SLC7A7, TTR, FRZB, RSPO3, GCNT1, DIO3, TMEM27, APOC1, EPHA4, C4orf18, GLIPR2, PCDH10, F10, BMP5, FMO5, STMN2, SLC5A9, PORCN, EGFLAM, RAB17, CST2, PNPLA3, MOSPD1, ELMO1, CHST9, SLC30A10, TMC7, C8orf49, CDH12, ST8SIA4, SLCO2A1, MANEA, LRIG3, HCN1, ADAMTS12, TMEM144, VTN, CAMK2N1, ABCC4, PCDH7, OR2T4, UNC93A, COL4A6, PAMR1, SLC1A1, PROS1, APOM, APOM, APOM, LYPD6B, TMEM88, ITLN2, BMPER, GPR141, VEGFA, DKK1, APOA2, AFP, APOB, FLRT3, KIAA1772, FBN2, FRZB, KLK6, FREM2, RSPO3, APOA1, APOA1, SEMA3D, KEL, BMP5, HEPH, STMN2, CACNB3, CHST9, TMC7, FAM171B, ATP5G1, FOLR1, ABCC2, EFNA5, CPT1C, MAGED1, RASA4, KDELR3, ST6GALNAC2, EDNRA, LRRC37B, ABCC4, CLCN5, MBOAT2, SLC44A5, NPC2, ELFN1, MMD, SLC5A3, IMPAD1, OSTC, DSC2, SLC31A1, SLC5A12, LRRN3, NPIPL3, GDPD1, NPIPL3, NPIPL3, LINGO2, LYPD6B, TRPA1, SLC40A1, SLC30A10, CCKBR, VIPR2, COLEC12, FLRT3, LGR5, GPR141, BST2, SLC5A9, GPR128, KEL, LRIG3, LYPD6B, FSHR, LIFR, FOLH1, CXCR4, ITGA5, AMOT, LY6E, SEMA6D, GJA5, PRTG, CD34, TMEM144, ROR2, GPR177, OR2T4, SLC7A7, KCNJ3, CLDN18, GPR151, SLC44A5, CDH10, TMEM27, SLC1A1, TMEM56, CD177, PLXNA2, SLC26A2, DSCAM, TMEM133, IL13RA1, ATP2B1, CD302, MEGF9, EDNRA, CDH2, GPR161, TYRO3, FLRT2, LRIT3, PCDH7, NRCAM, SMAGP, AMHR2, ELTD1, GRPR, EPHA4, and CD99, wherein upregulation above a predetermined threshold of an expression level of the marker positively associated with the definite endodermal cells as compared to the expression level of the marker in a reference cell, wherein said reference cell is an undifferentiated human embryonic stem cell (hESC), is indicative of a definite endodermal cell.

According to an aspect of some embodiments of the present invention there is provided a method of isolating definite endodermal cells, comprising: (a) identifying the definite endodermal cells according to the method of some embodiments of the invention, (b) isolating the definite endodermal cells identified according to step (a) to thereby obtain an isolated population of the definite endodermal cells, thereby isolating the definite endodermal cells.

According to an aspect of some embodiments of the present invention there is provided a method of qualifying a definite endodermal cell population, comprising: determining in a sample of the cell population a percentage of the definite endodermal cells which are identified according to the method of some embodiments of the invention out of the total cells in the sample, thereby qualifying the definite endodermal cell population.

According to an aspect of some embodiments of the present invention there is provided an isolated population of pancreatic progenitor cells obtained according to the method of some embodiments of the invention.

According to an aspect of some embodiments of the present invention there is provided an isolated population of pancreatic progenitor cells, comprising at least 75% of cells having a TROP-2+ and/or TROP-2+/GPR50+ expression pattern.

According to an aspect of some embodiments of the present invention there is provided an isolated population of definite endodermal cells obtained according to the method of some embodiments of the invention.

According to an aspect of some embodiments of the present invention there is provided an isolated population of definite endodermal cells, comprising at least 50% of cells having a SOX17+/SOX7+ expression pattern.

According to an aspect of some embodiments of the present invention there is provided a nucleic acid construct comprising a first polynucleotide encoding a reporter protein and a second polynucleotide which comprises a human endogenous SOX17 regulatory sequence, wherein the first polynucleotide being under transcriptional regulation of the SOX17 regulatory sequence, wherein the SOX17 regulatory sequence comprises an upstream sequence and a downstream sequence, wherein the upstream sequence comprises the nucleotide sequence set forth in SEQ ID NO:38; and wherein the downstream sequence comprises the nucleotide sequence set forth in SEQ ID NO:39.

According to an aspect of some embodiments of the invention there is provided a nucleic acid construct comprising a first polynucleotide encoding a reporter protein and a second polynucleotide which comprises a human endogenous PDX1 regulatory sequence, wherein the first polynucleotide being under transcriptional regulation of the PDX1 regulatory sequence, wherein the PDX1 regulatory sequence comprises an upstream sequence and a downstream sequence, wherein the upstream sequence comprises the nucleotide sequence set forth in SEQ ID NO:16; and wherein the downstream sequence comprises the nucleotide sequence set forth in SEQ ID NO:17.

According to an aspect of some embodiments of the present invention there is provided a cell comprising the nucleic acid construct of some embodiments of the invention.

According to an aspect of some embodiments of the present invention there is provided a method of screening for markers which differentiate a definite endodermal cell from an undifferentiated pluripotent stem cell, comprising comparing the expression level of markers between the undifferentiated pluripotent stem cell and the cell of some embodiments of the invention, wherein upregulation or downregulation in the expression level above a predetermined threshold indicates that the markers differentiate the definite endodermal cell from the undifferentiated pluripotent stem cell, thereby screening for markers which differentiate the definite endodermal cell from the undifferentiated pluripotent stem cell.

According to an aspect of some embodiments of the present invention there is provided a method of screening for compounds capable of inducing differentiation of undifferentiated pluripotent stem cells to definite endodermal cells, comprising: (a) contacting undifferentiated pluripotent stem cells which comprise the nucleic acid construct of some embodiments of the invention with at least one compound of a plurality of candidate compounds, and; (b) monitoring an expression level of the reporter protein in the cells following the contacting, wherein an increase above a predetermined level in the expression level of the reporter protein following the contacting as compared to the expression level prior to the contacting is indicative that the at least one compound is capable of inducing differentiation of the undifferentiated pluripotent stem cells to the definite endodermal cells, thereby screening for the compounds capable of inducing differentiation of undifferentiated pluripotent stem cells to definite endodermal cells.

According to an aspect of some embodiments of the present invention there is provided a cell comprising the nucleic acid construct of some embodiments of the invention.

According to an aspect of some embodiments of the present invention there is provided a method of screening for markers which differentiate a pancreatic progenitor cell from a definite endodermal cell, comprising comparing the expression level of markers between the cell of some embodiments of the invention and the cell of some embodiments of the invention, wherein upregulation or downregulation in the expression level above a predetermined threshold indicates that the markers differentiate the pancreatic progenitor cell from the definite endodermal cell, thereby screening for markers which differentiate the pancreatic progenitor cell from the definite endodermal cell.

According to an aspect of some embodiments of the present invention there is provided a method of screening for compounds capable of inducing differentiation of definite endodermal cells or undifferentiated pluripotent stem cells to pancreatic progenitor cells, comprising: (a) contacting definite endodermal cells or undifferentiated pluripotent stem cells which comprise the nucleic acid construct of some embodiments of the invention with at least one compound of a plurality of candidate compounds, and; (b) monitoring an expression level of the reporter protein in the cells following the contacting, wherein an increase above a predetermined level in the expression level of the reporter protein following the contacting as compared to the expression level prior to the contacting is indicative that the at least one compound is capable of inducing differentiation of the definite endodermal cells or undifferentiated pluripotent stem cells to the pancreatic progenitor cells, thereby screening for the compounds capable of inducing differentiation of definite endodermal cells or undifferentiated pluripotent stem cells to the pancreatic progenitor cells.

According to an aspect of some embodiments of the present invention there is provided a kit for screening for markers which differentiate a definite endodermal cell from a pluripotent stem cell, comprising the cell of some embodiments of the invention.

According to an aspect of some embodiments of the present invention there is provided a kit for screening for markers which differentiate a pancreatic progenitor cell from a definite endodermal cell, comprising the cell of some embodiments of the invention and the cell of some embodiments of the invention.

According to some embodiments of the invention, the method further comprising determining in a population of cells which comprises pancreatic progenitor cells at least one marker that is: (i) negatively associated with the pancreatic differentiation, the marker being selected from the group consisting of: CST1, CER1, ANKRD1, TRY6, HAS2, DKK1, PRSS2, HP, APOA2, RHOBTB3, BMP2, ACE2, STC1, PDZK1, HHEX, VIL1, PRDM1, EOMES, DNAJC15, TNIK, IGFBP5, RLBP1L2, ADAMTS9, EPSTI1, C5, ARHGAP24, TRY6, ANGPT2, TTR, MYL7, FST, KITLG, GATA3, ST8SIA4, CCDC141, TSPYL5, EGFLAM, TTN, LEFTY2, FOXA2, FAM184A, STMN2, DIO3, FN1, PRSS1, NPPB, and OTX2, wherein a downregulation above a predetermined threshold of an expression level of the marker negatively associated with the pancreatic differentiation as compared to the expression level of the marker in a reference cell, wherein said reference cell is a definite endodermal cell which expresses SOX17, is indicative of a pancreatic progenitor cell and alternatively or additionally; (ii) positively associated with the pancreatic differentiation, the marker being selected from the group consisting of: ALPPL2, DPPA5, H19, CRYZ, CXCL12, TYW3, ZYG11A, CRABP1, IDO1, POU5F1, HEY2, HIST1H1A, TFAP2C, DPPA2, ZFP42, LECT1, NECAB1, CKMT1A, SAMHD1, FGF2, PLA2G2A, PRDM14, POU5F1, GLI3, GSTT2, OLFML3, DAZL, GALNT3, SOX2, POU5F1B, ACTN3, CPT1A, DCLK1, EDIL3, NANOG, THUMPD3, VASH2, ATCAY, USP44, HIST1H4F, NANOG, PIM2, DNMT3B, ZNF483, FEZF1, SCARNA9L, and SILV, wherein an upregulation above a predetermined threshold of an expression level of the marker positively associated with the pancreatic differentiation as compared to the expression level of the marker in a reference cell, wherein said reference cell is a definite endodermal cell which expresses SOX17, is indicative of a pancreatic progenitor cell.

According to some embodiments of the invention, the method further comprising determining in a population of cells which comprises pancreatic progenitor cells at least one marker that is: (i) negatively associated with the pancreatic differentiation, the marker being selected from the group consisting of: TMPRSS11E, LGR5, SLC39A8, TM4SF18, CUZD1, GPC4, SLC22A3, CXCR4, NRN1, TMBIM4, THBS2, SLC7A5, TMEM47, NTS, CER1, CST1, NODAL, PRRX1, KGFLP1, NFIB, GCNT4, MIXL1, CAV1, LUM, RASGRF2, OXCT1, GLIPR1, VSNL1, FST, POSTN, GNA14, CBR1, TNIK, RGS5, KGFLP1, ETS1, MPPED2, ACTA2, SEMA3A, DACT1, ANXA1, COL12A1, KITLG, MMP2, DLEU2, ACE2, ACTG2, PUS7L, RNU5B-1, COL3A1, LEFTY2, NPPB, S1AE, AFP, ZFP42, HAS2, TRY6, NR5A2, and EBF1, wherein a downregulation above a predetermined threshold of an expression level of the marker negatively associated with the pancreatic differentiation as compared to the expression level of the marker in a reference cell, wherein said reference cell is an undifferentiated human embryonic stem cell (hESC), is indicative of a pancreatic progenitor cell, and alternatively or additionally; (ii) positively associated with the pancreatic differentiation, the marker being selected from the group consisting of: TACSTD2 (TROP-2), BST2 LRP2, LAMA1, GPC3, LGI1, FN1, LPHN3, FGB, KIT, DPP4, TRO, SPA17, ROBO2, DLK1, PRTG, DKK1, APOA2, AFP, APOB, FLRT3, KIAA1772, FBN2, FRZB, KLK6, FREM2, RSPO3, APOA1, APOA1, SEMA3D, KEL, BMP5, HEPH, STMN2, CACNB3, CHST9, TMC7, FAM171B, ATP5G1, FOLR1, ABCC2, EFNA5, CPT1C, MAGED1, RASA4, KDELR3, ST6GALNAC2, EDNRA, LRRC37B, ABCC4, CLCN5, MBOAT2, SLC44A5, NPC2, ELFN1, MMD, SLC5A3, IMPAD1, OSTC, DSC2, SLC31A1, SLC5A12, LRRN3, NPIPL3, GDPD1, NPIPL3, NPIPL3, LINGO2, LYPD6B, PRTG, LRP2, CACNG7, DLK1, CACNA2D2, CR1L, SCNN1G, HTR2C, LPAR3, THBS3, KCNG3, SDK2, HLA-DRA, SLC18A2, CXCL16, TMEM63C, SLC17A7, GFRA3, DPPA5, ALPPL2, CRYZ, H19, ZYG11A, TYW3, ARRDC4, TXNIP, FOS, NCRNA00173, GSTT2, C3, DDX43, B2M, PYGM, CYP4F22, ZNF578, ENO2, ZNF248, NLRP4, SNORD59B, SNORD113-4, CASZ1, MIR21, IFI16, ZNF560, TDRD1, ZNF680, HSPA1B, HSPA1A, EGR1, RASGRP2, ASMTL, CPT1C, ATCAY, ACSF2, PAMR1, SILV, ACTN3, HORMAD1, ACSM3, RNF157, SAMHD1, and RCN1, wherein an upregulation above a predetermined threshold of an expression level of the marker positively associated with the pancreatic differentiation as compared to the expression level of the marker in a reference cell, wherein said reference cell is an undifferentiated human embryonic stem cell (hESC), is indicative of a pancreatic progenitor cell.

According to some embodiments of the invention, the population of cells which comprises pancreatic progenitor cells express a transcription factor selected from the group consisting of PDX1, ngn3, pax4, hlxb9, nkx6.1, Hnf6, and sox9.

According to some embodiments of the invention, the definite endodermal cells are identified by a method comprising determining in a population of cells which comprises definite endodermal cells at least one marker that is: (i) negatively associated with definite endodermal cells, the marker being selected from the group consisting of: KDR, PCDHB5, FAT4, FLT1, NRN1, THBS2, PTPRZ1, SLC6A15, GPR176, SEMA6A, THBS1, CDH11, GRID2, SLC7A11, CDH1, LRFN5, EDNRB, THY1, NETO1, KCND2, TMPRSS11E, CD44, PDPN, SLC7A1, KAL1, KCNG3, GPM6B, FXYD5, PCDH18, ICAM3, MCTP1, TACR3, TMEM155, ZFP42, THUMPD3, ANXA1, SPP1, PRDM14, GNA14, EDIL3, CXCL12, PSMD5, PRRX1, NANOG, TRIM22, NANOG, RASGRF2, POU5F1B, POLR3G, HHLA1, POU5F1, VSNL1, SCG3, B3GALT1, LECT1, NTS, MBNL1, CKMT1A, NECAB1, FGF2, SFRP2, DCLK1, DACT1, CRABP1, TFAP2C, SCGB3A2, LRAT, CUZD1, GLB1L3, METTL7A, VAT1L, COL12A1, OLFML3, SOX2, USP44, HIST1H4F, KGFLP1, CPT1A, and DBC1, wherein downregulation above a predetermined threshold of an expression level of the marker negatively associated with definite endodermal cells as compared to the expression level of the marker in a reference cell, wherein said reference cell is an undifferentiated human embryonic stem cell (hESC), is indicative of a definite endodermal cell, and alternatively or additionally; (ii) positively associated with definite endodermal cells, the marker being selected from the group consisting of: FLRT3, FSHR, LIFR, ROR2, KEL, TRPA1, CD177, CCKBR, APOA1, APOA1, FZD5, FN1, BMP2, ADAMTS9, DPP4, FGA, COLEC12, GPR128, IGFBP5, FZD4, ITGA5, STC1, TNFSF4, CD177, IHH, LRP2, LAMA1, GPC3, LGI1, FN1, LPHN3, FGB, KIT, DPP4, TRO, SPA17, ROBO2, DLK1, CST1, DKK1, HAS2, APOA2, CDH2, LGR5, AFP, APOB, DGKK, HP, SYTL5, SLC7A7, TTR, FRZB, RSPO3, GCNT1, DIO3, TMEM27, APOC1, EPHA4, C4orf18, GLIPR2, PCDH10, F10, BMP5, FMO5, FOLR1, STMN2, SLC5A9, PORCN, EGFLAM, RAB17, CST2, PNPLA3, MOSPD1, ELMO1, CHST9, SLC30A10, TMC7, C8orf49, CDH12, ST8SIA4, SLCO2A1, MANEA, LRIG3, HCN1, ADAMTS12, FOLH1, TMEM144, VTN, CAMK2N1, ABCC4, PCDH7, OR2T4, UNC93A, COL4A6, PAMR1, SLC1A1, PROS1, APOM, APOM, APOM, LYPD6B, TMEM88, ITLN2, BMPER, GPR141, VEGFA, PRTG, DKK1, APOA2, AFP, APOB, FLRT3, KIAA1772, FBN2, FRZB, KLK6, FREM2, RSPO3, APOA1, APOA1, SEMA3D, KEL, BMP5, HEPH, STMN2, CACNB3, CHST9, TMC7, FAM171B, ATP5G1, FOLR1, ABCC2, EFNA5, CPT1C, MAGED1, RASA4, KDELR3, ST6GALNAC2, EDNRA, LRRC37B, ABCC4, CLCN5, MBOAT2, SLC44A5, NPC2, ELFN1, MMD, SLC5A3, IMPAD1, OSTC, DSC2, SLC31A1, SLC5A12, LRRN3, NPIPL3, GDPD1, NPIPL3, NPIPL3, LINGO2, SLC40A1, SLC30A10, CCKBR, VIPR2, COLEC12, FLRT3, LGR5, GPR141, BST2, SLC5A9, GPR128, KEL, LRIG3, LYPD6B, FSHR, LIFR, FOLH1, CXCR4, ITGA5, AMOT, LY6E, SEMA6D, GJA5, PRTG, CD34, TMEM144, ROR2, GPR177, OR2T4, SLC7A7, KCNJ3, CLDN18, GPR151, SLC44A5, CDH10, TMEM27, SLC1A1, TMEM56, CD177, PLXNA2, SLC26A2, DSCAM, TMEM133, IL13RA1, ATP2B1, CD302, MEGF9, EDNRA, CDH2, GPR161, TYRO3, FLRT2, LRIT3, PCDH7, NRCAM, SMAGP, AMHR2, ELTD1, GRPR, EPHA4, CD99, GATA3, SEMA3E, HHEX, ZNF280A, FAM184A, WDR72, PDZK1, RLBP1L2, SHISA2, VIL1, STMN2, APOA2, SERHL, PPFIBP2, DKK1, MUM1L1, IGFBP5, ST6GALNAC2, TSPYL5, STC1, SYTL5, EPSTI1, ANKRD1, ARHGAP24, KRT18P49, PRSS2, RHOBTB3, FRZB, RARB, ADAMTS9, ARL4D, PRDM1, HP, FZD5, TRY6, ATP6V0D2, ANGPT2, DENND2C, BMP5, FOXA2, HAS2, BMP2, S100A16, FOLH1B, FAM122C, and FZD4, wherein upregulation above a predetermined threshold of an expression level of the marker positively associated with definite endodermal cells as compared to the expression level of the marker in a reference cell, wherein said reference cell is an undifferentiated human embryonic stem cell (hESC), is indicative of a definite endodermal cell, thereby identifying the definite endodermal cells.

According to some embodiments of the invention, the at least one marker positively associated with pancreatic differentiation is selected from the group consisting of: TACSTD2 (TROP-2), GPR50, BST2, LGI1, VIPR2, SLC2A1, MUC15, MUC12, LPHN3, MUC16, VTCN1, MMP16, FZD3, ITGB6, GFRA3, ROBO1, NLGN1, MUC12, CNTFR, LPHN1, SULF1, ADAM23, SCUBE3, PLAU, CDON, SLIT2, C7orf68, PLXDC2, CD74, MUC15, GPR56, VTCN1, ITGB6, NTRK2, AREG, BOC, ITGA4, KLRK1, PRTG, PTPRZ1, GABRP, SILV, KIAA1772, PLP1, OVOS, HAPLN1, EPHA7, ENO2, PCDHB5, OVOS, SYT1, DCT, GPAM, SLITRK6, DCC, FREM2, SDK2, CGA, ATP1B2, SEMA3D, PCDHB15, CDH1, WNT8B, LPAR4, NPIPL3, FAM171B, PTN, ABCC2, ADAMTS3, RASA4, CPT1C, SLC6A6, PCDHB3, LRRC37B, RNFT2, KCNG3, TRPC1, ALPPL2, OR4F21, CCL2, KIF5A, OLFM2, CACNG7, MPHOSPH9, SLC13A4, MOXD1, C6orf186, SLC4A8, STX16, AMY2A, SPARCL1, MGP, A2M, DCN, ATP8B1, MMRN1, EMP1, PLA2G2A, PDE3A, TLR3, CYP1B1, PTGIS, RFTN2, PLEKHA2, SMOC1, STOM, JAM2, CHL1, SCG5, IGFBP7, NPR3, IFI6, CR1L, OR2A4, and OR2A7.

According to some embodiments of the invention, the at least one marker positively associated with pancreatic differentiation is selected from the group consisting of: TACSTD2 (TROP-2), BST2, GPR50, ROBO1, NTRK2, ITGA4, LGI1, VIPR2, SLC2A1, MUC15, MUC12, LPHN3, MUC16, VTCN1, MMP16, FZD3, ITGB6, GFRA3, NLGN1, MUC12, CNTFR, LPHN1, SULF1, ADAM23, SCUBE3, PLAU, CDON, SLIT2, C7orf68, PLXDC2, CD74, MUC15, GPR56, VTCN1, ITGB6, AREG, BOC, and KLRK1.

According to some embodiments of the invention, the at least one marker positively associated with pancreatic differentiation is TROP-2.

According to some embodiments of the invention, the at least one marker positively associated with pancreatic differentiation is GPR50.

According to some embodiments of the invention, the at least one marker positively associated with pancreatic differentiation comprises at least two markers, said at least two markers are TROP-2 and GPR50.

According to some embodiments of the invention, the at least one marker positively associated with pancreatic differentiation comprises at least three markers, said at least three markers comprise TROP-2, GPR50 and a marker selected from the group consisting of BST2, NTRK2, ITGA4, KDR, PTPRN, LGI1, VIPR2, SLC2A1, MUC15, MUC12, LPHN3, MUC16, VTCN1, MMP16, FZD3, ITGB6, GFRA3, ROBO1, NLGN1, MUC12, CNTFR, LPHN1, SULF1, ADAM23, SCUBE3, PLAU, CDON, SLIT2, C7orf68, PLXDC2, CD74, MUC15, GPR56, VTCN1, ITGB6, AREG, BOC, KLRK1, PRTG, PTPRZ1, GABRP, SILV, KIAA1772, PLP1, OVOS, HAPLN1, EPHA7, ENO2, PCDHB5, OVOS, SYT1, DCT, GPAM, SLITRK6, DCC, FREM2, SDK2, CGA, ATP1B2, SEMA3D, PCDHB15, CDH1, WNT8B, LPAR4, NPIPL3, FAM171B, PTN, ABCC2, ADAMTS3, RASA4, CPT1C, SLC6A6, PCDHB3, LRRC37B, RNFT2, KCNG3, TRPC1, ALPPL2, OR4F21, CCL2, KIF5A, OLFM2, CACNG7, MPHOSPH9, SLC13A4, MOXD1, C6orf186, SLC4A8, STX16, AMY2A, SPARCL1, MGP, A2M, DCN, ATP8B1, MMRN1, EMP1, PLA2G2A, PDE3A, TLR3, CYP1B1, PTGIS, RFTN2, PLEKHA2, SMOC1, STOM, JAM2, CHL1, SCG5, IGFBP7, NPR3, IFI6, CR1L, OR2A4, OR2A7, KCNG3, CACNG7, GRID2, CDH1, LPAR3, SEMA6A, PTPRZ1, ATP1A3, CAMKV, SCNN1G, SYT6, SLC18A2, PCDHB5, ABCG2, HLA-DRA, CR1L, HTR2C, EDNRB, PCDH11X, SLC17A7, SCNN1A, CD9, CXCL16, FXYD5, GABRQ, GFRA3, CACNA2D2, CLDN4, PLP1, PDPN, MMP24, SDK2, GPR176, GPR64, GPR160, PCDH11Y, NKAIN4, ATP1B2, SCN8A, THBS4, CR2, HLA-DQA1, HLA-DRA, HTR7, SLC2A1, HLA-DRA, KCNS3, SLC7A3, HLA-DPB2, CACNA1B, and GPR143

According to some embodiments of the invention, the at least one marker positively associated with definite endodermal cells is selected from the group consisting of: COLEC12, ROR2, FLRT3, LGR5, LIFR, KEL, FSHR, TRPA1, FOLR1, LRP2, FOLH1, CD177, CCKBR, ITGA5, FZD5, FN1, BMP2, ADAMTS9, DPP4, FGA, GPR128, IGFBP5, FZD4, STC1, TNFSF4, CD177, IHH, APOA1, LAMA1, GPC3, LGI1, FN1, LPHN3, FGB, KIT, DPP4, TRO, SPA17, ROBO2, DLK1, CST1, DKK1, HAS2, APOA2, CDH2, AFP, APOB, DGKK, HP, SYTL5, SLC7A7, TTR, FRZB, RSPO3, GCNT1, DIO3, TMEM27, APOC1, EPHA4, C4orf18, GLIPR2, PCDH10, F10, BMP5, FMO5, STMN2, SLC5A9, PORCN, EGFLAM, RAB17, CST2, PNPLA3, MOSPD1, ELMO1, CHST9, SLC30A10, TMC7, C8orf49, CDH12, ST8SIA4, SLCO2A1, MANEA, LRIG3, HCN1, ADAMTS12, TMEM144, VTN, CAMK2N1, ABCC4, PCDH7, OR2T4, UNC93A, COL4A6, PAMR1, SLC1A1, PROS1, APOM, APOM, APOM, LYPD6B, TMEM88, ITLN2, BMPER, GPR141, VEGFA, PRTG, DKK1, APOA2, AFP, APOB, FLRT3, KIAA1772, FBN2, FRZB, KLK6, FREM2, RSPO3, APOA1, APOA1, SEMA3D, KEL, BMP5, HEPH, STMN2, CACNB3, CHST9, TMC7, FAM171B, ATP5G1, FOLR1, ABCC2, EFNA5, CPT1C, MAGED1, RASA4, KDELR3, ST6GALNAC2, EDNRA, LRRC37B, ABCC4, CLCN5, MBOAT2, SLC44A5, NPC2, ELFN1, MMD, SLC5A3, IMPAD1, OSTC, DSC2, SLC31A1, SLC5A12, LRRN3, NPIPL3, GDPD1, NPIPL3, NPIPL3, LINGO2, and LYPD6B.

According to some embodiments of the invention, the at least one marker positively associated with definite endodermal cells is selected from the group consisting of: FSHR, LIFR, COLEC12, ROR2, ITGA5, CD177, CCKBR, APOA1, APOA1, FZD5, FN1, BMP2, ADAMTS9, DPP4, FGA, GPR128, IGFBP5, FZD4, STC1, TNFSF4, CD177, IHH, LRP2, LAMA1, GPC3, LGI1, FN1, LPHN3, FGB, KIT, DPP4, TRO, SPA17, ROBO2, and DLK1.

According to some embodiments of the invention, the cell sample comprises the isolated pancreatic progenitor cells of some embodiments of the invention.

According to some embodiments of the invention, presence of at least a predetermined percentage of the pancreatic progenitor cells in the cell sample indicates the suitability of the pancreatic progenitor cells for transplantation in a subject.

According to some embodiments of the invention, the method further comprising determining in the population of cells which comprises definite endodermal cells at least one marker that is positively associated with definite endodermal cells, the marker being selected from the group consisting of: FSHR, COLEC12, ROR2, LIFR, LIFR, FLRT3, KEL, LGR5, FOLR1, CD177, CCKBR, APOA1, APOA1, FZD5, FN1, BMP2, ADAMTS9, DPP4, FGA, GPR128, IGFBP5, FZD4, ITGA5, STC1, TNFSF4, CD177, IHH, LRP2, LAMA1, GPC3, LGI1, FN1, LPHN3, FGB, KIT, DPP4, TRO, SPA17, ROBO2, DLK1, CST1, DKK1, HAS2, APOA2, CDH2, AFP, APOB, DGKK, HP, SYTL5, SLC7A7, TTR, FRZB, RSPO3, GCNT1, TRPA1, DIO3, TMEM27, APOC1, EPHA4, C4orf18, GLIPR2, PCDH10, F10, BMP5, FMO5, STMN2, SLC5A9, PORCN, EGFLAM, RAB17, CST2, PNPLA3, MOSPD1, ELMO1, CHST9, SLC30A10, TMC7, C8orf49, CDH12, ST8SIA4, SLCO2A1, MANEA, LRIG3, HCN1, ADAMTS12, FOLH1, TMEM144, VTN, CAMK2N1, ABCC4, PCDH7, OR2T4, UNC93A, COL4A6, PAMR1, SLC1A1, PROS1, APOM, APOM, APOM, LYPD6B, TMEM88, ITLN2, BMPER, GPR141, VEGFA, PRTG, DKK1, APOA2, AFP, APOB, FLRT3, KIAA1772, FBN2, FRZB, KLK6, FREM2, RSPO3, APOA1, APOA1, SEMA3D, KEL, BMP5, HEPH, STMN2, CACNB3, CHST9, TMC7, FAM171B, ATP5G1, FOLR1, ABCC2, EFNA5, CPT1C, MAGED1, RASA4, KDELR3, ST6GALNAC2, EDNRA, LRRC37B, ABCC4, CLCN5, MBOAT2, SLC44A5, NPC2, ELFN1, MMD, SLC5A3, IMPAD1, OSTC, DSC2, SLC31A1, SLC5A12, LRRN3, NPIPL3, GDPD1, NPIPL3, NPIPL3, LINGO2, LYPD6B, TRPA1, SLC40A1, SLC30A10, CCKBR, VIPR2, COLEC12, FLRT3, LGR5, GPR141, BST2, SLC5A9, GPR128, KEL, LRIG3, LYPD6B, FSHR, LIFR, FOLH1, CXCR4, ITGA5, AMOT, LY6E, SEMA6D, GJA5, PRTG, CD34, TMEM144, ROR2, GPR177, OR2T4, SLC7A7, KCNJ3, CLDN18, GPR151, SLC44A5, CDH10, TMEM27, SLC1A1, TMEM56, CD177, PLXNA2, SLC26A2, DSCAM, TMEM133, IL13RA1, ATP2B1, CD302, MEGF9, EDNRA, CDH2, GPR161, TYRO3, FLRT2, LRIT3, PCDH7, NRCAM, SMAGP, AMHR2, ELTD1, GRPR, EPHA4, CD99, GATA3, SEMA3E, HHEX, ZNF280A, FAM184A, WDR72, PDZK1, RLBP1L2, SHISA2, VIL1, STMN2, APOA2, SERHL, PPFIBP2, DKK1, MUM1L1, IGFBP5, ST6GALNAC2, TSPYL5, STC1, SYTL5, EPSTI1, ANKRD1, ARHGAP24, KRT18P49, PRSS2, RHOBTB3, FRZB, RARB, ADAMTS9, ARL4D, PRDM1, HP, FZD5, TRY6, ATP6V0D2, ANGPT2, DENND2C, BMP5, FOXA2, HAS2, BMP2, S100A16, FOLH1B, FAM122C, and FZD4, wherein upregulation above a predetermined threshold of an expression level of the marker positively associated with definite endodermal cells as compared to the expression level of the marker in a reference cell, wherein said reference cell is an undifferentiated human embryonic stem cell (hESC), is indicative of a definite endodermal cell.

According to some embodiments of the invention, the method further comprising determining in the population of cells which comprises definite endodermal cells at least one marker that is positively associated with definite endodermal cells, the marker being selected from the group consisting of: GATA3, SEMA3E, HHEX, ZNF280A, FAM184A, WDR72, PDZK1, RLBP1L2, SHISA2, VIL1, STMN2, APOA2, SERHL, PPFIBP2, DKK1, MUM1L1, IGFBP5, ST6GALNAC2, TSPYL5, STC1, SYTL5, EPSTI1, ANKRD1, ARHGAP24, KRT18P49, PRSS2, RHOBTB3, FRZB, RARB, ADAMTS9, ARL4D, PRDM1, HP, FZD5, TRY6, ATP6V0D2, ANGPT2, DENND2C, BMP5, FOXA2, HAS2, BMP2, S100A16, FOLH1B, FAM122C, and FZD4, wherein upregulation above a predetermined threshold of an expression level of the definite endodermal cells as compared to the expression level of the marker in a reference cell, wherein said reference cell is an undifferentiated human embryonic stem cell (hESC), is indicative of a definite endodermal cell.

According to some embodiments of the invention, the method further comprising determining in the population of cells which comprises definite endodermal cells at least one marker that is negatively associated with definite endodermal cells, the marker being selected from the group consisting of: KDR, PCDHB5, FAT4, FLT1, NRN1, THBS2, PTPRZ1, SLC6A15, GPR176, SEMA6A, THBS1, CDH11, GRID2, SLC7A11, CDH1, LRFN5, EDNRB, THY1, NETO1, KCND2, TMPRSS11E, CD44, PDPN, SLC7A1, KAL1, KCNG3, GPM6B, FXYD5, PCDH18, ICAM3, MCTP1, TACR3, TMEM155, ZFP42, THUMPD3, ANXA1, SPP1, PRDM14, GNA14, EDIL3, CXCL12, PSMD5, PRRX1, NANOG, TRIM22, NANOG, RASGRF2, POU5F1B, POLR3G, HHLA1, POU5F1, VSNL1, SCG3, B3GALT1, LECT1, NTS, MBNL1, CKMT1A, NECAB1, FGF2, SFRP2, DCLK1, DACT1, CRABP1, TFAP2C, SCGB3A2, LRAT, CUZD1, GLB1L3, METTL7A, VAT1L, COL12A1, OLFML3, SOX2, USP44, HIST1H4F, KGFLP1, CPT1A, and DBC1, wherein downregulation above a predetermined threshold of an expression level of the marker negatively associated with definite endodermal cells as compared to the expression level of the marker in a reference cell, wherein said reference cell is an undifferentiated human embryonic stem cell (hESC), is indicative of a definite endodermal cell.

According to some embodiments of the invention, the at least one marker positively associated with definite endodermal cells is selected from the group consisting of: FSHR, COLEC12, ROR2 ITGA5, LRP2, CD177, CCKBR, TRPA1, KEL, FOLR1, FOLH1, APOA1, APOA1, FZD5, FN1, BMP2, ADAMTS9, DPP4, FGA, GPR128, IGFBP5, LIFR, FZD4, PRTG, STC1, TNFSF4, CD177, IHH, LAMA1, GPC3, LGI1, FN1, LPHN3, FGB, KIT, DPP4, TRO, SPA17, ROBO2, DLK1, CST1, DKK1, HAS2, APOA2, CDH2, LGR5, AFP, FLRT3, APOB, DGKK, HP, SYTL5, SLC7A7, TTR, FRZB, RSPO3, GCNT1, DIO3, TMEM27, APOC1, EPHA4, C4orf18, GLIPR2, PCDH10, F10, BMP5, FMO5, STMN2, SLC5A9, PORCN, EGFLAM, RAB17, CST2, PNPLA3, MOSPD1, ELMO1, CHST9, SLC30A10, TMC7, C8orf49, CDH12, ST8SIA4, SLCO2A1, MANEA, LRIG3, HCN1, ADAMTS12, TMEM144, VTN, CAMK2N1, ABCC4, PCDH7, OR2T4, UNC93A, COL4A6, PAMR1, SLC1A1, PROS1, APOM, APOM, APOM, LYPD6B, TMEM88, ITLN2, BMPER, GPR141, VEGFA, DKK1, APOA2, AFP, APOB, FLRT3, KIAA1772, FBN2, FRZB, KLK6, FREM2, RSPO3, APOA1, APOA1, SEMA3D, KEL, BMP5, HEPH, STMN2, CACNB3, CHST9, TMC7, FAM171B, ATP5G1, FOLR1, ABCC2, EFNA5, CPT1C, MAGED1, RASA4, KDELR3, ST6GALNAC2, EDNRA, LRRC37B, ABCC4, CLCN5, MBOAT2, SLC44A5, NPC2, ELFN1, MMD, SLC5A3, IMPAD1, OSTC, DSC2, SLC31A1, SLC5A12, LRRN3, NPIPL3, GDPD1, NPIPL3, NPIPL3, LINGO2, and LYPD6B.

According to some embodiments of the invention, the at least one marker positively associated with definite endodermal cells is selected from the group consisting of: FSHR, COLEC12, ROR2 ITGA5, LRP2, CD177, CCKBR, TRPA1, KEL, FOLR1, FOLH1, APOA1, APOA1, FZD5, FN1, BMP2, ADAMTS9, DPP4, FGA, GPR128, IGFBP5, LIFR, FZD4, PRTG, STC1, TNFSF4, CD177, IHH, LAMA1, GPC3, LGI1, FN1, LPHN3, FGB, KIT, DPP4, TRO, SPA17, ROBO2, and DLK1.

According to some embodiments of the invention, the at least one marker positively associated with definite endodermal cells is selected from the group consisting of: CD177, CCKBR, APOA1, APOA1, FSHR, FZD5, FN1, BMP2, ADAMTS9, DPP4, FGA, COLEC12, ROR2, GPR128, IGFBP5, LIFR, FZD4, ITGA5, STC1, TNFSF4, CD177 and IHH.

According to some embodiments of the invention, the isolated population of pancreatic progenitor cells are further characterized by an expression signature of PDX1+/ngn3+/pax4+/hlxb9+/nkx6.1+/Hnf6+/sox9+.

According to some embodiments of the invention, the pancreatic progenitor cells are genetically unmodified.

According to some embodiments of the invention, the definite endodermal cells are genetically unmodified.

According to some embodiments of the invention, the definite endodermal cells are characterized by a SOX17+/SOX7+ expression signature.

According to some embodiments of the invention, the definite endodermal cells are characterized by a SOX17+/SOX7+/GSC+/CER+/FOXA2+/CD34+/CXCR4+/NANOG− expression signature.

According to some embodiments of the invention, the definite endodermal cells are characterized by a SOX17+/SOX7+/GSC+/CER+/FOXA2+/CXCR4+/NANOG− expression signature.

According to some embodiments of the invention, the undifferentiated embryonic stem cells (ESCs) are characterized by Oct4+ expression pattern.

According to some embodiments of the invention, the pancreatic progenitor cells are obtained by differentiating stem cells into pancreatic progenitor cells.

According to some embodiments of the invention, the definite endodermal cells are obtained by differentiating stem cells into the definite endodermal cells.

According to some embodiments of the invention, the stem cells are undifferentiated pluripotent stem cells.

According to some embodiments of the invention, the stem cells are adult stem cells.

According to some embodiments of the invention, the stem cells are fetal stem cells.

According to some embodiments of the invention, the undifferentiated pluripotent stem cells are embryonic stem cells (ESCs).

According to some embodiments of the invention, the undifferentiated pluripotent stem cells are induced pluripotent stem cells (iPSCs).

According to some embodiments of the invention, the cells are human cells.

According to some embodiments of the invention, differentiating the undifferentiated pluripotent stem cells into the pancreatic progenitor cells is performed by: (a) differentiating the pluripotent stem cells into definite endodermal cells to thereby obtain a population of cells which comprises definite endodermal cells, and (b) differentiating the population of cells which comprises the definite endodermal cells into the pancreatic progenitor cells, thereby inducing the differentiation of the pluripotent stem cells into the pancreatic progenitor cells.

According to some embodiments of the invention, differentiating the undifferentiated pluripotent stem cells into the pancreatic progenitor cells is performed by differentiation of the pluripotent stem cells into embryoid bodies.

According to some embodiments of the invention, the embryoid bodies are differentiated until about day 7-21 of human EBs differentiation.

According to some embodiments of the invention, differentiating the undifferentiated pluripotent stem cells into the definite endodermal cells is performed by culturing the pluripotent stem cells in the presence of activin A, Wnt3A, a small molecule Induce Definitive Endoderm 1 (IDE1) and/or a small molecule Induce Definitive Endoderm 2 (IDE2).

According to some embodiments of the invention, differentiating the definite endodermal cells into the pancreatic progenitor cells is performed by culturing the definite endodermal cells in the presence of retinoic acid.

According to some embodiments of the invention, differentiating the definite endodermal cells into the pancreatic progenitor cells is performed by culturing the definite endodermal cells in the presence of bFGF, KGF, FGF10, noggin, cyclopamine, KAAD cyclopamine, B27, Indolactam V, nicotinamide and/or epidermal growth factor.

According to some embodiments of the invention, step (b) of the method of some embodiments of the invention is effected by an immunological isolation assay selected from the group consisting of fluorescent activated cell sorter (FACS), Magnetic-activated cell sorting (MACS) or immunopanning.

According to some embodiments of the invention, the nucleic acid construct is a bacterial artificial chromosome (BAC).

According to some embodiments of the invention, the cell is a stem cell.

According to some embodiments of the invention, the stem cell is an embryonic stem cell or an induced pluripotent stem cell.

According to some embodiments of the invention, the cell is a human cell.

According to some embodiments of the invention, the method further comprising synthesizing the compound capable of inducing differentiation of the undifferentiated pluripotent stem cells to the definite endodermal cells.

According to some embodiments of the invention, the undifferentiated pluripotent stem cell is an embryonic stem cell, an adult stem cell or an adult-derived stem cell such as induced pluripotent stem cells (iPSC).

According to some embodiments of the invention, the pluripotent stem cell is characterized by an Oct4+/SSEA4+/SSEA3+/TRA1-60+ expression signature.

According to some embodiments of the invention, the method further comprising synthesizing the compound capable of inducing differentiation of the definite endodermal cells or undifferentiated pluripotent stem cells to the pancreatic progenitor cells.

According to some embodiments of the invention, the kit further comprising a pluripotent stem cell.

According to some embodiments of the invention, the kit further comprising at least one agent suitable for detecting an expression level of a marker of interest.

According to some embodiments of the invention, the expression level is detected by an RNA detection method.

According to some embodiments of the invention, the expression level is detected by a protein detection method.

According to some embodiments of the invention, the kit further comprising a genetic micro array chip.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C depict generation and expression of the SOX17-GFP reporter constructs. EGFP and floxed neomycin resistance gene was knocked into the SOX17 gene locus in a human genomic BAC by replacing the coding sequence of SOX17 (ATG to TGA) with the coding sequence of GFP and also the floxed neomycin resistance gene. FIG. 1A schematically illustrates the structure of a recombinant SOX17-GFP BAC construct (using BAC RP11-53M11) according to some embodiments of the invention. FIG. 1B schematically illustrates the structure of a recombinant SOX17-GFP plasmid according to some embodiments of the invention. The positions of the 5′-arm, the 3′-arm in the plasmid construct are shown relative to nucleotide number 1 of the recombinant BAC RP11-53M11 SOX17-GFP (SEQ ID NO:1). In addition the positions of two non repetitive 3′ external screening probes are also shown with positions relative to nucleotide number 1 of the recombinant BAC RP11-53M11 SOX17-GFP (SEQ ID NO:1). “Ext.”=external. FIG. 1C schematically illustrates the structure of the recombinant RP11-47H10 SOX17-GFP BAC construct according to some embodiments of the invention.

FIGS. 2A-H depict expression of the SOX17-GFP reporter construct in human embryonic stem cells and cells differentiated therefrom. FIG. 2A-FACS (fluorescence-activated cell sorting) of GFP expressing cells in two day old embryoid bodies (EBs) of a SOX17-GFP clone grown in the presence of activin A (66 ng/ml). A hESC clone containing the SOX17 reporter BAC was allowed to differentiate to form EBs. At day 2, the cells were dissociated and subjected to sorting by FACS into GFP⁺ (GFP-positive cells; green cells in gate R2) and GFP⁻ (GFP-negative cells; red cells) cell populations. The GFP⁺ population represents a population of SOX17⁺ (SOX17-positive) endoderm progenitor cells. FIGS. 2B-2E are microscopic images depicting SOX17 GFP reporter cells observed in phase contrast (FIGS. 2B-C) or using fluorescent microscopy (FIGS. 2D-E). A SOX17 GFP reporter clone was grown in the presence of activin A (66 ng/ml) for three days, and GFP expression was observed using a fluorescent microscope. FIG. 2F—A microscopic image depicting SOX17 expression in a SOX17 reporter clone. An 80% confluent monolayer of the SOX17-GFP reporter ES cell line was treated for three days with activin A (66 ng/ml), following which immunohistochemistry was performed with anti-SOX17 (red staining) and anti-GFP (green staining) antibodies. Note the clear correlation between SOX17 and GFP expression. FIG. 2G—A microscopic image depicting double staining of the cells described in FIG. 2F with both OCT4 (green staining), which marks undifferentiated cells, and SOX17 (red staining), which marks the definitive endoderm population. Note that OCT4 positive cells are negative for SOX17. FIG. 2H—A histogram depicting relative gene expression levels in SOX17-GFP+ cells as compared to SOX17-GFP-cells. SOX17-GFP reporter cells were grown as a monolayer in the presence of activin A (66 ng/ml), were harvested and FACS-sorted into GFP⁺ and GFP⁻ cells. RNA was extracted from the sorted populations which was reverse transcribed and was analyzed by real-time qPCR. The relative expression of various genes in the SOX17-GFP⁻ population as compared to the SOX17-GFP⁻ population is presented. Note that while SOX17, SOX7, GSC, CER, FOXA2, CD34 and CXCR4 are highly expressed in SOX17-GFP⁺ cells as compared to the SOX17-GFP⁻ cells, the expression of NANOG, a marker of pluripotency, is higher in SOX17-GFP⁻ cells as compared to SOX17-GFP⁺ cells.

FIGS. 3A-E depict generation of PDX1-GFP reporter constructs according to some embodiments of the invention. FIG. 3A—a schematic illustration of the PDX1 ATG-GFP-knock in recombinant BAC construct of some embodiments of the invention. FIG. 3B—a schematic illustration with a nucleotide positions of the 5′-arm, GFP gene, floxed neomycin resistance gene and 3′-arm of the PDX1 ATG-GFP-knock in BAC construct of some embodiments of the invention. FIG. 3C—a schematic illustration of the PDX1 ATG-GFP-knock in plasmid construct (which includes the 5′-arm, the GFP gene, the floxed neomycin resistance gene and the 3′-arm) along with the relative position of the 3′-external probe (not included in the plasmid construct) in the recombinant BAC which was used to generate the recombinant plasmid construct; FIG. 3D—a schematic illustration of the PDX1-IRES-GFP plasmid construct according to some embodiments of the invention. FIG. 3E—a schematic illustration of the PDX1-mCherry BAC construct according to some embodiments of the invention.

FIGS. 4A-D depicts expression of the PDX1-GFP reporter BAC. FIGS. 4A-B—Dot plots depicting FACS analyses of 15 day-old EBs from PDX1 GFP reporter BAC clones. The PDX1 BAC reporter clone was allowed to form EBs for fifteen days. The EBs were then dissociated into single cells which were analysed by FACS. FIG. 4A—represents control cells from wild type hESCs that do not contain a reporter construct. FIG. 4B—represents GFP expression (R2) in EBs from PDX1-GFP reporter clone. Note that while in the control cells there are no cells which express GFP (R2), cells harbouring the PDX1-GFP construct express GFP (R2). FIG. 4C—A confocal microscopy image depicting 15 day-old EBs developed from a hESC harbouring the PDX1-GFP reporter clone that were subjected to immunostaining. Note that the EBs contain cells which express PDX1 in the nucleus (red staining) and GFP in the cytoplasm (green staining). The nuclei were counterstained with TO-PRO-3 (blue stain). FIG. 4D—A histogram depicting gene expression levels in PDX1-GFP⁺ cells and PDX1-GFP⁻ cells as compared to unsorted EBs. The PDX1 BAC reporter clone was allowed to form EBs for fifteen days after which they were dissociated into single cells and the PDX1-GFP⁺ cells and PDX1-GFP⁻ cells were sorted by FACS, and RNA was extracted from the GFP⁺ and GFP⁻ populations. qRT-PCR (quantitative RT-PCR) was performed to compare expression of various markers in the two different cell populations. Red bars=GFP⁺ cells; Blue bars=GFP− cells. Note that PDX1 is enriched in the GFP⁺ population. Also note the upregulation of markers of pancreatic differentiation HLXB9, HNF6, NGN3 and PAX4 in PDX1-GFP+ cells as compared to PDX1-GFP⁻ cells.

FIGS. 5A-E depict gene profiling of the SOX17⁺ and PDX1⁺ precursor cells. FIG. 5A—Schematic description of the strategy for identification of unique, stage specific expression markers. Each colored ellipse represents a different differentiation stage in pancreatic development from undifferentiated embryonic cells (OCT4 positive cells), through endodermal progenitor cells (SOX17 positive cells); pancreatic progenitor cells (PDX1 positive cells) to mature pancreatic cells (insulin positive cells). RNA from each cell group was extracted and expression profiling was performed. Comparison between each group enables stage specific identification. FIGS. 5B-E—graphs depicting clustering of gene expression profiles obtained by Affymetrix analysis of mRNA extracted from GFP positive cells that were isolated by FACS from SOX17 reporter clones or from PDX1 reporter clones, and compared with the gene expression profile of undifferentiated hESC and of mature pancreas (as reported in the Affymetrix site). FIG. 5B—“SOX17 only genes” (definite endodermal-associated genes, represented by the newly identified markers: TMEM45A, RELL2 and PPFIA4; FIG. 5C—PDX1 only genes, represented by SILV (Official gene symbol PMEL) and GPR50; FIG. 5D—SOX17 and PDX1 genes, represented by the STX16, DLK1, CPT1C, LRP2 and BST2; and FIG. 5E—PDX1 and Pancreases genes, represented by TROP-2 (also known as TROP2; official gene symbol TACSTD2).

FIGS. 6A-B depict cell sorting using antibodies which specifically bind GPR50 and TROP-2 in twenty five day old genetically-unmodified EBs and gene expression analysis of the various subgroups. FIG. 6A—Twenty five day old dissociated EBs were immunostained with GPR50 (FITC-labeled) and TROP-2 (APC, Allophycocyanin-labeled) antibodies. Four populations were separated by FACS: GPR50−/TROP-2−, GPR50−/TROP-2⁺, GPR50⁺/TROP-2− and GPR50⁺/TROP-2⁺. FIG. 6B—RT-qPCR was performed to compare expression of various pancreatic markers in the four different cell populations isolated by FACS as described in FIG. 6B.

FIG. 7 is a graph depicting gene clustering by Partek analysis. The x axis: cell types; The y axis: normalized expression means value. Purple, red, green and blue curves represent the specific genes for the SOX17⁺, SOX17⁺/PDX1⁺, PDX1⁺ and PDX1⁺/Pancreas cell populations, respectively.

FIG. 8 is a histogram depicting SOX17⁺ stage specific genes. The x axis: genes; The y axis: expression ±SE. Blue, red, green and purple bars represent HESC, SOX17, PDX1 and Pancreas expression, respectively.

FIG. 9 is a histogram depicting PDX1⁺ stage-specific genes. The x axis: genes; The y axis: expression ±SE. Blue, red, green and purple bars represent HESC, SOX17, PDX1 and Pancreas expression, respectively.

FIG. 10 is a histogram depicting SOX17⁺/PDX1⁺ stage specific genes. The x axis: genes; The y axis: expression ±SE. Blue, red, green and purple bars represent HESC, SOX17, PDX1 and Pancreas expression, respectively.

FIG. 11 is a histogram depicting PDX1⁺-Pancreas specific genes. The x axis: genes; The y axis: expression ±SE. Blue, red, green and purple bars represent HESC, SOX17, PDX1 and Pancreas expression, respectively.

FIGS. 12A-E are FACS analyses depicting the expression of the new endodermal surface markers BST (FIG. 12A), FLRT3 (FIG. 12B), COLEC12 (FIG. 12C), GPR49 (FIG. 12D) and LIFR (FIG. 12E) in cells of day-7 embryoid bodies. Embryoid bodies were prepared from H9.2 hESCs grown for 7 days and then separated to single cells with TrypLE Select. Next, the cells were incubated for 30 minutes at room temperature with one of the following antibodies: Rabbit anti BST-2 1:200; Rabbit anti FLRT-3 1:50; Goat anti CL-P1/COLEC12 1:40; Rabbit anti GPR-49/LGR5 1:100; Anti human LIF-R-PE 1:10 or control non treated cells, followed by secondary antibodies anti rabbit Cy3 (for BST2, FLRT-3 and GPR-49) or anti goat Cy3 (COLEC12) for 15 minutes at room temperature. The expression of these markers in the EBs (green) were compared to undifferentiated hESCs cells (red) by FACS calibrator (BD) using Flow-Jo analysis. Note that 43.3% of the EBs cells are BST+ (FIG. 12A), 70.19% of the EBs cells are COLEC12+ (FIG. 12C), 38.32% of the EBs cells are PGR49+ (FIG. 12D), and 16.13% of the EBs cells are LIFR+ (FIG. 12E).

FIGS. 13A-D are FACS analyses depicting BST2, KDR and CXCR4 expression in IDE treated cells. Undifferentiated H9.2 cells were treated with IDE1/2 and then stained with an anti-BST2 antibody (1:200), an anti-KDR antibody Fitc conjugated (1:10) or with a combination of both, followed by a cy3-conjugated secondary antibody; or stained with 1:10 anti-human CXCR4-PerCP and 1:10 anti-human VEGF R2/KDR-Fitc, and taken for FACS analysis. Note that incubation with an anti-BST2 antibody resulted in isolation of 11.9% BST2+ cells from the IDE-treated cells (FIG. 13A); incubation with an anti-KDR antibody resulted in 4.32% KDR+ cells from IDE-treated cells (FIG. 13B); incubation with an anti-BST2 and anti-KDR antibodies resulted in 10.7% Bst2⁺/KDR⁻ (BST2 positive and KDR negative) cells of the IDE-treated cells and 0.39% of KDR+/BST2− (KDR positive and BST2 negative) cells of the IDE-treated cells (FIG. 13C); and incubation with the anti-CXCR4 and anti-KDR antibodies resulted in 1.05% CXCR4+/KDR− (CXCR4 positive and KDR negative) cells of the IDE-treated cells (FIG. 13D).

FIGS. 14A-C are representative images of confocal microscopy depicting expression of PDX1 in day-28 of human embryoid bodies which were sorted with an anti-TROP-2 antibody, demonstrating enrichment of a pancreatic progenitor cells. Blue—DAPI, nuclear staining; Red—PDX1. Note that most of the cells presented in each of the microscopic fields express PDX1 in their cytoplasm.

### DETAILED DESCRIPTION OF THE INVENTION

The present invention, in some embodiments thereof, relates to methods of isolating pancreatic progenitor cells, definite endodermal cells and isolated cell population obtained thereby and, to nucleic acid constructs, cells and kit for identifying markers which characterize pancreatic progenitor cells or definite endodermal cells, and to methods and kits using same for identifying compounds involved in the differentiation of same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

In the quest for developing cutting edge therapeutics, a significant effort is put on the production of stem cells (hESC and/or iPS) derived treatments. A key step in a successful production of new insulin secreting cells is the identification, isolation and characterization of stage-specific progenitor cells. The present inventors used genetic labeling for screening stage specific genes, and examined the expression profiling of cells in specific stages of the pancreatic differentiation process.

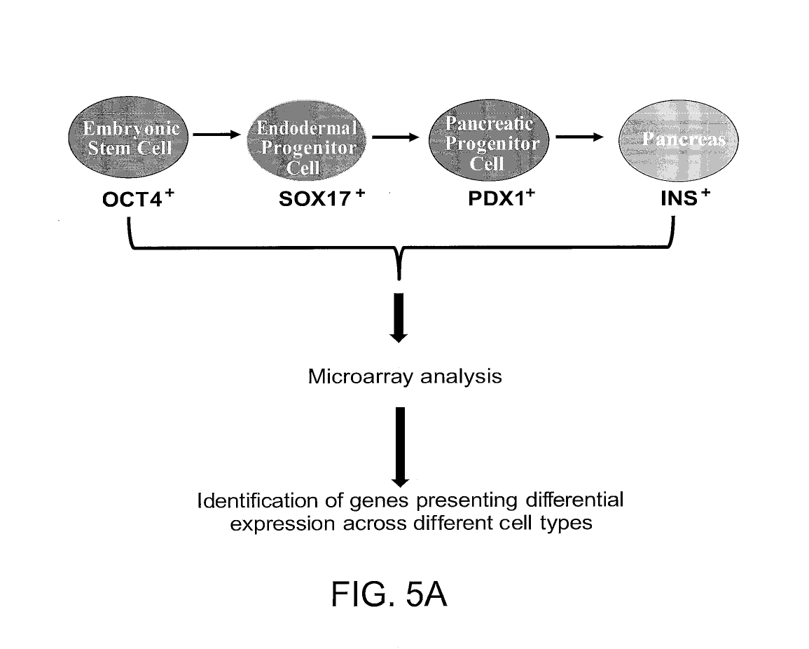

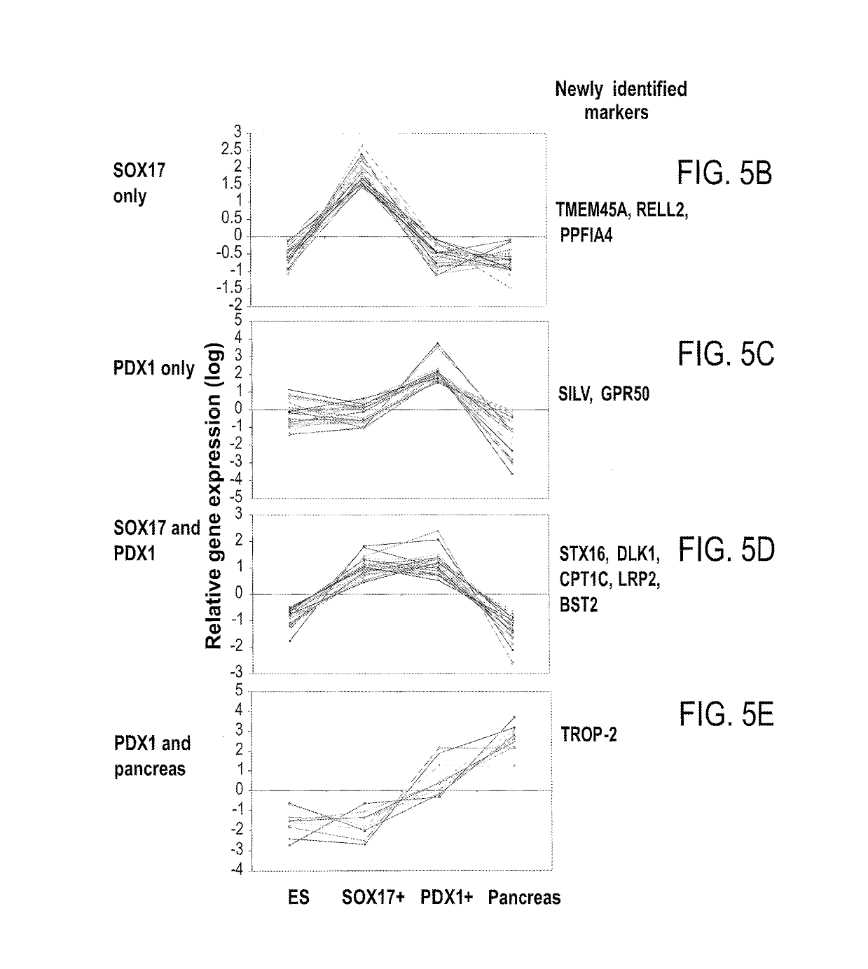

FIG. 5A depicts the general strategy used to identify markers presenting differential expression across different cell types. To achieve that goal, pluripotent stem cells were used for two major differentiation stages: (1) Definitive endoderm stage represented by SOX17 positive population; (2) Pancreatic progenitor cells represented by PDX1 positive population. Undifferentiated pluripotent stem cells (e.g., ESCs) were used as control for the experiment. In addition, DNA microarray data from pancreas cells was used for further comparisons. To enhance the reliability of the analysis, three replicas from each cell type were included in the experiment.

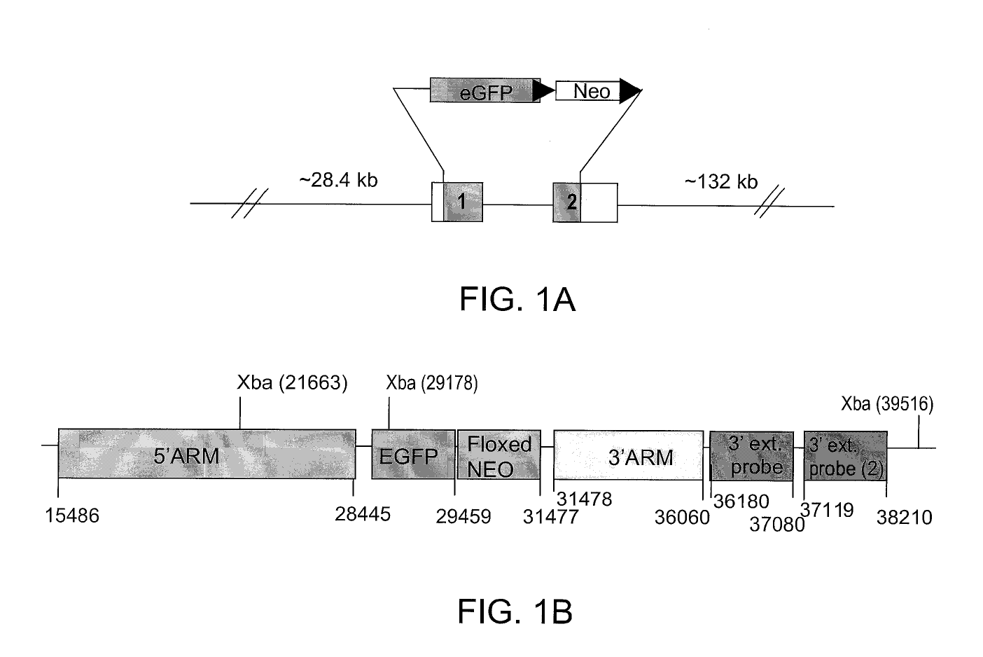

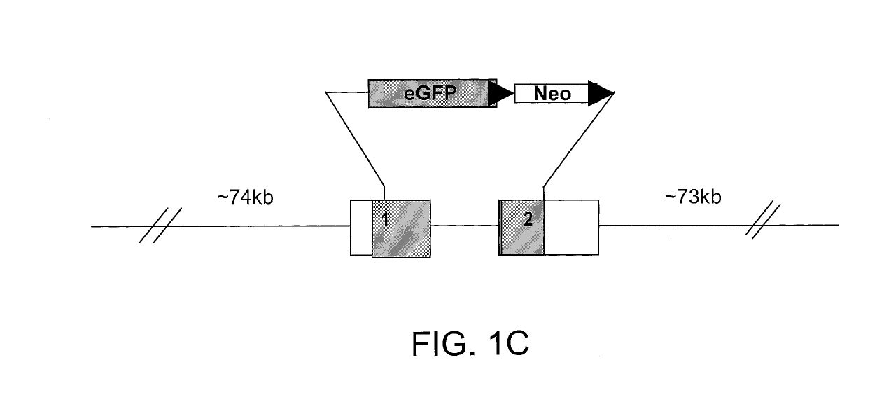

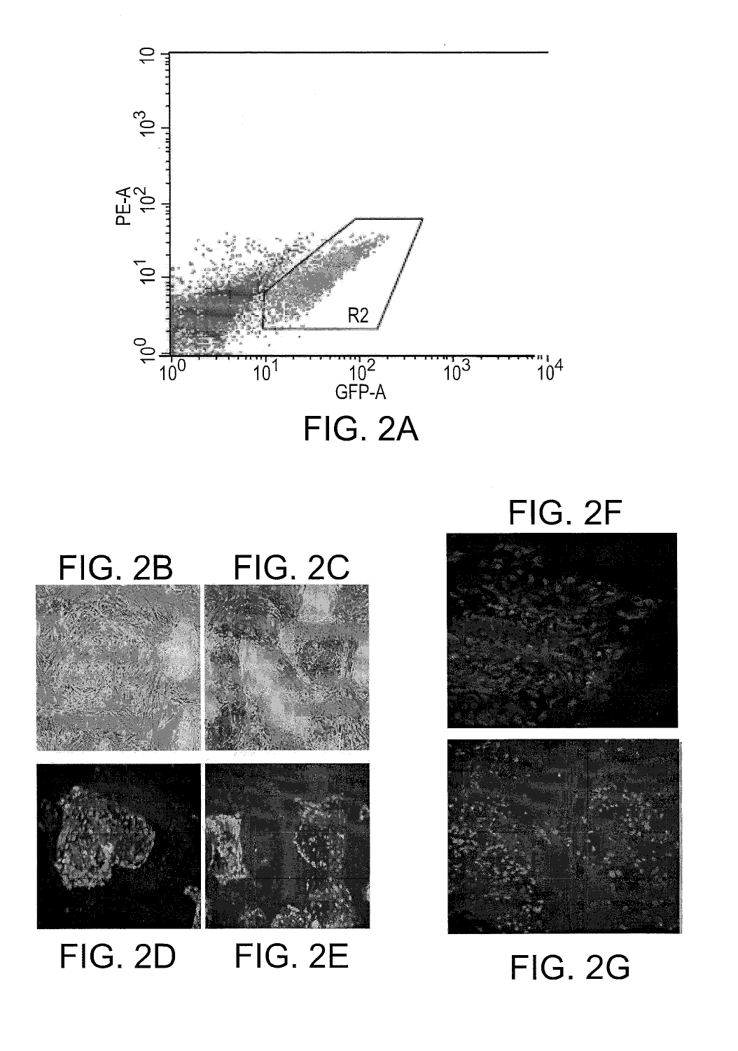

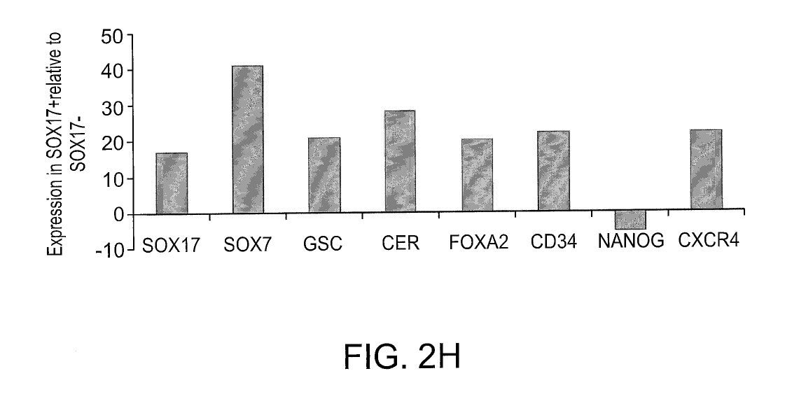

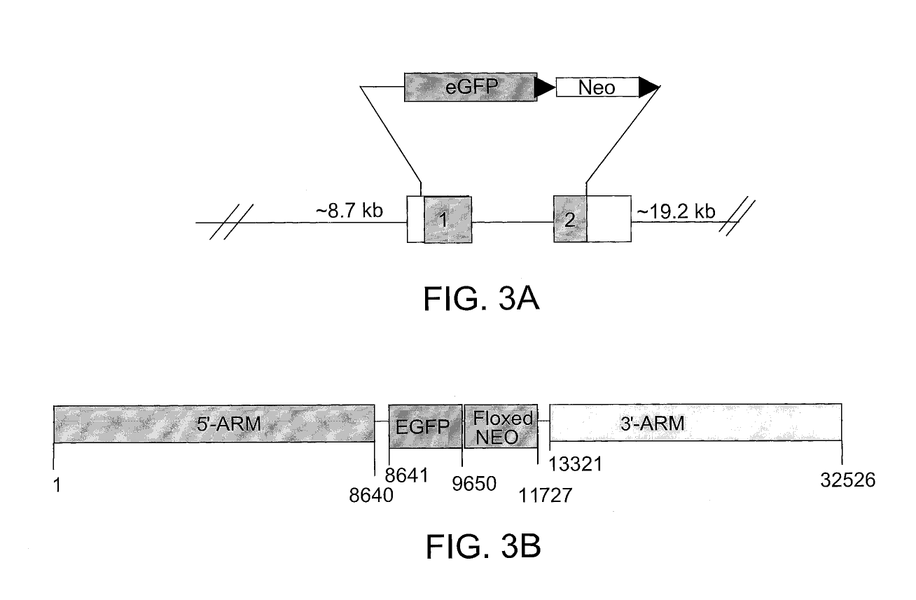

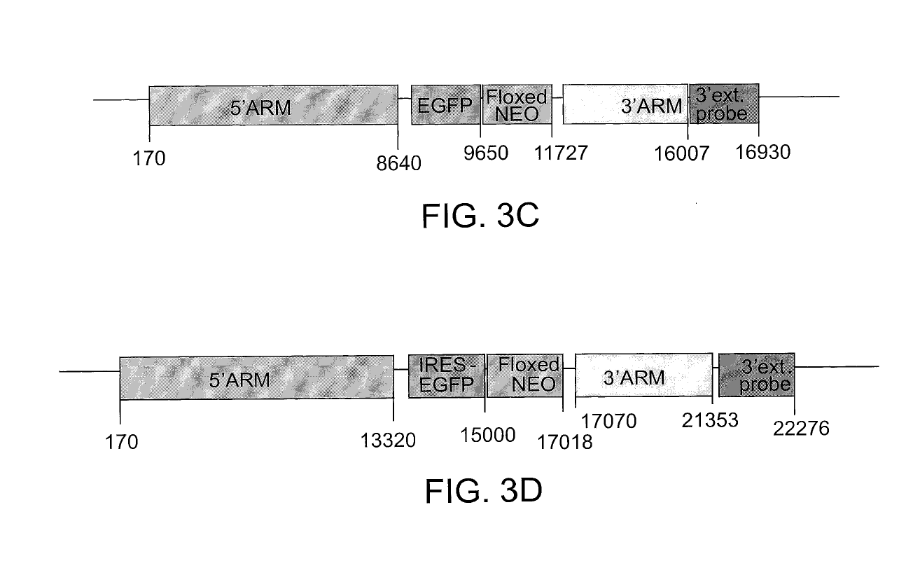

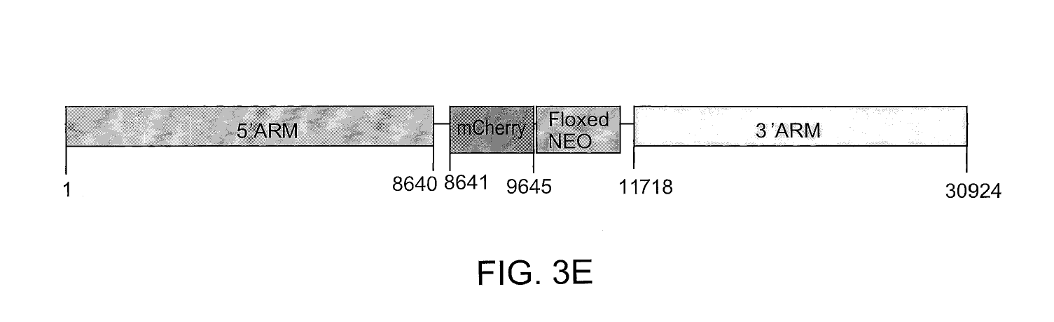

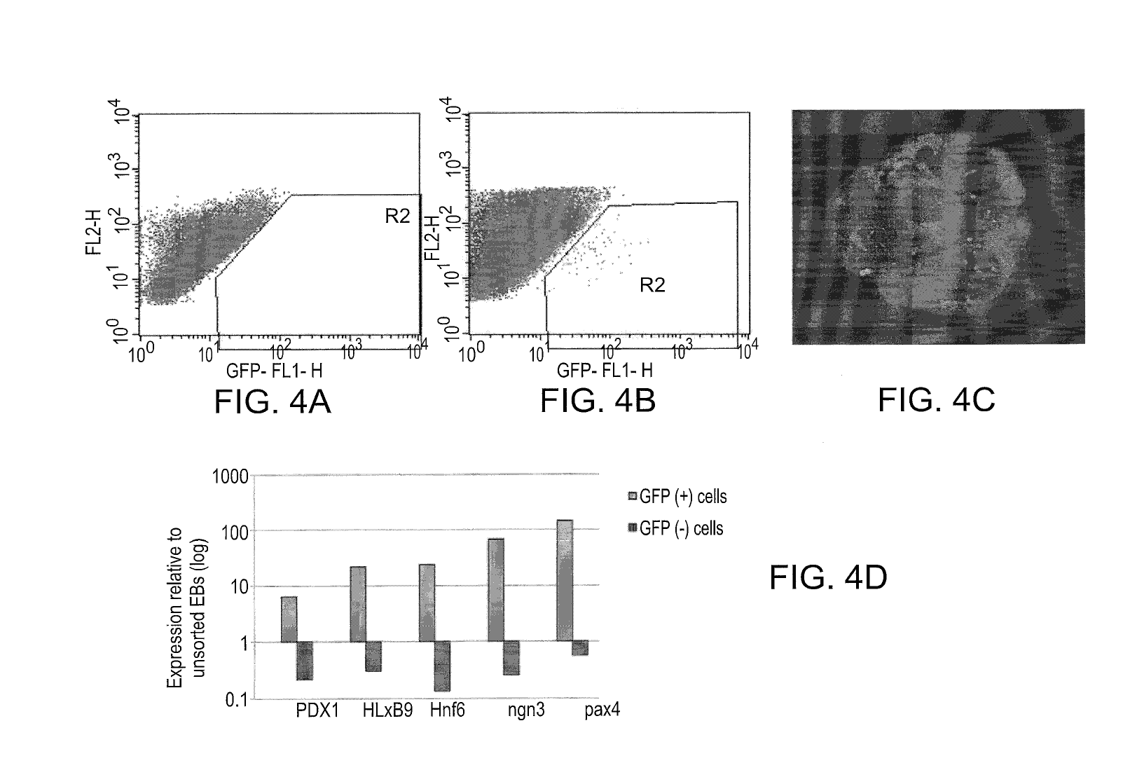

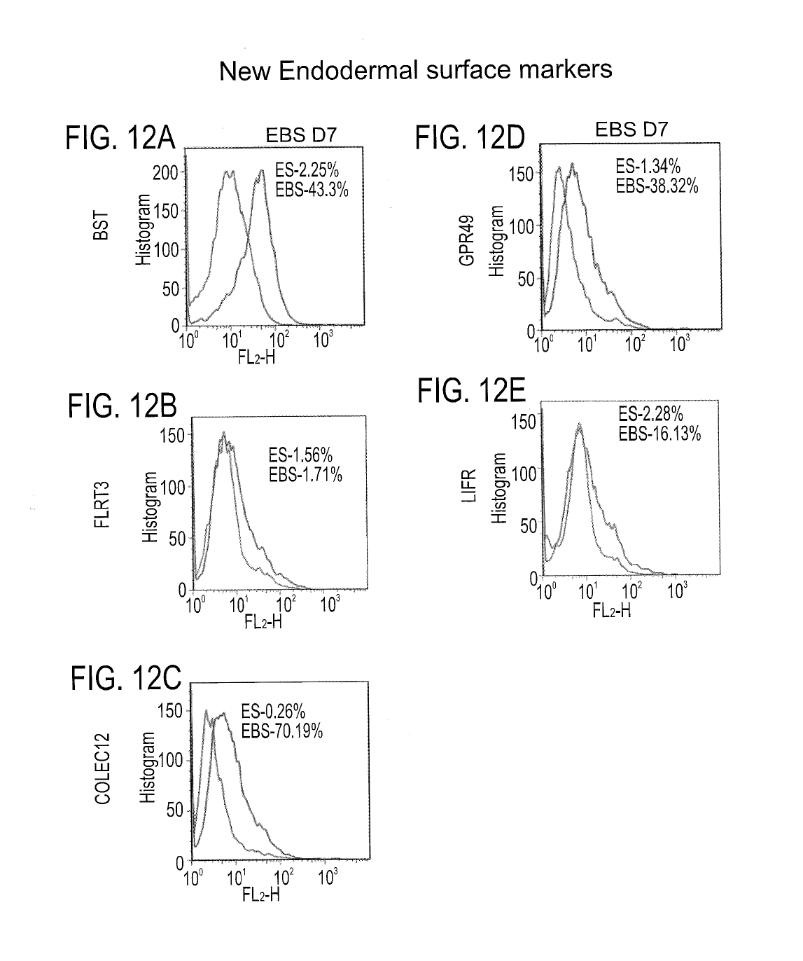

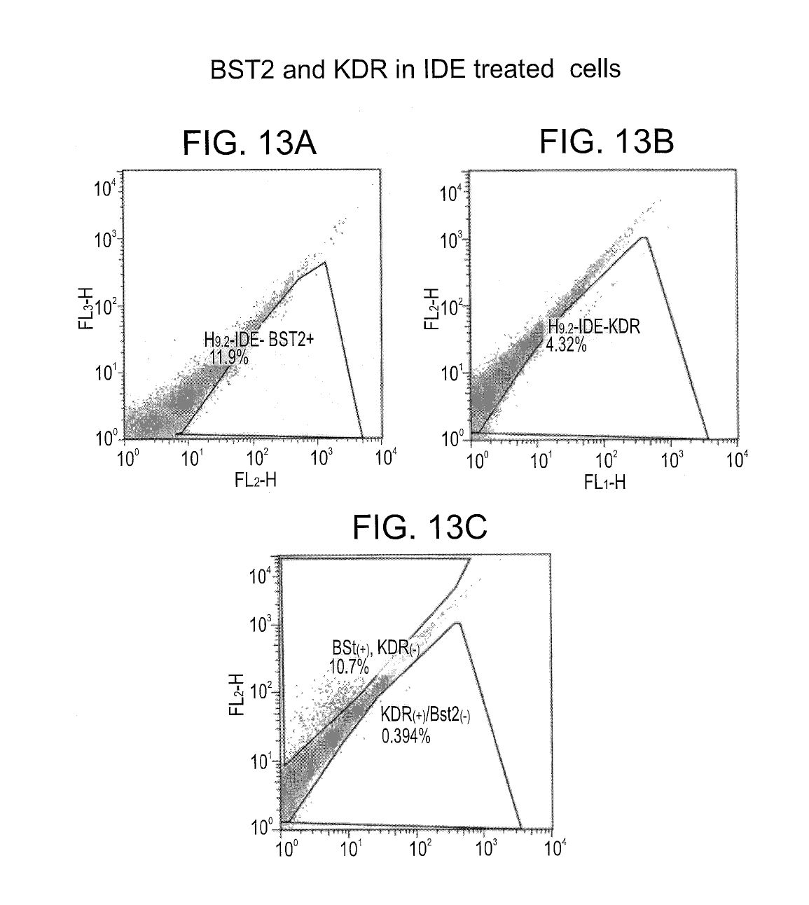

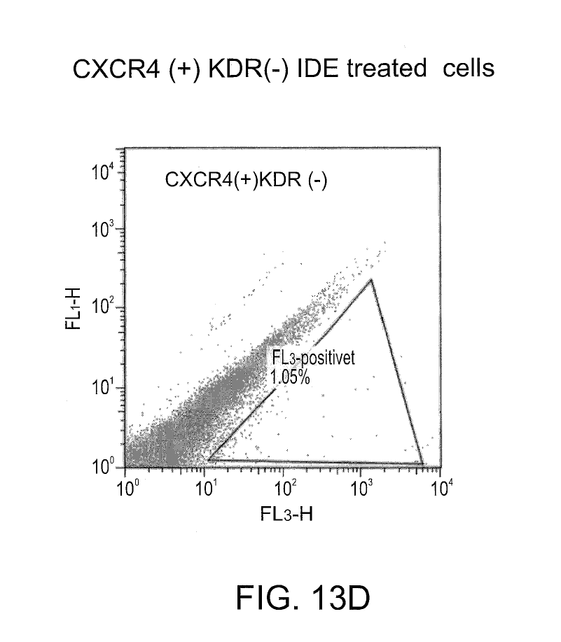

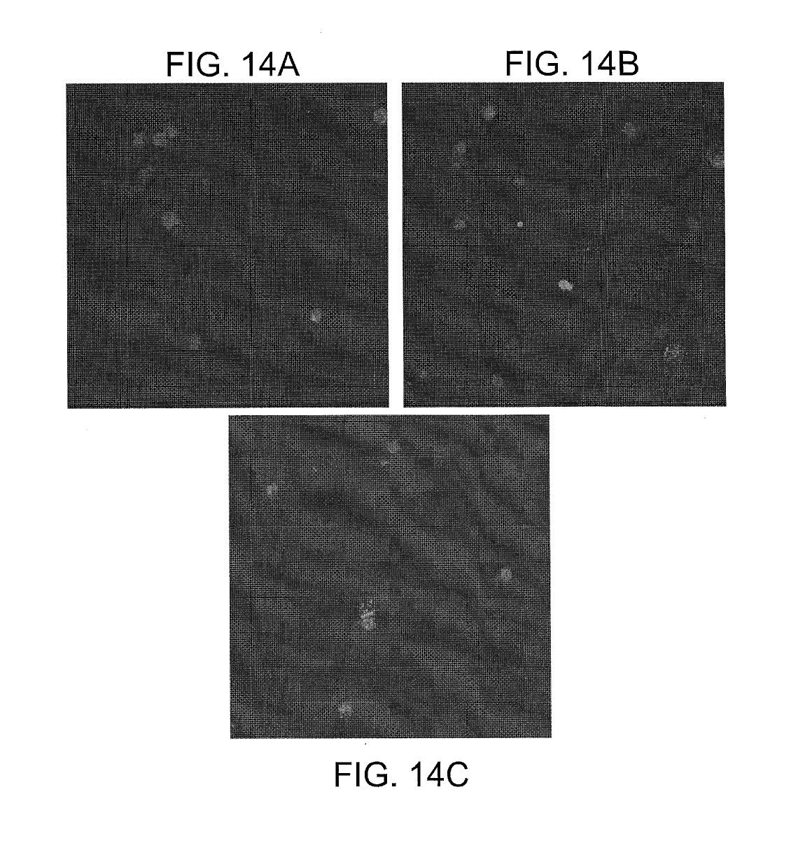

Thus, the present inventors have uncovered various markers which characterize the endoderm progenitor population and the pancreatic progenitor population, two intermediate stages along the differentiation pathway to insulin producing cells. As described in the Examples section which follows, the present inventors have generated human embryonic stem cells clones harboring SOX17 (definitive endoderm marker) regulatory sequences fused to a reporter gene (e.g., GFP) (FIGS. 1A-C, Example 1 of the Examples section which follows), and the PDX1 (pancreatic progenitor marker) regulatory sequences fused to a reporter gene (e.g., GFP or mCherry) (FIGS. 3A-E, Example 2 of the Examples section which follows), introduced the constructs into embryonic stem cells and identified subpopulations of GFP⁺ cells [FIG. 2A (SOX17+ cells) and FIG. 4B (PDX1+ cells); Example 1 and 2 of the Examples section which follows]. Using FACS-based purification, the present inventors identified and isolated stage-specific human progenitor cell populations. As is further shown in FIGS. 2H and 4D, Tables 1-12 and described in Examples 1-3 of the Examples section which follows, the present inventors have determined the expression level of various genes in the isolated SOX17+ or PDX1+ stage-specific populations and identified genes that are upregulated or downregulated at specific stages of differentiation and hence define those stages. The identified markers (genes) are involved in the differentiation process and can be used for optimizing conditions for ultimately producing beta cells for transplantation therapy, for selecting pancreatic progenitors from mixed populations of differentiating cells and for carrying out quality control of candidate islet replacement cells. In addition, as is shown in Tables 13-20 and described in Example 4 of the Examples section which follows, by employing the PARTEK cluster analysis the present inventors have identified additional genes/markers which can be used to identify pancreatic progenitor cells, definite endodermal cells, SOX17 and PDX1 expressing cells, and PDX1+ and pancreatic cells. Further experiments using some of the novel markers identified herein were used to isolate pancreatic progenitor cells. Thus, as shown in FIGS. 14A-C and described in Example 5 of the Examples section which follows, cells of embryoid bodies (day 28), which were sorted using an anti-TROP-2 antibody, were found to include at least 84% of PDX1-positive cells. In addition, as is shown in FIGS. 12A-E and described in Table 21 (Example 6 of the Examples section which follows), cells of 7-day-old EBs were found to express high levels of BST2, FLRT3, COLEC12, GPR-49/LGR5 and LIF-R as compared to undifferentiated hESCs. Moreover, induction of undifferentiated hESCs towards the definite endoderm stage by treatment with IDE1/IDE2 resulted in cells which are BST2+/KDR−, and cells which are CXCR4+/KDR− (FIGS. 13A-D, Example 7 of the Examples section which follows).

Thus, according to an aspect of some embodiments of the invention there is provided a method of identifying pancreatic progenitor cells. The method is effected by determining in a population of cells which comprises pancreatic progenitor cells at least one marker that is:

(i) positively associated with pancreatic differentiation, the marker being selected from the group consisting of: TACSTD2 (TROP-2), GPR50, BST2, NTRK2, ITGA4, KDR, PTPRN, LGI1, VIPR2, SLC2A1, MUC15, MUC12, LPHN3, MUC16, VTCN1, MMP16, FZD3, ITGB6, GFRA3, ROBO1, NLGN1, MUC12, CNTFR, LPHN1, SULF1, ADAM23, SCUBE3, PLAU, CDON, SLIT2, C7orf68, PLXDC2, CD74, MUC15, GPR56, VTCN1, ITGB6, AREG, BOC, KLRK1, PRTG, PTPRZ1, GABRP, SILV, KIAA1772, PLP1, OVOS, HAPLN1, EPHA7, ENO2, PCDHB5, OVOS, SYT1, DCT, GPAM, SLITRK6, DCC, FREM2, SDK2, CGA, ATP1B2, SEMA3D, PCDHB15, CDH1, WNT8B, LPAR4, NPIPL3, FAM171B, PTN, ABCC2, ADAMTS3, RASA4, CPT1C, SLC6A6, PCDHB3, LRRC37B, RNFT2, KCNG3, TRPC1, ALPPL2, OR4F21, CCL2, KIF5A, OLFM2, CACNG7, MPHOSPH9, SLC13A4, MOXD1, C6orf186, SLC4A8, STX16, AMY2A, SPARCL1, MGP, A2M, DCN, ATP8B1, MMRN1, EMP1, PLA2G2A, PDE3A, TLR3, CYP1B1, PTGIS, RFTN2, PLEKHA2, SMOC1, STOM, JAM2, CHL1, SCG5, IGFBP7, NPR3, IFI6, CR1L, OR2A4, OR2A7, KCNG3, CACNG7, GRID2, CDH1, LPAR3, SEMA6A, PTPRZ1, ATP1A3, CAMKV, SCNN1G, SYT6, SLC18A2, PCDHB5, ABCG2, HLA-DRA, CR1L, HTR2C, EDNRB, PCDH11X, SLC17A7, SCNN1A, CD9, CXCL16, FXYD5, GABRQ, GFRA3, CACNA2D2, CLDN4, PLP1, PDPN, MMP24, SDK2, GPR176, GPR64, GPR160, PCDH11Y, NKAIN4, ATP1B2, SCN8A, THBS4, CR2, HLA-DQA1, HLA-DRA, HTR7, SLC2A1, HLA-DRA, KCNS3, SLC7A3, HLA-DPB2, CACNA1B, and GPR143, wherein an upregulation above a predetermined threshold of an expression level of the marker positively associated with the pancreatic differentiation as compared to the expression level of the marker in a reference cell, wherein the reference cell is a definite endodermal cell which expresses SOX17, is indicative of a positive identification of a pancreatic progenitor cell, and alternatively or additionally

(ii) negatively associated with the pancreatic differentiation, the marker being selected from the group consisting of: LGR5, CCKBR, CXCR4, FLRT3, TRPA1, SLC40A1, LRIG3, COLEC12, EPSTI1, GPR128, CDH2, SLC5A9, FSHR, SLC30A10, IL13RA1, SLC7A7, PCDH10, SLC1A1, GPR141, LIFR, TMEM27, GPC4, LYPD6B, FOLH1, TRPC4, PCDH7, KEL, KCNJ3, OR2T4, VIPR2, FLRT2, CD34, SLC39A8, CLDN11, CXCR7, ITGA5, ITGAV, CALCR, CLDN18, SLC7A5, TMBIM4, SLCO2A1, CDH10, AMHR2, ASAM, CLDN1, DSCAM, TMEM88, PLXNA2, CD177, TMEM144, GPR37, GJA5, SEMA6D, NIPAL2, GPR151, MCC, TMEM136, KCNG1, LHFPL2, MOSPD1, SLC37A1, LRIT3, EPHA4, GPR177, and IL1R1, wherein a downregulation above a predetermined threshold of an expression level of the marker negatively associated with the pancreatic differentiation as compared to the expression level of the marker in a reference cell, wherein the reference cell is a definite endodermal cell which expresses SOX17, is indicative of a positive identification of a pancreatic progenitor cell,

thereby identifying the pancreatic progenitor cells.

As used herein the phrase “pancreatic progenitor cell” relates to a cell which express the duodenal homoebox factor-1 (PDX1) and which is not terminally differentiated but has the potential to differentiate to pancreatic endocrine, exocrine and/or duct cells.

As used herein the phrase “terminally differentiated” refers to a cell which cannot further differentiate in the lineage.

According to some embodiments of the invention, the pancreatic progenitor cell is capable of differentiating into at least two types of cells present in the pancreas.

It should be noted that the pancreas includes endocrine, exocrine and duct cells.

Pancreatic endocrine cells are present in the pancreatic islets of Langerhans, and produce hormones that underlie the endocrine functions of the pancreas, such as insulin, glucagon, and somatostatin. Pancreatic exocrine cells are present in the pancreas and secrete the pancreatic juice containing digestive enzymes that assist the absorption of nutrients and the digestion in the small intestine (e.g., proteases and peptidases, lipases, carbohydrases, and nucleases). Pancreatic duct cells transport bile and pancreatic enzymes from the pancreas to the hepatopancreatic ampulla.

According to some embodiments of the invention, the pancreatic progenitor cells are capable of differentiating into insulin-producing cells.

As used herein the term “identifying” refers to classifying a cell according the expression level of a marker characterizing the cell.

For example, a cell can be classified as being a pancreatic progenitor cell or as being a non-pancreatic progenitor cell based on the expression level of a marker characterizing the pancreatic progenitor cell.

As used herein the phrase “associated with pancreatic differentiation” refers to a marker which is upregulated (i.e., positively associated) or downregulated (i.e., negatively associated) when a cell is induced towards the pancreatic cell lineage, e.g., in a pancreatic progenitor cell.

As used herein the phrase “expression level” refers to the degree of gene expression and/or gene product activity in a specific cell. For example, up-regulation or down-regulation of various genes can affect the level of the gene product (i.e., RNA and/or protein) in a specific cell.

It should be noted that the level of expression can be determined in arbitrary absolute units, or in normalized units (relative to known expression levels of a control reference). For example, when using DNA chips, the expression levels are normalized according to the chips' internal controls or by using quantile normalization such as RMA (Robust Multichip Average).

As used herein the term “marker” refers to a gene or a gene product described using an accepted gene symbol.

According to some embodiments of the invention, the level of the marker is determined by the expression level of any of the polynucleotide transcripts which comprise the target nucleotide sequence identified by the Affymetrix probe set ID and/or any of the proteins which comprise the amino acid sequence encoded by the target nucleotide sequence identified by the Affymetrix probe set ID. The target nucleotide sequences identified by each of the Affymetrix probe set IDs are provided in Tables 1-20 in the Examples section which follows. The polynucleotides and polypeptides (which are identified by the Affynetrix probe set ID) are presented in Tables 1-20 by their sequence identifiers (SEQ ID NO:) and in some cases also by their GenBank Accession numbers. All polypeptides and polynucleotides are provided in the sequence listing of the application.

According to some embodiments of the invention, each of the polynucleotides or polypeptides, for which the expression level is determined, comprises the entire target sequence (with 100% sequence identity) or the amino acid sequence encoded by the target sequence, respectively.

The cells used by the method of some embodiments of the invention can be isolated cells (at least partially removed from a subject), cultured cells and/or non-cultured cells (e.g., primary cells obtained from a subject). The cells can be obtained from the subject by any known method including, but not limited to, tissue biopsy (e.g., pancreas biopsy) obtained using a surgical tool or a needle), fine needle aspiration, and the like. It should be noted that the cells may be isolated from the subject (e.g., for in vitro detection) or may optionally comprise a cell that has not been physically removed from the subject (e.g., in vivo detection).

According to some embodiments of the invention, the expression level of the marker in the test cell (e.g., the cell of the population of cells which is subject to the method of identifying pancreatic progenitor cells according to some embodiments of the invention) and in the reference cell (e.g., the definite endodermal cell which expresses SOX17) is determined under identical assay conditions.

According to some embodiments of the invention, the level of expression of the marker (gene) of the invention is determined using an RNA or a protein detection method.

According to some embodiments of the invention, the RNA or protein molecules are extracted from the cell of the subject.

Methods of extracting RNA or protein molecules from cells of a subject are well known in the art. Once obtained, the RNA or protein molecules can be characterized for the expression and/or activity level of various RNA and/or protein molecules using methods known in the arts.

Non-limiting examples of assays for detecting the expression level of RNA molecules in a cell sample include Northern blot analysis, RT-PCR, RNA in situ hybridization (using e.g., DNA or RNA probes to hybridize RNA molecules present in the cells or tissue sections), in situ RT-PCR (e.g., as described in Nuovo G J, et al. Am J Surg Pathol. 1993, 17: 683-90; Komminoth P, et al. Pathol Res Pract. 1994, 190: 1017-25), and oligonucleotide microarray (e.g., by hybridization of polynucleotide sequences derived from a sample to oligonucleotides attached to a solid surface [e.g., a glass wafer) with addressable location, such as Affymetrix microarray (Affymetrix®, Santa Clara, Calif.)].

Non-limiting examples of assays for detecting the expression level and/or activity of specific protein molecules in a cell sample include Enzyme linked immunosorbent assay (ELISA), Western blot analysis, radio-immunoassay (RIA), Fluorescence activated cell sorting (FACS), immunohistochemical analysis, in situ activity assay (using e.g., a chromogenic substrate applied on the cells containing an active enzyme), in vitro activity assays (in which the activity of a particular enzyme is measured in a protein mixture extracted from the cells). For example, in case the detection of the expression level of a secreted protein is desired, ELISA assay may be performed on a sample of fluid obtained from the subject (e.g., serum), which contains cell-secreted content.

According to some embodiments of the invention, the definite endodermal cell, which express SOX17, is characterized by a SOX17+/SOX7+ expression signature.

According to some embodiments of the invention, the definite endodermal cell is characterized by the SOX17+/SOX7+/GSC+/CER+/FOXA2+/CXCR4+/NANOG− expression signature.

According to some embodiments of the invention, the definite endodermal cell is characterized by the SOX17+/SOX7+/GSC+/CER+/FOXA2+/CXCR4+/NANOG− expression signature.

According to some embodiments of the invention, the definite endodermal cell is characterized by the SOX17+/SOX7+/GSC+/CER+/FOXA2+/CXCR4+/CD34+/NANOG− expression signature.

According to some embodiments of the invention, the expression level of the marker in the definite endodermal cell is determined from at least one definite endodermal cells, e.g., from at least 2, from at least 3, from at least 4, from at least 5, from at least 6, from at least 7, from at least 8, from at least 9, from at least 10, from at least 20, from at least 100, from at least 1000, from at least 1×10⁴, from at least 1×10⁵, e.g., from at least 1×10⁶ definite endodermal cells. It should be noted that when more than one definite endodermal cell is used, the expression level of the marker may comprise an average of the expression level of several or all cells, and those of skills in the art are capable of averaging expression levels from 2 or more cells, using e.g., normalized expression values.

As used herein the phrase “an upregulation above a predetermined threshold” refers to an increase in the level of expression in the cell relative to a reference cell (e.g., the definite endodermal cell) which is higher than a predetermined threshold such as a about 10%, e.g., higher than about 20%, e.g., higher than about 30%, e.g., higher than about 40%, e.g., higher than about 50%, e.g., higher than about 60%, higher than about 70%, higher than about 80%, higher than about 90%, higher than about 2 times, higher than about three times, higher than about four time, higher than about five times, higher than about six times, higher than about seven times, higher than about eight times, higher than about nine times, higher than about 20 times, higher than about 50 times, higher than about 100 times, higher than about 200 times of at least one reference cell (e.g., definite endodermal cell). The upregulation in the expression level can be also determined using logarithmic fold changes as shown in the Examples section which follows.

According to some embodiments of the invention, the method further comprising determining in a population of cells which comprises pancreatic progenitor cells at least one marker that is: (i) negatively associated with the pancreatic differentiation, the marker being selected from the group consisting of: CST1, CER1, ANKRD1, TRY6, HAS2, DKK1, PRSS2, HP, APOA2, RHOBTB3, BMP2, ACE2, STC1, PDZK1, HHEX, VIL1, PRDM1, EOMES, DNAJC15, TNIK, IGFBP5, RLBP1L2, ADAMTS9, EPSTI1, C5, ARHGAP24, TRY6, ANGPT2, TTR, MYL7, FST, KITLG, GATA3, ST8SIA4, CCDC141, TSPYL5, EGFLAM, TTN, LEFTY2, FOXA2, FAM184A, STMN2, DIO3, FN1, PRSS1, NPPB, RGSS, MANEA, OTX2, CFLAR, FZD5, LOC151009, IFLTD1, PPFIBP2, SYTL5, ARHGAP28, NTS, APOB, CST2, FOLH1B, TFF1, GPAM, RNF152, SEMA3E, OXCT1, DUSP4, RSPO3, EHHADH, DGKK, VEGFA, APOA1, RAB17, LYPD6B, TNNC1, ZNF280A, FRZB, APOA1, SOX17, SAMD3, BMP5, GATA4, MATN3, FAM122C, SPOCK3, PLOD2, NPL, ATP6V0D2, SERHL, SERPINE2, TGFB2, ELMO1, C8orf49, ENC1, COL5A2, FMO5, COL4A1, MGST2, GLUD2, CLIP4, UNC93A, GLIPR2, GSTA2, H2AFY2, ST6GALNAC2, DUSP6, NTN4, LHX1, PUS7L, TNC, BARB, HNF1B, SHISA2, NEXN, MSL3L2, IRAK4, S100A16, CDK6, FAM159B, ELL2, B3GALNT1, ARL4D, ETS2, MUM1L1, VTN, SEMA3A, ZNF611, VWA5A, ZNF518B, NUDT4P1, RASGEF1B, TPK1, ANXA3, MYL4, BMI1, ODC1, ARSE, PTPN13, ZNF321, NODAL, ANKMY2, LPGAT1, PROS1, KLF8, PAX6, GCNT1, S100Z, ZNF702P, CCDC129, LRRFIP1, TBC1D9, YPEL2, GCNT4, F10, ZNF585B, ELMOD2, GLT8D3, ETS1, SALL1, IAH1, RFC1, ZNF214, DPPA3, FGA, B3GNT5, COL4A6, DENND2C, FZD4, MYOCD, DDIT4L, PLCXD3, ELL2, SPOCK1, IGFBP6, SERHL2, SERPINI1, FAM26E, RNF128, RBM24, GALNT4, CTSL2, ALPK2, ANKRD20B, PLCE1, TAGLN, STAT4, SOAT1, MIXL1, HNF4A, IQCA1, KLHL14, MAML3, EGF, LEFTY1, ANKRD20B, RGS8, TAL2, ADI1, SPA17, KHDRBS2, and MCC, wherein a downregulation above a predetermined threshold of an expression level of the marker negatively associated with the pancreatic differentiation as compared to the expression level of the marker in a reference cell, wherein the reference cell is a definite endodermal cell which expresses SOX17 is indicative of a positive identification of a pancreatic progenitor cell and alternatively or additionally; (ii) positively associated with the pancreatic differentiation, the marker being selected from the group consisting of: ALPPL2, DPPA5, H19, CRYZ, CXCL12, TYW3, ZYG11A, CRABP1, IDO1, POU5F1, HEY2, HIST1H1A, TFAP2C, DPPA2, ZFP42, LECT1, NECAB1, CKMT1A, SAMHD1, FGF2, PLA2G2A, PRDM14, POU5F1, GLI3, GSTT2, OLFML3, DAZL, GALNT3, SOX2, POU5F1B, ACTN3, CPT1A, DCLK1, EDIL3, NANOG, THUMPD3, VASH2, ATCAY, USP44, HIST1H4F, NANOG, PIM2, DNMT3B, ZNF483, FEZF1, SCARNA9L, SCGB3A2, SILV, ENPP1, MYC, NLRP7, TDRD1, HHLA1, MT1G, RASL11B, PYGM, APOBEC3B, COL14A1, PTRF, HOXA1, CYP2S1, NRK, RASGRP2, FBXO2, KIF5A, PLEKHA2, HERC5, TRIML2, ARRDC4, HSPA2, ZNF248, NPTX2, MT1X, SPP1, PHC1, LRAT, NLRP4, GFPT2, ZNF680, MYO1E, B2M, DDX43, FABP3, GRHL2, ACOXL, CDCA7L, LDB2, KIAA1772, SFRP2, TRIM71, PLAU, AIM1, MT1E, STAT3, ZSCAN10, SCG3, VAT1L, HPDL, ZFP57, BNC2, CYP4F22, NOTCH1, LIX1, QPRT, ZNF398, RGS10, MT1G, GAA, PAX3, RHBDL3, POLR3G, FAM46B, MT1F, GLB1L3, RBM46, PRODH, ZNF300, STOM, PLA2G16, GAL, TOX, B3GALT1, MDGA2, MAD2L2, ATP6V0A4, LAMA2, HORMAD1, TRIM22, RNF157, ADD2, UPRT, PSMB8, ZNF562, FAM9C, UGT8, KIF5A, SNRPN, UNC13A, DOC2A, ZNF560, VENTX, RARRES2, CCDC109B, MT2A, ALDOC, STARD9, and AKAP1, wherein an upregulation above a predetermined threshold of an expression level of the marker positively associated with the pancreatic differentiation as compared to the expression level of the marker in a reference cell, wherein the reference cell is a definite endodermal cell which expresses SOX17, is indicative of a positive identification of a pancreatic progenitor cell.

According to an aspect of some embodiments of the invention, the method of identifying the pancreatic progenitor cells comprising determining in a population of cells which comprises pancreatic progenitor cells at least one marker that is: (i) negatively associated with the pancreatic differentiation, the marker being selected from the group consisting of: LGR5, CCKBR, CXCR4, FLRT3, TRPA1, SLC40A1, LRIG3, COLEC12, EPSTI1, GPR128, CDH2, SLC5A9, FSHR, SLC30A10, IL13RA1, SLC7A7, PCDH10, SLC1A1, GPR141, LIFR, TMEM27, GPC4, LYPD6B, FOLH1, TRPC4, PCDH7, KEL, KCNJ3, OR2T4, VIPR2, FLRT2, CD34, SLC39A8, CLDN11, CXCR7, ITGA5, ITGAV, CALCR, CLDN18, SLC7A5, TMBIM4, SLCO2A1, CDH10, AMHR2, ASAM, CLDN1, DSCAM, TMEM88, PLXNA2, CD177, TMEM144, GPR37, GJA5, SEMA6D, NIPAL2, GPR151, MCC, TMEM136, KCNG1, LHFPL2, MOSPD1, SLC37A1, LRIT3, EPHA4, GPR177, IL1R1, CST1, CER1, ANKRD1, TRY6, HAS2, DKK1, PRSS2, HP, APOA2, RHOBTB3, BMP2, ACE2, STC1, PDZK1, HHEX, VIL1, PRDM1, EOMES, DNAJC15, TNIK, IGFBP5, RLBP1L2, ADAMTS9, EPSTI1, C5, ARHGAP24, TRY6, ANGPT2, TTR, MYL7, FST, KITLG, GATA3, ST8SIA4, CCDC141, TSPYL5, EGFLAM, TTN, LEFTY2, FOXA2, FAM184A, STMN2, DIO3, FN1, PRSS1, NPPB, RGS5, MANEA, OTX2, CFLAR, FZD5, LOC151009, IFLTD1, PPFIBP2, SYTL5, ARHGAP28, NTS, APOB, CST2, FOLH1B, TFF1, GPAM, RNF152, SEMA3E, OXCT1, DUSP4, RSPO3, EHHADH, DGKK, VEGFA, APOA1, RAB17, LYPD6B, TNNC1, ZNF280A, FRZB, APOA1, SOX17, SAMD3, BMP5, GATA4, MATN3, FAM122C, SPOCK3, PLOD2, NPL, ATP6V0D2, SERHL, SERPINE2, TGFB2, ELMO1, C8orf49, ENC1, COL5A2, FMO5, COL4A1, MGST2, GLUD2, CLIP4, UNC93A, GLIPR2, GSTA2, H2AFY2, ST6GALNAC2, DUSP6, NTN4, LHX1, PUS7L, TNC, BARB, HNF1B, SHISA2, NEXN, MSL3L2, IRAK4, S100A16, CDK6, FAM159B, ELL2, B3GALNT1, ARL4D, ETS2, MUM1L1, VTN, SEMA3A, ZNF611, VWA5A, ZNF518B, NUDT4P1, RASGEF1B, TPK1, ANXA3, MYL4, BMI1, ODC1, ARSE, PTPN13, ZNF321, NODAL, ANKMY2, LPGAT1, PROS1, KLF8, PAX6, GCNT1, S100Z, ZNF702P, CCDC129, LRRFIP1, TBC1D9, YPEL2, GCNT4, F10, ZNF585B, ELMOD2, GLT8D3, ETS1, SALL1, IAH1, RFC1, ZNF214, DPPA3, FGA, B3GNT5, COL4A6, DENND2C, FZD4, MYOCD, DDIT4L, PLCXD3, ELL2, SPOCK1, IGFBP6, SERHL2, SERPINI1 FAM26E, RNF128, RBM24, GALNT4, CTSL2, ALPK2, ANKRD20B, PLCE1, TAGLN, STAT4, SOAT1, MIXL1, HNF4A, IQCA1, KLHL14, MAML3, EGF, LEFTY1, ANKRD20B, RGS8, TAL2, ADI1, SPA17, KHDRBS2, and MCC, wherein a downregulation above a predetermined threshold of an expression level of the marker negatively associated with the pancreatic differentiation as compared to the expression level of the marker in a reference cell, wherein the reference cell is a definite endodermal cell which expresses SOX17, is indicative of a positive identification of a pancreatic progenitor cell and alternatively or additionally; (ii) positively associated with the pancreatic differentiation, the marker being selected from the group consisting of: TACSTD2 (TROP-2), GPR50, BST2, NTRK2, ITGA4, KDR, PTPRN, LGI1, VIPR2, SLC2A1, MUC15, MUC12, LPHN3, MUC16, VTCN1, MMP16, FZD3, ITGB6, GFRA3, ROBO1, NLGN1, MUC12, CNTFR, LPHN1, SULF1, ADAM23, SCUBE3, PLAU, CDON, SLIT2, C7orf68, PLXDC2, CD74, MUC15, GPR56, VTCN1, ITGB6, AREG, BOC, KLRK1, PRTG, PTPRZ1, GABRP, SILV, KIAA1772, PLP1, OVOS, HAPLN1, EPHA7, ENO2, PCDHB5, OVOS, SYT1, DCT, GPAM, SLITRK6, DCC, FREM2, SDK2, CGA, ATP1B2, SEMA3D, PCDHB15, CDH1, WNT8B, LPAR4, NPIPL3, FAM171B, PTN, ABCC2, ADAMTS3, RASA4, CPT1C, SLC6A6, PCDHB3, LRRC37B, RNFT2, KCNG3, TRPC1, ALPPL2, OR4F21, CCL2, KIF5A, OLFM2, CACNG7, MPHOSPH9, SLC13A4, MOXD1, C6orf186, SLC4A8, STX16, AMY2A, SPARCL1, MGP, A2M, DCN, ATP8B1, MMRN1, EMP1, PLA2G2A, PDE3A, TLR3, CYP1B1, PTGIS, RFTN2, PLEKHA2, SMOC1, STOM, JAM2, CHL1, SCG5, IGFBP7, NPR3, IFI6, CR1L, OR2A4, OR2A7, KCNG3, CACNG7, GRID2, CDH1, LPAR3, SEMA6A, PTPRZ1, ATP1A3, CAMKV, SCNN1G, SYT6, SLC18A2, PCDHB5, ABCG2, HLA-DRA, CR1L, HTR2C, EDNRB, PCDH11X, SLC17A7, SCNN1A, CD9, CXCL16, FXYD5, GABRQ, GFRA3, CACNA2D2, CLDN4, PDPN, MMP24, SDK2, GPR176, GPR64, GPR160, PCDH11Y, NKAIN4, ATP1B2, SCN8A, THBS4, CR2, HLA-DQA1, HLA-DRA, HTR7, SLC2A1, HLA-DRA, KCNS3, SLC7A3, HLA-DPB2, CACNA1B, GPR143, ALPPL2, DPPA5, H19, CRYZ, CXCL12, TYW3, ZYG11A, CRABP1, IDO1, POU5F1, HEY2, HIST1H1A, TFAP2C, DPPA2, ZFP42, LECT1, NECAB1, CKMT1A, SAMHD1, FGF2, PLA2G2A, PRDM14, POU5F1, GLI3, GSTT2, OLFML3, DAZL, GALNT3, SOX2, POU5F1B, ACTN3, CPT1A, DCLK1, EDIL3, NANOG, THUMPD3, VASH2, ATCAY, USP44, HIST1H4F, NANOG, PIM2, DNMT3B, ZNF483, FEZF1, SCARNA9L, SCGB3A2, SILV, ENPP1, MYC, NLRP7, TDRD1, HHLA1, MT1G, RASL11B, PYGM, APOBEC3B, COL14A1, PTRF, HOXA1, CYP2S1, NRK, RASGRP2, FBXO2, KIF5A, PLEKHA2, HERC5, TRIML2, ARRDC4, HSPA2, ZNF248, NPTX2, MT1X, SPP1, PHC1, LRAT, NLRP4, GFPT2, ZNF680, MYO1E, B2M, DDX43, FABP3, GRHL2, ACOXL, CDCA7L, LDB2, KIAA1772, SFRP2, TRIM71, PLAU, AIM1, MT1E, STAT3, ZSCAN10, SCG3, VAT1L, HPDL, ZFP57, BNC2, CYP4F22, NOTCH1, LIX1, QPRT, ZNF398, RGS10, MT1G, GAA, PAX3, RHBDL3, POLR3G, FAM46B, MT1F, GLB1L3, RBM46, PRODH, ZNF300, STOM, PLA2G16, GAL, TOX, B3GALT1, MDGA2, MAD2L2, ATP6V0A4, LAMA2, HORMAD1, TRIM22, RNF157, ADD2, UPRT, PSMB8, ZNF562, FAM9C, UGT8, KIF5A, SNRPN, UNC13A, DOC2A, ZNF560, VENTX, RARRES2, CCDC109B, MT2A, ALDOC, STARD9, and AKAP1, wherein an upregulation above a predetermined threshold of an expression level of the marker positively associated with the pancreatic differentiation as compared to the expression level of the marker in a reference cell, wherein the reference cell is a definite endodermal cell which expresses SOX17 is indicative of a positive identification of a pancreatic progenitor cell.

According to some embodiments of the invention, the method further comprising determining in a population of cells which comprises pancreatic progenitor cells at least one marker that is: (i) negatively associated with the pancreatic differentiation, the marker being selected from the group consisting of: TMPRSS11E, LGR5, SLC39A8, TM4SF18, CUZD1, GPC4, SLC22A3, CXCR4, NRN1, TMBIM4, THBS2, SLC7A5, TMEM47, NTS, CER1, CST1, NODAL, PRRX1, KGFLP1, NFIB, GCNT4, MIXL1, CAV1, LUM, RASGRF2, OXCT1, GLIPR1, VSNL1, FST, POSTN, GNA14, CBR1, TNIK, RGS5, KGFLP1, ETS1, MPPED2, ACTA2, SEMA3A, DACT1, ANXA1, COL12A1, KITLG, MMP2, DLEU2, ACE2, ACTG2, PUS7L, RNU5B-1, COL3A1, LEFTY2, NPPB, S1AE, AFP, ZFP42, HAS2, TRY6, NR5A2, EBF1, RPPH1, NFIX, IAH1, RUNX1T1, COL5A2, VCAN, RNU5E, ADAMTS5, CDK6, LRRC17, DBC1, MYL4, ANKRD1, EOMES, LCP1, TAGLN, TNFAIP6, FAM159B, and PGM5P2, wherein a downregulation above a predetermined threshold of an expression level of the marker negatively associated with the pancreatic differentiation as compared to the expression level of the marker in a reference cell, wherein the reference cell is an undifferentiated embryonic stem cell (hESC), is indicative of a positive identification of a pancreatic progenitor cell, and alternatively or additionally; (ii) positively associated with the pancreatic differentiation, the marker being selected from the group consisting of: TACSTD2 (TROP-2), BST2 LRP2, LAMA1, GPC3, LGI1, FN1, LPHN3, FGB, KIT, DPP4, TRO, SPA17, ROBO2, DLK1, PRTG, DKK1, APOA2, AFP, APOB, FLRT3, KIAA1772, FBN2, FRZB, KLK6, FREM2, RSPO3, APOA1, APOA1, SEMA3D, KEL, BMP5, HEPH, STMN2, CACNB3, CHST9, TMC7, FAM171B, ATP5G1, FOLR1, ABCC2, EFNA5, CPT1C, MAGED1, RASA4, KDELR3, ST6GALNAC2, EDNRA, LRRC37B, ABCC4, CLCN5, MBOAT2, SLC44A5, NPC2, ELFN1, MMD, SLC5A3, IMPAD1, OSTC, DSC2, SLC31A1, SLC5A12, LRRN3, NPIPL3, GDPD1, NPIPL3, NPIPL3, LINGO2, LYPD6B, PRTG, LRP2, CACNG7, DLK1, CACNA2D2, CR1L, SCNN1G, HTR2C, LPAR3, THBS3, KCNG3, SDK2, HLA-DRA, SLC18A2, CXCL16, TMEM63C, SLC17A7, GFRA3, DPPA5, ALPPL2, CRYZ, H19, ZYG11A, TYW3, ARRDC4, TXNIP, FOS, NCRNA00173, GSTT2, C3, DDX43, B2M, PYGM, CYP4F22, ZNF578, ENO2, ZNF248, NLRP4, SNORD59B, SNORD113-4, CASZ1, MIR21, IFI16, ZNF560, TDRD1, ZNF680, HSPA1B, HSPA1A, EGR1, RASGRP2, ASMTL, CPT1C, ATCAY, ACSF2, PAMR1, SILV, ACTN3, HORMAD1, ACSM3, RNF157, SAMHD1, and RCN1, wherein an upregulation above a predetermined threshold of an expression level of the marker positively associated with the pancreatic differentiation as compared to the expression level of the marker in a reference cell, wherein the reference cell is an undifferentiated embryonic stem cell (ESC), is indicative of a positive identification of a pancreatic progenitor cell.

According to an aspect of some embodiments of the invention, the method of identifying pancreatic progenitor cells comprising determining in a population of cells which comprises pancreatic progenitor cells at least one marker that is: (i) negatively associated with pancreatic differentiation, the marker being selected from the group consisting of: LGR5, CCKBR, CXCR4, FLRT3, TRPA1, SLC40A1, LRIG3, COLEC12, EPSTI1, GPR128, CDH2, SLC5A9, FSHR, SLC30A10, IL13RA1, SLC7A7, PCDH10, SLC1A1, GPR141, LIFR, TMEM27, GPC4, LYPD6B, FOLH1, TRPC4, PCDH7, KEL, KCNJ3, OR2T4, VIPR2, FLRT2, CD34, SLC39A8, CLDN11, CXCR7, ITGA5, ITGAV, CALCR, CLDN18, SLC7A5, TMBIM4, SLCO2A1, CDH10, AMHR2, ASAM, CLDN1, DSCAM, TMEM88, PLXNA2, CD177, TMEM144, GPR37, GJA5, SEMA6D, NIPAL2, GPR151, MCC, TMEM136, KCNG1, LHFPL2, MOSPD1, SLC37A1, LRIT3, EPHA4, GPR177, and IL1R1, wherein downregulation above a predetermined threshold of an expression level of the marker negatively associated with the pancreatic differentiation as compared to the expression level of the marker in a reference cell, wherein the reference cell is a definite endodermal cell which expresses SOX17, is indicative of a positive identification of a pancreatic progenitor cell, and alternatively or additionally; (ii) positively associated with pancreatic differentiation, the marker being selected from the group consisting of: TACSTD2 (TROP-2), GPR50, BST2, NTRK2, ITGA4, KDR, PTPRN, LGI1, VIPR2, SLC2A1, MUC15, MUC12, LPHN3, MUC16, VTCN1, MMP16, FZD3, ITGB6, GFRA3, ROBO1, NLGN1, MUC12, CNTFR, LPHN1, SULF1, ADAM23, SCUBE3, PLAU, CDON, SLIT2, C7orf68, PLXDC2, CD74, MUC15, GPR56, VTCN1, ITGB6, AREG, BOC, KLRK1, PRTG, PTPRZ1, GABRP, SILV, KIAA1772, PLP1, OVOS, HAPLN1, EPHA7, ENO2, PCDHB5, OVOS, SYT1, DCT, GPAM, SLITRK6, DCC, FREM2, SDK2, CGA, ATP1B2, SEMA3D, PCDHB15, CDH1, WNT8B, LPAR4, NPIPL3, FAM171B, PTN, ABCC2, ADAMTS3, RASA4, CPT1C, SLC6A6, PCDHB3, LRRC37B, RNFT2, KCNG3, TRPC1, ALPPL2, OR4F21, CCL2, KIF5A, OLFM2, CACNG7, MPHOSPH9, SLC13A4, MOXD1, C6orf186, SLC4A8, STX16, AMY2A, SPARCL1, MGP, A2M, DCN, ATP8B1, MMRN1, EMP1, PLA2G2A, PDE3A, TLR3, CYP1B1, PTGIS, RFTN2, PLEKHA2, SMOC1, STOM, JAM2, CHL1, SCG5, IGFBP7, NPR3, IFI6, CR1L, OR2A4, OR2A7, KCNG3, CACNG7, GRID2, CDH1, LPAR3, SEMA6A, PTPRZ1, ATP1A3, CAMKV, SCNN1G, SYT6, SLC18A2, PCDHB5, ABCG2, HLA-DRA, CR1L, HTR2C, EDNRB, PCDH11X, SLC17A7, SCNN1A, CD9, CXCL16, FXYD5, GABRQ, GFRA3, CACNA2D2, CLDN4, PTPRN, PLP1, PDPN, MMP24, SDK2, GPR176, GPR64, GPR160, PCDH11Y, NKAIN4, ATP1B2, SCN8A, THBS4, CR2, HLA-DQA1, HLA-DRA, HTR7, SLC2A1, HLA-DRA, KCNS3, SLC7A3, HLA-DPB2, CACNA1B, and GPR143 wherein upregulation above a predetermined threshold of an expression level of the marker positively associated with the pancreatic differentiation as compared to the expression level of the marker in a reference cell, wherein the reference cell is a definite endodermal cell which expresses SOX17, is indicative of a positive identification of a pancreatic progenitor cells, and alternatively or additionally; (iii) negatively associated with pancreatic differentiation, the marker being selected from the group consisting of: TMPRSS11E, LGR5, SLC39A8, TM4SF18, CUZD1, GPC4, SLC22A3, CXCR4, NRN1, TMBIM4, THBS2, SLC7A5, and TMEM47, wherein downregulation above a predetermined threshold of an expression level of the marker negatively associated with pancreatic differentiation as compared to the expression level of the marker in a reference cell, wherein the reference cell is an undifferentiated embryonic stem cell (ESC), is indicative of a positive identification of a pancreatic progenitor cell, and alternatively or additionally; (iv) positively associated with pancreatic differentiation, the marker being selected from the group consisting of: TACSTD2 (TROP-2), LRP2, LAMA1, GPC3, LGI1, FN1, LPHN3, FGB, KIT, DPP4, TRO, SPA17, ROBO2, DLK1, BST2, PRTG, LRP2, CACNG7, DLK1, CACNA2D2, CR1L, SCNN1G, HTR2C, LPAR3, THBS3, KCNG3, SDK2, HLA-DRA, SLC18A2, CXCL16, TMEM63C, SLC17A7, GFRA3, PRTG, DKK1, APOA2, AFP, APOB, FLRT3, KIAA1772, FBN2, FRZB, KLK6, FREM2, RSPO3, APOA1, APOA1, SEMA3D, KEL, BMP5, HEPH, STMN2, CACNB3, CHST9, TMC7, FAM171B, ATP5G1, FOLR1, ABCC2, EFNA5, CPT1C, MAGED1, RASA4, KDELR3, ST6GALNAC2, EDNRA, LRRC37B, ABCC4, CLCN5, MBOAT2, SLC44A5, NPC2, ELFN1, MMD, SLC5A3, IMPAD1, OSTC, DSC2, SLC31A1, SLC5A12, LRRN3, NPIPL3, GDPD1, NPIPL3, NPIPL3, LINGO2, and LYPD6B, wherein upregulation above a predetermined threshold of an expression level of the marker positively associated with pancreatic differentiation as compared to the expression level of the marker in a reference cell, wherein the reference cell is an undifferentiated ESC, is indicative of a positive identification of a pancreatic progenitor cell; thereby identifying the pancreatic progenitor cells.

As shown in FIG. 4D and described in Example 2 of the Examples section which follows, pancreatic progenitor cells identified according to the method of some embodiments of the invention express typical pancreatic progenitor cell markers, such as PDX1, hlxb9, Hnf6, ngn3, and Pax4.

According to some embodiments of the invention, the population of cells which comprises pancreatic progenitor cells express a transcription factor selected from the group consisting of PDX1, ngn3, pax4, hlxb9, nkx6.1, Hnf6, and sox9.

According to some embodiments of the invention, the definite endodermal cells are identified by a method comprising determining in a population of cells which comprises definite endodermal cells at least one marker that is: (i) negatively associated with definite endodermal cells, the marker being selected from the group consisting of: KDR, PCDHB5, FAT4, FLT1, NRN1, THBS2, PTPRZ1, SLC6A15, GPR176, SEMA6A, THBS1, CDH11, GRID2, SLC7A11, CDH1, LRFN5, EDNRB, THY1, NETO1, KCND2, TMPRSS11E, CD44, PDPN, SLC7A1, KAL1, KCNG3, GPM6B, FXYD5, PCDH18, ICAM3, MCTP1, TACR3, TMEM155, ZFP42, THUMPD3, ANXA1, SPP1, PRDM14, GNA14, EDIL3, CXCL12, PSMD5, PRRX1, NANOG, TRIM22, NANOG, RASGRF2, POU5F1B, POLR3G, HHLA1, POU5F1, VSNL1, SCG3, B3GALT1, LECT1, NTS, MBNL1, CKMT1A, NECAB1, FGF2, SFRP2, DCLK1, DACT1, CRABP1, TFAP2C, SCGB3A2, LRAT, CUZD1, GLB1L3, METTL7A, VAT1L, COL12A1, OLFML3, SOX2, USP44, HIST1H4F, KGFLP1, CPT1A, DBC1, CHAC1, CAV1, MT1G, NFIX, FERMT1, GLIPR1, TOX, SNRPN (SNORD116-6), HEY2, T1MP4, IDO1, MT2A, NR5A2, UPRT, MYC, CCDC109B, GAL, ZNF483, RND3, BNC2, COL3A1, LUM, LDB2, MT1E, SNRPN (SNORD116-23), SNRPN (SNORD116-27), GALNT3, PIPOX, PDK1, PREX2, CYP2S1, NRK, VAV3, TNFAIP6, ENPP1, ADD2, SNRPN (SNORD116-24), SNRPN (SNORD109A), STC2, SNORA22, MPPED2, ZNF562, GAP43, FOXB1, TSHZ3, HPGD, ZDHHC22, ACOXL, GLI3, CDCA7L, ZSCAN10, GFPT2, PLP2, HIST1H1A, CAMKV, HERC5, MT1X, TERF1, RAB31, SNRPN (SNORD116-13), ETV1, MT1G, ACTA1, SNRPN (SNORD116-20), NFIB, ZEB2, CBR1, ATXN7L1, SNRPN (SNORD116-1), MT1F, SNRPN (SNORD116-29), AP1M2, ACTG2, CYP2B6, SERPINE1, GRHL2, SLIT2, PIM2, SMARCA2, and RPPH1, wherein downregulation above a predetermined threshold of an expression level of the marker negatively associated with definite endodermal cells as compared to the expression level of the marker in a reference cell, wherein the reference cell is an undifferentiated embryonic stem cell (hESC), is indicative of a positive identification of a definite endodermal cell, and alternatively or additionally; (ii) positively associated with definite endodermal cells, the marker being selected from the group consisting of: FLRT3, FSHR, LIFR, ROR2, KEL, TRPA1, CD177, CCKBR, APOA1, APOA1, FZD5, FN1, BMP2, ADAMTS9, DPP4, FGA, COLEC12, GPR128, IGFBP5, FZD4, ITGA5, STC1, TNFSF4, CD177, IHH, LRP2, LAMA1, GPC3, LGI1, FN1, LPHN3, FGB, KIT, DPP4, TRO, SPA17, ROBO2, DLK1, CST1, DKK1, HAS2, APOA2, CDH2, LGR5, AFP, APOB, DGKK, HP, SYTL5, SLC7A7, TTR, FRZB, RSPO3, GCNT1, DIO3, TMEM27, APOC1, EPHA4, C4orf18, GLIPR2, PCDH10, F10, BMP5, FMO5, FOLR1, STMN2, SLC5A9, PORCN, EGFLAM, RAB17, CST2, PNPLA3, MOSPD1, ELMO1, CHST9, SLC30A10, TMC7, C8orf49, CDH12, ST8SIA4, SLCO2A1, MANEA, LRIG3, HCN1, ADAMTS12, FOLH1, TMEM144, VTN, CAMK2N1, ABCC4, PCDH7, OR2T4, UNC93A, COL4A6, PAMR1, SLC1A1, PROS1, APOM, APOM, APOM, LYPD6B, TMEM88, ITLN2, BMPER, GPR141, VEGFA, PRTG, DKK1, APOA2, AFP, APOB, FLRT3, KIAA1772, FBN2, FRZB, KLK6, FREM2, RSPO3, APOA1, APOA1, SEMA3D, KEL, BMP5, HEPH, STMN2, CACNB3, CHST9, TMC7, FAM171B, ATP5G1, FOLR1, ABCC2, EFNA5, CPT1C, MAGED1, RASA4, KDELR3, ST6GALNAC2, EDNRA, LRRC37B, ABCC4, CLCN5, MBOAT2, SLC44A5, NPC2, ELFN1, MMD, SLC5A3, IMPAD1, OSTC, DSC2, SLC31A1, SLC5A12, LRRN3, NPIPL3, GDPD1, NPIPL3, NPIPL3, LINGO2, TRPA1, SLC40A1, SLC30A10, CCKBR, VIPR2, COLEC12, FLRT3, LGR5, GPR141, BST2, SLC5A9, GPR128, KEL, LRIG3, LYPD6B, FSHR, LIFR, FOLH1, CXCR4, ITGA5, AMOT, LY6E, SEMA6D, GJA5, PRTG, CD34, TMEM144, ROR2, GPR177, OR2T4, SLC7A7, KCNJ3, CLDN18, GPR151, SLC44A5, CDH10, TMEM27, SLC1A1, TMEM56, CD177, PLXNA2, SLC26A2, DSCAM, TMEM133, IL13RA1, ATP2B1, CD302, MEGF9, EDNRA, CDH2, GPR161, TYRO3, FLRT2, LRIT3, PCDH7, NRCAM, SMAGP, AMHR2, ELTD1, GRPR, EPHA4, CD99, GATA3, SEMA3E, HHEX, ZNF280A, FAM184A, WDR72, PDZK1, RLBP1L2, SHISA2, VIL1, STMN2, APOA2, SERHL, PPFIBP2, DKK1, MUM1L1, IGFBP5, ST6GALNAC2, TSPYL5, STC1, SYTL5, EPSTI1, ANKRD1, ARHGAP24, KRT18P49, PRSS2, RHOBTB3, FRZB, RARB, ADAMTS9, ARL4D, PRDM1, HP, FZD5, TRY6, ATP6V0D2, ANGPT2, DENND2C, BMP5, FOXA2, HAS2, BMP2, S100A16, FOLH1B, FAM122C, FZD4, C5, S100A14, VEGFA, CLIP4, GPAM, HNF1B, APOA1, CFLAR, RBM24, RNF152, TTR, TTN, EGFLAM, APOB, DIO3, IFLTD1, ABCC4, CCDC141, ENC1, NEK2, ELMO1, SPOCK3, SERPINI1, ACSL1, GATM, EHHADH, NUDT4, CST1, GLUD2, NPL, ZNF702P, TRY6, SPOCK1, AGL, TFF1, DGKK, SALL1, MANEA, KIT, KRT19, TNNC1, SEPP1, ST8SIA4, YPEL2, ANKMY2, DNAJC15, RNF128, PTPN13, F10, SAMD3, GCNT1, IPP, PROS1, SV2B, PLOD2, MAGEH1, CHST9, ZNF518B, TMEM106C, SERHL2, NTN4, SOX17, FRRS1, OTX2, RNASEL, ELMOD2, MYCT1, PAX6, MGST2, BBS5, MTSS1, VTN, WBP5, DUB4, CCDC92, BTG2, LPGAT1, FN1, TBX3, PLCE1, KRT19P2, IFI16, PORCN, PRSS1, MYL7, DUSP4, PROS1, ANKRD20B, CTSL2, FMO5, USP27X, LAMA1, ADAM28, ZNF611, ANKRD20B, ZNF137, S100Z, GPSM2, TGFB2, and ARHGAP28, wherein upregulation above a predetermined threshold of an expression level of the marker positively associated with definite endodermal cells as compared to the expression level of the marker in a reference cell, wherein the reference cell is an undifferentiated embryonic stem cell (hESC), is indicative of a positive identification of a definite endodermal cell, thereby identifying the definite endodermal cells.

According to some embodiments of the invention the at least one marker positively associated with pancreatic differentiation is selected from the group consisting of: TACSTD2 (TROP-2), GPR50, BST2, LGI1, VIPR2, SLC2A1, MUC15, MUC12, LPHN3, MUC16, VTCN1, MMP16, FZD3, ITGB6, GFRA3, ROBO1, NLGN1, MUC12, CNTFR, LPHN1, SULF1, ADAM23, SCUBE3, PLAU, CDON, SLIT2, C7orf68, PLXDC2, CD74, MUC15, GPR56, VTCN1, ITGB6, NTRK2, AREG, BOC, ITGA4, KLRK1, PRTG, PTPRZ1, GABRP, SILV, KIAA1772, PLP1, OVOS, HAPLN1, EPHA7, ENO2, PCDHB5, OVOS, SYT1, DCT, GPAM, SLITRK6, DCC, FREM2, SDK2, CGA, ATP1B2, SEMA3D, PCDHB15, CDH1, WNT8B, LPAR4, NPIPL3, FAM171B, PTN, ABCC2, ADAMTS3, RASA4, CPT1C, SLC6A6, PCDHB3, LRRC37B, RNFT2, KCNG3, TRPC1, ALPPL2, OR4F21, CCL2, KIF5A, OLFM2, CACNG7, MPHOSPH9, SLC13A4, MOXD1, C6orf186, SLC4A8, STX16, AMY2A, SPARCL1, MGP, A2M, DCN, ATP8B1, MMRN1, EMP1, PLA2G2A, PDE3A, TLR3, CYP1B1, PTGIS, RFTN2, PLEKHA2, SMOC1, STOM, JAM2, CHL1, SCG5, IGFBP7, NPR3, IFI6, CR1L, OR2A4, and OR2A7.

According to some embodiments of the invention the at least one marker positively associated with pancreatic differentiation is selected from the group consisting of: TACSTD2 (TROP-2), BST2, GPR50, ROBO1, NTRK2, ITGA4, LGI1, VIPR2, SLC2A1, MUC15, MUC12, LPHN3, MUC16, VTCN1, MMP16, FZD3, ITGB6, GFRA3, NLGN1, MUC12, CNTFR, LPHN1, SULF1, ADAM23, SCUBE3, PLAU, CDON, SLIT2, C7orf68, PLXDC2, CD74, MUC15, GPR56, VTCN1, ITGB6, AREG, BOC, and KLRK1.

According to some embodiments of the invention the at least one marker positively associated with pancreatic differentiation is TROP-2.

According to some embodiments of the invention the at least one marker positively associated with pancreatic differentiation is GPR50.

According to some embodiments of the invention the at least one marker positively associated with pancreatic differentiation comprises at least two markers, said at least two markers are TROP-2 and GPR50.

According to some embodiments of the invention the at least one marker positively associated with pancreatic differentiation comprises at least three markers, said at least three markers comprise TROP-2, GPR50 and at least one marker selected from the group consisting of TACSTD2 (TROP-2), GPR50, BST2, NTRK2, ITGA4, KDR, PTPRN, LGI1, VIPR2, SLC2A1, MUC15, MUC12, LPHN3, MUC16, VTCN1, MMP16, FZD3, ITGB6, GFRA3, ROBO1, NLGN1, MUC12, CNTFR, LPHN1, SULF1, ADAM23, SCUBE3, PLAU, CDON, SLIT2, C7orf68, PLXDC2, CD74, MUC15, GPR56, VTCN1, ITGB6, AREG, BOC, KLRK1, PRTG, PTPRZ1, GABRP, SILV, KIAA1772, PLP1, OVOS, HAPLN1, EPHA7, ENO2, PCDHB5, OVOS, SYT1, DCT, GPAM, SLITRK6, DCC, FREM2, SDK2, CGA, ATP1B2, SEMA3D, PCDHB15, CDH1, WNT8B, LPAR4, NPIPL3, FAM171B, PTN, ABCC2, ADAMTS3, RASA4, CPT1C, SLC6A6, PCDHB3, LRRC37B, RNFT2, KCNG3, TRPC1, ALPPL2, OR4F21, CCL2, KIF5A, OLFM2, CACNG7, MPHOSPH9, SLC13A4, MOXD1, C6orf186, SLC4A8, STX16, AMY2A, SPARCL1, MGP, A2M, DCN, ATP8B1, MMRN1, EMP1, PLA2G2A, PDE3A, TLR3, CYP1B1, PTGIS, RFTN2, PLEKHA2, SMOC1, STOM, JAM2, CHL1, SCG5, IGFBP7, NPR3, IFI6, CR1L, OR2A4, OR2A7, KCNG3, CACNG7, GRID2, CDH1, LPAR3, SEMA6A, PTPRZ1, ATP1A3, CAMKV, SCNN1G, SYT6, SLC18A2, PCDHB5, ABCG2, HLA-DRA, CR1L, HTR2C, EDNRB, PCDH11X, SLC17A7, SCNN1A, CD9, CXCL16, FXYD5, GABRQ, GFRA3, CACNA2D2, CLDN4, PLP1, PDPN, MMP24, SDK2, GPR176, GPR64, GPR160, PCDH11Y, NKAIN4, ATP1B2, SCN8A, THBS4, CR2, HLA-DQA1, HLA-DRA, HTR7, SLC2A1, HLA-DRA, KCNS3, SLC7A3, HLA-DPB2, CACNA1B, and GPR143.

According to some embodiments of the invention, the at least one marker positively associated with definite endodermal cells is selected from the group consisting of: COLEC12, ROR2, FLRT3, LGR5, LIFR, KEL, FSHR, TRPA1, FOLR1, LRP2, FOLH1, CD177, CCKBR, ITGA5, FZD5, FN1, BMP2, ADAMTS9, DPP4, FGA, GPR128, IGFBP5, FZD4, STC1, TNFSF4, CD177, IHH, APOA1, APOA1, LAMA1, GPC3, LGI1, FN1, LPHN3, FGB, KIT, DPP4, TRO, SPA17, ROBO2, DLK1, CST1, DKK1, HAS2, APOA2, CDH2, AFP, APOB, DGKK, HP, SYTL5, SLC7A7, TTR, FRZB, RSPO3, GCNT1, DIO3, TMEM27, APOC1, EPHA4, C4orf18, GLIPR2, PCDH10, F10, BMP5, FMO5, STMN2, SLC5A9, PORCN, EGFLAM, RAB17, CST2, PNPLA3, MOSPD1, ELMO1, CHST9, SLC30A10, TMC7, C8orf49, CDH12, ST8SIA4, SLCO2A1, MANEA, LRIG3, HCN1, ADAMTS12, TMEM144, VTN, CAMK2N1, ABCC4, PCDH7, OR2T4, UNC93A, COL4A6, PAMR1, SLC1A1, PROS1, APOM, APOM, APOM, LYPD6B, TMEM88, ITLN2, BMPER, GPR141, VEGFA, PRTG, DKK1, APOA2, AFP, APOB, FLRT3, KIAA1772, FBN2, FRZB, KLK6, FREM2, RSPO3, APOA1, APOA1, SEMA3D, KEL, BMP5, HEPH, STMN2, CACNB3, CHST9, TMC7, FAM171B, ATP5G1, FOLR1, ABCC2, EFNA5, CPT1C, MAGED1, RASA4, KDELR3, ST6GALNAC2, EDNRA, LRRC37B, ABCC4, CLCN5, MBOAT2, SLC44A5, NPC2, ELFN1, MMD, SLC5A3, IMPAD1, OSTC, DSC2, SLC31A1, SLC5A12, LRRN3, NPIPL3, GDPD1, NPIPL3, NPIPL3, LINGO2, and LYPD6B.

According to some embodiments of the invention, the at least one marker positively associated with definite endodermal cells is selected from the group consisting of: FSHR, LIFR, COLEC12, ROR2, ITGA5, CD177, CCKBR, APOA1, APOA1, FZD5, FN1, BMP2, ADAMTS9, DPP4, FGA, GPR128, IGFBP5, FZD4, STC1, TNFSF4, CD177, IHH, LRP2, LAMA1, GPC3, LGI1, FN1, LPHN3, FGB, KIT, DPP4, TRO, SPA17, ROBO2, and DLK1.

The phrase “embryonic stem cell” refers to an embryonic cell capable of differentiating into cells of all three embryonic germ layers (i.e., endoderm, ectoderm and mesoderm), or remaining in an undifferentiated state.

According to some embodiments of the invention, the ESC which is used as reference cell is a human ESC obtained from the embryonic tissue formed after gestation (e.g., blastocyst) before implantation (i.e., a pre-implantation blastocyst).

The embryonic stem cells of the invention can be obtained using well-known cell-culture methods. For example, human embryonic stem cells can be isolated from human blastocysts. Human blastocysts are typically obtained from human in vivo preimplantation embryos or from in vitro fertilized (IVF) embryos. Alternatively, a single cell human embryo can be expanded to the blastocyst stage. For the isolation of human ES cells the zona pellucida is removed from the blastocyst and the inner cell mass (ICM) is isolated by immunosurgery, in which the trophectoderm cells are lysed and removed from the intact ICM by gentle pipetting. The ICM is then plated in a tissue culture flask containing the appropriate medium which enables its outgrowth. Following 9 to 15 days, the ICM derived outgrowth is dissociated into clumps either by a mechanical dissociation or by an enzymatic degradation and the cells are then re-plated on a fresh tissue culture medium. Colonies demonstrating undifferentiated morphology are individually selected by micropipette, mechanically dissociated into clumps, and re-plated. Resulting ES cells are then routinely split every 4-7 days. For further details on methods of preparation human ES cells see Thomson et al., [U.S. Pat. No. 5,843,780; Science 282: 1145, 1998; Curr. Top. Dev. Biol. 38: 133, 1998; Proc. Natl. Acad. Sci. USA 92: 7844, 1995]; Bongso et al., [Hum Reprod 4: 706, 1989]; and Gardner et al., [Fertil. Steril. 69: 84, 1998].

It will be appreciated that commercially available stem cells can also be used with this aspect of the present invention. Human ES cells can be purchased from the NIH human embryonic stem cells registry (www.escr.nih.gov). Non-limiting examples of commercially available embryonic stem cell lines are BG01, BG02, BG03, BG04, CY12, CY30, CY92, CY10, TE03, TE04 and TE06.

According to the method of some embodiments of the invention, the hESCs are undifferentiated hESCs.

According to some embodiments of the invention, the undifferentiated hESCs are characterized by an Oct4+ expression pattern.

According to an aspect of some embodiments of the invention, there is provided a method of isolating pancreatic progenitor cells, comprising: (a) identifying the pancreatic progenitor cells according to the method of some embodiments of the invention, and (b) isolating the pancreatic progenitor cells identified according to step (a) to thereby obtain isolated pancreatic progenitor cells, thereby isolating the pancreatic progenitor cells.

As used herein the term “isolating” refers to at least partially separating from the natural environment e.g., the population of cells.

Isolating the pancreatic progenitor cells from the cell population can be performed by any immunological based method which results in the physical isolation of pancreatic progenitor cells having a specific cell surface marker using an antibody or an antibody fragment which specifically recognizes the marker. Examples include, but are not limited to isolation by fluorescence-activated cell sorting (FACS) using the specific antibodies, magnetic beads coated by the specific antibodies [Magnetic-activated cell sorting (MACS)], columns coated by the specific antibodies or immunopanning.

According to some embodiments of the invention, step (b) of the method of some embodiments of the invention is effected by an immunological isolation assay selected from the group consisting of fluorescent activated cell sorter (FACS), Magnetic-activated cell sorting (MACS) or immunopanning.

For example, for isolation using fluorescence-activated cell sorting, the cells are labeled with a fluorescent antibody (e.g., PE-conjugated anti TROP-2 antibody, or PE-conjugated anti GPR50 antibody) and then inserted into a cell sorter (e.g., FACS Aria sorter).

For isolation using magnetic beads, the cells are labeled with a magnetic bead conjugated antibody anti TROP-2 antibody (Miltenyi Biotec) or anti GPR antibody; alternatively, the cells can be labeled with a non-conjugated antibody and followed by incubation with a match isotype bead conjugated secondary antibody (anti mouse IgG1 bead conjugated). Isolation can be performed using magnetic cell separation column such as MAX (Miltenyi Biotec).

According to an aspect of some embodiments of the invention, there is provided an isolated population of pancreatic progenitor cells obtained according to the method of some embodiments of the invention.

According to some embodiments of the invention, the isolated pancreatic progenitor cell population is characterized by TROP-2+ expression pattern.

According to some embodiments of the invention, the isolated pancreatic progenitor cell population is characterized by TROP-2+/GPR50+ expression pattern.

According to some embodiments of the invention, the isolated pancreatic progenitor cell population is characterized by an ngn3+/pax4+/hlxb9+/nkx6.1+/Hnf6+/sox9+/PDX1+ expression signature.

According to an aspect of some embodiments of the invention, there is provided an isolated population of pancreatic progenitor cells, comprising at least about 50%, at least about 60%, e.g., at least about 75% (e.g., 75%), e.g., at least about 80% (e.g., 80%), e.g., at least about 85% (e.g., 85%), e.g., at least about 90% (e.g., 90%), e.g., at least about 95% (e.g., 95%), e.g., at least about 96%, e.g., at least about 97%, e.g., at least about 98%, e.g., at least about 99%, e.g., 100% of cells having a TROP-2+ and/or TROP-2+/GPR50+ expression pattern.

According to some embodiments of the invention, the isolated population of pancreatic progenitor cells is characterized by a TROP-2+/GPR50+/ngn3+/pax4+/hlxb9+/nkx6.1+/Hnf6+/sox9+/PDX1+ expression signature.

According to an aspect of some embodiments of the invention, there is provided an isolated population of pancreatic progenitor cells, comprising at least about 50%, at least about 60%, e.g., at least about 75% (e.g., 75%), e.g., at least about 80% (e.g., 80%), e.g., at least about 85% (e.g., 85%), e.g., at least about 90% (e.g., 90%), e.g., at least about 95% (e.g., 95%), e.g., at least about 96%, e.g., at least about 97%, e.g., at least about 98%, e.g., at least about 99%, e.g., 100% of cells PDX1+.

According to some embodiments of the invention, the isolated population of pancreatic progenitor cells is characterized by a TROP-2+/ngn3+/pax4+/hlxb9+/nkx6.1+/Hnf6+/sox9+/PDX1+ expression signature.

According to some embodiments of the invention, the pancreatic progenitor cells are genetically unmodified (i.e., were not subjected to genetic manipulation, using e.g., recombinant DNA techniques).

According to some embodiments of the invention, the isolated population of pancreatic progenitor cells can be further expanded to produce an expanded population of cells.

Following are non-limiting protocols for culturing and expanding the pancreatic progenitor cells isolated according to some embodiments of the invention, e.g., the TROP-2+/GPR50+ cells:

Protocol 1: The pancreatic progenitor cells (e.g., TROP-2+/GPR50+ cells) are plated on adherent tissue culture plates and cultured in the presence of the DMEM medium (Invitrogen) supplemented with 1% B27 (Invitrogen) [For further details see Kroon 2008 (Nat Biotechnol. 2008; 26:443-52. Epub 2008 Feb. 20) and D'Amour 2006 (Nat Biotechnol. 2006, 24:1392-401. Epub 2006 Oct. 19), each of which is fully incorporated herein by reference]. The medium can be further supplemented with retinoic acid (RA, all trans retinoic acid at a concentration of 2 μM), CYC (KAAD cyclopamine 0.25 μM), and Nog (noggin 50 ng/ml). Additionally or alternatively, the medium can be supplemented with 2 μM RA, 0.25 μM CYC, and 50 ng/m FGF10. Additionally or alternatively, the medium can be supplemented with 50 ng/ml Exendin 4, with or without 1 μM DAPT. Additionally or alternatively, the medium can be CMRL (Invitrogen) supplemented with 1% BSA, 50 ng/ml IGF-1 and 50 ng/ml HGF with or without 50 ng/ml Exendin 4. Additionally or alternatively, the cells can be cultured in DMEM/F12 (Invitrogen), supplemented with N2 (Invitrogen), BSA (2 mg/ml) and bFGF (10 ng/ml) for 4 days, followed by 8 days of culturing in the presence of nicotinamide (10 mM), or alternatively culturing in DMEM/F12 (Invitrogen) supplemented with nicotinamide (10 mM) for 8 days.

Protocol 2: The pancreatic progenitor cells (e.g., TROP-2+/GPR50+ cells) are plated on adherent tissue culture plates and cultured in the presence of the DMEM medium supplemented with 1% B27, with or without Indolactam V (e.g., at a concentration of 330 mM). Additionally or alternatively, the pancreatic progenitor cells are plated on adherent tissue culture plates and cultured in the presence of the DMEM medium supplemented with 1% B27, 2 μM RA, 50 ng/ml FGF10, 0.25 μM KAAD-cyclopamine and cultured for up to 3 days, followed by culturing the cells in the DMEM medium, in the presence of 1% B27, 50 ng/ml FGF10 with or without ILV (e.g., at a concentration of 300 nM) for up to 4 days [for further details see Borowiak 2009 (Cell Stem Cell. 2009; 4: 348-58) and Chen 2009 (Nat Chem Biol. 2009; 5:258-65. Epub 2009 Mar. 15), each of which is fully incorporated herein by reference].

According to an aspect of some embodiments of the invention, there is provided a method of qualifying a pancreatic progenitor cell population, comprising: determining in a sample of the cell population a percentage of the pancreatic progenitor cells which are identified according to the method of some embodiments of the invention out of the total cells in the sample, thereby qualifying the pancreatic progenitor cell population.

According to some embodiments of the invention, pancreatic progenitor cell population has been isolated according to the method of some embodiments of the invention.

According to some embodiments of the invention, presence of at least a predetermined percentage of the pancreatic progenitor cells in the cell sample indicates the suitability of the pancreatic progenitor cells for transplantation in a subject.

According to some embodiments of the invention, the predetermined percentage of the pancreatic progenitor cells comprises at least about 80%, e.g., at least 81%, 82%, 83%, 84%, 85%, 86%, 87%, 88%, 89%, 90%, 91%, 92%, 93%, 94%, 95%, 96%, 97%, 98%, 99% or 100% pancreatic progenitor cells.

According to an aspect of some embodiments of the invention, there is provided a method of isolating insulin producing cells, comprising culturing the pancreatic progenitor cells isolated by the method of some embodiments of the invention, or the pancreatic progenitor cell population qualified according to the method of some embodiments of the invention under conditions suitable for maturation of the pancreatic progenitor cells into beta cells, thereby isolating insulin producing cells.

Methods and conditions suitable for maturation of the pancreatic progenitor cells into beta cells are known in the art and described in Kroon 2008 (Nat Biotechnol. 2008; 26:443-52), D'Amour 2006 (Nat Biotechnol. 2006, 24:1392-401), Borowiak 2009 (Cell Stem Cell. 2009; 4: 348-58) and Chen 2009 (Nat Chem Biol. 2009; 5:258-65. Epub 2009 Mar. 15), each of which is fully incorporated herein by reference in its entirety.

According to an aspect of some embodiments of the invention, there is provided a method of transplanting pancreatic progenitor cells in a subject, (a) qualifying the pancreatic progenitor cells according to the method of some embodiments of the invention, wherein presence of at least a predetermined percentage of the pancreatic progenitor cells in the cell sample indicates the suitability of the pancreatic progenitor cells for transplantation in a subject, to thereby obtain a pancreatic progenitor cell population being suitable for transplantation in a subject, (b) transplanting in the subject the pancreatic progenitor cell population or cells derived therefrom being suitable for transplantation in a subject, thereby transplanting the pancreatic progenitor cells in the subject.

As used herein the phrase “cells derived therefrom” refers to cells which are differentiated from the pancreatic progenitor cells.

According to some embodiments of the invention, the cells which are differentiated from the pancreatic progenitor cells are capable of producing insulin.

According to some embodiments of the invention, the pancreatic progenitor cell population or cells derived therefrom can be encapsulated prior to transplantation in the subject.

The primary goal in encapsulation as a cell therapy is to protect allogeneic and xenogeneic cell transplants from destruction by the host immune system, thereby eliminating or reducing the need for immuno-suppressive drug therapy. Techniques for macro and microencapsulation of cells are known to those of skill in the art (see, for example, Chang, P. et al. 1999; Matthew, H. W. et al. 1991; Yanagi, K. et al. 1989; Cai Z. H. et al. 1988; Chang, T. M. 1992).

Encapsulation techniques are generally classified as microencapsulation, involving small spherical vehicles and macroencapsulation, involving larger flat-sheet and hollow-fiber membranes (Uludag, H. et al. Technology of mammalian cell encapsulation. Adv Drug Deliv Rev. 2000; 42: 29-64).

Methods of preparing microcapsules are known in the arts and include for example those disclosed by Lu M Z, et al., Cell encapsulation with alginate and alpha-phenoxycinnamylidene-acetylated poly(allylamine) Biotechnol Bioeng. 2000, 70: 479-83, Chang T M and Prakash S. Procedures for microencapsulation of enzymes, cells and genetically engineered microorganisms. Mol Biotechnol. 2001, 17: 249-60, and Lu M Z, et al., A novel cell encapsulation method using photosensitive poly(allylamine alpha-cyanocinnamylideneacetate). J. Microencapsul. 2000, 17: 245-51.

For example, microcapsules are prepared by complexing modified collagen with a ter-polymer shell of 2-hydroxyethyl methylacrylate (HEMA), methacrylic acid (MAA) and methyl methacrylate (MMA), resulting in a capsule thickness of 2-5 μm. Such microcapsules can be further encapsulated with additional 2-5 μm ter-polymer shells in order to impart a negatively charged smooth surface and to minimize plasma protein absorption (Chia, S. M. et al. Multi-layered microcapsules for cell encapsulation Biomaterials. 2002 23: 849-56).

Other microcapsules are based on alginate, a marine polysaccharide (Sambanis, A. Encapsulated islets in diabetes treatment. Diabetes Thechnol. Ther. 2003, 5: 665-8) or its derivatives. For example, microcapsules can be prepared by the polyelectrolyte complexation between the polyanions sodium alginate and sodium cellulose sulphate with the polycation poly(methylene-co-guanidine) hydrochloride in the presence of calcium chloride.

It will be appreciated that cell encapsulation is improved when smaller capsules are used. Thus, the quality control, mechanical stability, diffusion properties, and in vitro activities of encapsulated cells improved when the capsule size was reduced from 1 mm to 400 μm (Canaple L. et al., Improving cell encapsulation through size control. J Biomater Sci Polym Ed. 2002; 13: 783-96). Moreover, nanoporous biocapsules with well-controlled pore size as small as 7 nm, tailored surface chemistries and precise microarchitectures were found to successfully immunoisolate microenvironments for cells (Williams D. Small is beautiful: microparticle and nanoparticle technology in medical devices. Med Device Technol. 1999, 10: 6-9; Desai, T. A. Microfabrication technology for pancreatic cell encapsulation. Expert Opin Biol Ther. 2002, 2: 633-46).

Additional methods of cell encapsulation are well known in the art, such as those described in European Patent Publication No. 301,777 or U.S. Pat. Nos. 4,353,888; 4,744,933; 4,749,620; 4,814,274; 5,084,350; 5,089,272; 5,578,442; 5,639,275; and 5,676,943, each of which is incorporated herein by reference. Other methods are described U.S. Pat. No. 6,281,341; Desai 2002 Exp. Opin. Biol. Hortelano et al. 1996 Blood 87:5095-5103; Pelegrin et al. 1998 Gene Ther. 5:828-834; Lohr et al. 2001 Lancet 357:1591-1592; Cirone et al. Hum. Gene Ther. 13: 1157-1166, each of which is hereby incorporated by reference in it's entirety.

The ordinary skilled artisan will select a biocompatible, as well as a mechanically and chemically stable membrane of a suitable permeability cut-off value that provides immune protection to the implant, functional performance, biosafety and long term survival of the graft.

Encapsulated cells generated according to the present teachings can be used in a myriad of research and clinical applications.

As used herein the term “subject” includes mammals, preferably human beings at any age which may benefit from transplantation of pancreatic cells.

According to some embodiments of the invention, the subject suffers from insulin insufficiency.

According to some embodiments of the invention, the subject has diabetes.

According to some embodiments of the invention, the subject has insulin-dependent diabetes such as type I or type II diabetes.

According to some embodiments of the invention, the subject has loss or reduced of insulin production. For example, the subject has an injured pancreas (e.g., due to a trauma), suffers from pancreatitis and/or suffers from a pancreatic tumor.

According to some embodiments of the invention, the subject has pancreatic cancer and being treated with an agent which kills or reduces the number of beta cells, such as Zanosar (streptozotocin).

According to an aspect of some embodiments of the invention, there is provided a method of identifying definite endodermal cells, comprising determining in a population of cells which comprises definite endodermal cells at least one marker that is negatively associated with definite endodermal cells, the marker being selected from the group consisting of: KDR, PCDHB5, FAT4, FLT1, NRN1, THBS2, PTPRZ1, SLC6A15, GPR176, SEMA6A, THBS1, CDH11, GRID2, SLC7A11, CDH1, LRFN5, EDNRB, THY1, NETO1, KCND2, TMPRSS11E, CD44, PDPN, SLC7A1, KAL1, KCNG3, GPM6B, FXYD5, PCDH18, ICAM3, MCTP1, TACR3, and TMEM155, wherein downregulation above a predetermined threshold of an expression level of the marker negatively associated with the definite endodermal cells as compared to the expression level of the marker in a reference cell, wherein the reference cell is an undifferentiated human ESC, is indicative of a positive identification of a definite endodermal cell.

According to some embodiments of the invention, the method further comprising determining in the population of cells which comprises definite endodermal cells at least one marker that is positively associated with definite endodermal cells, the marker being selected from the group consisting of: FSHR, COLEC12, ROR2, LIFR, LIFR, FLRT3, KEL, LGR5, FOLR1, CD177, CCKBR, APOA1, APOA1, FZD5, FN1, BMP2, ADAMTS9, DPP4, FGA, GPR128, IGFBP5, FZD4, ITGA5, STC1, TNFSF4, CD177, IHH, LRP2, LAMA1, GPC3, LGI1, FN1, LPHN3, FGB, KIT, DPP4, TRO, SPA17, ROBO2, DLK1, CST1, DKK1, HAS2, APOA2, CDH2, AFP, APOB, DGKK, HP, SYTL5, SLC7A7, TTR, FRZB, RSPO3, GCNT1, TRPA1, DIO3, TMEM27, APOC1, EPHA4, C4orf18, GLIPR2, PCDH10, F10, BMP5, FMO5, STMN2, SLC5A9, PORCN, EGFLAM, RAB17, CST2, PNPLA3, MOSPD1, ELMO1, CHST9, SLC30A10, TMC7, C8orf49, CDH12, ST8SIA4, SLCO2A1, MANEA, LRIG3, HCN1, ADAMTS12, FOLH1, TMEM144, VTN, CAMK2N1, ABCC4, PCDH7, OR2T4, UNC93A, COL4A6, PAMR1, SLC1A1, PROS1, APOM, APOM, APOM, LYPD6B, TMEM88, ITLN2, BMPER, GPR141, VEGFA, PRTG, DKK1, APOA2, AFP, APOB, FLRT3, KIAA1772, FBN2, FRZB, KLK6, FREM2, RSPO3, APOA1, APOA1, SEMA3D, KEL, BMP5, HEPH, STMN2, CACNB3, CHST9, TMC7, FAM171B, ATP5G1, FOLR1, ABCC2, EFNA5, CPT1C, MAGED1, RASA4, KDELR3, ST6GALNAC2, EDNRA, LRRC37B, ABCC4, CLCN5, MBOAT2, SLC44A5, NPC2, ELFN1, MMD, SLC5A3, IMPAD1, OSTC, DSC2, SLC31A1, SLC5A12, LRRN3, NPIPL3, GDPD1, NPIPL3, NPIPL3, LINGO2, LYPD6B, TRPA1, SLC40A1, SLC30A10, CCKBR, VIPR2, COLEC12, FLRT3, LGR5, GPR141, BST2, SLC5A9, GPR128, KEL, LRIG3, LYPD6B, FSHR, LIFR, FOLH1, CXCR4, ITGA5, AMOT, LY6E, SEMA6D, GJA5, PRTG, CD34, TMEM144, ROR2, GPR177, OR2T4, SLC7A7, KCNJ3, CLDN18, GPR151, SLC44A5, CDH10, TMEM27, SLC1A1, TMEM56, CD177, PLXNA2, SLC26A2, DSCAM, TMEM133, IL13RA1, ATP2B1, CD302, MEGF9, EDNRA, CDH2, GPR161, TYRO3, FLRT2, LRIT3, PCDH7, NRCAM, SMAGP, AMHR2, ELTD1, GRPR, EPHA4, CD99, GATA3, SEMA3E, HHEX, ZNF280A, FAM184A, WDR72, PDZK1, RLBP1L2, SHISA2, VIL1, STMN2, APOA2, SERHL, PPFIBP2, DKK1, MUM1L1, IGFBP5, ST6GALNAC2, TSPYL5, STC1, SYTL5, EPSTI1, ANKRD1, ARHGAP24, KRT18P49, PRSS2, RHOBTB3, FRZB, RARB, ADAMTS9, ARL4D, PRDM1, HP, FZD5, TRY6, ATP6V0D2, ANGPT2, DENND2C, BMP5, FOXA2, HAS2, BMP2, S100A16, FOLH1B, FAM122C, FZD4, C5, S100A14, VEGFA, CLIP4, GPAM, HNF1B, APOA1, CFLAR, RBM24, RNF152, TTR, TTN, EGFLAM, APOB, DIO3, IFLTD1, ABCC4, CCDC141, ENC1, NEK2, ELMO1, SPOCK3, SERPINI1, ACSL1, GATM, EHHADH, NUDT4, CST1, GLUD2, NPL, ZNF702P, TRY6, SPOCK1, AGL, TFF1, DGKK, SALL1, MANEA, KIT, KRT19, TNNC1, SEPP1, ST8SIA4, YPEL2, ANKMY2, DNAJC15, RNF128, PTPN13, F10, SAMD3, GCNT1, IPP, PROS1, SV2B, PLOD2, MAGEH1, CHST9, ZNF518B, TMEM106C, SERHL2, NTN4, SOX17, FRRS1, OTX2, RNASEL, ELMOD2, MYCT1, PAX6, MGST2, BBS5, MTSS1, VTN, WBP5, DUB4, CCDC92, BTG2, LPGAT1, FN1, TBX3, PLCE1, KRT19P2, IFI16, PORCN, PRSS1, MYL7, DUSP4, PROS1, ANKRD20B, CTSL2, FMO5, USP27X, LAMA1, ADAM28, ZNF611, ANKRD20B, ZNF137, S100Z, GPSM2, TGFB2, and ARHGAP28, wherein upregulation above a predetermined threshold of an expression level of the marker positively associated with definite endodermal cells as compared to the expression level of the marker in a reference cell, wherein the reference cell is an undifferentiated hESC, is indicative of a positive identification of a definite endodermal cell.

According to an aspect of some embodiments of the invention, there is provided a method of identifying definite endodermal cells, comprising determining in a population of cells which comprises definite endodermal cells at least one marker that is positively associated with definite endodermal cells, the marker being selected from the group consisting of: FSHR, COLEC12, ROR2 ITGA5, LRP2, CD177, CCKBR, TRPA1, KEL, FOLR1, FOLH1, APOA1, APOA1, FZD5, FN1, BMP2, ADAMTS9, DPP4, FGA, GPR128, IGFBP5, LIFR, FZD4, PRTG, STC1, TNFSF4, CD177, IHH, LAMA1, GPC3, LGI1, FN1, LPHN3, FGB, KIT, DPP4, TRO, SPA17, ROBO2, DLK1, CST1, DKK1, HAS2, APOA2, CDH2, LGR5, AFP, FLRT3, APOB, DGKK, HP, SYTL5, SLC7A7, TTR, FRZB, RSPO3, GCNT1, DIO3, TMEM27, APOC1, EPHA4, C4orf18, GLIPR2, PCDH10, F10, BMP5, FMO5, STMN2, SLC5A9, PORCN, EGFLAM, RAB17, CST2, PNPLA3, MOSPD1, ELMO1, CHST9, SLC30A10, TMC7, C8orf49, CDH12, ST8SIA4, SLCO2A1, MANEA, LRIG3, HCN1, ADAMTS12, TMEM144, VTN, CAMK2N1, ABCC4, PCDH7, OR2T4, UNC93A, COL4A6, PAMR1, SLC1A1, PROS1, APOM, APOM, APOM, LYPD6B, TMEM88, ITLN2, BMPER, GPR141, VEGFA, DKK1, APOA2, AFP, APOB, FLRT3, KIAA1772, FBN2, FRZB, KLK6, FREM2, RSPO3, APOA1, APOA1, SEMA3D, KEL, BMP5, HEPH, STMN2, CACNB3, CHST9, TMC7, FAM171B, ATP5G1, FOLR1, ABCC2, EFNA5, CPT1C, MAGED1, RASA4, KDELR3, ST6GALNAC2, EDNRA, LRRC37B, ABCC4, CLCN5, MBOAT2, SLC44A5, NPC2, ELFN1, MMD, SLC5A3, IMPAD1, OSTC, DSC2, SLC31A1, SLC5A12, LRRN3, NPIPL3, GDPD1, NPIPL3, NPIPL3, LINGO2, LYPD6B, TRPA1, SLC40A1, SLC30A10, CCKBR, VIPR2, COLEC12, FLRT3, LGR5, GPR141, BST2, SLC5A9, GPR128, KEL, LRIG3, LYPD6B, FSHR, LIFR, FOLH1, CXCR4, ITGA5, AMOT, LY6E, SEMA6D, GJA5, PRTG, CD34, TMEM144, ROR2, GPR177, OR2T4, SLC7A7, KCNJ3, CLDN18, GPR151, SLC44A5, CDH10, TMEM27, SLC1A1, TMEM56, CD177, PLXNA2, SLC26A2, DSCAM, TMEM133, IL13RA1, ATP2B1, CD302, MEGF9, EDNRA, CDH2, GPR161, TYRO3, FLRT2, LRIT3, PCDH7, NRCAM, SMAGP, AMHR2, ELTD1, GRPR, EPHA4, and CD99, wherein upregulation above a predetermined threshold of an expression level of the marker positively associated with the definite endodermal cells as compared to the expression level of the marker in a reference cell, wherein the reference cell is an undifferentiated hESCs, is indicative of a positive identification of a definite endodermal cell.

According to some embodiments of the invention, the method further comprising determining in the population of cells which comprises definite endodermal cells at least one marker that is positively associated with definite endodermal cells, the marker being selected from the group consisting of: GATA3, SEMA3E, HHEX, ZNF280A, FAM184A, WDR72, PDZK1, RLBP1L2, SHISA2, VIL1, STMN2, APOA2, SERHL, PPFIBP2, DKK1, MUM1L1, IGFBP5, ST6GALNAC2, TSPYL5, STC1, SYTL5, EPSTI1, ANKRD1, ARHGAP24, KRT18P49, PRSS2, RHOBTB3, FRZB, RARB, ADAMTS9, ARL4D, PRDM1, HP, FZD5, TRY6, ATP6V0D2, ANGPT2, DENND2C, BMP5, FOXA2, HAS2, BMP2, S100A16, FOLH1B, FAM122C, FZD4, C5, S100A14, VEGFA, CLIP4, GPAM, HNF1B, APOA1, CFLAR, RBM24, RNF152, TTR, TTN, EGFLAM, APOB, DIO3, IFLTD1, ABCC4, CCDC141, ENC1, NEK2, ELMO1, SPOCK3, SERPINI1, ACSL1, GATM, EHHADH, NUDT4, CST1, GLUD2, NPL, ZNF702P, TRY6, SPOCK1, AGL, TFF1, DGKK, SALL1, MANEA, KIT, KRT19, TNNC1, SEPP1, ST8SIA4, YPEL2, ANKMY2, DNAJC15, RNF128, PTPN13, F10, SAMD3, GCNT1, IPP, PROS1, SV2B, PLOD2, MAGEH1, CHST9, ZNF518B, TMEM106C, SERHL2, NTN4, SOX17, FRRS1, OTX2, RNASEL, ELMOD2, MYCT1, PAX6, MGST2, BBS5, MTSS1, VTN, WBP5, DUB4, CCDC92, BTG2, LPGAT1, FN1, TBX3, PLCE1, KRT19P2, IFI16, PORCN, PRSS1, MYL7, DUSP4, PROS1, ANKRD20B, CTSL2, FMO5, USP27X, LAMA1, ADAM28, ZNF611, ANKRD20B, ZNF137, S100Z, GPSM2, TGFB2, and ARHGAP28, wherein upregulation above a predetermined threshold of an expression level of the marker positively associated with the definite endodermal cells as compared to the expression level of the marker in a reference cell, wherein the reference cell is an undifferentiated hESC, is indicative of a positive identification of a definite endodermal cell.

According to some embodiments of the invention, the at least one marker positively associated with definite endodermal cells is selected from the group consisting of: FSHR, COLEC12, ROR2 ITGA5, LRP2, CD177, CCKBR, TRPA1, KEL, FOLR1, FOLH1, APOA1, APOA1, FZD5, FN1, BMP2, ADAMTS9, DPP4, FGA, GPR128, IGFBP5, LIFR, FZD4, PRTG, STC1, TNFSF4, CD177, IHH, LAMA1, GPC3, LGI1, FN1, LPHN3, FGB, KIT, DPP4, TRO, SPA17, ROBO2, DLK1, CST1, DKK1, HAS2, APOA2, CDH2, LGR5, AFP, FLRT3, APOB, DGKK, HP, SYTL5, SLC7A7, TTR, FRZB, RSPO3, GCNT1, DIO3, TMEM27, APOC1, EPHA4, C4orf18, GLIPR2, PCDH10, F10, BMP5, FMO5, STMN2, SLC5A9, PORCN, EGFLAM, RAB17, CST2, PNPLA3, MOSPD1, ELMO1, CHST9, SLC30A10, TMC7, C8orf49, CDH12, ST8SIA4, SLCO2A1, MANEA, LRIG3, HCN1, ADAMTS12, TMEM144, VTN, CAMK2N1, ABCC4, PCDH7, OR2T4, UNC93A, COL4A6, PAMR1, SLC1A1, PROS1, APOM, APOM, APOM, LYPD6B, TMEM88, ITLN2, BMPER, GPR141, VEGFA, DKK1, APOA2, AFP, APOB, FLRT3, KIAA1772, FBN2, FRZB, KLK6, FREM2, RSPO3, APOA1, APOA1, SEMA3D, KEL, BMP5, HEPH, STMN2, CACNB3, CHST9, TMC7, FAM171B, ATP5G1, FOLR1, ABCC2, EFNA5, CPT1C, MAGED1, RASA4, KDELR3, ST6GALNAC2, EDNRA, LRRC37B, ABCC4, CLCN5, MBOAT2, SLC44A5, NPC2, ELFN1, MMD, SLC5A3, IMPAD1, OSTC, DSC2, SLC31A1, SLC5A12, LRRN3, NPIPL3, GDPD1, NPIPL3, NPIPL3, LINGO2, and LYPD6B.

According to some embodiments of the invention, the at least one marker positively associated with definite endodermal cells is selected from the group consisting of: FSHR, COLEC12, ROR2 ITGA5, LRP2, CD177, CCKBR, TRPA1, KEL, FOLR1, FOLH1, APOA1, APOA1, FZD5, FN1, BMP2, ADAMTS9, DPP4, FGA, GPR128, IGFBP5, LIFR, FZD4, PRTG, STC1, TNFSF4, CD177, IHH, LAMA1, GPC3, LGI1, FN1, LPHN3, FGB, KIT, DPP4, TRO, SPA17, ROBO2, and DLK1.

According to some embodiments of the invention, the at least one marker positively associated with definite endodermal cells is selected from the group consisting of: CD177, CCKBR, APOA1, APOA1, FSHR, FZD5, FN1, BMP2, ADAMTS9, DPP4, FGA, COLEC12, ROR2, GPR128, IGFBP5, LIFR, FZD4, ITGA5, STC1, TNFSF4, CD177 and IHH.

According to an aspect of some embodiments of the invention, there is provided a method of identifying definite endodermal cells. The method is effected by determining in a population of cells which comprises definite endodermal cells at least one marker that is positively associated with definite endodermal cells, the marker being selected from the group consisting of: TRPA1, SLC40A1, SLC30A10, CCKBR, VIPR2, COLEC12, FLRT3, LGR5, GPR141, BST2, SLC5A9, GPR128, KEL, LRIG3, LYPD6B, FSHR, LIFR, FOLH1, CXCR4, ITGA5, AMOT, LY6E, SEMA6D, GJA5, PRTG, CD34, TMEM144, ROR2, GPR177, OR2T4, SLC7A7, KCNJ3, CLDN18, GPR151, SLC44A5, CDH10, TMEM27, SLC1A1, TMEM56, CD177, PLXNA2, SLC26A2, DSCAM, TMEM133, IL13RA1, ATP2B1, CD302, MEGF9, EDNRA, CDH2, GPR161, TYRO3, FLRT2, LRIT3, PCDH7, NRCAM, SMAGP, AMHR2, ELTD1, GRPR, EPHA4, and CD99, wherein upregulation above a predetermined threshold of an expression level of the marker positively associated with the definite endodermal cells as compared to the expression level of the marker in a reference cell, wherein the reference cell is an undifferentiated hESC, is indicative of a positive identification of a definite endodermal cell.

According to some embodiments of the invention, the method further comprising determining in the population of cells which comprises definite endodermal cells at least one marker that is positively associated with definite endodermal cells, the marker being selected from the group consisting of: GATA3, SEMA3E, HHEX, ZNF280A, FAM184A, WDR72, PDZK1, RLBP1L2, SHISA2, VIL1, STMN2, APOA2, SERHL, PPFIBP2, DKK1, MUM1L1, IGFBP5, ST6GALNAC2, TSPYL5, STC1, SYTL5, EPSTI1, ANKRD1, ARHGAP24, KRT18P49, PRSS2, RHOBTB3, FRZB, RARB, ADAMTS9, ARL4D, PRDM1, HP, FZD5, TRY6, ATP6V0D2, ANGPT2, DENND2C, BMP5, FOXA2, HAS2, BMP2, S100A16, FOLH1B, FAM122C, FZD4, C5, S100A14, VEGFA, CLIP4, GPAM, HNF1B, APOA1, CFLAR, RBM24, RNF152, TTR, TTN, EGFLAM, APOB, DIO3, IFLTD1, ABCC4, CCDC141, ENC1, NEK2, ELMO1, SPOCK3, SERPINI1, ACSL1, GATM, EHHADH, NUDT4, CST1, GLUD2, NPL, ZNF702P, TRY6, SPOCK1, AGL, TFF1, DGKK, SALL1, MANEA, KIT, KRT19, TNNC1, SEPP1, ST8SIA4, YPEL2, ANKMY2, DNAJC15, RNF128, PTPN13, F10, SAMD3, GCNT1, IPP, PROS1, SV2B, PLOD2, MAGEH1, CHST9, ZNF518B, TMEM106C, SERHL2, NTN4, SOX17, FRRS1, OTX2, RNASEL, ELMOD2, MYCT1, PAX6, MGST2, BBS5, MTSS1, VTN, WBP5, DUB4, CCDC92, BTG2, LPGAT1, FN1, TBX3, PLCE1, KRT19P2, IFI16, PORCN, PRSS1, MYL7, DUSP4, PROS1, ANKRD20B, CTSL2, FMO5, USP27X, LAMA1, ADAM28, ZNF611, ANKRD20B, ZNF137, S100Z, GPSM2, TGFB2, and ARHGAP28, wherein upregulation above a predetermined threshold of an expression level of the marker positively associated with the definite endodermal cells as compared to the expression level of the marker in a reference cell, wherein the reference cell is an undifferentiated hESC, is indicative of a positive identification of a definite endodermal cell.

According to some embodiments of the invention, the method further comprising determining in the population of cells which comprises definite endodermal cells at least one marker that is negatively associated with definite endodermal cells, the marker being selected from the group consisting of: KDR, PCDHB5, FAT4, FLT1, NRN1, THBS2, PTPRZ1, SLC6A15, GPR176, SEMA6A, THBS1, CDH11, GRID2, SLC7A11, CDH1, LRFN5, EDNRB, THY1, NETO1, KCND2, TMPRSS11E, CD44, PDPN, SLC7A1, KAL1, KCNG3, GPM6B, FXYD5, PCDH18, ICAM3, MCTP1, TACR3, TMEM155, ZFP42, THUMPD3, ANXA1, SPP1, PRDM14, GNA14, EDIL3, CXCL12, PSMD5, PRRX1, NANOG, TRIM22, NANOG, RASGRF2, POU5F1B, POLR3G, HHLA1, POU5F1, VSNL1, SCG3, B3GALT1, LECT1, NTS, MBNL1, CKMT1A, NECAB1, FGF2, SFRP2, DCLK1, DACT1, CRABP1, TFAP2C, SCGB3A2, LRAT, CUZD1, GLB1L3, METTL7A, VAT1L, COL12A1, OLFML3, SOX2, USP44, HIST1H4F, KGFLP1, CPT1A, DBC1, CHAC1, CAV1, MT1G, NFIX, FERMT1, GLIPR1, TOX, SNRPN (SNORD116-6), HEY2, T1MP4, IDO1, MT2A, NR5A2, UPRT, MYC, CCDC109B, GAL, ZNF483, RND3, BNC2, COL3A1, LUM, LDB2, MT1E, SNRPN (SNORD116-23), SNRPN (SNORD116-27), GALNT3, PIPOX, PDK1, PREX2, CYP2S1, NRK, VAV3, TNFAIP6, ENPP1, ADD2, SNRPN (SNORD116-24), SNRPN (SNORD109A), STC2, SNORA22, MPPED2, ZNF562, GAP43, FOXB1, TSHZ3, HPGD, ZDHHC22, ACOXL, GLI3, CDCA7L, ZSCAN10, GFPT2, PLP2, HIST1H1A, CAMKV, HERC5, MT1X, TERF1, RAB31, SNRPN (SNORD116-13), ETV1, MT1G, ACTA1, SNRPN (SNORD116-20), NFIB, ZEB2, CBR1, ATXN7L1, SNRPN (SNORD116-1), MT1F, SNRPN (SNORD116-29), AP1M2, ACTG2, CYP2B6, SERPINE1, GRHL2, SLIT2, PIM2, SMARCA2, and RPPH1, wherein downregulation above a predetermined threshold of an expression level of the marker negatively associated with definite endodermal cells as compared to the expression level of the marker in a reference cell, wherein the reference cell is an undifferentiated hESC, is indicative of a positive identification of a definite endodermal cell.

According to an aspect of some embodiments of the invention, there is provided a method of isolating definite endodermal cells. The method is effected by (a) identifying the definite endodermal cells according to the method of some embodiments of the invention, (b) isolating the definite endodermal cells identified according to step (a) to thereby obtain an isolated population of the definite endodermal cells, thereby isolating the definite endodermal cells.

According to an aspect of some embodiments of the invention, there is provided an isolated population of definite endodermal cells obtained according to the method of some embodiments of the invention.

According to some embodiments of the invention, the isolated cell population of definite endodermal cells is characterized by a SOX17+/SOX7+ expression pattern.

According to an aspect of some embodiments of the invention, there is provided an isolated population of definite endodermal cells, comprising at least about 20%, at least about 25%, at least about 30%, at least about 35%, at least about 40%, at least about 45%, at least about 50% (e.g., 50%), at least about 55% (e.g., 55%), at least about 60% (e.g., 60%), at least about 65% (e.g., 65%), at least about 70% (e.g., 70%), at least about 75% (e.g., 75%), at least about 80% (e.g., 80%), at least about 85% (e.g., 85%), at least about 90% (e.g., 90%), at least about 95% (e.g., 95%), at least about 96% (e.g., 96%), at least about 97% (e.g., 97%), at least about 98% (e.g., 98%), at least about 99% (e.g., 99%), e.g., 100% cells having a SOX17+/SOX7+ expression pattern.

According to an aspect of some embodiments of the invention, there is provided a method qualifying a definite endodermal cell population. The method is effected by determining in a sample of the cell population a percentage of the definite endodermal cells which are identified according to the method of some embodiments of the invention out of the total cells in the sample, thereby qualifying the definite endodermal cell population.

According to some embodiments of the invention, the pancreatic progenitor cells or the definite endodermal cells are obtained by differentiating stem cells into pancreatic progenitor cells.

According to some embodiments of the invention, the stem cells are undifferentiated pluripotent stem cells.

As used herein the phrase “pluripotent stem cells” refers to cells which are capable of differentiating into cells of all three embryonic germ layers (i.e., endoderm, ectoderm and mesoderm). The phrase “pluripotent stem cells” may read on embryonic stem cells (ESCs) and/or induced pluripotent stem cells (iPS cells).

The ESCs which are used to generate the pancreatic progenitor cells or the definite endodermal cells can be obtained from the embryonic tissue formed after gestation (e.g., blastocyst) before implantation (i.e., a pre-implantation blastocyst); extended blastocyst cells (EBCs) which are obtained from a post-implantation/pre-gastrulation stage blastocyst (see WO2006/040763]; and/or embryonic germ (EG) cells which are obtained from the genital tissue of a fetus any time during gestation, preferably before 10 weeks of gestation.

Extended blastocyst cells (EBCs) can be obtained from a blastocyst of at least nine days post fertilization at a stage prior to gastrulation. Prior to culturing the blastocyst, the zona pellucida is digested [for example by Tyrode's acidic solution (Sigma Aldrich, St Louis, Mo., USA)] so as to expose the inner cell mass. The blastocysts are then cultured as whole embryos for at least nine and no more than fourteen days post fertilization (i.e., prior to the gastrulation event) in vitro using standard embryonic stem cell culturing methods.

Embryonic germ (EG) cells are prepared from the primordial germ cells obtained from fetuses of about 8-11 weeks of gestation (in the case of a human fetus) using laboratory techniques known to anyone skilled in the arts. The genital ridges are dissociated and cut into small chunks which are thereafter disaggregated into cells by mechanical dissociation. The EG cells are then grown in tissue culture flasks with the appropriate medium. The cells are cultured with daily replacement of medium until a cell morphology consistent with EG cells is observed, typically after 7-30 days or 1-4 passages. For additional details on methods of preparation human EG cells see Shamblott et al., [Proc. Natl. Acad. Sci. USA 95: 13726, 1998] and U.S. Pat. No. 6,090,622.

The phrase “induced pluripotent stem (iPS) cell” (or embryonic-like stem cell) as used herein refers to a proliferative and pluripotent stem cell which is obtained by de-differentiation of a somatic cell (e.g., an adult somatic cell).

According to some embodiments of the invention, the iPS cell is characterized by a proliferative capacity which is similar to that of ESCs and thus can be maintained and expanded in culture for an almost unlimited time.

IPS cells can be endowed with pluripotency by genetic manipulation which re-program the cell to acquire embryonic stem cells characteristics. For example, the iPS cells of the invention can be generated from somatic cells by induction of expression of Oct-4, SOX2, Kfl4 and c-Myc in a somatic cell essentially as described in Takahashi and Yamanaka, 2006, Takahashi et al, 2007, Meissner et al, 2007, and Okita K., et al, 2007, Nature 448: 313-318). Additionally or alternatively, the iPS cells of the invention can be generated from somatic cells by induction of expression of Oct4, SOX2, Nanog and Lin28 essentially as described in Yu et al, 2007, and Nakagawa et al, 2008. It should be noted that the genetic manipulation (re-programming) of the somatic cells can be performed using any known method such as using plasmids or viral vectors, or by derivation without any integration to the genome [Yu J, et al., Science. 2009, 324: 797-801].

The iPS cells of the invention can be obtained by inducing de-differentiation of embryonic fibroblasts [Takahashi and Yamanaka, 2006; Meissner et al, 2007], fibroblasts formed from hESCs [Park et al, 2008], Fetal fibroblasts [Yu et al, 2007; Park et al, 2008], foreskin fibroblast [Yu et al, 2007; Park et al, 2008], adult dermal and skin tissues [Hanna et al, 2007; Lowry et al, 2008], b-lymphocytes [Hanna et al 2007] adult liver and stomach cells [Aoi et al, 2008] and beta-cells derived iPSCs (Bar-Nur O., et al., 2011m Cell Stem Cell 9: 1-7).

IPS cell lines are also available via cell banks such as the WiCell bank. Non-limiting examples of commercially available iPS cell lines include the iPS foreskin clone 1 [WiCell Catalogue No. iPS(foreskin)-1-DL-1], the iPSIMR90 clone 1 [WiCell Catalogue No. iPS(IMR90)-1-DL-1], and the iPSIMR90 clone 4 [WiCell Catalogue No. iPS(IMR90)-4-DL-1].

According to some embodiments of the invention, the induced pluripotent stem cells are human induced pluripotent stem cells.

According to some embodiments of the invention, the stem cells are adult stem cells.

The phrase “adult stem cells” (also called “tissue stem cells” or a stem cell from a somatic tissue) refers to any stem cell derived from a somatic tissue [of either a postnatal or prenatal animal (especially the human)]. The adult stem cell is generally thought to be a multipotent stem cell, capable of differentiation into multiple cell types. Adult stem cells can be derived from any adult, neonatal or fetal tissue such as adipose tissue, skin, kidney, liver, prostate, pancreas, intestine, bone marrow and placenta. Non-limiting examples include mesenchymal stem cells (MSCs), cord blood stem cells, fetal stem cells and the like.

Hematopoietic stem cells, which may also referred to as adult tissue stem cells, include stem cells obtained from blood or bone marrow tissue of an individual at any age or from cord blood of a newborn individual. Preferred stem cells according to this aspect of some embodiments of the invention are embryonic stem cells, preferably of a human or primate (e.g., monkey) origin.

Placental and cord blood stem cells may also be referred to as “young stem cells”.

According to some embodiments of the invention, the cells are human cells.

According to some embodiments of the invention, differentiating the undifferentiated pluripotent stem cells into the pancreatic progenitor cells is performed by: (a) differentiating the pluripotent stem cells into definite endodermal cells to thereby obtain a population of cells which comprises definite endodermal cells, and (b) differentiating the population of cells which comprises the definite endodermal cells into the pancreatic progenitor cells, thereby inducing the differentiation of the pluripotent stem cells into the pancreatic progenitor cells.

According to some embodiments of the invention, differentiating the undifferentiated pluripotent stem cells into the definite endodermal cells is performed by culturing the pluripotent stem cells in the presence of activin A, Wnt3A, a small molecule Induce Definitive Endoderm 1 (IDE1) and/or a small molecule Induce Definitive Endoderm 2 (IDE2) as described in the Examples section which follows and in Jiang J., et al., 2007 (Generation of insulin-producing islet-like clusters from human embryonic stem cells. Stem Cells 25:1940-1953) and/or in D'Amour et al., 2006 (Nat Biotechnol 24:1392-1401), each of which is fully incorporated herein by reference in its entirety.

Differentiation of the definite endodermal cells into the pancreatic progenitor cells can be performed by culturing the definite endodermal cells in the presence of bFGF, KGF, FGF10, noggin, cyclopamine, KAAD cyclopamine, B27, Indolactam V, nicotinamide and/or epidermal growth factor.

According to some embodiments of the invention, differentiating the definite endodermal cells into the pancreatic progenitor cells is performed by culturing the definite endodermal cells in the presence of bFGF, noggin and epidermal growth factor (additional description is provided in Jiang J, Stem Cells. 2007; 25(8):1940-53, which is fully incorporated herein by reference in its entirety.

According to some embodiments of the invention, differentiating the undifferentiated pluripotent stem cells into the pancreatic progenitor cells is performed by differentiation of the pluripotent stem cells into embryoid bodies.

As used herein the phrase “embryoid bodies” (EBs) refers to three dimensional multicellular aggregates of differentiated and undifferentiated cells derivatives of three embryonic germ layers.

Embryoid bodies are formed upon the removal of embryonic stem cells (ESCs) or induced pluripotent stem cells (iPSCs) from feeder layers or feeder cells-free culture systems. ESCs and/or iPSCs removal can be effected using type IV Collagenase treatment for a limited time. Following dissociation from the culturing surface, the cells are transferred to tissue culture plates containing a culture medium supplemented with serum and amino acids.

During the culturing period, EBs are further monitored for their differentiation state. Cell differentiation can be determined upon examination of cell or tissue-specific markers which are known to be indicative of differentiation. For example, EB-derived-differentiated cells may express the neurofilament 68 KD which is a characteristic marker of the ectoderm cell lineage.

The differentiation level of the EB cells can be monitored by following the loss of expression of Oct-4, and the increased expression level of other markers such as α-fetoprotein, NF-68 kDa, α-cardiac and albumin. Methods useful for monitoring the expression level of specific genes are well known in the art and include RT-PCR, semi-quantitative RT-PCR, Northern blot, RNA in situ hybridization, Western blot analysis and immunohistochemistry.

Embryoid bodies can be generated from various primates and mammals such as human, monkeys and rodents (e.g., mouse, rat).

According to some embodiments of the invention, the embryoid bodies are obtained from human embryoid bodies.

According to some embodiments of the invention, the embryoid bodies are obtained by spontaneous differentiation of pluripotent stem cells.

According to some embodiments of the invention, for differentiation into pancreatic progenitor cells, the embryoid bodies are differentiated until about day 7-21 of human EBs differentiation.

According to some embodiments of the invention, for differentiation into definite endodermal cells, the embryoid bodies are differentiated until about day 3-7 of human EBs differentiation.

According to an aspect of some embodiments of the invention there is provided a nucleic acid construct comprising a first polynucleotide encoding a reporter protein and a second polynucleotide which comprises a human endogenous SOX17 regulatory sequence, wherein the first polynucleotide being under transcriptional regulation of the SOX17 regulatory sequence, wherein the SOX17 regulatory sequence comprises an upstream sequence and a downstream sequence.

According to some embodiments of the invention, expression of the reporter protein is under the transcriptional regulation of the SOX17 regulatory sequences.

As used herein the phrase “reporter protein” refers to any polypeptide which can be detected in a cell. According to some embodiments of the invention, the reporter polypeptide can be directly detected in the cell (no need for a detectable moiety with an affinity to the reporter) by exerting a detectable signal which can be viewed in living cells (e.g., using a fluorescent microscope). Non-limiting examples of a nucleic acid sequence encoding a reporter polypeptide according to this aspect of the present invention include the red fluorescent protein (RFP), the green fluorescent protein (GFP) (e.g., SEQ ID NO:2) or mCherry (e.g., SEQ ID NO:33).

Alternatively, the reporter polypeptide can be indirectly detected such as when the reporter polypeptide is an epitope tag. Indirect detection can be effected by introducing a detectable moiety (labeled antibody) having an affinity to the reporter or when the reporter is an enzyme by introducing a labeled substrate. For example, the reporter polypeptide can be an antigen which is recognized by and binds to a specific antibody. Preferably, when such a reporter polypeptide is utilized the antibody or the polypeptide capable of binding the reporter protein is labeled (e.g., by covalently attaching to a label such as a fluorescent dye).

Non-limiting examples of suitable SOX17 upstream regulatory sequences which can be used in the nucleic acid construct of some embodiments of the invention include the sequences comprising SEQ ID NO: 7, 36, and 38.

Non-limiting examples of suitable SOX17 downstream regulatory sequences which can be used in the nucleic acid construct of some embodiments of the invention include the sequences comprising SEQ ID NO: 8, 37 and 39.

According to an aspect of some embodiments of the invention there is provided a nucleic acid construct comprising a first polynucleotide encoding a reporter protein and a second polynucleotide which comprises a human endogenous SOX17 regulatory sequence, wherein the first polynucleotide being under transcriptional regulation of the SOX17 regulatory sequence, wherein the SOX17 regulatory sequence comprises an upstream sequence and a downstream sequence, wherein the upstream sequence comprises the nucleotide sequence set forth in SEQ ID NO:38; and wherein the downstream sequence comprises the nucleotide sequence set forth in SEQ ID NO:39.

According to an aspect of some embodiments of the present invention there is provided a nucleic acid construct comprising a first polynucleotide encoding a reporter protein and a second polynucleotide which comprises a human endogenous PDX1 regulatory sequence, wherein the first polynucleotide being under transcriptional regulation of the PDX1 regulatory sequence, wherein the PDX1 regulatory sequence comprises an upstream sequence and a downstream sequence.

Non-limiting examples of suitable PDX1 upstream regulatory sequences which can be used in the nucleic acid construct of some embodiments of the invention include the sequences comprising SEQ ID NO: 16, 19, 23, and 31.

Non-limiting examples of suitable PDX1 downstream regulatory sequences which can be used in the nucleic acid construct of some embodiments of the invention include the sequences comprising SEQ ID NO: 17, 20, 24, and 32.

According to an aspect of some embodiments of the present invention there is provided a nucleic acid construct comprising a first polynucleotide encoding a reporter protein and a second polynucleotide which comprises a human endogenous PDX1 regulatory sequence, wherein the first polynucleotide being under transcriptional regulation of the PDX1 regulatory sequence, wherein the PDX1 regulatory sequence comprises an upstream sequence and a downstream sequence, wherein the upstream sequence comprises the nucleotide sequence set forth in SEQ ID NO:16 (corresponds to nucleotides 1 to 8640 of AL353195.14); and wherein the downstream sequence comprises the nucleotide sequence set forth in SEQ ID NO:17 (corresponds to nucleotides 13321 to 32526 of AL353195.14 (SEQ ID NO:34)). According to some embodiments of the invention, the nucleic acid construct is a bacterial artificial chromosome (BAC).

Non-limiting examples of suitable BACs which comprise the SOX17 genomic sequence include the RP11-53M11 BAC (SEQ ID NO:35; GenBank Accession No. AC091076.7), and the RP11-47H10 BAC (SEQ ID NO:40).

A non-limiting example of a suitable BACs which comprise the PDX1 genomic sequence include the RP11-322P28 (SEQ ID NO:34; GenBank Accession No. AL353195.14).

According to an aspect of some embodiments of the invention there is provided a cell comprising the nucleic acid construct of some embodiments of the invention.

According to some embodiments of the invention, the cell is a stem cell.

According to some embodiments of the invention, the stem cell is an embryonic stem cell or an induced pluripotent stem cell.

According to some embodiments of the invention, the cell is a human cell.

According to an aspect of some embodiments of the invention there is provided a method of screening for markers which differentiate a definite endodermal cell from an undifferentiated pluripotent stem cell, comprising comparing the expression level of markers between the undifferentiated pluripotent stem cell and the cell of some embodiments of the invention (e.g., the cell which comprises the reporter protein under the transcriptional regulation of the SOX17 upstream and downstream regulatory sequence), wherein upregulation or downregulation in the expression level above a predetermined threshold indicates that the markers differentiate the definite endodermal cell from the undifferentiated pluripotent stem cell, thereby screening for markers which differentiate the definite endodermal cell from the undifferentiated pluripotent stem cell.

According to an aspect of some embodiments of the invention there is provided a method of screening for compounds capable of inducing differentiation of undifferentiated pluripotent stem cells to definite endodermal cells, comprising: (a) contacting undifferentiated pluripotent stem cells which comprise the nucleic acid construct of some embodiments of the invention (e.g., the construct which comprises the reporter protein under the transcriptional regulation of the SOX17 upstream and downstream regulatory sequence) with at least one compound of a plurality of candidate compounds, and; (b) monitoring an expression level of the reporter protein in the cells following the contacting, wherein an increase above a predetermined level in the expression level of the reporter protein following the contacting as compared to the expression level prior to the contacting is indicative that the at least one compound is capable of inducing differentiation of the undifferentiated pluripotent stem cells to the definite endodermal cells, thereby screening for the compounds capable of inducing differentiation of undifferentiated pluripotent stem cells to definite endodermal cells.

According to some embodiments of the invention, the method further comprising synthesizing the compound capable of inducing differentiation of the undifferentiated pluripotent stem cells to the definite endodermal cells.

According to an aspect of some embodiments of the invention there is provided a method of screening for markers which differentiate a pancreatic progenitor cell from a definite endodermal cell, comprising comparing the expression level of markers between the cell which comprises the reporter protein under the transcriptional regulation of the SOX17 upstream and downstream regulatory sequence of some embodiments of the invention and the cell which comprises the reporter protein under the transcriptional regulation of the PDX1 upstream and downstream regulatory sequence of some embodiments of the invention, wherein upregulation or downregulation in the expression level above a predetermined threshold indicates that the markers differentiate the pancreatic progenitor cell from the definite endodermal cell, thereby screening for markers which differentiate the pancreatic progenitor cell from the definite endodermal cell.

According to an aspect of some embodiments of the invention there is provided a method of screening for compounds capable of inducing differentiation of definite endodermal cells or undifferentiated pluripotent stem cells to pancreatic progenitor cells, comprising: (a) contacting definite endodermal cells or undifferentiated pluripotent stem cells which comprise the nucleic acid construct of some embodiments of the invention (which comprises the reporter protein under the transcriptional regulation of the PDX1 upstream and downstream regulatory sequences) with at least one compound of a plurality of candidate compounds, and; (b) monitoring an expression level of the reporter protein in the cells following the contacting, wherein an increase above a predetermined level in the expression level of the reporter protein following the contacting as compared to the expression level prior to the contacting is indicative that the at least one compound is capable of inducing differentiation of the definite endodermal cells or undifferentiated pluripotent stem cells to the pancreatic progenitor cells, thereby screening for the compounds capable of inducing differentiation of definite endodermal cells or undifferentiated pluripotent stem cells to the pancreatic progenitor cells.

According to some embodiments of the invention the method further comprising synthesizing the compound capable of inducing differentiation of the definite endodermal cells or undifferentiated pluripotent stem cells to the pancreatic progenitor cells.

The agents of some embodiments of the invention which are described hereinabove for screening for markers which differentiate a definite endodermal cell from an undifferentiated pluripotent stem cell, for screening for compounds capable of inducing differentiation of undifferentiated pluripotent stem cells to definite endodermal cells, for screening for markers which differentiate a pancreatic progenitor cell from a definite endodermal cell, and/or for screening for compounds capable of inducing differentiation of definite endodermal cells or undifferentiated pluripotent stem cells to pancreatic progenitor cells may be included in a diagnostic kit/article of manufacture preferably along with appropriate instructions for use and labels indicating FDA approval for the above described use.

Such a kit can include, for example, at least one container including at least one of the above described agents (e.g., the nucleic acid constructs, the cells comprising same) and an imaging reagent packed in another container (e.g., enzymes, antibodies, buffers, chromogenic substrates, fluorogenic material). The kit may also include appropriate buffers and preservatives for improving the shelf-life of the kit.

According to an aspect of some embodiments of the invention there is provided a kit for screening for markers which differentiate a definite endodermal cell from a pluripotent stem cell, comprising the cell of some embodiments of the invention.

According to some embodiments of the invention the kit further comprising a pluripotent stem cell.

According to an aspect of some embodiments of the invention there is provided a kit for screening for markers which differentiate a pancreatic progenitor cell from a definite endodermal cell, comprising the cell of some embodiments of the invention and the cell of some embodiments of the invention.

According to some embodiments of the invention the kit further comprising at least one agent suitable for detecting an expression level of a marker of interest.

According to some embodiments of the invention the expression level is detected by an RNA detection method.

According to some embodiments of the invention the expression level is detected by a protein detection method.

According to some embodiments of the invention the kit further comprising a genetic micro array chip.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Methods

hESC Culture and Differentiation—

H9 (4), I3, I6 (13) and CSES2 (14, 15) cells were routinely cultured on mouse embryonic fibroblasts (MEFs) that have been mitotically inactivated by mitomycin C. The cells were grown in ES cell medium consisting of: 80% knockout Dulbeco's Modified Eagle's Medium (Invitrogen, Paisley, UK), 20% knockout serum replacement (Invitrogen), 1 mM glutamine (Biological Industries, Israel) and 1% non-essential amino acids (Biological Industries, Israel), 0.1 mM 2-mercaptoethanol (Sigma Chemical Co.) and 4 ng/ml basic fibroblast growth factor (bFGF) (PeproTech, Rehovot, Israel). Karyotyping of hESCs and transfected clones was performed as described (16). Embryonic bodies (EBs) were formed by harvesting the cells with a trypsin solution which contains calcium and magnesium (Biological Industries, Israel). When cell clusters began to detach from the MEF feeders, the cells were collected, centrifuged and transferred to plastic non coated UV-irradiated bacteriological Petri dishes in EB medium in order to allow their aggregation. The EB medium used is essentially the same as the ES cell medium except bFGF was not included. The EB medium consisted of: 80% knockout Dulbecco's Modified Eagle's Medium (Invitrogen, Paisley, UK), 20% knockout serum replacement (Invitrogen), 1 mM glutamine (Biological Industries, Israel) and 1% non-essential amino acids (Biological Industries, Israel).

Transfections

BAC Transgenesis of a SOX17-GFP Reporter BAC—

The SOX17 BAC reporter construct was built using recombineering (recombination-mediated genetic engineering) (17) in which the coding sequence of the SOX17 gene was replaced by the coding sequence of the GFP gene together with a floxed neomycin resistance gene. BACs were obtained from the Australian Genome Research Facility and specifically RP11-53M11 and RP11-47H10 were modified. The modified BACs were electroporated into hESCs essentially according to the protocol of Zwaka and Thomson (18). Briefly, hESCs cells were harvested from the plate with a trypsin solution containing calcium and magnesium and were centrifuged and the pellet was resuspended in 500 microliters of ES cell medium. Fifty micrograms of BAC DNA were mixed with PBS to a final volume of 300 microliters and the DNA solution was mixed with the cell suspension and was transferred to a 4 mm cuvette (Bio-Rad, Richmond, Calif.). The cells were electroporated using a Bio-Rad Gene Pulser at 320V and 200 mF. After electroporation the cuvette was allowed to stand for ten minutes and then the contents were transferred to a plate of MEFs in ES cell medium. For selection of resistant colonies, the hESCs were cultured on MEFs harboring the neomycin resistance gene (derived from DR4 mice; Jackson Laboratories USA). To obtain optimal survival of the cells after electroporation, Rho-associated kinase (ROCK) Inhibitor (Alexis Biochemicals, San Diego, Calif., USA) was added to the medium at a concentration of 10 mM. Two days later, selection with G418 (40 microgram/ml effective concentration) was initiated and selection medium was changed every two or three days. At about day 14, G418 resistant clones were isolated and expanded.

The various clones were allowed to form embryoid bodies (EBs) in the presence of activin A (66 ng/ml) (Peprotech) and two days later, the EBs were dissociated into single cells with TrypLE select (Invitrogen) and were analysed by FACS.

BAC Transgenesis of PDX1-GFP Reporter BAC—

A PDX1 BAC reporter construct was built in which the coding sequence of the PDX1 gene was replaced by the coding sequence of the GFP gene together with a floxed Neo gene in a BAC spanning the genomic region of the PDX1 gene (RP11-328P22, GenBank Accession No. AL353195.14 (SEQ ID NO:34)). The BAC included about 8.6 kb of upstream promoter sequence of the PDX1 gene (SEQ ID NO:16) and about 19.2 Kb of the downstream sequence of the PDX1 gene (SEQ ID NO:17).

Similarly to the SOX17-BAC, various hESC clones harbouring the PDX1-GFP reporter BAC were isolated and were allowed to differentiate by forming EBs. At day 14, the EBs were dissociated into single cells and were analysed by FACS.

Cell Culture and Genetic Labeling—

Human Embryonic Stem Cells (HESC) clones carrying either SOX17 or PDX1 BAC GFP reporter protein were generated for the screening. Cells were grown in a tissue culture facility and received standard treatments and medium replacement. Differentiations to definitive endoderm (SOX17⁺) and to pancreatic cells (PDX1⁺) were achieved by applying the appropriate protocols as described below. Positive monitoring of SOX17-GFP⁺ cells and PDX1-GFP⁺ cells enables the isolation of semi-homogenous population for the expression analysis.

Differentiation of Pluripotent Stem Cells into Definite Endodermal Cells—

To differentiate hESCs toward definitive endoderm, the present inventors used the protocol described by D'Amour (D'Amour 2006, Nat Biotechnol 24:1392-1401) which includes culturing the cells in the presence of Activin A or its substitute IDE2 (Borowiak M, 2009 (Cell Stem Cell 4, 348-358)). In brief, 80-90% confluent hESCs were cultured for 1 day in RPMI (Invitrogen) medium supplemented with 1 mM glutamine and either 25 ng/ml Wnt3a (R&D) and 100 ng/ml Activin A (Peprotec), or 5 μM IDE2 (Enzo Life Sciences Inc, Lausen, Switzerland). The medium was then changed to RPMI with 0.2% Foetal bovine serum (FBS) (Invitrogen), 1 mM glutamine and 100 ng/ml Activin A or 5 μM IDE2 for culturing of 2 additional days.

Differentiation of Definite Endodermal Cells into Pancreatic Progenitor Cells—

Definite endodermal cells can be further differentiated into pancreatic progenitor cells by following the differentiation protocol(s) described by D'Amour 2006 (Nat Biotechnol 24:1392-1401), Kroon 2008 (Nat Biotechnol. 2008; 26:443-52. Epub 2008 Feb. 20) and/or Borowiak 2009 (Cell Stem Cell 4, 348-358, Apr. 3, 2009).

Microarray—

RNA was extracted from FACS sorted cell populations using Qiagen micro RNA isolation kit. The integrity of the RNA was confirmed using an RNA Pico Bioanalyzer and microarray analysis was performed on the Human Gene ST1.0 chip (Affymetrix, Santa Clara, Calif.). Three replicas from each differentiation stage (SOX17⁺ and PDX1⁺ cells) were loaded on microarray chips. Analysis was performed on the Human Gene ST1.0 chip (Affymetrix, Santa Clara, Calif.). Evaluation of changes in gene expression was performed by comparing array results to a previous Human Embryonic Stem Cells (HESC) microarray data (3 replicas) and to Pancreas microarray results (3 replicas).

Data Analysis—

In accordance with the aim of the current study, an emphasis was put on genes with known surface characteristics. The Gene Ontology (GO) Cellular Component Term information from the Affymetrix database was analyzed, enabling an efficient prioritization of the relevant genes and sequences. The present inventors used the Partek® Genomics Suite™ (Partek Incorporated, St. Louis, Mo., U.S.A) for the bioinformatic analysis. Following the first analysis, a refinement of each selected gene was done by crossing the results with the comprehensive GeneCards database (http://www(dot)genecards(dot)org/). Following the cross check, genes were divided into two groups: cell surface genes; and putative surface and membranal genes.

Statistical Analysis—

The Fold Change (FC) threshold was set on FC=2.0. Expression comparisons were calculated by using Student's test. Significance was taken as P<0.05.

Selection of PDX1 Expressing Cells—

14 day-old EBs or older EBs were used to select for TROP-2 and GPR50 expressing cells.

FACS Sorting—

FACS sorting was performed on single cells that had been dissociated using TrypLE select (Invitrogen) using a FACS ARIA (Becton Dickinson, Bedford, Mass.). GFP positive (GFP+) and GFP negative (GFP−) cells were isolated. FACS sorting was also performed using allophycocyanin (APC) conjugated mouse monoclonal anti human TROP-2 (1:100) and mouse monoclonal anti human GPR50 (1:200) (both from R&D Systems Inc., Minneapolis, Minn.) followed by secondary anti-mouse immunoglobulin G (IgG) fluorescence isothiocyanate (FITC) conjugated antibody 1:100 (Chemicon/Millipore, Billerica, Mass.).

RT-PCR—

Total RNA was isolated from differentiated hESCs using RNeasy micro kit (Qiagen, Hilden, GmbH) according to the manufacturer's recommended protocol. cDNA was synthesized using Superscript reverse transcriptase (Invitrogen). Quantitative Real Time (QRT) PCR (RT-qPCR) analysis was performed in triplicate and normalized to the internal endogene GAPDH gene expression. The reaction was performed in an ABI Prism 7000 (Applied Biosystems, Warrington, UK) with the TaqMan Universal PCR Master Mix (Applied Biosystems) using Taqman probes (Applied Biosystems) and analyzed using the relative Quantification (RQ) study in the Sequence Detection Software (V. 1.2; Applied Biosystems).

Immunofluorescence—

The cells were seeded on 13 mm glass cover slides in E-well culture plates. Forty-eight hours after seeding, cells were fixed for 20 minutes in 4% paraformaldehyde in PBS; permeabilized using 0.5% TritonX-100 in PBS/1% fetal bovine serum, and incubated overnight with the primary antibodies: goat anti-SOX17 1:400 (R&D), goat anti-OCT4 1:200 (Santa Cruz), rabbit anti-green florescent protein (GFP) polyclonal antibody 1:100 (Chemicon/Millipore, Billerica, Mass.), or goat anti-PDX1 1:10,000 (Abcam plc, Cambridge, UK). After rinsing, secondary anti-mouse or anti-goat IgG Cy3 and anti-rabbit IgG Cy2 (Jackson Laboratories, West Grove, Pa.) were added to the samples, which were then incubated for an additional hour. Finally, the cells were rinsed once more and mounted with mounting media (VECTASHIELD, Vector Lab, CA). The slides were analyzed using a confocal microscope (Bio-Rad MRC 1024, Richmond Calif., USA).

### Example 1

The differentiation of hESCs into pancreatic beta cells is a stepwise process by which the initially pluripotent cell gradually becomes more committed towards the final cell fate of a functional insulin-producing cell. Initially, the pluripotent stem cells differentiate via mesendoderm into definitive endoderm. The definitive endoderm then commits towards a pancreatic cell fate, and these cells in turn differentiate towards an endocrine pancreatic cell fate, after which they commit to beta cells. FIG. 5A depicts a schematic illustration of differentiation of stem cells into pancreatic cells.

In order to fully characterize the differentiation of hESCs into insulin-producing cells it is important to identify and isolate these stage specific progenitor cells and characterize their properties on the molecular level. By obtaining a transcriptional profile of these cells and by identifying transcription factors that they express, a clearer picture of the differentiation process can be obtained. The present inventors took the approach of genetically labeling hESCs with stage specific fluorescent reporter constructs, thus generating hESCs reporter clones. These provide a simple, accurate and sensitive readout of the appearance of the progenitors thus enabling optimization of conditions for inducing their differentiation. The fluorescent reporter lines also provide valuable tools for isolating progenitor cells by FACS and further studying their gene profile.

The present inventors chose two key transcription factors expressed during differentiation towards beta cells: SOX17 and PDX1. SOX17 is expressed early at the definitive endoderm stage while PDX1 is expressed at a critical point in the pathway at which the cells decide to differentiate into pancreas or non-pancreas endodermal derivatives. Using genetically modified hESCs expressing EGFP under the control of SOX17 or PDX1 promoters, the present inventors were able to isolate the stage specific cells by FACS and characterize their transcriptional profile by qPCR and microarray analysis.

Experimental Results

BAC Transgenesis of a SOX17-GFP Reporter BAC

The simplest method to generate reporter cells is to introduce a reporter plasmid expressing EGFP under a minimal promoter. The problem with this approach is that the random integration of a plasmid is subject to position effects, and also that the minimal promoter might not mimic the exact in vivo expression pattern of the gene. A different approach that alleviates some of these problems is to modify a BAC that spans the genomic region of the gene by inserting IRES-EGFP into the 3′UTR of the gene or by replacing the coding sequence of the gene with the coding sequence of EGFP. In such a way, EGFP is expressed under the control of a more extensive genomic sequence that presumably contains all or most of the critical transcriptional regulatory elements of the gene. Furthermore, the large size of the BAC may, to some extent, buffer the reporter construct from position effects.

A SOX17-BAC reporter construct was built in which the coding sequence of the SOX17 gene was replaced by the coding sequence of the GFP gene together with a floxed neo gene.

Preparation of RP11-53M11 BAC SOX17-Reporter:

EGFP and floxed neo was knocked into the SOX17 gene locus in a human genomic BAC RP11-53M11 by replacing the coding sequence of SOX17 (ATG to TGA) with the coding sequence of GFP and also the floxed neo. FIG. 1A schematically illustrates the structure of the recombinant construct. The BAC includes approximately 28.3 kb of SOX17 upstream sequence (SEQ ID NO:36) and approximately 132 kb of the SOX17 downstream sequence (SEQ ID NO:37). The sequence of the recombinant BAC (SEQ ID NO:1) includes the GFP coding sequence (SEQ ID NO:2), along with the SV40 poly A sequence (SEQ ID NO:27), a loxP site (SEQ ID NO:4), the neomycin resistance gene sequence (SEQ ID NO:3) with the SV40 early promoter/enhancer (SEQ ID NO:28), and the TK polyadenylation site (SEQ ID NO:29), a second loxP site (SEQ ID NO:4), encompassed by the human endogenous SOX17 upstream and downstream regulatory sequences.

Preparation of SOX17-GFP Knock in (KI) Plasmid Construct—

The present inventors have then subcloned out a region of the recombinant RP11-53M11 BAC SOX17-reporter BAC into a plasmid in order to generate a gene targeting vector (SEQ ID NO:6). The genomic sequence encompassing the SOX17-GFP sequence (from nucleotide 15487 of SEQ ID NO:1 to nucleotide 32492 of SEQ ID NO:1) was subcloned into the Bluescript plasmid (Stratagene) (FIG. 1B, the sequence marked as 5′-arm, EGFP, Floxed Neo and 3′-arm). The 5′-arm includes about 9 kb of SOX17 upstream regulatory sequence (SEQ ID NO:7) and the 3′-arm includes about 5 kb of SOX17 downstream regulatory sequence (SEQ ID NO:8).

Two 3′-external probes were prepared (SEQ ID NOs:9 and 10) in order to detect correct targeting of the vector into the genomic DNA of the host cell. For example, Southern blot screening of targeting at the 3′ end, the 3′-external probe (2) (SEQ ID NO:10) is used on XbaI digested genomic DNA (gDNA). The expected wild type band is 16.7 kb and the expected targeted band is 10.3 kb. The vector can be linearized with NruI. The targeted clone should be ampicillin and kanamycin resistant.

Preparation of BAC SOX17-Reporter RP11-47H10:

EGFP and floxed neo was knocked into the SOX17 gene locus in a human genomic BAC RP11-47H10 by replacing the coding sequence of SOX17 (ATG to TGA) with the coding sequence of GFP and the floxed neo. FIG. 1C schematically illustrates the structure of the recombinant construct. There is approximately 74 kb upstream of the SOX17 upstream regulatory sequence (SEQ ID NO:38) and approximately 73 kb of the downstream regulatory sequence (SEQ ID NO:39). The sequence of the recombinant BAC (SEQ ID NO:5) includes the GFP coding sequence (SEQ ID NO:2), along with the SV40 poly A sequence (SEQ ID NO:27), a loxP site (SEQ ID NO:4), the neomycin resistance gene sequence (SEQ ID NO:3) with the SV40 early promoter/enhancer (SEQ ID NO:28), and the TK polyadenylation site (SEQ ID NO:29), a second loxP site (SEQ ID NO:4), encompassed by the human endogenous SOX17 upstream (SEQ ID NO:38) and downstream (SEQ ID NO:39) regulatory sequences.

Preparation of SOX17-GFP Knock-in hESC Clones and Embryoid Bodies Differentiated Therefrom—

The RP11-47H10 GFP reporter BAC was electroporated into hESCs and G418 resistant clones were isolated. The various clones were allowed to form EBs in the presence of activin A (66 ng/ml) and at day 2, the EBs were dissociated into single cells and were analysed by FACS (FIG. 2A) and by fluorescent microscopy (FIGS. 2B-2E). It was previously shown by several groups that high concentrations of activin A induced hESCs to differentiate into definitive endoderm. SOX17 is one of the endodermal genes that are induced using such a protocol, which initiates the path toward insulin producing cell differentiation (19, 20). As shown, there was a clear population of GFP expressing cells in the different clones which varied from about 5% to about 40% (FIG. 2A). Next, an 80% confluent monolayer of the SOX17-GFP reporter line was treated with activin A (66 ng/ml) for three days after which immunohistochemistry was performed. The staining was carried out with anti-SOX17 (red) and anti-GFP (green) and demonstrated a clear correlation between SOX17 and GFP expression (FIG. 2E) thus confirming that the GFP⁺ cells indeed represent the SOX17⁺ population. The present inventors further stained the cells for both OCT4 (green), which marks undifferentiated cells, and SOX17 (red), marking the definitive endoderm population (FIG. 2G). Cells expressing OCT4 indicating the pluripotent state, were not positive for SOX17 and conversely, cells expressing SOX17 had turned off their OCT4 expression.

Gene Expression Analysis of SOX17+ Cells—

The SOX17-GFP reporter cells were grown as a monolayer in the presence of activin A for two days and then they were FACS-sorted into GFP⁺ and GFP⁻ populations. RNA was extracted from both populations and cDNA was synthesized. qPCR was performed comparing the relative expression of various genes between the GFP⁺ and the GFP⁻ cell populations (FIG. 2H). The present inventors were able to demonstrate that the GFP⁺ cells were enriched for SOX17 expression as compared to the GFP⁻ cell population, thus confirming that the GFP positive cells indeed represent a SOX17 positive cell population. Moreover, in the GFP⁺ cell population, an upregulation of genes which are related to definitive endoderm or mesendoderm such as chemokine receptor 4 (CXCR4), Cerebrus (CER), Goosecoid (GSC), CD34, and hepatocyte nuclear factor 3 beta (FOXA2) was observed, while expression of genes, such as NANOG which is primarily a marker of undifferentiated cells, was clearly decreased in the GFP⁺ population. These results further validate that the GFP+ sorted population indeed represent definitive endoderm or mesendoderm.

Transcriptional Profiling of SOX17⁺ Cells—

Microarray analysis was performed on the SOX17⁺ fraction using an Affymetrix Human Gene ST1.0 chip to obtain a transcriptional profile of this precursor population. Tables 1-2 hereinbelow summarize the findings of the microarray analysis, with information regarding membrane-associated genes (membranal and cell surface markers) which are upregulated (Table 1) and downregulated (Table 2) in the SOX17+ subpopulation of cells as compared to the genetically un-modified hESCs. Tables 3-4 hereinbelow summarize the findings of the microarray analysis, with information regarding non-membrane-associated genes (e.g., non-membranal, intracellular, secreted, transcription factors and the like) which are upregulated (Table 3) and downregulated (Table 4) in the SOX17+ subpopulation of cells as compared to the genetically un-modified hESCs. Examples of transcription factors and growth factors that were enriched in the SOX17⁺ expressing cells include FOXA2, bone morphogenetic protein 2 (BMP2), frizzled homolog 5 (FZD5), HNF1 homeobox B (HNF1b), GATA binding protein 3 (GATA3), paired box 3 (PAX3), vascular endothelial growth factor A (VEGFA) and Kruppel-like factor 8 (KLF8). Examples of secreted proteins that were enriched in the SOX17+ expressing cells include insulin-like growth factor binding protein 5 (IGFBP5), Apolipoprotein A-I (ApoA1) and apolipoprotein B (APOB). Examples of transmembrane proteins enriched in this population include CXCR4 (as was previously reported), CLAUDIN 18, cholecystokinin B receptor (CCKBR), leukemia inhibitory factor receptor alpha (LIFR), G protein-coupled receptor 141 (GPR141), G protein-coupled receptor 128 (GPR128), follicle stimulating hormone receptor (FSHR), CD34, and bone marrow stromal cell antigen 2 (BST2).

TABLE 1
Membranal genes which are upregulated in SOX17+ cells as compared to genetically un-modified hESCs

| | | | | SEQ ID | | |
|---|---|---|---|---|---|---|
| | | | | NO: (Of | | |
| | | Affy. target nucl. | | Rep. Public | | Log 2 fold change |
| Gene Symbol | Probe Set ID | SEQ ID NO: | Rep. Public ID | ID) | Polyn. SEQ ID NOs: rep. by target | [SOX17-HESC] |
| TRPA1 | 8151341 | 44 | NM_007332 | 107 | 4060, 8269, 5433, | +4.18 |
| SLC40A1 | 8057677 | 45 | NM_014585 | 108 | 3154, 4368, 6969, 5406, 7045, | +3.69 |
| | | | | | 8144, 8627, 7674, 6595, 6989, | |
| SLC30A10 | 7924342 | 46 | NM_018713 | 109 | 3493, 3718, 6268, 6130, 8273, | +3.65 |
| | | | | | 8216, | |
| CCKBR | 7938090 | 47 | NM_176875 | 110 | 2809, 5307, 5676, | +3.50 |
| VIPR2 | 8165658 | 48 | X59268 | 111 | 2553, 3426, 3422, 6269, | +3.45 |
| COLEC12 | 8021946 | 49 | NM_130386 | 112 | 2891, 4601, 6579, | +3.12 |
| FLRT3 | 8065071 | 50 | NM_198391 | 113 | 2812, 5172, 5173, 5918, 7056, | +3.11 |
| | | | | | 6380, | |
| LGR5 | 7957140 | 51 | NM_003667 | 114 | 2732, 3193, 6795, 5509, | +3.09 |
| GPR141 | 8132347 | 52 | NM_181791 | 115 | 3313, 3318, 6342, 5701, 6692, | +3.04 |
| BST2 | 8035304 | 53 | NM_004335 | 116 | 3090, 2805, 5403, 7124, | +3.02 |
| SLC5A9 | 7901316 | 54 | NM_001011547 | 117 | 4682, 4626, 4627, 6649, 6403, | +2.94 |
| | | | | | 7403, 7829, 6781, 6361, | |
| GPR128 | 8081298 | 55 | NM_032787 | 118 | 3995, 4847, 5470, 7733, 8308, | +2.93 |
| | | | | | 8445, | |
| KEL | 8143534 | 56 | NM_000420 | 119 | 2886, 2987, 6934, 8187, 8288, | +2.91 |
| | | | | | 6818, 6144, 8559, | |
| LRIG3 | 7964602 | 57 | NM_153377 | 120 | 3457, 3537, 4639, 5656, 6683, | +2.89 |
| | | | | | 7048, | |
| LYPD6B | 8045664 | 58 | NM_177964 | 121 | 3039, 3538, 8714, 5464, 7286, | +2.83 |
| | | | | | 6455, 7123, 7806, 7494, 8341, | |
| | | | | | 7067, 6613, | |
| FSHR | 8052072 | 59 | NM_000145 | 122 | 4265, 5021, 5022, 6252, 5713, | +2.73 |
| | | | | | 7612, 7221, 6397, | |
| LIFR | 8111677 | 60 | NM_001127671 | 123 | 2551, 4403, 4404, 5491, 7187, | +2.72 |
| | | | | | 7919, 8314, 7758, | |
| FOLH1 | 7948058 | 61 | NM_153696 | 124 | 3060, 3615, 2779, 2843, 3871, | +2.70 |
| | | | | | 2598, 4703, 4705, 4140, 4138, | |
| | | | | | 2773, 3026, 3280, 5177, 5176, | |
| | | | | | 2766, 5178, 3821, 6173, 5921, | |
| | | | | | 6307, 5425, 6469, 6968, | |
| CXCR4 | 8055465 | 62 | NM_001008540 | 125 | 2782, 3775, 3776, 5384, 6834, | +2.69 |
| | | | | | 7645, | |
| ITGA5 | 7963786 | 63 | NM_002205 | 126 | 3341, 3761, 5661, 6880, | +2.58 |
| AMOT | 8174576 | 64 | NM_001113490 | 127 | 2826, 4332, 3837, 7904, 5933, | +2.55 |
| | | | | | 6751, 5641, 6093, 6295, | |
| LY6E | 8148572 | 65 | NM_001127213 | 128 | 2668, 4370, 4371, 8116, 5582, | +2.47 |
| | | | | | 7083, 8656, 8711, 8353, 7871, | |
| | | | | | 7878, | |
| SEMA6D | 7983527 | 66 | NM_024966 | 129 | 3078, 3079, 3080, 3081, 3082, | +2.46 |
| | | | | | 4192, 4279, 3184, 3186, 3187, | |
| | | | | | 3185, 3188, 3183, 6040, 7595, | |
| | | | | | 5709, 6809, 6877, 7470, 6289, | |
| | | | | | 5994, | |
| GJA5 | 7919340 | 67 | NM_005266 | 130 | 3364, 4104, 4105, 7691, 6103, | +2.46 |
| | | | | | 6550, 5474, | |
| PRTG | 7989073 | 68 | NM_173814 | 131 | 3710, 4778, 6379, | +2.42 |
| CD34 | 7923978 | 69 | NM_001773 | 132 | 2563, 3891, 3854, 5956, 6086, | +2.40 |
| | | | | | 5785, 6059, 8087, | |
| TMEM144 | 8098041 | 70 | NM_018342 | 133 | 2802, 3163, 3170, 3261, 3319, | +2.40 |
| | | | | | 3506, 3267, 4584, 8697, 8424, | |
| | | | | | 5686, 8259, 8744, 8370, 7977, | |
| | | | | | 8192, 7923, 7849, 8039, 7954, | |
| | | | | | 8413, 7830, | |
| ROR2 | 8162283 | 71 | NM_004560 | 134 | 3804, 4102, 2608, 3151, 3287, | +2.35 |
| | | | | | 5241, 6393, 7306, 6756, 8135, | |
| GPR177 | 7916862 | 72 | NM_024911 | 135 | 3473, 5169, 5171, 5170, 6985, | +2.33 |
| | | | | | 7944, 8040, 5423, 6217, 5957, | |
| | | | | | 7720, 6063, 8128, 6006, | |
| OR2T4 | 7911273 | 73 | NM_001004696 | 136 | 3717, 6107, | +2.33 |
| SLC7A7 | 7977786 | 74 | NM_001126105 | 137 | 2789, 5292, 5291, 7158, 7255, | +2.32 |
| | | | | | 5870, 8255, 6471, 7642, | |
| KCNJ3 | 8045795 | 75 | NM_002239 | 138 | 2667, 3201, 5594, 8178, | +2.31 |
| CLDN18 | 8082928 | 76 | NM_016369 | 139 | 4183, 3788, 3787, 8686, 5317, | +2.29 |
| | | | | | 6211, | |
| TMEM106C | 7955063 | 77 | NM_024056 | 140 | 3447, 4690, 4693, 4691, 4692, | +2.22 |
| | | | | | 5419, 6591, 7332, | |
| GPR151 | 8114896 | 78 | NM_194251 | 141 | 3379, 3780, 5572, | +2.21 |
| SLC44A5 | 7917052 | 79 | NM_152697 | 142 | 3153, 4453, 4454, 7183, 6087, | +2.18 |
| | | | | | 6160, | |
| CDH10 | 8111255 | 80 | NM_006727 | 143 | 2788, 5142, 8497, 5507, 8253, | +2.18 |
| | | | | | 8371, | |
| TMEM27 | 8171472 | 81 | NM_020665 | 144 | 2960, 4942, 6430, | +2.17 |
| SLC1A1 | 8154135 | 82 | NM_004170 | 145 | 3070, 5025, 5401, 7218, 8331, | +2.13 |
| TMEM56 | 7903162 | 83 | NM_152487 | 146 | 4133, 4607, 6302, 7020, 6966, | +2.11 |
| | | | | | 6405, | |
| CD177 | 8037298 | 84 | NM_020406 | 147 | 3744, 4005, 6408, 7480, 7666, | +2.09 |
| PLXNA2 | 7923991 | 85 | NM_025179 | 148 | 4111, 4029, 6303, 8403, 6499, | +2.08 |
| SLC26A2 | 8109194 | 86 | NM_000112 | 149 | 3484, 3972, 5711, 7134, | +2.05 |
| DSCAM | 8070421 | 87 | NM_001389 | 150 | 2796, 3591, 6842, | +2.04 |
| TMEM133 | 7943369 | 88 | NM_032021 | 151 | 2860, 3175, 5703, | +2.02 |
| IL13RA1 | 8169580 | 89 | NM_001560 | 152 | 2683, 3219, 6256, 6220, 8730, | +2.00 |
| | | | | | 6320, | |
| ATP2B1 | 7965359 | 90 | NM_001001323 | 153 | 2655, 3664, 3663, 6331, 5399, | +1.92 |
| | | | | | 6047, 6021, 6425, | |
| CD302 | 8056102 | 91 | NM_014880 | 154 | 3314, 3316, 3317, 3215, 2603, | +1.92 |
| | | | | | 4536, 3431, 3435, 4256, 3004, | |
| | | | | | 4660, 5416, 6685, 8428, 8482, | |
| | | | | | 7955, | |
| MEGF9 | 8163775 | 92 | NM_001080497 | 155 | 4704, 5185, 6235, 6965, | +1.91 |
| EDNRA | 8097692 | 93 | NM_001957 | 156 | 2600, 4920, 4921, 4922, 8628, | +1.89 |
| | | | | | 5591, 5960, 8235, 5962, 8220, | |
| | | | | | 6628, 7962, | |
| CDH2 | 8022674 | 94 | NM_001792 | 157 | 3134, 4668, 5546, | +1.88 |
| GPR161 | 7922108 | 95 | NM_153832 | 158 | 3044, 3194, 7409, 6232, 7729, | +1.86 |
| | | | | | 6084, 8332, 5530, 5999, | |
| TYRO3 | 7982938 | 96 | NM_006293 | 159 | 2646, 5109, 5565, | +1.85 |
| FLRT2 | 7976073 | 97 | NM_013231 | 160 | 4059, 3816, 5667, | +1.82 |
| LRIT3 | 8096839 | 98 | NM_198506 | 161 | 3439, 4440, 5693, 7042, 6600, | +1.82 |
| PCDH7 | 8094520 | 99 | NM_032457 | 162 | 2723, 5014, 5015, 2937, 5262, | +1.80 |
| | | | | | 5768, 5971, | |
| NRCAM | 8142270 | 100 | NM_001037132 | 163 | 3565, 3859, 3993, 3989, 3309, | +1.79 |
| | | | | | 3102, 3441, 4484, 5180, 5181, | |
| | | | | | 5182, 5183, 5184, 8267, 7609, | |
| | | | | | 7127, 6412, 6732, 7550, 6919, | |
| | | | | | 8165, 7672, 6704, 6312, 7407, | |
| | | | | | 7189, 7606, 6991, 7159, 6420, | |
| | | | | | 6482, 6987, | |
| SMAGP | 7963280 | 101 | NM_001033873 | 164 | 3242, 3886, 3885, 6421, 7267, | +1.73 |
| AMHR2 | 7955797 | 102 | NM_020547 | 165 | 4056, 4904, 4903, 4905, 7400, | +1.73 |
| | | | | | 5395, | |
| ELTD1 | 7917182 | 103 | NM_022159 | 166 | 3027, 4175, 6126, 7630, | +1.73 |
| GRPR | 8166202 | 104 | NM_005314 | 167 | 2579, 3797, 6967, | +1.73 |
| EPHA4 | 8059279 | 105 | NM_004438 | 168 | 3926, 3577, 6427, 7497, 5543, | +1.72 |
| CD99 | 8165794 | 106 | NM_001122898 | 169 | 3639, 2591, 2693, 3374, 2887, | +1.70 |
| | | | | | 3376, 3010, 3030, 4292, 4358, | |
| | | | | | 3378, 8134, 6730, 8768, 6667, | |
| | | | | | 6606, 7512, 7524, | |

Table 1. Provided are the description of the membranal genes along with the sequence identifiers of the Affymetrix probes, the target sequence identified by the Affymetrix probe (Affy. target nucl. SEQ ID NO:), and the mRNA sequences represented by the target sequences. Also shown are the log 2 fold change in expression levels of the indicated genes between the SOX17 GFP cells and the genetically unmodified HESCs. The (+) or (−) sign means that the genes are upregulated or downregulated, respectively, in the SOX17-GFP cells as compared to the genetically unmodified HESCs.

TABLE 2
Membranal genes which are downregulated in SOX17+ cells as compared to genetically un-modified ESCs

| | | Affy. | | | | Log 2 fold |
|---|---|---|---|---|---|---|
| | | target | | SEQ ID NO: | | change |
| Gene | | nucl. SEQ | | (of Rep. | | [SOX17- |
| Symbol | Probe Set ID | ID NO: | Rep. Public ID | Public ID) | Polyn. SEQ ID NOs: rep. by target | HESC] |
| KDR | 8100393 | 170 | NM_002253 | 204 | 2708, 4588, 7921, 5498, 8228, | −4.30 |
| PCDHB5 | 8108697 | 171 | NM_015669 | 205 | 2872, 2931, 5379, | −3.42 |
| FAT4 | 8097288 | 172 | NM_024582 | 206 | 3507, 4331, 5697, 7491, | −2.99 |
| FLT1 | 7970763 | 173 | NM_001159920 | 207 | 3763, 4816, 4818, 4819, 4817, 5510, | −2.88 |
| NRN1 | 8123739 | 174 | NM_016588 | 208 | 3230, 3454, 7755, 5387, | −2.87 |
| THBS2 | 8130867 | 175 | NM_003247 | 209 | 4184, 3540, 8063, 7661, 5992, 6904, | −2.85 |
| PTPRZ1 | 8135774 | 176 | NM_002851 | 210 | 2599, 3943, 7457, 8030, 7815, 8011, 8237, 7646, | −2.79 |
| SLC6A15 | 7965206 | 177 | NM_001146335 | 211 | 3618, 4807, 4688, 4689, 5536, 5746, 5761, 7293, | −2.77 |
| GPR176 | 7987439 | 178 | NM_007223 | 212 | 3580, 2783, 5852, | −2.72 |
| SEMA6A | 8113666 | 179 | NM_020796 | 213 | 2844, 4722, 3122, 3148, 4310, 4145, 8137, 8152, | −2.68 |
| | | | | | 7900, 5499, 8724, 8667, 8467, 8111, 5437, 8321, | |
| | | | | | 7896, 5978, 8764, | |
| THBS1 | 7982597 | 180 | NM_003246 | 214 | 4759, 3539, 7996, 5434, 8028, 7563, 6593, 8344, | −2.63 |
| CDH11 | 8001800 | 181 | NM_001797 | 215 | 2639, 2970, 5559, 6928, | −2.51 |
| GRID2 | 8096440 | 182 | NM_001510 | 216 | 2719, 4235, 8429, 5557, 7933, 8154, | −2.51 |
| SLC7A11 | 8102800 | 183 | NM_014331 | 217 | 2916, 3899, 8531, 5555, | −2.46 |
| CDH1 | 7996837 | 184 | NM_004360 | 218 | 4149, 4182, 2614, 4278, 4556, 2794, 2795, 4415, | −2.46 |
| | | | | | 4294, 4315, 4347, 5506, 7453, 5404, 7133, | |
| LRFN5 | 7974109 | 185 | NM_152447 | 219 | 4563, 4583, 5622, | −2.45 |
| EDNRB | 7972157 | 186 | NM_003991 | 220 | 2606, 5247, 5246, 5248, 5597, 7819, 7369, | −2.44 |
| THY1 | 7952268 | 187 | NM_006288 | 221 | 3096, 4729, 5694, | −2.36 |
| NETO1 | 8023828 | 188 | NM_138999 | 222 | 3269, 3045, 5252, 5646, 6813, 5692, | −2.28 |
| KCND2 | 8135705 | 189 | NM_012281 | 223 | 3907, 3225, 5604, 8488, | −2.28 |
| TMPRSS11E | 8095380 | 190 | NM_014058 | 224 | 3472, 4222, 5579, 8653, | −2.21 |
| TMPRSS11E | 8095364 | 191 | NM_014058 | 225 | 3472, 4222, 5579, 8653, | −2.19 |
| CD44 | 7939341 | 192 | NM_000610 | 226 | 3067, 3068, 2739, 3276, 4538, 2566, 2586, 2564, | −2.17 |
| | | | | | 2565, 2660, 4282, 4552, 4163, 4631, 2890, 3277, | |
| | | | | | 3579, 3658, 3659, 3660, 3661, 3662, 6650, 7581, | |
| | | | | | 7244, 6542, 7096, 5461, 6031, 7241, 6225, 7493, | |
| | | | | | 6898, 5515, 6679, 5545, 5516, | |
| PDPN | 7898057 | 193 | NM_001006624 | 227 | 3236, 2746, 2747, 3084, 3519, 2965, 3133, 4305, | −2.08 |
| | | | | | 4306, 3747, 3748, 3745, 3746, 8458, 8786, 8213, | |
| | | | | | 5840, 7528, 8392, 8050, 7659, 7822, 8446, | |
| SLC7A1 | 7970810 | 194 | NM_003045 | 228 | 3517, 4636, 6881, | −2.08 |
| KAL1 | 8171248 | 195 | NM_000216 | 229 | 2607, 4088, 5560, 8200, | −2.07 |
| KCNG3 | 8051785 | 196 | NM_133329 | 230 | 3647, 5227, 5228, 5783, 7561, | −2.05 |
| GPM6B | 8171359 | 197 | NM_005278 | 231 | 4714, 3686, 3685, 3687, 3684, 6150, 8294, 6043, | −2.04 |
| | | | | | 5590, 7105, | |
| FXYD5 | 8027778 | 198 | NM_144779 | 232 | 3228, 3470, 2828, 3419, 3286, 4528, 3008, 3388, | −1.96 |
| | | | | | 4895, 4896, 4898, 4897, 6381, 7395, 6565, 5906, | |
| | | | | | 7311, 8512, | |
| PCDH18 | 8102792 | 199 | NM_019035 | 233 | 4262, 4225, 5888, 7284, | −1.93 |
| ICAM3 | 8033987 | 200 | NM_002162 | 234 | 3247, 4340, 5318, | −1.92 |
| MCTP1 | 8113130 | 201 | NM_024717 | 235 | 3688, 3689, 4710, 3372, 4621, 4622, 8584, 8536, | −1.89 |
| | | | | | 8020, 8738, 8599, 7737, 8789, 5624, 7945, 7981, | |
| | | | | | 7997, 8146, 6686, 7715, 8769, 8630, 7018, | |
| TACR3 | 8102127 | 202 | NM_001059 | 236 | 2592, 5166, 5672, | −1.82 |
| TMEM155 | 8102636 | 203 | NM_152399 | 237 | 3259, 4659, 4024, 8590, 7444, 8029, 8685, 7009, | −1.79 |
| | | | | | 7201, 8783, 5815, 7282, | |

Table 2. Provided are the description of the membranal genes along with the sequence identifiers of the Affymetrix probes, the target sequence identified by the Affymetrix probe, and the mRNA sequences represented by the target sequences. Also shown are the log 2 fold change in expression levels of the indicated genes between the SOX17 GFP cells and the genetically unmodified HESCs. The (+) or (−) sign means that the genes are upregulated or downregulated, respectively, in the SOX17-GFP cells as compared to the genetically unmodified HESCs.

TABLE 3
Non-membranal genes which are up-regulated in SOX17+ cells as compared to genetically un-modified ESCs

| | | Affy. target | | SEQ ID NO: | | Log 2 fold |
|---|---|---|---|---|---|---|
| Gene | Probe | nucl. SEQ | Rep. | (Of Rep. | Polyn. SEQ ID NOs: | change |
| Symbol | Set ID | ID NO: | Public ID | Public ID) | rep. by target | [SOX17-HESC] |
| GATA3 | 7926105 | 238 | NM_002051 | 530 | 2915, 3695, 3696, 6780, 6049, 7381, | +4.48 |
| SEMA3E | 8140650 | 239 | NM_012431 | 531 | 4218, 5112, 5113, 5625, 7193, | +4.11 |
| HHEX | 7929282 | 240 | NM_002729 | 532 | 2961, 4124, 7867, 5797, 8612, | +3.94 |
| ZNF280A | 8074853 | 241 | NM_080740 | 533 | 3304, 3570, 5866, | +3.88 |
| FAM184A | 8129231 | 242 | NM_024581 | 534 | 3503, 4208, 4209, 8021, 7766, 5900, 8495, | +3.82 |
| | | | | | 6262, | |
| WDR72 | 7988990 | 243 | NM_182758 | 535 | 3418, 4240, 6598, 6055, | +3.80 |
| PDZK1 | 7904843 | 244 | NM_002614 | 536 | 3336, 5243, 4204, 7461, 7260, 6532, 6226, | +3.75 |
| | | | | | 7090, 6900, 7707, 7472, 6465, | |
| RLBP1L2 | 8121803 | 245 | NM_001010852 | 537 | 3116, 3869, 5483, 6248, | +3.66 |
| SHISA2 | 7970676 | 246 | NM_001007538 | 538 | 4110, 3758, 5588, | +3.57 |
| VIL1 | 8048319 | 247 | NM_007127 | 539 | 4477, 4578, 5439, | +3.51 |
| STMN2 | 8147030 | 248 | NM_007029 | 540 | 4711, 5225, 5342, | +3.51 |
| APOA2 | 7921834 | 249 | NM_001643 | 541 | 2901, 2758, 6253, 8161, 7460, 7113, 8544, | +3.44 |
| | | | | | 6696, 7530, 7963, 6627, 6449, | |
| SERHL | 8073585 | 250 | NM_014509 | 542 | 4046, 3113, 3203, 4870, 6789, 7257, 7696, | +3.42 |
| | | | | | 6190, 7885, 6148, 8055, 5575, | |
| PPFIBP2 | 7938231 | 251 | NM_003621 | 543 | 3011, 4355, 5626, | +3.39 |
| DKK1 | 7927631 | 252 | NM_012242 | 544 | 3471, 3796, 7543, 8463, 8242, 5959, | +3.38 |
| MUM1L1 | 8169145 | 253 | NM_152423 | 545 | 3142, 4991, 4993, 6316, 5894, 5576, | +3.33 |
| IGFBP5 | 8058857 | 254 | NM_000599 | 546 | 2583, 4357, 8723, 5360, | +3.28 |
| ST6GALNAC2 | 8018761 | 255 | NM_006456 | 547 | 2638, 4437, 5345, | +3.27 |
| TSPYL5 | 8151931 | 256 | NM_033512 | 548 | 3241, 3675, 5757, | +3.26 |
| STC1 | 8149825 | 257 | NM_003155 | 549 | 2725, 3793, 5771, | +3.25 |
| SYTL5 | 8166747 | 258 | NM_001163335 | 550 | 3411, 4887, 4885, 4886, 5912, 6888, 5629, | +3.19 |
| EPSTI1 | 7971296 | 259 | NM_033255 | 551 | 3367, 3694, 3693, 5738, 5732, 7664, 7742, | +3.18 |
| ANKRD1 | 7934979 | 260 | NM_014391 | 552 | 2995, 3511, 5932, | +3.18 |
| ARHGAP24 | 8096160 | 261 | NM_001025616 | 553 | 3856, 4013, 4014, 4012, 8287, 7220, 7970, | +3.08 |
| | | | | | 6456, 8329, 8596, 5517, 8693, 8490, | |
| KRT18P49 | 8169233 | 262 | ENST00000427083 | 554 | 7599, | +3.07 |
| PRSS2 | 8136790 | 263 | — | | | +3.05 |
| RHOBTB3 | 8106986 | 264 | NM_014899 | 555 | 3394, 4041, 7121, 8530, 7782, 8683, 8248, | +3.05 |
| | | | | | 7828, 8218, 8489, | |
| FRZB | 8057506 | 265 | NM_001463 | 556 | 2679, 5106, 5695, | +3.05 |
| RARB | 8078286 | 266 | NM_016152 | 557 | 2681, 3505, 4596, 4598, 8684, 7033, 8333, | +3.03 |
| | | | | | 6707, 5820, 6360, 7180, | |
| ADAMTS9 | 8088560 | 267 | NM_182920 | 558 | 4100, 3333, 8622, 8439, 8457, 6636, 7795, | +3.01 |
| | | | | | 8233, 8784, | |
| ARL4D | 8007493 | 268 | NM_001661 | 559 | 3037, 3901, 5663, | +3.00 |
| PRDM1 | 8121257 | 269 | NM_182907 | 560 | 3253, 5303, 4361, 6228, 6168, 8184, 8624, | +2.96 |
| | | | | | 7700, | |
| HP | 7997188 | 270 | NM_005143 | 561 | 3893, 4016, 4018, 2580, 2622, 2977, 2992, | +2.96 |
| | | | | | 3453, 3617, 4365, 4366, 6921, 6310, 6000, | |
| | | | | | 6001, | |
| FZD5 | 8058498 | 271 | NM_003468 | 562 | 2934, 4015, 5838, | +2.95 |
| TRY6 | 8136807 | 272 | — | | | +2.94 |
| ATP6V0D2 | 8147145 | 273 | NM_152565 | 563 | 3548, 3146, 7959, 8150, 5593, | +2.93 |
| ANGPT2 | 8149071 | 274 | NM_001147 | 564 | 2696, 4344, 4345, 4346, 5744, 7577, | +2.92 |
| DENND2C | 7918768 | 275 | NM_198459 | 565 | 3586, 4103, 6796, 6943, 8236, 8046, 6164, | +2.88 |
| | | | | | 7594, | |
| BMP5 | 8127193 | 276 | NM_021073 | 566 | 2611, 3202, 5997, 7150, | +2.87 |
| FOXA2 | 8065344 | 277 | NM_021784 | 567 | 2770, 4577, 4466, 5823, 7393, 7364, 6571, | +2.83 |
| HAS2 | 8152617 | 278 | NM_005328 | 568 | 2676, 4348, 5843, 6409, | +2.80 |
| BMP2 | 8060850 | 279 | NM_001200 | 569 | 3900, 6567, | +2.79 |
| S100A16 | 7920291 | 280 | NM_080388 | 570 | 3782, 2999, 5985, 6066, 6285, 6298, 8082, | +2.79 |
| FOLH1B | 7942998 | 281 | NM_153696 | 571 | 3060, 2843, 3280, 5177, 2766, 5178, 6173, | +2.78 |
| | | | | | 5921, 6307, 5425, 6469, | |
| FAM122C | 8169995 | 282 | NM_001170779 | 572 | 4513, 4955, 4956, 4957, 4958, 4959, 4960, | +2.77 |
| | | | | | 4954, 6451, 7031, 8034, 6865, 6135, 8059, | |
| | | | | | 8191, 6912, 8587, 6236, | |
| FZD4 | 7950885 | 283 | NM_012193 | 573 | 2784, 5289, | +2.77 |
| C5 | 8163839 | 284 | NM_001735 | 574 | 3997, 3497, 8045, 5344, 6954, 6376, | +2.77 |
| S100A14 | 7920297 | 285 | NM_020672 | 575 | 2827, 2832, 8484, 5892, 5948, 6180, 7538, | +2.76 |
| | | | | | 6036, | |
| VEGFA | 8119898 | 286 | NM_001025366 | 576 | 3808, 4966, 4967, 4968, 4969, 4970, 4971, | +2.74 |
| | | | | | 4972, 4973, 4974, 4975, 4976, 4977, 4978, | |
| | | | | | 4979, 4980, 4981, 8712, 5968, 6175, 7640, | |
| | | | | | 5961, 5690, 7243, 8549, 6759, 6136, 7716, | |
| | | | | | 8136, 8355, 8159, 8018, 7079, 8270, 8120, | |
| | | | | | 7866, 8507, 8066, 7946, | |
| CLIP4 | 8041179 | 287 | NM_024692 | 577 | 4055, 4666, 6910, 6677, 7982, 7631, 5684, | +2.72 |
| | | | | | 7192, 6576, 6387, 7420, 8380, | |
| GPAM | 8165663 | 288 | AK172782 | 578 | 3609, | +2.70 |
| HNF1B | 8014591 | 289 | NM_000458 | 579 | 2989, 4226, 4915, 5347, 6709, 6537, | +2.69 |
| APOA1 | 7951865 | 290 | NM_000039 | 580 | 3902, 2762, 6306, 5375, 6654, 6990, | +2.68 |
| CFLAR | 8047381 | 291 | NM_003879 | 581 | 2698, 2700, 2701, 2702, 2703, 2697, 2727, | +2.67 |
| | | | | | 2728, 2729, 2714, 2715, 2716, 2717, 2718, | |
| | | | | | 3809, 3282, 4540, 4723, 2706, 2695, 2707, | |
| | | | | | 4275, 4518, 2881, 4319, 5254, 5255, 7718, | |
| | | | | | 7181, 7213, 6901, 7210, 7050, 6868, 7797, | |
| | | | | | 7093, 6246, 6301, 6416, 6026, 8088, 7173, | |
| | | | | | 6972, 6669, 7157, 7540, 7179, 6019, 5721, | |
| RBM24 | 8117045 | 292 | NM_153020 | 582 | 3092, 4733, 4735, 4734, 6592, 6373, 5606, | +2.66 |
| | | | | | 8008, 8572, 8110, | |
| RNF152 | 8023598 | 293 | NM_173557 | 583 | 3123, 3946, 5605, | +2.66 |
| TTR | 8020795 | 294 | NM_000371 | 584 | 2722, 4730, 6739, 5362, | +2.66 |
| TTN | 8057056 | 295 | NM_133378 | 585 | 3308, 2656, 3424, 4106, 4107, 3344, 3048, | +2.63 |
| | | | | | 5008, 5009, 5011, 5007, 5010, 7368, 5887, | |
| | | | | | 5945, 7339, 6830, 8194, 6950, 6315, 6321, | |
| | | | | | 6481, 7366, 6697, 6793, 7546, 6131, 5681, | |
| EGFLAM | 8105013 | 296 | NM_152403 | 586 | 3530, 5277, 5276, 5275, 6119, 5859, 8466, | +2.62 |
| | | | | | 5742, 6783, 8384, 6717, 7825, 8629, 8422, | |
| | | | | | 7942, 6265, | |
| APOB | 8050619 | 297 | NM_000384 | 587 | 2558, 3976, 5351, | +2.62 |
| DIO3 | 7976858 | 298 | NM_001362 | 588 | 2990, 4161, 8265, 6088, | +2.61 |
| IFLTD1 | 7961875 | 299 | NM_152590 | 589 | 4498, 4781, 4782, 4784, 4785, 4783, 5790, | +2.60 |
| | | | | | 7143, 6544, 6853, | |
| ABCC4 | 7972297 | 300 | NM_005845 | 590 | 3298, 4237, 4238, 6825, 6506, 6826, 6736, | +2.58 |
| | | | | | 7298, | |
| CCDC141 | 8057377 | 301 | NM_173648 | 591 | 3425, 5152, 5914, 5666, 7929, | +2.58 |
| ENC1 | 8112615 | 302 | NM_003633 | 592 | 2731, 4418, 7756, 5853, 8481, | +2.57 |
| NEK2 | 7924096 | 303 | NM_002497 | 593 | 3232, 5258, 5965, 7142, 6196, | +2.57 |
| ELMO1 | 8139057 | 304 | NM_014800 | 594 | 2968, 3692, 4045, 2861, 3438, 4258, 4520, | +2.56 |
| | | | | | 4479, 2801, 2880, 3542, 5280, 5279, 8359, | |
| | | | | | 7086, 5936, 6939, 6791, 6584, 7972, 7197, | |
| | | | | | 7051, 6279, 5770, 6974, 6770, 7488, 7401, | |
| | | | | | 7549, 6438, 7349, | |
| SPOCK3 | 8103544 | 305 | NM_016950 | 595 | 3468, 4487, 4491, 4493, 4496, 4500, 4505, | +2.54 |
| | | | | | 4506, 4522, 2959, 3354, 2879, 3351, 3951, | |
| | | | | | 3952, 7390, 8151, 7745, 8674, 7862, 8555, | |
| | | | | | 8291, 8542, 7775, 8670, 8212, 7911, 5944, | |
| | | | | | 8680, 8411, 8508, 6200, 8249, 7909, 8528, | |
| | | | | | 8673, 7951, 8415, 8750, 7722, | |
| SERPINI1 | 8083779 | 306 | NM_005025 | 596 | 4280, 4350, 4351, 7428, 8534, 7747, 5615, | +2.54 |
| | | | | | 7839, 7903, | |
| ACSL1 | 8103951 | 307 | NM_001995 | 597 | 2601, 4521, 2572, 4269, 4707, 4709, 3264, | +2.52 |
| | | | | | 3180, 3549, 8277, 8532, 8386, 8421, 8369, | |
| | | | | | 8009, 8062, 6545, 8440, 5566, 8524, 7826, | |
| | | | | | 7554, | |
| GATM | 7988414 | 308 | NM_001482 | 598 | 3346, 4123, 6982, | +2.52 |
| EHHADH | 8092523 | 309 | NM_001166415 | 599 | 3206, 4927, 4926, 5368, 8762, | +2.52 |
| NUDT4 | 7957536 | 310 | NM_199040 | 600 | 3968, 3967, | +2.50 |
| CST1 | 8065412 | 311 | NM_001898 | 601 | 3369, 3033, 5734, 6511, | +2.50 |
| GLUD2 | 8169711 | 312 | NM_012084 | 602 | 2899, 3400, 5864, | +2.47 |
| NPL | 7908003 | 313 | NM_030769 | 603 | 3446, 5244, 8301, 8566, 7329, 6079, 6297, | +2.47 |
| | | | | | 6185, 6208, 6540, 5453, 5873, 5974, 6575, | |
| ZNF702P | 8039025 | 314 | NR_003578 | 604 | 3075, 4190, 5550, | +2.45 |
| TRY6 | 8136801 | 315 | NM_002770 | 605 | 3326, 3327, 4043, 4044, 3896, 4082, 2633, | +2.45 |
| | | | | | 3056, 4031, 3057, 4349, 5199, 5294, 5200, | |
| | | | | | 4875, 7372, 5743, 6222, 8379, | |
| SPOCK1 | 8114287 | 316 | NM_004598 | 606 | 2804, 3903, 8553, 8505, 6817, 7957, 5469, | +2.41 |
| | | | | | 8014, 7754, | |
| AGL | 7903239 | 317 | NM_000028 | 607 | 2687, 4063, 4068, 4064, 4066, 4065, 4067, | +2.40 |
| | | | | | 6243, 5987, 6127, 5817, 5995, 5972, 8735, | |
| | | | | | 6008, | |
| TFF1 | 8070579 | 318 | NM_003225 | 608 | 2552, 3668, 5847, | +2.39 |
| DGKK | 8172670 | 319 | NM_001013742 | 609 | 3778, 5274, 6444, | +2.37 |
| SALL1 | 8001387 | 320 | NM_001127892 | 610 | 4497, 4411, 4078, 6646, 5432, 7163, | +2.36 |
| MANEA | 8121144 | 321 | NM_024641 | 611 | 4179, 5290, 6156, 5952, 8006, | +2.35 |
| KIT | 8095110 | 322 | NM_000222 | 612 | 3646, 4153, 4152, 5648, 8162, | +2.35 |
| KRT19 | 8015349 | 323 | NM_002276 | 613 | 3524, 4126, 7422, 8625, 5885, 6794, 6946, | +2.35 |
| TNNC1 | 8087925 | 324 | NM_003280 | 614 | 3054, 4613, 5365, 7708, | +2.35 |
| SEPP1 | 8111915 | 325 | NM_001093726 | 615 | 3112, 3121, 2969, 3211, 3248, 3487, 4155, | +2.33 |
| | | | | | 4154, 3820, 8621, 7799, 8519, 8258, 7809, | |
| | | | | | 8140, 7833, 8546, 8284, 8435, 8309, 7973, | |
| | | | | | 7743, 7917, 8097, | |
| ST8SIA4 | 8113358 | 326 | NM_005668 | 616 | 3302, 4599, 3244, 7055, 7738, 5386, | +2.32 |
| YPEL2 | 8008819 | 327 | NM_001005404 | 617 | 3716, 3768, 5719, | +2.32 |
| ANKMY2 | 8138370 | 328 | NM_020319 | 618 | 3159, 5132, 7035, 7445, 7172, 5729, | +2.31 |
| DNAJC15 | 7968872 | 329 | NM_013238 | 619 | 3842, 3846, 7013, 8073, | +2.31 |
| RNF128 | 8169174 | 330 | NM_024539 | 620 | 3527, 3478, 3479, 5441, 5712, 6719, | +2.31 |
| PTPN13 | 8096176 | 331 | NM_006264 | 621 | 2621, 4579, 4580, 4581, 4582, 6886, 7770, | +2.31 |
| | | | | | 7790, 7383, 8720, 7840, 6014, 5863, 7589, | |
| F10 | 7970241 | 332 | NM_000504 | 622 | 2567, 2573, 3210, 3246, 4308, 4309, 3935, | +2.30 |
| | | | | | 7894, 6670, 7728, 6624, 7953, 7352, 6493, | |
| SAMD3 | 8129482 | 333 | NM_001017373 | 623 | 3098, 4120, 4122, 7473, 7107, 6192, 5825, | +2.30 |
| | | | | | 7041, 6688, | |
| GCNT1 | 8155930 | 334 | NM_001490 | 624 | 2578, 4156, 4157, 4158, 4159, 4160, 8460, | +2.29 |
| | | | | | 6349, 8001, 7226, 6641, | |
| IPP | 7915775 | 335 | NM_016486 | 625 | 2772, 4084, 4775, 4774, 7978, 7647, 6202, | +2.28 |
| | | | | | 6163, | |
| PROS1 | 8089015 | 336 | NM_000313 | 626 | 2597, 4770, 6690, 8232, | +2.27 |
| SV2B | 7986195 | 337 | NM_001167580 | 627 | 3448, 4936, 4935, 5668, 6488, | +2.26 |
| PLOD2 | 8091283 | 338 | NM_182943 | 628 | 3144, 3823, 3824, 7950, 7025, 6032, 8398, | +2.24 |
| | | | | | 5670, 7845, | |
| MAGEH1 | 8167887 | 339 | NM_014061 | 629 | 2950, 3007, 6141, | +2.24 |
| CHST9 | 8022666 | 340 | NM_031422 | 630 | 3460, 4937, 5865, | +2.23 |
| ZNF518B | 8099364 | 341 | NM_053042 | 631 | 3218, 3781, 5718, 8226, 8286, 7880, 7968, | +2.23 |
| TMEM106C | 7955063 | 342 | NM_024056 | 632 | 3447, 4690, 4693, 4691, 4692, 5419, 6591, | +2.22 |
| | | | | | 7332, | |
| SERHL2 | 8073596 | 343 | NM_014509 | 633 | 3645, 4046, 4023, 4076, 4075, 4492, 4499, | +2.20 |
| | | | | | 3203, 7885, 6148, 6161, 6501, 7434, 5747, | |
| | | | | | 5575, | |
| NTN4 | 7965573 | 344 | NM_021229 | 634 | 2846, 3957, 6056, 5886, | +2.20 |
| SOX17 | 8146462 | 345 | NM_022454 | 635 | 3009, 4141, 5778, | +2.20 |
| FRRS1 | 7917954 | 346 | NM_001013660 | 636 | 3386, 4150, 5573, 8650, 7934, 6212, 6764, | +2.20 |
| OTX2 | 7979357 | 347 | NM_021728 | 637 | 3073, 3223, 3224, 6604, 6242, | +2.19 |
| RNASEL | 7922707 | 348 | NM_021133 | 638 | 3790, 5239, 6286, 6523, | +2.19 |
| ELMOD2 | 8097529 | 349 | NM_153702 | 639 | 3416, 5196, 8425, 7800, 5802, 8102, 8387, | +2.18 |
| | | | | | 8257, 8391, | |
| MYCT1 | 8122860 | 350 | NM_025107 | 640 | 3129, 4215, 6145, | +2.17 |
| PAX6 | 7947338 | 351 | NM_000280 | 641 | 3805, 4400, 4401, 4402, 6733, 7304, 6876, | +2.16 |
| | | | | | 7135, 7188, 7299, 6953, 7333, 7281, 8690, | |
| | | | | | 7478, 7234, 8432, 7017, 6745, 7558, 5383, | |
| MGST2 | 8097513 | 352 | NM_002413 | 642 | 3025, 5261, 8016, 7820, 8094, 5528, 8127, | +2.15 |
| BBS5 | 8046147 | 353 | NM_152384 | 643 | 3608, 3739, 8230, 6450, 7499, 7824, 5869, | +2.14 |
| | | | | | 6872, | |
| MTSS1 | 8152764 | 354 | NM_014751 | 644 | 2790, 4171, 7149, 7038, 8535, 8296, 7813, | +2.13 |
| | | | | | 8241, 8289, 8581, 5899, | |
| VTN | 8013606 | 355 | NM_000638 | 645 | 2897, 3932, 5348, | +2.13 |
| WBP5 | 8169022 | 356 | NM_001006613 | 646 | 3375, 3752, 3755, 3753, 3751, 6158, 6146, | +2.12 |
| DUB4 | 8094134 | 357 | AY533200 | 647 | 3572, 3865, 4780, 7780, 7776, 8514, 8351, | +2.12 |
| CCDC92 | 7967486 | 358 | NM_025140 | 648 | 3339, 2893, 5390, | +2.12 |
| BTG2 | 7908917 | 359 | NM_006763 | 649 | 2680, 3257, 8005, 5749, | +2.12 |
| LPGAT1 | 7924107 | 360 | NM_014873 | 650 | 3152, 4614, 7821, 6002, 6174, | +2.11 |
| FN1 | 8058765 | 361 | NM_054034 | 651 | 3732, 3409, 3983, 4677, 4679, 3284, 3310, | +2.11 |
| | | | | | 3403, 3404, 3405, 3406, 4727, 3110, 4551, | |
| | | | | | 4571, 4572, 4576, 3822, 3494, 2852, 3620, | |
| | | | | | 3621, 3622, 3623, 3624, 3625, 5996, 7280, | |
| | | | | | 7649, 6915, 8364, 8529, 6768, 7398, 5846, | |
| | | | | | 6424, 8736, 8430, 7036, 5993, 8153, 8487, | |
| | | | | | 6324, 6771, 6223, 6205, 5539, 6620, 6121, | |
| | | | | | 6206, 8147, 5687, | |
| TBX3 | 7966690 | 362 | NM_016569 | 652 | 3024, 3627, 3626, 6215, 5414, 6072, | +2.11 |
| PLCE1 | 7929388 | 363 | NM_016341 | 653 | 4217, 4071, 4918, 6138, 5440, 6045, 6266, | +2.11 |
| KRT19P2 | 7957608 | 364 | NM_002276 | 654 | 4928, 4126, 5201, 5885, 6917, | +2.10 |
| IFI16 | 7906400 | 365 | NM_005531 | 655 | 3138, 2735, 2792, 2582, 3114, 4519, 2986, | +2.10 |
| | | | | | 4028, 5903, 7297, 7065, 5990, 6176, 8454, | |
| | | | | | 7984, 8195, 7186, 7588, 5913, 5739, | |
| PORCN | 8167287 | 366 | NM_203473 | 656 | 2854, 2855, 2856, 2857, 4728, 2616, 4468, | +2.09 |
| | | | | | 2979, 2983, 3366, 3574, 3575, 3573, 3576, | |
| | | | | | 5975, 8479, 5883, 6961, 5689, 8317, 8547, | |
| | | | | | 6074, 7887, 5982, 7832, 6053, 6535, | |
| PRSS1 | 8136795 | 367 | NM_002769 | 657 | 4044, 4083, 5224, 7372, 8647, 5627, 7412, | +2.09 |
| | | | | | 7969, | |
| MYL7 | 8139307 | 368 | NM_021223 | 658 | 3043, 3698, 7148, 5334, 7843, 7318, 7526, | +2.08 |
| DUSP4 | 8150076 | 369 | NM_001394 | 659 | 2649, 5265, 5266, 5393, | +2.06 |
| PROS1 | 8089011 | 370 | NM_000313 | 660 | 2597, 4770, 6690, | +2.06 |
| ANKRD20B | 8020349 | 371 | ENST00000400842 | 661 | 0 | +2.05 |
| CTSL2 | 8162652 | 372 | NM_001333 | 662 | 3462, 5253, 8071, 5450, | +2.05 |
| FMO5 | 7919314 | 373 | NM_001461 | 663 | 3169, 4740, 4742, 4741, 5452, 5926, 6568, | +2.05 |
| | | | | | 7792, 6924, | |
| USP27X | 8167601 | 374 | NM_001145073 | 664 | 4754, | +2.04 |
| LAMA1 | 8022176 | 375 | NM_005559 | 665 | 4685, 5278, 8564, 7980, 8761, 6083, | +2.04 |
| | | | | | 7657, | |
| ADAM28 | 8145293 | 376 | NM_014265 | 666 | 4384, 3969, 3970, 7848, 8743, 8310, 7519, | +2.03 |
| | | | | | 7029, 7897, 7794, 7884, 5486, 8614, | |
| ZNF611 | 8038981 | 377 | NM_030972 | 667 | 3136, 4861, 4862, 4863, 4864, 5722, | +2.03 |
| ANKRD20B | 7970381 | 378 | AK057820 | 668 | 2984, 4169, 6891, 6432, 6336, 7704, 6356, | +2.02 |
| | | | | | 7272, 7347, | |
| ZNF137 | 8030954 | 379 | U09414 | 669 | 2628, 4364, | +2.02 |
| S100Z | 8106411 | 380 | NM_130772 | 670 | 3017, 4191, 8752, 5748, | +2.01 |
| GPSM2 | 7903565 | 381 | NM_013296 | 671 | 3143, 4876, 3650, 2672, 4515, 3370, 4289, | +2.01 |
| | | | | | 7669, 5503, 6737, 6476, 6581, 7296, 6065, | |
| | | | | | 7166, 5954, 5917, 5991, | |
| TGFB2 | 7909789 | 382 | NM_003238 | 672 | 2610, 5188, 5189, 7936, 5882, 8554, 5876, | +2.00 |
| ARHGAP28 | 8019964 | 383 | NM_001010000 | 673 | 2803, 5198, 5415, 6442, 5664, 6551, 7534, | +2.00 |
| | | | | | 7585, | |

Table 3. Provided are the description of the non-membranal genes along with the sequence identifiers of the Affymetrix probes, the target sequence identified by the Affymetrix probe, and the mRNA sequences represented by the target sequences. Also shown are the log 2 fold change in expression levels of the indicated genes between the SOX17 GFP cells and the genetically unmodified HESCs. The (+) or (−) sign means that the genes are upregulated or downregulated, respectively, in the SOX17-GFP cells as compared to the genetically unmodified HESCs.

TABLE 4
Non-membranal genes which are down-regulated in SOX17+ cells as compared to genetically un-modified ESCs

| | | Affy. target | | SEQ ID NO: | | Log 2 fold |
|---|---|---|---|---|---|---|
| Gene | Probe | nucl. SEQ | Rep. | (Of Rep. | Polyn. SEQ ID NOs: | change |
| Symbol | Set ID | ID NO: | Public ID | Public ID) | rep. by target | [SOX17-HESC] |
| ZFP42 | 8098690 | 384 | NM_174900 | 674 | 3498, 3937, 5793, 8300, | −5.87 |
| THUMPD3 | 8077513 | 385 | NM_015453 | 675 | 2882, 2944, 4333, 4334, 6458, 6573, 7246, 8510, | −4.50 |
| | | | | | 6238, 6855, 6555, 8394, 7095, 6527, | |
| ANXA1 | 8155849 | 386 | NM_000700 | 676 | 3181, 2757, 6327, 5394, 7974, 6460, 7578, 7227, | −4.42 |
| | | | | | 8129, 8580, | |
| SPP1 | 8096301 | 387 | NM_001040058 | 677 | 4706, 3942, 3501, 3944, 8278, 7992, 5370, 6716, | −4.21 |
| | | | | | 8452, 8651, 6109, 6075, 7831, 7801, | |
| PRDM14 | 8151240 | 388 | NM_024504 | 678 | 2858, 3552, 5534, | −4.18 |
| GNA14 | 8161892 | 389 | NM_004297 | 679 | 2753, 4750, 6030, 7575, | −4.16 |
| EDIL3 | 8112980 | 390 | NM_005711 | 680 | 2720, 3295, 8700, 8201, 7504, 5630, | −4.05 |
| CXCL12 | 7933194 | 391 | NM_199168 | 681 | 2665, 5012, 5013, 5117, 5003, 6588, 8491, 7914, | −3.84 |
| | | | | | 6051, 6656, 6859, | |
| PSMD5 | 8163795 | 392 | NM_005047 | 682 | 4517, 4254, 2800, 2963, 3212, 8729, 8252, 5958, | −3.78 |
| | | | | | 6078, 8378, 5320, 5967, | |
| PRRX1 | 7907222 | 393 | NM_022716 | 683 | 4004, 3771, 3772, 5361, 6117, 8431, 7764, 8243, | −3.77 |
| | | | | | 8582, | |
| NANOG | 7953675 | 394 | NM_024865 | 684 | 3571, 3299, 4206, 5371, | −3.72 |
| TRIM22 | 7938035 | 395 | NM_006074 | 685 | 2821, 2822, 2823, 4283, 4533, 4544, 4481, 3016, | −3.56 |
| | | | | | 3162, 4213, 4312, 4320, 4326, 5229, 7088, 6634, | |
| | | | | | 6762, 8083, 6459, 8567, 6533, 6911, 6474, 7269, | |
| | | | | | 7353, | |
| NANOG | 7987365 | 396 | NM_024865 | 686 | 3571, 3299, 4206, 5371, | −3.53 |
| RASGRF2 | 8106660 | 397 | NM_006909 | 687 | 4058, 5302, 8643, 5518, 8502, 8537, 8352, | −3.52 |
| POU5F1B | 8148315 | 398 | NM_001159542 | 688 | 5084, 4815, 6578, 6639, | −3.50 |
| POLR3G | 8106820 | 399 | NM_006467 | 689 | 4507, 4049, 6583, 8418, | −3.44 |
| HHLA1 | 8152938 | 400 | NM_001145095 | 690 | 2750, 4755, 7527, 8041, | −3.36 |
| POU5F1 | 8124889 | 401 | NM_203289 | 691 | 2865, 2866, 2867, 2868, 3988, 3986, 4587, 5026, | −3.36 |
| | | | | | 5027, 5028, 5029, 5030, 5031, 5032, 5033, 5034, | |
| | | | | | 5035, 5036, 5037, 5038, 5039, 5040, 5041, 5042, | |
| | | | | | 5043, 5044, 5045, 5046, 5047, 5048, 5049, 5050, | |
| | | | | | 5051, 5052, 5053, 5054, 5055, 5056, 5057, 5058, | |
| | | | | | 5059, 5060, 5061, 5062, 5063, 5064, 5065, 5066, | |
| | | | | | 5067, 5068, 5069, 5070, 5071, 5072, 5073, 5074, | |
| | | | | | 5075, 5076, 5077, 5078, 5079, 5080, 5081, 5082, | |
| | | | | | 5083, 5084, 5085, 5086, 5087, 5088, 5089, 5090, | |
| | | | | | 5091, 5092, 5093, 5094, 5095, 5096, 5097, 5098, | |
| | | | | | 5099, 5100, 5101, 5102, 5103, 5104, 4227, 4228, | |
| | | | | | 4229, 4230, 4231, 3958, 3959, 4369, 5016, 5017, | |
| | | | | | 4052, 5175, 5179, 8740, 7043, 6463, 8204, 6434, | |
| | | | | | 6854, 6431, 8068, 6882, 7219, 7760, 8266, 8247, | |
| | | | | | 6495, 7073, 7834, 7275, 7155, 8483, 6801, 7986, | |
| | | | | | 6354, 8509, 6963, 7415, 7877, 6325, 7358, 5405, | |
| | | | | | 7117, 6852, 8575, 6970, 7641, 7552, | |
| VSNL1 | 8040430 | 402 | NM_003385 | 692 | 3384, 3838, 6777, 6629, 8659, 5837, 6389, 6436, | −3.33 |
| SCG3 | 7983718 | 403 | NM_001165257 | 693 | 3014, 4909, 4908, 5333, | −3.33 |
| B3GALT1 | 8046078 | 404 | NM_020981 | 694 | 2763, 5191, 6587, 5756, | −3.28 |
| LECT1 | 7971838 | 405 | NM_007015 | 695 | 3028, 3784, 3785, 7153, 6673, | −3.25 |
| NTS | 7957458 | 406 | NM_006183 | 696 | 2948, 5242, 5429, | −3.19 |
| MBNL1 | 8083429 | 407 | NM_021038 | 697 | 3270, 3597, 3598, 3599, 3600, 3601, 3602, 3603, | −3.18 |
| | | | | | 6944, 7767, 5777, 8388, 7338, 5574, 7958, 5685, | |
| | | | | | 5890, 6259, 8038, 5830, | |
| CKMT1A | 7983239 | 408 | NM_020990 | 698 | 4017, 4725, 3845, 3844, 5618, 8459, 7658, 6700, | −3.18 |
| | | | | | 6805, 7001, 6390, 7746, 7418, 6964, 8012, 7084, | |
| | | | | | 7264, 7777, 8717, 7315, 6930, 7503, 6473, 7092, | |
| | | | | | 7417, 7902, 7582, | |
| NECAB1 | 8147244 | 409 | NM_022351 | 699 | 3290, 4737, 6800, 7785, 7823, 8669, 7989, 8696, | −3.14 |
| FGF2 | 8097256 | 410 | NM_198041 | 700 | 2576, 5304, 4197, 5305, 5478, 5980, 5822, | −3.12 |
| SFRP2 | 8103254 | 411 | NM_003013 | 701 | 2933, 3724, 5471, | −3.07 |
| DCLK1 | 7970954 | 412 | NM_004734 | 702 | 4221, 5192, 5193, 5194, 5195, 5448, 7317, 6024, | −3.07 |
| | | | | | 6492, 7076, | |
| DACT1 | 7974689 | 413 | NM_016651 | 703 | 2815, 4080, 4079, | −3.06 |
| CRABP1 | 7985159 | 414 | NM_004378 | 704 | 3015, 4439, 5645, | −3.02 |
| TFAP2C | 8063536 | 415 | NM_003222 | 705 | 3168, 3531, 5324, | −3.01 |
| SCGB3A2 | 8108995 | 416 | NM_054023 | 706 | 3022, 4995, 8754, 8215, 5614, | −2.89 |
| LRAT | 8097920 | 417 | NM_004744 | 707 | 3566, 3595, 8748, 8069, 7763, 8070, 5620, | −2.88 |
| CUZD1 | 7936798 | 418 | NM_022034 | 708 | 3275, 5260, 6276, 6229, 6120, 6165, 6557, 6134, | −2.86 |
| GLB1L3 | 7945296 | 419 | NM_001080407 | 709 | 3430, 4288, 6702, 8356, 8044, 7094, 7185, 6706, | −2.86 |
| METTL7A | 7955441 | 420 | NM_014033 | 710 | 3523, 3936, 5745, 6365, | −2.85 |
| VAT1L | 7997336 | 421 | NM_020927 | 711 | 3166, 3189, 5813, | −2.81 |
| COL12A1 | 8127563 | 422 | NM_080645 | 712 | 4761, 3954, 3953, 5758, 6009, 7798, 8346, 8170, | −2.80 |
| | | | | | 5484, 6823, | |
| OLFML3 | 7904158 | 423 | NM_020190 | 713 | 3467, 3697, 6147, 5677, 8035, 6986, | −2.78 |
| SOX2 | 8084165 | 424 | NM_003106 | 714 | 3338, 5264, 5632, | −2.76 |
| USP44 | 7965565 | 425 | NM_032147 | 715 | 3062, 3978, 3979, 6775, 5436, | −2.72 |
| HIST1H4F | 8117422 | 426 | NM_003540 | 716 | 3825, 3059, 7626, | −2.70 |
| KGFLP1 | 8067839 | 427 | NM_002009 | 717 | 3477, 2587, 4695, 4203, 5525, 7263, | −2.69 |
| CPT1A | 7949971 | 428 | NM_001876 | 718 | 2870, 4398, 4399, 7075, 5473, | −2.69 |
| DBC1 | 8163716 | 429 | NM_014618 | 719 | 2726, 4469, 4474, 3012, 3546, 3649, 4164, 6292, | −2.64 |
| | | | | | 6191, 5480, | |
| CHAC1 | 7982868 | 430 | NM_024111 | 720 | 3003, 4671, 4672, 7389, 6822, 8727, | −2.63 |
| CAV1 | 8135594 | 431 | NM_001753 | 721 | 4248, 4999, 5000, 5001, 5002, 6085, 6753, 8406, | −2.63 |
| MT1G | 8001531 | 432 | NM_005950 | 722 | 3006, 2840, 6392, 7114, 7170, 6815, | −2.63 |
| NFIX | 8026139 | 433 | NM_002501 | 723 | 4021, 4022, 3743, 3307, 2629, 2644, 2645, 4501, | −2.62 |
| | | | | | 4543, 4907, 3764, 5981, 6837, 5556, 6879, 5925, | |
| FERMT1 | 8064904 | 434 | NM_017671 | 724 | 3567, 4061, 7556, 7678, 8349, 5970, 5336, | −2.59 |
| GLIPR1 | 7957260 | 435 | NM_007043 | 725 | 2953, 3451, 4074, 4007, 6328, 5493, 5382, | −2.59 |
| TOX | 8150962 | 436 | NM_014729 | 726 | 2740, 3709, 6856, 6017, | −2.58 |
| SNRPN // | 7981960 | 437 | NR_003321 | 727 | 4118, | −2.57 |
| SNORD116-6 | | | | | | |
| HEY2 | 8121850 | 438 | NM_012259 | 728 | 4276, 3975, 5989, 6023, | −2.57 |
| TIMP4 | 8085360 | 439 | NM_003256 | 729 | 2945, 5251, 5824, | −2.56 |
| IDO1 | 8146092 | 440 | NM_002164 | 730 | 2584, 5259, 5421, 8523, 8757, 8169, 8149, 8114, | −2.53 |
| | | | | | 8319, 7915, 8778, | |
| MT2A | 7995783 | 441 | NM_005953 | 731 | 2919, 4617, 5366, | −2.52 |
| NR5A2 | 7908597 | 442 | NM_003822 | 732 | 2737, 3582, 3583, 6058, 5356, 8618, 7477, 6293, | −2.52 |
| | | | | | 5367, | |
| UPRT | 8168438 | 443 | NM_145052 | 733 | 3674, 4272, 2966, 4297, 4940, 4941, 7089, 8335, | −2.50 |
| | | | | | 6323, 6105, 6157, | |
| MYC | 8148317 | 444 | NM_002467 | 734 | 2869, 4851, 6776, 7274, | −2.48 |
| CCDC109B | 8096808 | 445 | NM_032992 | 735 | 3120, 3873, 3874, 4445, 8500, 7859, 7100, 6201, | −2.48 |
| GAL | 7942064 | 446 | NM_015973 | 736 | 3055, 3933, 5477, | −2.47 |
| ZNF483 | 8157193 | 447 | NM_133464 | 737 | 4219, 4428, 4429, 7118, 6162, 5698, | −2.45 |
| RND3 | 8055688 | 448 | NM_005168 | 738 | 3040, 3678, 3679, 3283, 3713, 4557, 4566, 2954, | −2.45 |
| | | | | | 4291, 4318, 4304, 3770, 7689, 7080, 7920, 6797, | |
| | | | | | 7108, 7603, 8181, 5569, 8123, | |
| BNC2 | 8160260 | 449 | NM_017637 | 739 | 3551, 4555, 3100, 4545, 4032, 4033, 4034, 4035, | −2.45 |
| | | | | | 4036, 4037, 2799, 3963, 6528, 6375, 7529, 7058, | |
| | | | | | 6914, 7266, 7235, 7564, 8608, 7382, 8765, | |
| COL3A1 | 8046922 | 450 | NM_000090 | 740 | 3042, 4000, 7681, 5702, 7724, 5577, | −2.42 |
| LUM | 7965403 | 451 | NM_002345 | 741 | 2920, 3798, 5519, | −2.42 |
| LDB2 | 8099524 | 452 | NM_001290 | 742 | 2733, 4590, 4591, 8679, 8166, 7837, 8661, 8298, | −2.41 |
| | | | | | 5669, 8605, 7719, 7814, 8032, 8473, 7455, 8734, | |
| MT1E | 7995797 | 453 | NM_175617 | 743 | 3381, 3905, 5861, 5789, | −2.40 |
| SNRPN // | 7981994 | 454 | NR_003337 | 744 | 4131, | −2.37 |
| SNORD116-23 | | | | | | |
| SNRPN // | 7982002 | 455 | NR_003341 | 745 | 4127, | −2.37 |
| SNORD116-27 | | | | | | |
| GALNT3 | 8056408 | 456 | NM_004482 | 746 | 3996, 3990, 3410, 3332, 4301, 4196, 6671, 7016, | −2.36 |
| | | | | | 6517, 7277, 7565, 6510, 6479, 6804, 6507, | |
| PIPOX | 8006005 | 457 | NM_016518 | 747 | 3342, 3789, 7690, 7748, 5638, 6426, 6464, | −2.35 |
| PDK1 | 8046408 | 458 | NM_002610 | 748 | 3198, 3485, 7350, 7468, 6847, 5563, 6903, | −2.34 |
| PREX2 | 8146794 | 459 | NM_024870 | 749 | 3640, 3643, 3644, 6885, 8788, 6153, 5651, | −2.33 |
| CYP2S1 | 8028991 | 460 | NM_030622 | 750 | 3461, 3715, 5647, 5708, | −2.33 |
| NRK | 8169115 | 461 | NM_198465 | 751 | 3311, 4081, 5354, | −2.33 |
| VAV3 | 7918157 | 462 | NM_006113 | 752 | 2741, 4095, 4094, 8118, 7879, 6112, 7502, 5897, | −2.32 |
| | | | | | 6080, 6942, | |
| TNFAIP6 | 8045688 | 463 | NM_007115 | 753 | 3052, 5234, 5352, 6810, | −2.29 |
| ENPP1 | 8122099 | 464 | NM_006208 | 754 | 3492, 4353, 5924, 6245, 8263, 8565, 7205, | −2.27 |
| ADD2 | 8052882 | 465 | NM_001617 | 755 | 3312, 2549, 4252, 4541, 3221, 3555, 5136, 5137, | −2.26 |
| | | | | | 5138, 5139, 5140, 7591, 8079, 5513, 5527, 8334, | |
| | | | | | 8155, 6282, 7652, 6883, 7881, 7694, 6520, 6309, | |
| | | | | | 7614, 7098, | |
| SNRPN // | 7981996 | 466 | NR_003338 | 756 | 4129, | −2.25 |
| SNORD116-24 | | | | | | |
| SNRPN // | 7981947 | 467 | NR_001289 | 757 | 3262, 3274, | −2.25 |
| SNORD109A | | | | | | |
| STC2 | 8115851 | 468 | NM_003714 | 758 | 3356, 3794, 8437, 5481, | −2.22 |
| SNORA22 | 8133106 | 469 | NR_002961 | 759 | 3945, | −2.22 |
| MPPED2 | 7947274 | 470 | NM_001584 | 760 | 2671, 4776, 4777, 6358, 6060, | −2.21 |
| ZNF562 | 8033801 | 471 | NM_001130031 | 761 | 4039, 4450, 4451, 4452, 6833, 5584, 6691, | −2.20 |
| GAP43 | 8081810 | 472 | NM_002045 | 762 | 2575, 4459, 4460, 5773, | −2.20 |
| FOXB1 | 7984011 | 473 | NM_012182 | 763 | 4249, 3974, 6779, | −2.19 |
| TSHZ3 | 8035896 | 474 | NM_020856 | 764 | 4257, 4125, 5381, | −2.18 |
| HPGD | 8103769 | 475 | NM_000860 | 765 | 2662, 2663, 2690, 4708, 4570, 4471, 4475, 3508, | −2.17 |
| | | | | | 3534, 4794, 4795, 4796, 7891, 7937, 8399, 8639, | |
| | | | | | 8591, 5581, 7846, 5659, 8577, 7975, 6419, 7812, | |
| ZDHHC22 | 7980344 | 476 | NM_174976 | 766 | 4047, 4139, 5735, | −2.17 |
| ACOXL | 8044353 | 477 | NM_001142807 | 767 | 4470, 4673, 6005, 7357, 6866, 6663, 6681, | −2.17 |
| GLI3 | 8139212 | 478 | NM_000168 | 768 | 3991, 3982, 2577, 4553, 3392, 4592, 6480, 7171, | −2.16 |
| | | | | | 6577, 6439, 8751, 6708, | |
| CDCA7L | 8138489 | 479 | NM_018719 | 769 | 3585, 4801, 4802, 4803, 7510, 6116, 7011, 6541, | −2.14 |
| | | | | | 6183, 8558, | |
| ZSCAN10 | 7998921 | 480 | NM_032805 | 770 | 3149, 2932, 5400, | −2.13 |
| GFPT2 | 8116418 | 481 | NM_005110 | 771 | 2751, 4135, 8471, 5407, 8222, 8775, 7999, | −2.13 |
| PLP2 | 8167449 | 482 | NM_002668 | 772 | 2555, 4965, 7651, 6875, 7509, | −2.13 |
| HIST1H1A | 8124380 | 483 | NM_005325 | 773 | 3881, 4053, 5377, | −2.12 |
| CAMKV | 8087530 | 484 | NM_024046 | 774 | 3407, 4516, 4542, 4547, 4568, 3355, 2883, 3337, | −2.12 |
| | | | | | 3368, 3000, 3165, 3910, 8007, 8299, 6547, 8504, | |
| | | | | | 8573, 8756, 8739, 7861, 8075, 7731, 6769, 7182, | |
| | | | | | 8603, 8099, 7254, 5602, | |
| HERC5 | 8096361 | 485 | NM_016323 | 775 | 3489, 4008, 8649, 5458, 8138, 8244, | −2.12 |
| MT1X | 7995838 | 486 | NM_005952 | 776 | 3371, 4455, 6391, | −2.12 |
| TERF1 | 8102789 | 487 | NM_003218 | 777 | 2691, 4416, 4417, 6010, 5544, 5494, | −2.12 |
| RAB31 | 8020110 | 488 | NM_006868 | 778 | 2829, 4352, 5595, 6339, | −2.12 |
| SNRPN // | 7981974 | 489 | NR_003328 | 779 | 4128, | −2.10 |
| SNORD116-13 | | | | | | |
| ETV1 | 8138289 | 490 | NM_004956 | 780 | 3858, 3887, 3888, 4724, 2640, 2658, 4696, 4698, | −2.10 |
| | | | | | 4701, 4715, 3245, 4328, 4314, 4877, 4878, 4879, | |
| | | | | | 4880, 4881, 4882, 4883, 6616, 7632, 7308, 7476, | |
| | | | | | 7190, 7710, 7373, 7697, 7749, 8199, 6672, 6566, | |
| | | | | | 5364, 6410, 7966, 7584, 8722, 7292, 7655, 6209, | |
| | | | | | 7888, 8053, 8570, 6394, | |
| MT1G | 8162531 | 491 | ENST00000396160 | 781 | 7570, | −2.10 |
| ACTA1 | 7924910 | 492 | NM_001100 | 782 | 3288, 3669, 3677, 4719, 2955, 3612, 5805, 6231, | −2.10 |
| | | | | | 5889, 6187, 6159, | |
| SNRPN // | 7981988 | 493 | AF241255 | 783 | 2839, 4130, | −2.10 |
| SNORD116-20 | | | | | | |
| NFIB | 8160138 | 494 | NM_005596 | 784 | 3285, 2688, 2685, 4244, 4562, 2874, 3417, 5146, | −2.09 |
| | | | | | 5145, 5147, 7613, 7638, 6784, 7684, 7327, 6514, | |
| | | | | | 7007, 8444, 7463, | |
| ZEB2 | 8055624 | 495 | NM_014795 | 785 | 3737, 3738, 4072, 4073, 4702, 2907, 3889, 3164, | −2.08 |
| | | | | | 3616, 3504, 4311, 4982, 4983, 4984, 7620, 7739, | |
| | | | | | 7312, 6428, 7804, 5578, 7411, 7695, 7432, 6411, | |
| | | | | | 6906, 7751, 8052, 7712, 6862, 6562, 7983, 7245, | |
| | | | | | 7643, 6372, 7130, 7002, 6978, 7523, | |
| CBR1 | 8068401 | 496 | NM_001757 | 786 | 3890, 3754, 7622, 5848, | −2.08 |
| ATXN7L1 | 8142087 | 497 | NM_138495 | 787 | 4574, 4661, 4286, 7346, 7677, 7827, | −2.07 |
| SNRPN // | 7981949 | 498 | NR_003316 | 788 | 4117, | −2.06 |
| SNORD116-1 | | | | | | |
| MT1F | 7995825 | 499 | NM_005949 | 789 | 3051, 4447, 5801, 6580, | −2.06 |
| SNRPN // | 7982006 | 500 | NR_003360 | 790 | 4121, | −2.06 |
| SNORD116-29 | | | | | | |
| AP1M2 | 8034084 | 501 | NM_005498 | 791 | 2896, 4739, 5398, | −2.05 |
| ACTG2 | 8042788 | 502 | NM_001615 | 792 | 2956, 3832, 6377, 7341, 6703, 6599, 7930, 5891, | −2.05 |
| CYP2B6 | 8028963 | 503 | NM_000767 | 793 | 2569, 3046, 5598, 5779, | −2.05 |
| SERPINE1 | 8135069 | 504 | NM_000602 | 794 | 2947, 4910, 4911, 5338, | −2.03 |
| GRHL2 | 8147697 | 505 | NM_024915 | 795 | 4098, 4097, 4270, 4529, 3637, 3606, 3638, 4299, | −2.03 |
| | | | | | 4354, 5422, 7907, 7656, 8209, 8281, 7516, | |
| SLIT2 | 8094301 | 506 | NM_004787 | 796 | 2748, 2765, 3985, 4678, 3436, 2744, 2767, 8366, | −2.03 |
| | | | | | 7786, 6676, 7740, 5522, 8033, 7772, 7956, 8641, | |
| | | | | | 8725, | |
| PIM2 | 8172471 | 507 | NM_006875 | 797 | 2994, 4637, 7671, 8024, 6884, | −2.02 |
| SMARCA2 | 8154059 | 508 | NM_139045 | 798 | 2631, 2619, 3109, 4525, 4559, 4564, 4569, 3205, | −2.02 |
| | | | | | 4322, 3654, 3655, 7236, 8645, 6437, 7334, 7214, | |
| | | | | | 5617, 7026, 6958, 5795, 5880, 7544, 7653, 6932, | |
| | | | | | 6870, 6536, 7608, 5988, 7032, | |
| RPPH1 | 7977507 | 509 | NR_002312 | 799 | 3839, | −2.01 |

Table 4. Provided are the description of the non-membranal genes along with the sequence identifiers of the Affymetrix probes, the target sequence identified by the Affymetrix probe, and the mRNA sequences represented by the target sequences. Also shown are the log 2 fold change in expression levels of the indicated genes between the SOX17 GFP cells and the genetically unmodified HESCs. The (+) or (−) sign means that the genes are upregulated or downregulated, respectively, in the SOX17-GFP cells as compared to the genetically unmodified HESCs.

### Example 2

### Generation of a PDX1-GFP BAC Construct and Isolation of PDX1-Expressing ESCS

Experimental Results

BAC Transgenesis of PDX1-GFP Reporter BAC—

An important gene in the commitment of definitive endoderm cells towards pancreas is PDX1. Thus, a fluorescent reporter construct was built to study precursor cells at this later stage of the differentiation process. The present inventors generated PDX1-reporter constructs in which the coding sequence of the PDX1 gene was replaced by the coding sequence of the EGFP gene together with a floxed Neo gene, as follows.

Preparation of PDX1 ATG-GFP-Knockin Construct:

EGFP and floxed neo was knocked into the PDX1 gene locus in BAC BAC RP11-322P28 by replacing the coding sequence of PDX1 (ATG to TGA) with the coding sequence of GFP and the floxed neomycin resistance gene. FIGS. 3A-B schematically illustrate the structure of the recombinant BAC RP11-322P28 PDX1-GFP construct (SEQ ID NO:15), which includes the PDX1 upstream regulatory sequence (SEQ ID NO:16, about 8.7 kb), the GFP coding sequence (SEQ ID NO:2) followed by the SV40 polyA sequence (SEQ ID NO:27), a loxP site (SEQ ID NO:4), the neomycin resistance gene sequence (SEQ ID NO:3) with the SV40 early promoter/enhancer (SEQ ID NO:28), and the TK polyadenylation site (SEQ ID NO:29), a second loxP site (SEQ ID NO:4), the PDX1 downstream regulatory sequence (SEQ ID NO:17, about 19.5 kb).

Preparation of PDX1 ATG-GFP-Knockin Plasmid Construct:

Part of the BAC PDX1-GFP construct (SEQ ID NO:15) was subcloned into the Bluescript plasmid (Stratagene) to generate the recombinant PDX1-GFP plasmid construct (SEQ ID NO:18) as follows. FIG. 3C schematically illustrates the structure of the recombinant plasmid PDX1-GFP construct (SEQ ID NO:18), which includes the 5′-arm (SEQ ID NO:19), the GFP coding sequence (SEQ ID NO:2) followed by the SV40 polyA sequence (SEQ ID NO:27), a loxP site (SEQ ID NO:4), the neomycin resistance gene sequence (SEQ ID NO:3) with the SV40 early promoter/enhancer (SEQ ID NO:28), and the TK polyadenylation site (SEQ ID NO:29), a second loxP site (SEQ ID NO:4), and a 3′-arm (SEQ ID NO:20).

A 3′-external probe was prepared (SEQ ID NO:21) in order to detect correct targeting of the vector into the genomic DNA of the host cell.

Preparation of PSC1x: PDX1 IRES-GFP-Knockin Plasmid Construct—

IRES-eGFP and floxed neo was knocked into the 3′ UTR of the PDX1 gene. FIG. 3D schematically illustrates the structure of the recombinant plasmid construct (SEQ ID NO:22), which includes the 5′-arm (SEQ ID NO:23) which includes the PDX1 exons, the PDX1 5′-UTR (5′-untranslated region), the 3′-UTR, an IRES sequence (SEQ ID NO:25), the GFP coding sequence (SEQ ID NO:2) followed by the SV40 polyA sequence (SEQ ID NO:27), a loxP site (SEQ ID NO:4), the neomycin resistance gene sequence (SEQ ID NO:3) with the SV40 early promoter/enhancer (SEQ ID NO:28), and the TK polyadenylation site (SEQ ID NO:29), a second loxP site (SEQ ID NO:4), and a 3′-arm (SEQ ID NO:24).

A 3′-external probe was prepared (SEQ ID NO:26) in order to detect correct targeting of the vector into the genomic DNA of the host cell.

Preparation of PDX1 ATG-mCherry-Knockin BAC—

mCherry and a floxed neo was knocked into the PDX1 gene locus in BAC RP11-322P28. FIG. 3D schematically illustrates the structure of the recombinant BAC construct (SEQ ID NO:30), which includes the 5′-arm (SEQ ID NO: 31) which includes PDX1 upstream regulatory sequence, the mCherry coding sequence (SEQ ID NO:33) followed by the SV40 polyA sequence (SEQ ID NO:27), a loxP site (SEQ ID NO:4), the neomycin resistance gene sequence (SEQ ID NO:3) with the SV40 early promoter/enhancer (SEQ ID NO:28), and the TK polyadenylation site (SEQ ID NO:29), a second loxP site (SEQ ID NO:4), and a 3′-arm (SEQ ID NO:32).

Transfection of the Recombinant Construct of Some Embodiments of the Invention into Human ESCs—

Various hESC clones harbouring the PDX1 GFP reporter BAC (SEQ ID NO:15) were isolated and were allowed to differentiate by forming EBs. At day 14, the EBs were dissociated into single cells and were analysed by FACS. The number of GFP+ cells in the different clones varied from about 0.5% to about 3% (FIG. 4C). 14 day-old EBs from a reporter clone were seen to contain clusters of GFP⁺ cells. The EBs were dissociated into single cells and were put onto cover slips using the cytospin, fixed and immunostained for PDX1. The cells clearly co-expressed PDX1 and GFP demonstrating that the GFP⁺ population indeed represent PDX1 positive cells (FIG. 4D).

Next, the PDX1⁺ cells were sorted by FACS and RNA was extracted from the GFP⁺ and GFP⁻ populations. RT-qPCR was performed to compare expression of various markers in the two different cell populations. As can be seen in FIG. 4E, PDX1 and other pancreatic differentiation markers were enriched in the GFP⁺ population. Some of the enriched genes include NGN3, paired box 4 (PAX4), hepatocyte nuclear factor 6 (HNF6) and homeobox gene HB9 (HLXB9)). These results further validate the identity of the PDX+ population as pancreatic progenitor cells.

TABLE 5
Membranal genes which are upregulated in PDX1 cells as compared to genetically un-modified ESCs

| | Affy. target | | SEQ ID NO: | | | Log 2 fold |
|---|---|---|---|---|---|---|
| Probe | nucl. SEQ | Rep. | (Of Rep. | Polyn. SEQ ID NOs: | Gene | change |
| Set ID | ID NO: | Public ID | Public ID) | rep. by target | Symbol | [PDX1-HESC] |
| 8035304 | 510 | NM_004335 | 800 | 3090, 2805, 5403, 7124, | BST2 | 3.99 |
| 7989073 | 511 | NM_173814 | 801 | 3710, 4778, 6379, | PRTG | 3.28 |
| 8056611 | 512 | NM_004525 | 802 | 2684, 4119, 5456, 8262, | LRP2 | 2.83 |
| 8031076 | 513 | NM_031896 | 803 | 3827, 5226, 7212, 5339, | CACNG7 | 2.71 |
| 7976783 | 514 | NM_003836 | 804 | 4273, 4994, 6774, 5796, 6319, | DLK1 | 2.66 |
| 8087691 | 515 | NM_001174051 | 805 | 2721, 2711, 2712, 4220, 4298, 5019, 3735, | CACNA2D2 | 2.56 |
| | | | | 3736, 6362, 6812, 5540, 6239, 8681, 7132, | | |
| | | | | 7985, 7734, 6957, 7200, | | |
| 7909390 | 516 | NM_175710 | 806 | 3897, 4185, 8470, 6548, 5851, | CR1L | 2.56 |
| 7994058 | 517 | NM_001039 | 807 | 3481, 4177, 5784, | SCNN1G | 2.54 |
| 8169447 | 518 | NM_000868 | 808 | 2609, 4287, 5920, 5564, 5986, | HTR2C | 2.44 |
| 7917276 | 519 | NM_012152 | 809 | 2780, 4363, 7551, 8539, 6011, | LPAR3 | 2.27 |
| 7920664 | 520 | NM_007112 | 810 | 2652, 3541, 6070, 7928, 8701, 6699, 6720, | THBS3 | 2.18 |
| | | | | 8067, 8010, 7294, 6846, | | |
| 8051785 | 521 | NM_133329 | 811 | 3647, 5227, 5228, 5783, 7561, | KCNG3 | 2.15 |
| 8018114 | 522 | NM_001144952 | 812 | 2814, 4748, 7774, 5765, 6851, 6326, | SDK2 | 2.13 |
| 8178193 | 523 | NM_019111 | 813 | 3652, 4537, 2590, 2589, 4532, 4478, 4721, | HLA-DRA | 2.10 |
| | | | | 4482, 3074, 3666, 5174, 6840, 8271, 7495, | | |
| | | | | 7301, 6340, 7125, 7667, 6973, 6698, 6694, | | |
| | | | | 7895, 6462, 7070, 8427, 7321, 6682, 7906, | | |
| | | | | 7421, 7648, 7487, 6525, | | |
| 7930837 | 524 | NM_003054 | 814 | 3141, 5107, 7960, 5655, | SLC18A2 | 2.09 |
| 8011713 | 525 | NM_022059 | 815 | 2851, 4210, 4211, 5728, | CXCL16 | 1.96 |
| 7916584 | 526 | NM_002353 | 816 | 2675, 4337, 6094, | TACSTD2 | 1.91 |
| 7975932 | 527 | NM_020431 | 817 | 4392, 4432, 5791, | TMEM63C | 1.85 |
| 8038367 | 528 | NM_020309 | 818 | 3483, 4743, 5332, | SLC17A7 | 1.83 |
| 8114415 | 529 | NM_001496 | 819 | 3167, 3584, 7363, 5508, | GFRA3 | 1.83 |

Table 5: Provided are the description of the membranal genes along with the sequence identifiers of the Affymetrix probes, the target sequence identified by the Affymetrix probe, and the mRNA sequences represented by the target sequences. Also shown are the log 2 fold change in expression levels of the indicated genes between the PDX1-GFP cells and the genetically unmodified HESCs. The (+) or (−) sign means that the genes are upregulated or downregulated, respectively, in the PDX1-GFP cells as compared to the genetically unmodified HESCs.

TABLE 6
Membranal genes which are downregulated in PDX1 cells as compared to genetically un-modified ESCs

| | Affy. target | | SEQ ID NO: | | | Log 2 fold |
|---|---|---|---|---|---|---|
| Probe | nucl. SEQ | Rep. | (Of Rep. | Polyn. SEQ ID NOs: | Gene | change |
| Set ID | ID NO: | Public ID | Public ID) | rep. by target | Symbol | [PDX1-HESC] |
| 8095364 | 820 | NM_014058 | 1411 | 3472, 4222, 5579, 8653, | TMPRSS11E | −3.58 |
| 8095380 | 821 | NM_014058 | 1412 | 3472, 4222, 5579, 8653, | TMPRSS11E | −3.60 |
| 7957140 | 822 | NM_003667 | 1413 | 2732, 3193, 6795, 5509, | LGR5 | −3.03 |
| 8101992 | 823 | NM_001135146 | 1414 | 2875, 4623, 4624, 3783, 4625, 7838, 6311, 8464, | SLC39A8 | −2.82 |
| | | | | 6341, 7047, | | |
| 8091402 | 824 | NM_001184723 | 1415 | 2962, 5118, 5119, 5621, 7779, 6927, 6623, 8441, | TM4SF18 | −2.73 |
| 7936798 | 825 | NM_022034 | 1416 | 3275, 5260, 6276, 6229, 6120, 6165, 6557, 6134, | CUZD1 | −2.27 |
| 8175217 | 826 | NM_001448 | 1417 | 2738, 3072, 6154, | GPC4 | −2.20 |
| 8123246 | 827 | NM_021977 | 1418 | 3195, 5535, 7120, | SLC22A3 | −2.20 |
| 8055465 | 828 | NM_001008540 | 1419 | 2782, 3775, 3776, 5384, 6834, 7645, | CXCR4 | −2.19 |
| 8123739 | 829 | NM_016588 | 1420 | 3230, 3454, 7755, 5387, | NRN1 | −2.16 |
| 7964745 | 830 | NM_016056 | 1421 | 3443, 2942, 4069, 7063, 6210, 5607, 7161, | TMBIM4 | −2.11 |
| 8130867 | 831 | NM_003247 | 1422 | 4184, 3540, 8063, 7661, 5992, 6904, | THBS2 | −2.05 |
| 8003298 | 832 | NM_003486 | 1423 | 2755, 3870, 5397, | SLC7A5 | −1.92 |
| 8172022 | 833 | NM_031442 | 1424 | 3197, 4343, 5466, | TMEM47 | −1.90 |

Table 6: Provided are the description of the membranal genes along with the sequence identifiers of the Affymetrix probes, the target sequence identified by the Affymetrix probe, and the mRNA sequences represented by the target sequences. Also shown are the log 2 fold change in expression levels of the indicated genes between the PDX1-GFP cells and the genetically unmodified HESCs. The (+) or (−) sign means that the genes are upregulated or downregulated, respectively, in the PDX1-GFP cells as compared to the genetically unmodified HESCs.

TABLE 7
Non-membranal genes which are upregulated in PDX1 cells as compared to genetically un-modified ESCs

| | Affy. target | | SEQ ID NO: | | | Log 2 fold |
|---|---|---|---|---|---|---|
| Probe | nucl. SEQ | Rep. | (Of Rep. | Polyn. SEQ ID NOs: | Gene | change |
| Set ID | ID NO: | Public ID | Public ID) | rep. by target | Symbol | [PDX1-HESC] |
| 8127522 | 834 | NM_001025290 | 1425 | 4388, 5232, 5916, | DPPA5 | 5.21 |
| 8049128 | 835 | NM_031313 | 1426 | 2557, 4233, 5696, | ALPPL2 | 5.18 |
| 7917037 | 836 | NM_001889 | 1427 | 3420, 2605, 3199, 3619, 4456, 4457, 4458, | CRYZ | 4.64 |
| | | | | 4612, 6114, 6052, 6863, 6288, 6530, 6018, | | |
| | | | | 8189, | | |
| 7945680 | 837 | NR_030533 | 1428 | 3526, 4930, 3779, 6619, 7610, 6652, 6726, | H19 | 4.26 |
| | | | | 6561, 7131, 6743, 7010, 6680, 6895, 7340, | | |
| | | | | 7012, 6491, | | |
| 7901497 | 838 | NM_001004339 | 1429 | 3328, 4808, 6317, 5872, | ZYG11A | 3.74 |
| 7902345 | 839 | NM_001162916 | 1430 | 3648, 4873, 4874, 4872, 8405, 6496, 6385, | TYW3 | 3.33 |
| | | | | 8689, 7110, 8780, 6273, | | |
| 7986350 | 840 | NM_183376 | 1431 | 3385, 4436, 5479, | ARRDC4 | 3.28 |
| 7904726 | 841 | NM_006472 | 1432 | 2648, 4356, 6092, 8025, 8646, 8023, | TXNIP | 3.08 |
| 7975779 | 842 | NM_005252 | 1433 | 3348, 4888, 5787, | FOS | 3.07 |
| 7959016 | 843 | NR_027345 | 1434 | 4799, 4800, 7778, 8557, | NCRNA00173 | 2.72 |
| 8071809 | 844 | NM_000854 | 1435 | 2877, 3667, 4143, 4144, 5682, 5316, | GSTT2 | 2.63 |
| 8074962 | 845 | NM_000854 | 1436 | 2877, 3667, 4143, 4144, 5682, 5316, | GSTT2 | 2.63 |
| 8033257 | 846 | NM_000064 | 1437 | 4194, 4038, 7636, 8397, 8664, 8376, 5358, | C3 | 2.60 |
| | | | | 6890, 6959, 7466, 8745, | | |
| 8120679 | 847 | NM_018665 | 1438 | 2820, 2957, 4749, 6125, 7145, 8551, 6076, | DDX43 | 2.58 |
| 7983360 | 848 | NM_004048 | 1439 | 3076, 3490, 6485, 6270, | B2M | 2.53 |
| 7949124 | 849 | NM_001164716 | 1440 | 2730, 4906, 4290, 8065, 5323, | PYGM | 2.47 |
| 8026424 | 850 | NM_173483 | 1441 | 3826, 4241, 5547, | CYP4F22 | 2.46 |
| 8030944 | 851 | NM_001099694 | 1442 | 4546, 4201, | ZNF578 | 2.44 |
| 7953532 | 852 | NM_001975 | 1443 | 3363, 2973, 5374, | ENO2 | 2.44 |
| 7933092 | 853 | NM_021045 | 1444 | 4114, 3320, 8456, 7285, 8408, 7542, 7283, | ZNF248 | 2.43 |
| | | | | 7447, 6137, | | |
| 8031576 | 854 | NM_134444 | 1445 | 3031, 4444, 5611, 5951, | NLRP4 | 2.39 |
| 7964246 | 855 | NR_003046 | 1446 | 3964, | SNORD59B | 2.34 |
| 7976812 | 856 | NR_003232 | 1447 | 4042, | SNORD113-4 | 2.34 |
| 7912347 | 857 | NM_001079843 | 1448 | 3894, 4091, 8716, 7616, | CASZ1 | 2.28 |
| 8008885 | 858 | NR_029493 | 1449 | 3773, 4929, | MIR21 | 2.27 |
| 7906400 | 859 | NM_005531 | 1450 | 3138, 2735, 2792, 2582, 3114, 4519, 2986, | IFI16 | 2.24 |
| | | | | 4028, 5903, 7297, 7065, 5990, 6176, 8454, | | |
| | | | | 7984, 8195, 7186, 7588, 5913, 5739, | | |
| 8033767 | 860 | NM_152476 | 1451 | 2982, 4116, 5683, | ZNF560 | 2.22 |
| 7930631 | 861 | NM_198795 | 1452 | 3518, 3513, 6133, 6452, 5445, 6004, 5919, | TDRD1 | 2.22 |
| 8139820 | 862 | NM_178558 | 1453 | 3061, 4448, 4449, 8320, 6601, 8790, 5658, | ZNF680 | 2.20 |
| 8118314 | 863 | NM_005346 | 1454 | 2997, 3529, 4339, 4461, 7351, 7433, 6971, | HSPA1B | 2.15 |
| | | | | 6486, 6386, 6633, 6951, 7233, 6772, 7683, | | |
| | | | | 6519, 6662, | | |
| 8118310 | 864 | NM_005345 | 1455 | 4504, 3360, 4461, 6563, 7351, 6971, 6630, | HSPA1A | 2.14 |
| | | | | 7449, 7498, 6386, 6951, 6668, 6772, 7683, | | |
| | | | | 7557, 6519, | | |
| 8108370 | 865 | NM_001964 | 1456 | 2571, 3296, 5376, 7583, | EGR1 | 2.09 |
| 7949104 | 866 | NM_001098670 | 1457 | 2786, 2787, 2742, 3987, 2749, 3104, 3106, | RASGRP2 | 2.07 |
| | | | | 3428, 4186, 4187, 3200, 6996, 7253, 7251, | | |
| | | | | 6422, 6110, 6605, 7167, 6721, 6400, 7783, | | |
| | | | | 7408, 7101, 6992, 8419, 6612, 7675, 7336, | | |
| | | | | 6494, 7993, 7692, 6747, 7413, | | |
| 8177011 | 867 | NM_004192 | 1458 | 3533, 5004, 5005, 5006, 7168, 8058, 7484, | ASMTL | 2.06 |
| | | | | 7559, 6621, 7587, | | |
| 8030448 | 868 | NM_001136052 | 1459 | 3049, 5236, 5237, 7371, 6022, 7446, 5780, | CPT1C | 2.05 |
| | | | | 5691, | | |
| 8024712 | 869 | NM_033064 | 1460 | 3258, 3101, 4560, 3035, 4405, 5929, 7505, | ATCAY | 2.05 |
| | | | | 5849, 7325, | | |
| 8008321 | 870 | NM_025149 | 1461 | 4485, 4216, 8568, 8657, 7865, 8078, 8132, | ACSF2 | 2.05 |
| | | | | 8703, 8080, 8098, 8518, 8485, 5631, 8002, | | |
| | | | | 8231, 8654, 8238, 8203, 8085, | | |
| 7947512 | 871 | NM_015430 | 1462 | 3786, 3701, 3702, 6790, 5537, 6920, | PAMR1 | 2.04 |
| 7963970 | 872 | NM_006928 | 1463 | 3105, 5245, 6941, 6287, | SILV | 2.03 |
| 7941662 | 873 | NM_001104 | 1464 | 3432, 2761, 8043, 7856, 8550, | ACTN3 | 2.03 |
| 7919787 | 874 | NM_032132 | 1465 | 3665, 3069, 3254, 3722, 5240, 8304, 6029, | HORMAD1 | 2.03 |
| | | | | 6113, 5998, 7030, 5874, 5867, 8731, 6466, | | |
| | | | | 5973, | | |
| 7993756 | 875 | NM_005622 | 1466 | 4281, 2813, 3634, 3635, 4670, 6949, 5609, | ACSM3 | 2.02 |
| | | | | 7003, 6291, | | |
| 8018652 | 876 | NM_052916 | 1467 | 4746, 5562, 5570, 5845, | RNF157 | 2.02 |
| 8066117 | 877 | NM_015474 | 1468 | 3449, 5023, 3830, 5349, 5541, 7106, 6100, | SAMHD1 | 2.01 |
| 7968928 | 878 | NM_002901 | 1469 | 3429, 3373, 4745, 4663, 7615, 8433, 8540, | RCN1 | 2.01 |
| | | | | 8791, 5816, 7507, 7679, 7940, 5326, | | |

Table 7: Provided are the description of the non-membranal genes along with the sequence identifiers of the Affymetrix probes, the target sequence identified by the Affymetrix probe, and the mRNA sequences represented by the target sequences. Also shown are the log 2 fold change in expression levels of the indicated genes between the PDX1-GFP cells and the genetically unmodified HESCs. The (+) or (−) sign means that the genes are upregulated or downregulated, respectively, in the PDX1-GFP cells as compared to the genetically unmodified HESCs.

TABLE 8
Non-membranal genes which are down regulated in PDX1 cells as compared to genetically un-modified ESCs

| | Affymetrix | | | | | Log 2 |
|---|---|---|---|---|---|---|
| | target nucl. | | SEQ ID | Polyn. | | fold change |
| Probe | sequence | Rep. | NO: (Of Rep. | SEQ ID NOs: | Gene | [PDX1- |
| Set ID | SEQ ID NO: | Public ID | Public ID) | rep. by target | Symbol | HESC] |
| 7957458 | 879 | NM_006183 | 1470 | 2948, 5242, 5429, | NTS | −6.28 |
| 8160163 | 880 | NM_005454 | 1471 | 3880, 3883, 6763, | CER1 | −5.61 |
| 8065412 | 881 | NM_001898 | 1472 | 3369, 3033, 5734, 6511, | CST1 | −5.17 |
| 7934156 | 882 | NM_018055 | 1473 | 3088, 4747, 7082, 5642, | NODAL | −4.21 |
| 7907222 | 883 | NM_022716 | 1474 | 4004, 3771, 3772, 5361, 6117, 8431, 7764, | PRRX1 | −3.87 |
| | | | | 8243, 8582, | | |
| 8067839 | 884 | NM_002009 | 1475 | 3477, 2587, 4695, 4203, 5525, 7263, | KGFLP1 | −3.72 |
| 8160138 | 885 | NM_005596 | 1476 | 3285, 2688, 2685, 4244, 4562, 2874, 3417, | NFIB | −3.49 |
| | | | | 5146, 5145, 5147, 7613, 7638, 6784, 7684, | | |
| | | | | 7327, 6514, 7007, 8444, 7463, | | |
| 8112668 | 886 | NM_016591 | 1477 | 2806, 4600, 5633, | GCNT4 | −3.42 |
| 7910134 | 887 | NM_031944 | 1478 | 2853, 2923, 6314, | MIXL1 | −3.41 |
| 8135594 | 888 | NM_001753 | 1479 | 4248, 4999, 5000, 5001, 5002, 6085, 6753, | CAV1 | −3.37 |
| | | | | 8406, | | |
| 7965403 | 889 | NM_002345 | 1480 | 2920, 3798, 5519, | LUM | −3.36 |
| 8106660 | 890 | NM_006909 | 1481 | 4058, 5302, 8643, 5518, 8502, 8537, 8352, | RASGRF2 | −3.34 |
| 8111892 | 891 | NM_000436 | 1482 | 2674, 4026, 8292, 5313, 8538, 8361, 8084, | OXCT1 | −3.25 |
| | | | | 7753, | | |
| 7957260 | 892 | NM_007043 | 1483 | 2953, 3451, 4074, 4007, 6328, 5493, 5382, | GLIPR1 | −3.25 |
| 8040430 | 893 | NM_003385 | 1484 | 3384, 3838, 6777, 6629, 8659, 5837, 6389, | VSNL1 | −3.24 |
| | | | | 6436, | | |
| 8105302 | 894 | NM_013409 | 1485 | 3343, 5282, 5281, 7924, 7913, 6382, 5408, | FST | −3.15 |
| 7971077 | 895 | NM_006475 | 1486 | 2617, 4640, 4641, 4642, 4649, 7522, 8474, | POSTN | −2.97 |
| | | | | 8449, 7103, 8183, 7375, 6642, | | |
| 8161892 | 896 | NM_004297 | 1487 | 2753, 4750, 6030, 7575, | GNA14 | −2.96 |
| 8068401 | 897 | NM_001757 | 1488 | 3890, 3754, 7622, 5848, | CBR1 | −2.95 |
| 8092095 | 898 | NM_015028 | 1489 | 4193, 4852, 4853, 4854, 4855, 4856, 4857, | TNIK | −2.95 |
| | | | | 4858, 4859, 4860, 8054, 5939, 6549, 7883, | | |
| | | | | 5603, 6786, 7448, 7215, 8447, 8382, 7882, | | |
| | | | | 6219, | | |
| 7921916 | 899 | NM_001195303 | 1490 | 3306, 5190, 5110, 7452, 5828, 6057, | RGS5 | −2.93 |
| 8155487 | 900 | NM_002009 | 1491 | 3476, 2587, 2975, 4695, 4203, 5525, 7263, | KGFLP1 | −2.89 |
| | | | | 7112, 6748, | | |
| 7952601 | 901 | NM_001162422 | 1492 | 3402, 4871, 4686, 4687, 7425, 5612, 5931, | ETS1 | −2.88 |
| 7947274 | 902 | NM_001584 | 1493 | 2671, 4776, 4777, 6358, 6060, | MPPED2 | −2.79 |
| 7934906 | 903 | NM_001141945 | 1494 | 3670, 3812, 3774, 2556, 3442, 4717, 4473, | ACTA2 | −2.79 |
| | | | | 2836, 3380, 4664, 4665, 5340, 8239, 7483, | | |
| | | | | 8264, 7450, 7803, 6754, | | |
| 8140668 | 904 | NM_006080 | 1495 | 4019, 3973, 5554, 7046, 6980, | SEMA3A | −2.72 |
| 7974689 | 905 | NM_016651 | 1496 | 2815, 4080, 4079, | DACT1 | −2.67 |
| 8155849 | 906 | NM_000700 | 1497 | 3181, 2757, 6327, 5394, 7974, 6460, 7578, | ANXA1 | −2.66 |
| | | | | 7227, 8129, 8580, | | |
| 8127563 | 907 | NM_080645 | 1498 | 4761, 3954, 3953, 5758, 6009, 7798, 8346, | COL12A1 | −2.59 |
| | | | | 8170, 5484, 6823, | | |
| 7965322 | 908 | NM_000899 | 1499 | 4054, 5149, 5148, 6216, 5328, 6318, 7574, | KITLG | −2.58 |
| 7995681 | 909 | NM_004530 | 1500 | 3362, 4409, 4410, 5329, 6518, | MMP2 | −2.57 |
| 7971653 | 910 | NR_002612 | 1501 | 2849, 3911, 7223, 6445, 7650, 7147, 7126, | DLEU2 | −2.57 |
| | | | | 7028, 6609, | | |
| 8171449 | 911 | NM_021804 | 1502 | 3847, 4462, 6787, 7949, 7759, 7328, 5427, | ACE2 | −2.56 |
| 8042788 | 912 | NM_001615 | 1503 | 2956, 3832, 6377, 7341, 6703, 6599, 7930, | ACTG2 | −2.56 |
| | | | | 5891, | | |
| 7962427 | 913 | NM_031292 | 1504 | 3592, 4173, 4172, 4174, 6664, 6050, 6684, | PUS7L | −2.52 |
| 7984259 | 914 | NR_002757 | 1505 | 4615, | RNU5B-1 | −2.40 |
| 8046922 | 915 | NM_000090 | 1506 | 3042, 4000, 7681, 5702, 7724, 5577, | COL3A1 | −2.37 |
| 7924682 | 916 | NM_003240 | 1507 | 3558, 4989, 4988, 8493, 7506, 6104, | LEFTY2 | −2.35 |
| 7912520 | 917 | NM_002521 | 1508 | 3029, 3909, 7941, 6388, | NPPB | −2.35 |
| 7944867 | 918 | NM_170601 | 1509 | 3593, 5256, 5538, | SIAE | −2.30 |
| 8095646 | 919 | NM_001134 | 1510 | 3041, 2756, 7886, 7273, 8047, 8272, | AFP | −2.29 |
| 8098690 | 920 | NM_174900 | 1511 | 3498, 3937, 5793, 8300, | ZFP42 | −2.29 |
| 8152617 | 921 | NM_005328 | 1512 | 2676, 4348, 5843, 6409, | HAS2 | −2.28 |
| 8136807 | 922 | — | | | TRY6 | −2.28 |
| 7908597 | 923 | NM_003822 | 1513 | 2737, 3582, 3583, 6058, 5356, 8618, 7477, | NR5A2 | −2.27 |
| | | | | 6293, 5367, | | |
| 8115543 | 924 | NM_024007 | 1514 | 3190, 4342, 8173, 8061, 8377, 5613, 8417, | EBF1 | −2.27 |
| | | | | 6926, 7796, 8496, 7892, | | |
| 7977507 | 925 | NR_002312 | 1515 | 3839, | RPPH1 | −2.26 |
| 8026139 | 926 | NM_002501 | 1516 | 4021, 4022, 3743, 3307, 2629, 2644, 2645, | NFIX | −2.21 |
| | | | | 4501, 4543, 4907, 3764, 5981, 6837, 5556, | | |
| | | | | 6879, 5925, | | |
| 8040163 | 927 | NM_001039613 | 1517 | 3912, 3107, 4261, 4549, 3207, 3482, 3588, | IAH1 | −2.20 |
| | | | | 3934, 6534, 8330, 8409, 8205, 6128, 8770, | | |
| | | | | 8556, 7901, 8340, 8747, 8755, 7721, 8381, | | |
| | | | | 8091, 7817, 5835, | | |
| 8151768 | 928 | NM_004349 | 1518 | 2705, 3651, 2626, 2602, 2625, 2624, 2654, | RUNX1T1 | −2.20 |
| | | | | 4142, 3323, 2686, 4712, 2904, 3581, 5209, | | |
| | | | | 5210, 5211, 5212, 5213, 5214, 5215, 5216, | | |
| | | | | 5217, 5218, 5219, 5220, 5221, 5222, 5223, | | |
| | | | | 8013, 6461, 5898, 7238, 8282, 8648, 8373, | | |
| | | | | 8771, 8423, 8210, 8363, 8306, 8545, 8741, | | |
| | | | | 8410, 8779, 8219, 7874, 8339, 8400, 8131, | | |
| | | | | 8197, 8357, 7910, 8124, 8636, 7994, 7908, | | |
| | | | | 7863, 8687, 8792, 8763, 7842, 8594, 8665, | | |
| | | | | 8145, 8274, 8404, 8092, 8089, 8443, 7757, | | |
| | | | | 8719, 5496, 6644, | | |
| 8057620 | 929 | NM_000393 | 1519 | 3762, 3939, 7320, 7380, 7573, | COL5A2 | −2.18 |
| 8106743 | 930 | NM_004385 | 1520 | 2643, 4889, 4890, 4891, 4892, 6221, 7925, | VCAN | −2.17 |
| | | | | 8699, 7773, 8196, 5495, 8365, 6091, | | |
| 7897801 | 931 | M77839 | 1521 | 2554, 4136, | RNU5E | −2.14 |
| 8069689 | 932 | NM_007038 | 1522 | 2778, 4585, 5725, | ADAMTS5 | −2.14 |
| 8140955 | 933 | NM_001145306 | 1523 | 3279, 4773, 4772, 5552, 8113, 8064, 7668, | CDK6 | −2.11 |
| | | | | 6346, | | |
| 8135218 | 934 | NM_005824 | 1524 | 2666, 4848, 4849, 5940, 5451, 7410, 8602, | LRRC17 | −2.11 |
| 8163716 | 935 | NM_014618 | 1525 | 2726, 4469, 4474, 3012, 3546, 3649, 4164, | DBC1 | −2.11 |
| | | | | 6292, 6191, 5480, | | |
| 8007921 | 936 | NM_001002841 | 1526 | 3053, 3703, 2998, 6007, 6931, | MYL4 | −2.10 |
| 7934979 | 937 | NM_014391 | 1527 | 2995, 3511, 5932, | ANKRD1 | −2.09 |
| 8085946 | 938 | NM_005442 | 1528 | 3171, 3140, 5868, 7137, 7635, | EOMES | −2.07 |
| 7971461 | 939 | NM_002298 | 1529 | 2593, 2594, 3108, 3126, 3127, 4472, 2928, | LCP1 | −2.05 |
| | | | | 2972, 4589, 6343, 5723, 6824, 7508, 6347, | | |
| | | | | 6695, 8668, 6816, | | |
| 7944082 | 940 | NM_003186 | 1530 | 2895, 3656, 3657, 3038, 7198, 5531, 6894, | TAGLN | −2.04 |
| | | | | 5715, | | |
| 8045688 | 941 | NM_007115 | 1531 | 3052, 5234, 5352, 6810, | TNFAIP6 | −2.04 |
| 8105607 | 942 | NM_001164442 | 1532 | 4893, 7805, 7948, 7918, 8707, 6538, | FAM159B | −2.03 |
| 8161520 | 943 | NM_021965 | 1533 | 3077, 4232, 3852, 6909, 6516, | | |

Table 8: Provided are the description of the non-membranal genes along with the sequence identifiers of the Affymetrix probes, the target sequence identified by the Affymetrix probe, and the mRNA sequences represented by the target sequences. Also shown are the log 2 fold change in expression levels of the indicated genes between the PDX1-GFP cells and the genetically unmodified HESCs. The (+) or (−) sign means that the genes are upregulated or downregulated, respectively, in the PDX1-GFP cells as compared to the genetically unmodified HESCs.

### Example 3

### Genes which are Upregulated or Downregulated in Pancreatic Progenitor (PDX1+) Versus Definite Endoderm (SOX17+) Cells

TABLE 9
Membranal genes which are upregulated in PDX1 cells as compared to SOX17 cells

| | | | SEQ ID | | | |
|---|---|---|---|---|---|---|
| | Affy. target | | NO: (Of | | | Log 2 |
| Probe | nucl. SEQ | Rep. | Rep. Pub- | Polyn. SEQ ID NOs: | Gene | fold change |
| Set ID | ID NO: | Public ID | lic ID) | rep. by target | Symbol | [PDX-SOX] |
| 8100393 | 944 | NM_002253 | 1534 | 2708, 4588, 7921, 5498, 8228, | KDR | 4.62 |
| 8051785 | 945 | NM_133329 | 1535 | 3647, 5227, 5228, 5783, 7561, | KCNG3 | 4.19 |
| 8031076 | 946 | NM_031896 | 1536 | 3827, 5226, 7212, 5339, | CACNG7 | 3.80 |
| 8096440 | 947 | NM_001510 | 1537 | 2719, 4235, 8429, 5557, 7933, 8154, | GRID2 | 3.63 |
| 7996837 | 948 | NM_004360 | 1538 | 4149, 4182, 2614, 4278, 4556, 2794, 2795, | CDH1 | 3.45 |
| | | | | 4415, 4294, 4315, 4347, 5506, 7453, 5404, | | |
| | | | | 7133, | | |
| 7917276 | 949 | NM_012152 | 1539 | 2780, 4363, 7551, 8539, 6011, | LPAR3 | 3.33 |
| 8113666 | 950 | NM_020796 | 1540 | 2844, 4722, 3122, 3148, 4310, 4145, 8137, | SEMA6A | 3.29 |
| | | | | 8152, 7900, 5499, 8724, 8667, 8467, 8111, | | |
| | | | | 5437, 8321, 7896, 5978, 8764, | | |
| 8135774 | 951 | NM_002851 | 1541 | 2599, 3943, 7457, 8030, 7815, 8011, 8237, | PTPRZ1 | 3.15 |
| | | | | 7646, | | |
| 8037079 | 952 | NM_152296 | 1542 | 3345, 3672, 8109, 6417, 5616, 7816, | ATP1A3 | 3.09 |
| 8087530 | 953 | NM_024046 | 1543 | 3407, 4516, 4542, 4547, 4568, 3355, 2883, | CAMKV | 3.04 |
| | | | | 3337, 3368, 3000, 3165, 3910, 8007, 8299, | | |
| | | | | 6547, 8504, 8573, 8756, 8739, 7861, 8075, | | |
| | | | | 7731, 6769, 7182, 8603, 8099, 7254, 5602, | | |
| 7994058 | 954 | NM_001039 | 1544 | 3481, 4177, 5784, | SCNN1G | 3.00 |
| 7918716 | 955 | NM_205848 | 1545 | 2981, 3239, 4010, 6234, 6167, 7976, 6283, | SYT6 | 2.92 |
| | | | | 6937, 6744, 6657, | | |
| 7930837 | 956 | NM_003054 | 1546 | 3141, 5107, 7960, 5655, | SLC18A2 | 2.87 |
| 8108697 | 957 | NM_015669 | 1547 | 2872, 2931, 5379, | PCDHB5 | 2.84 |
| 8101675 | 958 | NM_004827 | 1548 | 3811, 3819, 7943, 5391, | ABCG2 | 2.61 |
| 8178193 | 959 | NM_019111 | 1549 | 3652, 4537, 2590, 2589, 4532, 4478, 4721, | HLA-DRA | 2.60 |
| | | | | 4482, 3074, 3666, 5174, 6840, 8271, 7495, | | |
| | | | | 7301, 6340, 7125, 7667, 6973, 6698, 6694, | | |
| | | | | 7895, 6462, 7070, 8427, 7321, 6682, 7906, | | |
| | | | | 7421, 7648, 7487, 6525, | | |
| 7916584 | 960 | NM_002353 | 1550 | 2675, 4337, 6094, | TACSTD2 | 2.57 |
| 7909390 | 961 | NM_175710 | 1551 | 3897, 4185, 8470, 6548, 5851, | CR1L | 2.54 |
| 8169447 | 962 | NM_000868 | 1552 | 2609, 4287, 5920, 5564, 5986, | HTR2C | 2.48 |
| 7972157 | 963 | NM_003991 | 1553 | 2606, 5247, 5246, 5248, 5597, 7819, 7369, | EDNRB | 2.47 |
| 8168657 | 964 | NM_001168362 | 1554 | 3001, 2888, 4950, 4951, 2807, 4945, 4946, | PCDH11X | 2.45 |
| | | | | 4947, 4948, 4949, 2939, 2940, 2938, 5762, | | |
| | | | | 6189, 5311, 6640, 6106, 6184, 6241, 5877, | | |
| | | | | 7081, 5601, 6247, 8389, | | |
| 8038367 | 965 | NM_020309 | 1555 | 3483, 4743, 5332, | SLC17A7 | 2.44 |
| 7960529 | 966 | NM_001159575 | 1556 | 3514, 4813, 4814, 4812, 5353, 6068, 6294, | SCNN1A | 2.35 |
| | | | | 6143, 6414, | | |
| 7953291 | 967 | NM_001769 | 1557 | 3817, 5250, 7598, 7601, 7771, 7211, 6952, | CD9 | 2.35 |
| | | | | 5319, | | |
| 8011713 | 968 | NM_022059 | 1558 | 2851, 4210, 4211, 5728, | CXCL16 | 2.29 |
| 8027778 | 969 | NM_144779 | 1559 | 3228, 3470, 2828, 3419, 3286, 4528, 3008, | FXYD5 | 2.25 |
| | | | | 3388, 4895, 4896, 4898, 4897, 6381, 7395, | | |
| | | | | 6565, 5906, 7311, 8512, | | |
| 8170538 | 970 | NM_018558 | 1560 | 2811, 4939, 6037, 5850, | GABRQ | 2.25 |
| 8114415 | 971 | NM_001496 | 1561 | 3167, 3584, 7363, 5508, | GFRA3 | 2.23 |
| 8087691 | 972 | NM_001174051 | 1562 | 2721, 2711, 2712, 4220, 4298, 5019, 3735, | CACNA2D2 | 2.23 |
| | | | | 3736, 6362, 6812, 5540, 6239, 8681, 7132, | | |
| | | | | 7985, 7734, 6957, 7200, | | |
| 8133360 | 973 | NM_001305 | 1563 | 3437, 3401, 7342, 6124, 7250, | CLDN4 | 2.21 |
| 8059186 | 974 | NM_002846 | 1564 | 2647, 5233, 5755, 7545, 6502, 6839, 8004, | PTPRN | 2.20 |
| | | | | 8090, 8318, 8312, 7916, | | |
| 8169061 | 975 | NM_001128834 | 1565 | 3671, 3836, 3741, 3742, 2596, 2595, 2588, | PLP1 | 2.20 |
| | | | | 4267, 4502, 4503, 2878, 4324, 4329, 4438, | | |
| | | | | 3554, 3553, 8469, 6590, 7115, 7231, 7458, | | |
| | | | | 6933, 7196, 7209, 8108, 8781, 8623, 8141, | | |
| | | | | 8486, 7855, 5832, 8385, 5923, 8694, 6335, | | |
| | | | | 7709, 6348, 8343, 7178, 7629, | | |
| 7898057 | 976 | NM_001006624 | 1566 | 3236, 2746, 2747, 3084, 3519, 2965, 3133, | PDPN | 2.13 |
| | | | | 4305, 4306, 3747, 3748, 3745, 3746, 8458, | | |
| | | | | 8786, 8213, 5840, 7528, 8392, 8050, 7659, | | |
| | | | | 7822, 8446, | | |
| 8062123 | 977 | NM_006690 | 1567 | 2775, 3872, 5355, | MMP24 | 2.13 |
| 8018114 | 978 | NM_001144952 | 1568 | 2814, 4748, 7774, 5765, 6851, 6326, | SDK2 | 2.12 |
| 7987439 | 979 | NM_007223 | 1569 | 3580, 2783, 5852, | GPR176 | 2.10 |
| 8171624 | 980 | NM_005756 | 1570 | 3176, 3177, 3178, 3179, 3216, 3862, 3994, | GPR64 | 2.09 |
| | | | | 3992, 4253, 3172, 3173, 3174, 3093, 5123, | | |
| | | | | 5124, 5125, 5126, 5127, 5128, 5129, 5130, | | |
| | | | | 5131, 6034, 6617, 6614, 8617, 6938, 6296, | | |
| | | | | 6048, 6115, 6013, 6956, 6659, 6099, | | |
| 8083839 | 981 | NM_014373 | 1571 | 2885, 4317, 4609, 8290, 8552, 8316, 8057, | GPR160 | 2.07 |
| | | | | 7844, 7988, 6244, 7922, | | |
| 8176400 | 982 | NM_001168362 | 1572 | 3001, 2889, 4950, 2807, 4945, 4947, 2939, | PCDH11Y | 2.07 |
| | | | | 2940, 2938, 5762, 6189, 5311, 6640, 6184, | | |
| | | | | 6241, 5877, 7081, | | |
| 8067602 | 983 | NM_152864 | 1573 | 3226, 4323, 4809, 6602, 6260, 6027, 8666, | NKAIN4 | 2.06 |
| | | | | 7541, 6149, 6332, 8610, 6274, | | |
| 8004545 | 984 | NM_001678 | 1574 | 2604, 3681, 5449, | ATP1B2 | 2.05 |
| 7955502 | 985 | NM_001177984 | 1575 | 2769, 5108, 4009, 6054, 6237, 6251, | SCN8A | 2.03 |
| 8106573 | 986 | NM_003248 | 1576 | 3271, 5018, 8753, 6398, 8163, 6313, | THBS4 | 2.01 |
| 7909350 | 987 | NM_001006658 | 1577 | 4488, 4916, 4917, 6275, 5909, 8223, 8585, | CR2 | 2.01 |
| | | | | 6171, 6814, | | |
| 8118556 | 988 | NM_002122 | 1578 | 3810, 3721, 7313, 8208, 7034, 6552, 7074, | HLA-DQA1 | 1.98 |
| | | | | 5946, 8475, | | |
| 8118548 | 989 | NM_019111 | 1579 | 3652, 4537, 2590, 2589, 4532, 4478, 4721, | HLA-DRA | 1.97 |
| | | | | 4482, 3074, 3666, 5174, 6840, 8271, 7495, | | |
| | | | | 7301, 6340, 7125, 7667, 6973, 6698, 6694, | | |
| | | | | 7895, 6462, 7070, 8427, 7321, 6682, 7906, | | |
| | | | | 7421, 7648, 7487, 6525, | | |
| 7934970 | 990 | NM_000872 | 1580 | 3256, 4604, 4605, 4606, 5950, 6214, 5736, | HTR7 | 1.92 |
| | | | | 5523, | | |
| 7915472 | 991 | NM_006516 | 1581 | 4268, 4338, 5901, 6869, 6399, 8613, 6344, | SLC2A1 | 1.92 |
| | | | | 6364, 5884, 7600, | | |
| 8179481 | 992 | NM_019111 | 1582 | 3652, 4537, 2590, 2589, 4532, 4478, 4721, | HLA-DRA | 1.92 |
| | | | | 4482, 3074, 3666, 5174, 6840, 8271, 7495, | | |
| | | | | 7301, 6340, 7125, 7667, 6973, 6698, 6694, | | |
| | | | | 7895, 6462, 7070, 8427, 7321, 6682, 7906, | | |
| | | | | 7421, 7648, 7487, 6525, | | |
| 8040458 | 993 | NM_002252 | 1583 | 3347, 3213, 6867, 6984, 8229, 5665, 7423, | KCNS3 | 1.91 |
| 8173414 | 994 | NM_032803 | 1584 | 3087, 5135, 5134, 5854, 7474, | SLC7A3 | 1.86 |
| 8118607 | 995 | NR_001435 | 1585 | 4480, 2993, 3281, 6755, 8176, 8031, 7000, | HLA-DPB2 | 1.86 |
| | | | | 5720, 7237, 8785, 7391, 8295, 6363, 7378, | | |
| | | | | 8526, 7569, 6929, 7023, | | |
| 8159734 | 996 | NM_000718 | 1586 | 2559, 2560, 5298, 6089, 5532, 5955, 6280, | CACNA1B | 1.85 |
| | | | | 6151, 5911, 5558, 6198, | | |
| 8171284 | 997 | NM_000273 | 1587 | 3596, 4944, 6448, 6849, 7049, 7224, | | |

Table 9: Provided are the description of the membranal genes along with the sequence identifiers of the Affymetrix probes, the target sequence identified by the Affymetrix probe, and the mRNA sequences represented by the target sequences. Also shown are the log 2 fold change in expression levels of the indicated genes between the PDX1-GFP cells and the SOX17-GFP cells (PDX1-SOX17). The (+) or (−) sign means that the genes are upregulated or downregulated, respectively, in the PDX1-GFP cells as compared to the SOX17-GFP cells.

TABLE 10
Membranal genes which are downregulated in PDX1 cells as compared to SOX17 cells

| | | | SEQ ID | | | Log 2 |
|---|---|---|---|---|---|---|
| | Affy. target | | NO: (Of | | | fold change |
| Probe Set | nucl. SEQ | Rep. | Rep. Pub- | Polyn. SEQ ID NOs: | Gene | [PDX- |
| ID | ID NO: | Public ID | lic ID) | rep. by target | Symbol | SOX17] |
| 7957140 | 998 | NM_003667 | 1588 | 2732, 3193, 6795, 5509, | LGR5 | −6.13 |
| 7938090 | 999 | NM_176875 | 1589 | 2809, 5307, 5676, | CCKBR | −4.88 |
| 8055465 | 1000 | NM_001008540 | 1590 | 2782, 3775, 3776, 5384, 6834, 7645, | CXCR4 | −4.88 |
| 8065071 | 1001 | NM_198391 | 1591 | 2812, 5172, 5173, 5918, 7056, 6380, | FLRT3 | −4.44 |
| 8151341 | 1002 | NM_007332 | 1592 | 4060, 8269, 5433, | TRPA1 | −4.19 |
| 8057677 | 1003 | NM_014585 | 1593 | 3154, 4368, 6969, 5406, 7045, 8144, 8627, 7674, 6595, | SLC40A1 | −4.12 |
| | | | | 6989, | | |
| 7964602 | 1004 | NM_153377 | 1594 | 3457, 3537, 4639, 5656, 6683, 7048, | LRIG3 | −3.98 |
| 8021946 | 1005 | NM_130386 | 1595 | 2891, 4601, 6579, | COLEC12 | −3.86 |
| 7971296 | 1006 | NM_033255 | 1596 | 3367, 3694, 3693, 5738, 5732, 7664, 7742, | EPSTI1 | −3.83 |
| 8081298 | 1007 | NM_032787 | 1597 | 3995, 4847, 5470, 7733, 8308, 8445, | GPR128 | −3.63 |
| 8022674 | 1008 | NM_001792 | 1598 | 3134, 4668, 5546, | CDH2 | −3.38 |
| 7901316 | 1009 | NM_001011547 | 1599 | 4682, 4626, 4627, 6649, 6403, 7403, 7829, 6781, 6361, | SLC5A9 | −3.36 |
| 8052072 | 1010 | NM_000145 | 1600 | 4265, 5021, 5022, 6252, 5713, 7612, 7221, 6397, | FSHR | −3.33 |
| 7924342 | 1011 | NM_018713 | 1601 | 3493, 3718, 6268, 6130, 8273, 8216, | SLC30A10 | −3.27 |
| 8169580 | 1012 | NM_001560 | 1602 | 2683, 3219, 6256, 6220, 8730, 6320, | IL13RA1 | −3.20 |
| 7977786 | 1013 | NM_001126105 | 1603 | 2789, 5292, 5291, 7158, 7255, 5870, 8255, 6471, 7642, | SLC7A7 | −3.14 |
| 8097449 | 1014 | NM_032961 | 1604 | 2914, 2936, 2935, 7099, 5512, 8691, | PCDH10 | −3.11 |
| 8154135 | 1015 | NM_004170 | 1605 | 3070, 5025, 5401, 7218, 8331, | SLC1A1 | −3.07 |
| 8132347 | 1016 | NM_181791 | 1606 | 3313, 3318, 6342, 5701, 6692, | GPR141 | −3.05 |
| 8111677 | 1017 | NM_001127671 | 1607 | 2551, 4403, 4404, 5491, 7187, 7919, 8314, 7758, | LIFR | −2.98 |
| 8171472 | 1018 | NM_020665 | 1608 | 2960, 4942, 6430, | TMEM27 | −2.95 |
| 8175217 | 1019 | NM_001448 | 1609 | 2738, 3072, 6154, | GPC4 | −2.93 |
| 8045664 | 1020 | NM_177964 | 1610 | 3039, 3538, 8714, 5464, 7286, 6455, 7123, 7806, 7494, | LYPD6B | −2.91 |
| | | | | 8341, 7067, 6613, | | |
| 7948058 | 1021 | NM_153696 | 1611 | 3060, 3615, 2779, 2843, 3871, 2598, 4703, 4705, 4140, | FOLH1 | −2.90 |
| | | | | 4138, 2773, 3026, 3280, 5177, 5176, 2766, 5178, 3821, | | |
| | | | | 6173, 5921, 6307, 5425, 6469, 6968, | | |
| 7971104 | 1022 | NM_016179 | 1612 | 3415, 4643, 4644, 4645, 4646, 4647, 4648, 7469, 7085, | TRPC4 | −2.88 |
| | | | | 8143, 6069, 7054, 6227, 6264, 7998, 7627, 6564, 7066, | | |
| 8094520 | 1023 | NM_032457 | 1613 | 2723, 5014, 5015, 2937, 5262, 5768, 5971, | PCDH7 | −2.88 |
| 8143534 | 1024 | NM_000420 | 1614 | 2886, 2987, 6934, 8187, 8288, 6818, 6144, 8559, | KEL | −2.78 |
| 8045795 | 1025 | NM_002239 | 1615 | 2667, 3201, 5594, 8178, | KCNJ3 | −2.72 |
| 7911273 | 1026 | NM_001004696 | 1616 | 3717, 6107, | OR2T4 | −2.71 |
| 8165658 | 1027 | X59268 | 1617 | 2553, 3426, 3422, 6269, | VIPR2 | −2.68 |
| 7976073 | 1028 | NM_013231 | 1618 | 4059, 3816, 5667, | FLRT2 | −2.66 |
| 7923978 | 1029 | NM_001773 | 1619 | 2563, 3891, 3854, 5956, 6086, 5785, 6059, 8087, | CD34 | −2.64 |
| 8101992 | 1030 | NM_001135146 | 1620 | 2875, 4623, 4624, 3783, 4625, 7838, 6311, 8464, 6341, | SLC39A8 | −2.63 |
| | | | | 7047, | | |
| 8083887 | 1031 | NM_001185056 | 1621 | 2958, 5141, 4593, 7870, 5315, 6845, 7064, 7947, 8104, | CLDN11 | −2.57 |
| 8049471 | 1032 | NM_020311 | 1622 | 3396, 4030, 5457, | CXCR7 | −2.52 |
| 7963786 | 1033 | NM_002205 | 1623 | 3341, 3761, 5661, 6880, | ITGA5 | −2.48 |
| 8046861 | 1034 | NM_002210 | 1624 | 2615, 4762, 4763, 4764, 8678, 6893, 8003, 7665, 5446, | ITGAV | −2.44 |
| | | | | 6625, | | |
| 8140995 | 1035 | NM_001742 | 1625 | 2561, 4912, 4913, 4914, 6611, 6073, 7384, 7536, 6539, | CALCR | −2.43 |
| 8082928 | 1036 | NM_016369 | 1626 | 4183, 3788, 3787, 8686, 5317, 6211, | CLDN18 | −2.41 |
| 8003298 | 1037 | NM_003486 | 1627 | 2755, 3870, 5397, | SLC7A5 | −2.32 |
| 7964745 | 1038 | NM_016056 | 1628 | 3443, 2942, 4069, 7063, 6210, 5607, 7161, | TMBIM4 | −2.32 |
| 8090823 | 1039 | NM_005630 | 1629 | 2724, 4396, 7489, 6355, 8401, 8414, 8074, 5640, | SLCO2A1 | −2.29 |
| 8111255 | 1040 | NM_006727 | 1630 | 2788, 5142, 8497, 5507, 8253, 8371, | CDH10 | −2.29 |
| 7955797 | 1041 | NM_020547 | 1631 | 4056, 4904, 4903, 4905, 7400, 5395, | AMHR2 | −2.28 |
| 7952341 | 1042 | NM_024769 | 1632 | 3321, 3556, 7605, 6402, | ASAM | −2.26 |
| 8092726 | 1043 | NM_021101 | 1633 | 2752, 5133, 5652, 7810, | CLDN1 | −2.26 |
| 8070421 | 1044 | NM_001389 | 1634 | 2796, 3591, 6842, | DSCAM | −2.15 |
| 8004691 | 1045 | NM_203411 | 1635 | 3456, 3564, 5821, | TMEM88 | −2.15 |
| 7923991 | 1046 | NM_025179 | 1636 | 4111, 4029, 6303, 8403, 6499, | PLXNA2 | −2.14 |
| 8037298 | 1047 | NM_020406 | 1637 | 3744, 4005, 6408, 7480, 7666, | CD177 | −2.14 |
| 8098041 | 1048 | NM_018342 | 1638 | 2802, 3163, 3170, 3261, 3319, 3506, 3267, 4584, 8697, | TMEM144 | −2.13 |
| | | | | 8424, 5686, 8259, 8744, 8370, 7977, 8192, 7923, 7849, | | |
| | | | | 8039, 7954, 8413, 7830, | | |
| 8142687 | 1049 | NM_005302 | 1639 | 3208, 3301, 5833, | GPR37 | −2.09 |
| 7919340 | 1050 | NM_005266 | 1640 | 3364, 4104, 4105, 7691, 6103, 6550, 5474, | GJA5 | −2.09 |
| 7983527 | 1051 | NM_024966 | 1641 | 3078, 3079, 3080, 3081, 3082, 4192, 4279, 3184, 3186, | SEMA6D | −2.08 |
| | | | | 3187, 3185, 3188, 3183, 6040, 7595, 5709, 6809, 6877, | | |
| | | | | 7470, 6289, 5994, | | |
| 8151952 | 1052 | NM_024759 | 1642 | 4112, 2892, 8672, 8517, 8125, 6122, 8051, 8637, | NIPAL2 | −2.08 |
| 8114896 | 1053 | NM_194251 | 1643 | 3379, 3780, 5572, | GPR151 | −2.07 |
| 8113551 | 1054 | NM_002387 | 1644 | 3412, 4147, 4148, 8175, 8246, 5792, 7893, | MCC | −2.00 |
| 7944554 | 1055 | NM_174926 | 1645 | 4534, 5202, 5203, 5204, 5205, 5206, 5207, 5208, 6407, | TMEM136 | −1.98 |
| | | | | 5814, | | |
| 8067029 | 1056 | NM_002237 | 1646 | 2710, 3445, 4245, 2913, 3399, 4162, 8728, 6738, 7204, | KCNG1 | −1.94 |
| | | | | 6263, 7217, 6415, | | |
| 8112803 | 1057 | NM_005779 | 1647 | 3315, 4380, 4381, 3977, 8589, 8081, 8592, 8563, 6988, | LHFPL2 | −1.89 |
| | | | | 8101, | | |
| 8175288 | 1058 | NM_019556 | 1648 | 2903, 2824, 6997, 8283, 6003, 6290, 7788, 7752, 5881, | MOSPD1 | −1.87 |
| 8068810 | 1059 | NM_018964 | 1649 | 2845, 3683, 6723, 7548, 6976, 5984, 7385, 8095, | SLC37A1 | −1.86 |
| 8096839 | 1060 | NM_198506 | 1650 | 3439, 4440, 5693, 7042, 6600, | LRIT3 | −1.86 |
| 8059279 | 1061 | NM_004438 | 1651 | 3926, 3577, 6427, 7497, 5543, | EPHA4 | −1.84 |
| 7916862 | 1062 | NM_024911 | 1652 | 3473, 5169, 5171, 5170, 6985, 7944, 8040, 5423, 6217, | GPR177 | −1.83 |
| | | | | 5957, 7720, 6063, 8128, 6006, | | |
| 8043995 | 1063 | NM_000877 | 1653 | 2585, 2550, 2838, 3587, 3589, 3590, 3704, 3705, 4327, | IL1R1 | −1.80 |
| | | | | 3237, 7104, 5389, 7004, 6741, 7479, 7102, 7053, 7439, | | |
| | | | | 6674, 6715, 7705, 7500, 6803, 6643, | | |

Table 10: Provided are the description of the membranal genes along with the sequence identifiers of the Affymetrix probes, the target sequence identified by the Affymetrix probe, and the mRNA sequences represented by the target sequences. Also shown are the log 2 fold change in expression levels of the indicated genes between the PDX1-GFP cells and the SOX17-GFP cells (PDX1-SOX17). The (+) or (−) sign means that the genes are upregulated or downregulated, respectively, in the PDX1-GFP cells as compared to the SOX17-GFP cells.

TABLE 11
Non-membranal genes which are upregulated in PDX1 cells as compared to SOX17 cells

| | | | SEQ ID | | | Log 2 |
|---|---|---|---|---|---|---|
| | Affy. target | | NO: (Of | | | fold change |
| Probe | nucl. SEQ | Rep. | Rep. Pub- | Polyn. SEQ ID NOs: | Gene | [PDX- |
| Set ID | ID NO: | Public ID | lic ID) | rep. by target | Symbol | SOX17] |
| 8049128 | 1064 | NM_031313 | 1654 | 2557, 4233, 5696, | ALPPL2 | 5.61 |
| 8127522 | 1065 | NM_001025290 | 1655 | 4388, 5232, 5916, | DPPA5 | 5.51 |
| 7945680 | 1066 | NR_030533 | 1656 | 3526, 4930, 3779, 6619, 7610, 6652, 6726, 6561, 7131, | H19 | 5.26 |
| | | | | 6743, 7010, 6680, 6895, 7340, 7012, 6491, | | |
| 7917037 | 1067 | NM_001889 | 1657 | 3420, 2605, 3199, 3619, 4456, 4457, 4458, 4612, 6114, | CRYZ | 4.85 |
| | | | | 6052, 6863, 6288, 6530, 6018, 8189, | | |
| 7933194 | 1068 | NM_199168 | 1658 | 2665, 5012, 5013, 5117, 5003, 6588, 8491, 7914, 6051, | CXCL12 | 4.67 |
| | | | | 6656, 6859, | | |
| 7902345 | 1069 | NM_001162916 | 1659 | 3648, 4873, 4874, 4872, 8405, 6496, 6385, 8689, 7110, | TYW3 | 4.48 |
| | | | | 8780, 6273, | | |
| 7901497 | 1070 | NM_001004339 | 1660 | 3328, 4808, 6317, 5872, | ZYG11A | 4.16 |
| 7985159 | 1071 | NM_004378 | 1661 | 3015, 4439, 5645, | CRABP1 | 3.92 |
| 8146092 | 1072 | NM_002164 | 1662 | 2584, 5259, 5421, 8523, 8757, 8169, 8149, 8114, 8319, | IDO1 | 3.92 |
| | | | | 7915, 8778, | | |
| 8178470 | 1073 | NM_203289 | 1663 | 2865, 2866, 2867, 2868, 3988, 3986, 4587, 5026, 5027, | POU5F1 | 3.76 |
| | | | | 5028, 5029, 5030, 5031, 5032, 5033, 5034, 5035, 5036, | | |
| | | | | 5037, 5038, 5039, 5040, 5041, 5042, 5043, 5044, 5045, | | |
| | | | | 5046, 5047, 5048, 5049, 5050, 5051, 5052, 5053, 5054, | | |
| | | | | 5055, 5056, 5057, 5058, 5059, 5060, 5061, 5062, 5063, | | |
| | | | | 5064, 5065, 5066, 5067, 5068, 5069, 5070, 5071, 5072, | | |
| | | | | 5073, 5074, 5075, 5076, 5077, 5078, 5079, 5080, 5081, | | |
| | | | | 5082, 5083, 5084, 5085, 5086, 5087, 5088, 5089, 5090, | | |
| | | | | 5091, 5092, 5093, 5094, 5095, 5096, 5097, 5098, 5099, | | |
| | | | | 5100, 5101, 5102, 5103, 5104, 4227, 4228, 4229, 4230, | | |
| | | | | 4231, 3958, 3959, 4369, 5016, 5017, 4052, 5175, 5179, | | |
| | | | | 8740, 7043, 6463, 8204, 6434, 6854, 6431, 8068, 6882, | | |
| | | | | 7219, 7760, 8266, 8247, 6495, 7073, 7834, 7275, 7155, | | |
| | | | | 8483, 6801, 7986, 6354, 8509, 6963, 7415, 7877, 6325, | | |
| | | | | 7358, 5405, 7117, 6852, 8575, 6970, 7641, 7552, | | |
| 8121850 | 1074 | NM_012259 | 1664 | 4276, 3975, 5989, 6023, | HEY2 | 3.74 |
| 8124380 | 1075 | NM_005325 | 1665 | 3881, 4053, 5377, | HIST1H1A | 3.70 |
| 8063536 | 1076 | NM_003222 | 1666 | 3168, 3531, 5324, | TFAP2C | 3.69 |
| 8089438 | 1077 | NM_138815 | 1667 | 3289, 4865, 8060, | DPPA2 | 3.63 |
| 8098690 | 1078 | NM_174900 | 1668 | 3498, 3937, 5793, 8300, | ZFP42 | 3.59 |
| 7971838 | 1079 | NM_007015 | 1669 | 3028, 3784, 3785, 7153, 6673, | LECT1 | 3.54 |
| 8147244 | 1080 | NM_022351 | 1670 | 3290, 4737, 6800, 7785, 7823, 8669, 7989, 8696, | NECAB1 | 3.48 |
| 7983239 | 1081 | NM_020990 | 1671 | 4017, 4725, 3845, 3844, 5618, 8459, 7658, 6700, 6805, | CKMT1A | 3.47 |
| | | | | 7001, 6390, 7746, 7418, 6964, 8012, 7084, 7264, 7777, | | |
| | | | | 8717, 7315, 6930, 7503, 6473, 7092, 7417, 7902, 7582, | | |
| 8066117 | 1082 | NM_015474 | 1672 | 3449, 5023, 3830, 5349, 5541, 7106, 6100, | SAMHD1 | 3.46 |
| 8097256 | 1083 | NM_198041 | 1673 | 2576, 5304, 4197, 5305, 5478, 5980, 5822, | FGF2 | 3.44 |
| 7913216 | 1084 | NM_000300 | 1674 | 2906, 4866, 4867, 4868, 4869, 6945, 6892, 8598, 8048, | PLA2G2A | 3.42 |
| | | | | 8615, 6396, 7663, | | |
| 8151240 | 1085 | NM_024504 | 1675 | 2858, 3552, 5534, | PRDM14 | 3.39 |
| 8124889 | 1086 | NM_203289 | 1676 | 2865, 2866, 2867, 2868, 3988, 3986, 4587, 5026, 5027, | POU5F1 | 3.39 |
| | | | | 5028, 5029, 5030, 5031, 5032, 5033, 5034, 5035, 5036, | | |
| | | | | 5037, 5038, 5039, 5040, 5041, 5042, 5043, 5044, 5045, | | |
| | | | | 5046, 5047, 5048, 5049, 5050, 5051, 5052, 5053, 5054, | | |
| | | | | 5055, 5056, 5057, 5058, 5059, 5060, 5061, 5062, 5063, | | |
| | | | | 5064, 5065, 5066, 5067, 5068, 5069, 5070, 5071, 5072, | | |
| | | | | 5073, 5074, 5075, 5076, 5077, 5078, 5079, 5080, 5081, | | |
| | | | | 5082, 5083, 5084, 5085, 5086, 5087, 5088, 5089, 5090, | | |
| | | | | 5091, 5092, 5093, 5094, 5095, 5096, 5097, 5098, 5099, | | |
| | | | | 5100, 5101, 5102, 5103, 5104, 4227, 4228, 4229, 4230, | | |
| | | | | 4231, 3958, 3959, 4369, 5016, 5017, 4052, 5175, 5179, | | |
| | | | | 8740, 7043, 6463, 8204, 6434, 6854, 6431, 8068, 6882, | | |
| | | | | 7219, 7760, 8266, 8247, 6495, 7073, 7834, 7275, 7155, | | |
| | | | | 8483, 6801, 7986, 6354, 8509, 6963, 7415, 7877, 6325, | | |
| | | | | 7358, 5405, 7117, 6852, 8575, 6970, 7641, 7552, | | |
| 8139212 | 1087 | NM_000168 | 1677 | 3991, 3982, 2577, 4553, 3392, 4592, 6480, 7171, 6577, | GLI3 | 3.38 |
| | | | | 6439, 8751, 6708, | | |
| 8071809 | 1088 | NM_000854 | 1678 | 2877, 3667, 4143, 4144, 5682, 5316, | GSTT2 | 3.36 |
| 7904158 | 1089 | NM_020190 | 1679 | 3467, 3697, 6147, 5677, 8035, 6986, | OLFML3 | 3.31 |
| 8085676 | 1090 | NM_001351 | 1680 | 3383, 5153, 5154, 6778, | DAZL | 3.30 |
| 8056408 | 1091 | NM_004482 | 1681 | 3996, 3990, 3410, 3332, 4301, 4196, 6671, 7016, 6517, | GALNT3 | 3.29 |
| | | | | 7277, 7565, 6510, 6479, 6804, 6507, | | |
| 8084165 | 1092 | NM_003106 | 1682 | 3338, 5264, 5632, | SOX2 | 3.28 |
| 8148315 | 1093 | NM_001159542 | 1683 | 5084, 4815, 6578, 6639, | POU5F1B | 3.27 |
| 7941662 | 1094 | NM_001104 | 1684 | 3432, 2761, 8043, 7856, 8550, | ACTN3 | 3.27 |
| 7949971 | 1095 | NM_001876 | 1685 | 2870, 4398, 4399, 7075, 5473, | CPT1A | 3.26 |
| 7970954 | 1096 | NM_004734 | 1686 | 4221, 5192, 5193, 5194, 5195, 5448, 7317, 6024, 6492, | DCLK1 | 3.26 |
| | | | | 7076, | | |
| 8112980 | 1097 | NM_005711 | 1687 | 2720, 3295, 8700, 8201, 7504, 5630, | EDIL3 | 3.23 |
| 7953675 | 1098 | NM_024865 | 1688 | 3571, 3299, 4206, 5371, | NANOG | 3.23 |
| 8077513 | 1099 | NM_015453 | 1689 | 2882, 2944, 4333, 4334, 6458, 6573, 7246, 8510, 6238, | THUMPD3 | 3.22 |
| | | | | 6855, 6555, 8394, 7095, 6527, | | |
| 7909642 | 1100 | NM_001136475 | 1690 | 3791, 4510, 4251, 3161, 3278, 3303, 4655, 4653, 4654, | VASH2 | 3.19 |
| | | | | 6082, 5905, 6123, 6062, 6304, 5489, 8315, 6152, 8644, | | |
| | | | | 8692, | | |
| 8024712 | 1101 | NM_033064 | 1691 | 3258, 3101, 4560, 3035, 4405, 5929, 7505, 5849, 7325, | ATCAY | 3.18 |
| 7965565 | 1102 | NM_032147 | 1692 | 3062, 3978, 3979, 6775, 5436, | USP44 | 3.18 |
| 8117422 | 1103 | NM_003540 | 1693 | 3825, 3059, 7626, | HIST1H4F | 3.12 |
| 7987365 | 1104 | NM_024865 | 1694 | 3571, 3299, 4206, 5371, | NANOG | 3.09 |
| 8172471 | 1105 | NM_006875 | 1695 | 2994, 4637, 7671, 8024, 6884, | PIM2 | 3.08 |
| 8061746 | 1106 | NM_006892 | 1696 | 3002, 3249, 3250, 3251, 5288, 6284, 6608, 5831, 5310, | DNMT3B | 3.05 |
| | | | | 6240, 7520, 7394, 6299, | | |
| 8157193 | 1107 | NM_133464 | 1697 | 4219, 4428, 4429, 7118, 6162, 5698, | ZNF483 | 3.04 |
| 8142580 | 1108 | NM_001024613 | 1698 | 4758, 4836, 4837, 7698, 6675, 7454, 5751, | FEZF1 | 3.04 |
| 8171758 | 1109 | NR_023358 | 1699 | 4408, | SCARNA9L | 3.04 |
| 8108995 | 1110 | NM_054023 | 1700 | 3022, 4995, 8754, 8215, 5614, | SCGB3A2 | 3.00 |
| 7963970 | 1111 | NM_006928 | 1701 | 3105, 5245, 6941, 6287, | SILV | 2.98 |
| 8122099 | 1112 | NM_006208 | 1702 | 3492, 4353, 5924, 6245, 8263, 8565, 7205, | ENPP1 | 2.94 |
| 8148317 | 1113 | NM_002467 | 1703 | 2869, 4851, 6776, 7274, | MYC | 2.90 |
| 8039280 | 1114 | NM_139176 | 1704 | 4509, 4372, 4373, 4374, 7316, 5964, 5724, 7405, 7354, | NLRP7 | 2.88 |
| | | | | 7602, | | |
| 7930631 | 1115 | NM_198795 | 1705 | 3518, 3513, 6133, 6452, 5445, 6004, 5919, | TDRD1 | 2.86 |
| 8152938 | 1116 | NM_001145095 | 1706 | 2750, 4755, 7527, 8041, | HHLA1 | 2.86 |
| 8001531 | 1117 | NM_005950 | 1707 | 3006, 2840, 6392, 7114, 7170, 6815, | MT1G | 2.85 |
| 8095043 | 1118 | NM_023940 | 1708 | 3792, 3377, 8732, 5428, 8096, | RASL11B | 2.79 |
| 7949124 | 1119 | NM_001164716 | 1709 | 2730, 4906, 4290, 8065, 5323, | PYGM | 2.78 |
| 8073062 | 1120 | NM_004900 | 1710 | 2835, 3155, 5168, 5750, 5753, | APOBEC3B | 2.78 |
| 8148070 | 1121 | NM_021110 | 1711 | 4389, 2612, 2613, 2692, 2964, 3160, 4321, 3750, 8105, | COL14A1 | 2.71 |
| | | | | 5673, 5636, 7386, 8362, 7397, 5443, | | |
| 8015635 | 1122 | NM_012232 | 1712 | 3561, 4938, 6271, 5910, | PTRF | 2.69 |
| 8138708 | 1123 | NM_005522 | 1713 | 3389, 3921, 3920, 5895, 5930, | HOXA1 | 2.67 |
| 8028991 | 1124 | NM_030622 | 1714 | 3461, 3715, 5647, 5708, | CYP2S1 | 2.66 |
| 8169115 | 1125 | NM_198465 | 1715 | 3311, 4081, 5354, | NRK | 2.65 |
| 7949104 | 1126 | NM_001098670 | 1716 | 2786, 2787, 2742, 3987, 2749, 3104, 3106, 3428, 4186, | RASGRP2 | 2.64 |
| | | | | 4187, 3200, 6996, 7253, 7251, 6422, 6110, 6605, 7167, | | |
| | | | | 6721, 6400, 7783, 7408, 7101, 6992, 8419, 6612, 7675, | | |
| | | | | 7336, 6494, 7993, 7692, 6747, 7413, | | |
| 7912473 | 1127 | NM_183412 | 1717 | 3680, 3848, 3023, 4316, 3818, 5249, 8328, 6632, 6172, | FBXO2 | 2.62 |
| | | | | 7087, 6489, 6785, 5411, | | |
| 7956488 | 1128 | NM_004984 | 1718 | 4336, 3578, 5671, 7555, | KIF5A | 2.62 |
| 8145977 | 1129 | NM_021623 | 1719 | 4259, 4207, 8302, 7202, 6843, 7732, 8390, | PLEKHA2 | 2.61 |
| 8096361 | 1130 | NM_016323 | 1720 | 3489, 4008, 8649, 5458, 8138, 8244, | HERC5 | 2.61 |
| 8104107 | 1131 | NM_173553 | 1721 | 3502, 3928, 3929, 4676, 4718, 3234, 7761, 8133, 7793, | TRIML2 | 2.60 |
| | | | | 5580, 8122, | | |
| 7986350 | 1132 | NM_183376 | 1722 | 3385, 4436, 5479, | ARRDC4 | 2.60 |
| 7975076 | 1133 | NM_021979 | 1723 | 3158, 4151, 7440, 5369, | HSPA2 | 2.59 |
| 7933092 | 1134 | NM_021045 | 1724 | 4114, 3320, 8456, 7285, 8408, 7542, 7283, 7447, 6137, | ZNF248 | 2.59 |
| 8134463 | 1135 | NM_002523 | 1725 | 3340, 4771, 5502, 6922, | NPTX2 | 2.57 |
| 7995838 | 1136 | NM_005952 | 1726 | 3371, 4455, 6391, | MT1X | 2.55 |
| 8096301 | 1137 | NM_001040058 | 1727 | 4706, 3942, 3501, 3944, 8278, 7992, 5370, 6716, 8452, | SPP1 | 2.55 |
| | | | | 8651, 6109, 6075, 7831, 7801, | | |
| 7963869 | 1138 | NM_002355 | 1728 | 3673, 5287, 3831, 7078, 5809, 6441, 5420, 7521, 5321, | PHC1 | 2.53 |
| 8097920 | 1139 | NM_004744 | 1729 | 3566, 3595, 8748, 8069, 7763, 8070, 5620, | LRAT | 2.51 |
| 8031576 | 1140 | NM_134444 | 1730 | 3031, 4444, 5611, 5951, | NLRP4 | 2.49 |
| 8116418 | 1141 | NM_005110 | 1731 | 2751, 4135, 8471, 5407, 8222, 8775, 7999, | GFPT2 | 2.47 |
| 8139820 | 1142 | NM_178558 | 1732 | 3061, 4448, 4449, 8320, 6601, 8790, 5658, | ZNF680 | 2.47 |
| 7989277 | 1143 | NM_004998 | 1733 | 3855, 3757, 5660, | MYO1E | 2.45 |
| 7983360 | 1144 | NM_004048 | 1734 | 3076, 3490, 6485, 6270, | B2M | 2.44 |
| 8120679 | 1145 | NM_018665 | 1735 | 2820, 2957, 4749, 6125, 7145, 8551, 6076, | DDX43 | 2.44 |
| 7914342 | 1146 | NM_004102 | 1736 | 2918, 3829, 8327, 8609, 8516, 5937, | FABP3 | 2.44 |
| 8147697 | 1147 | NM_024915 | 1737 | 4098, 4097, 4270, 4529, 3637, 3606, 3638, 4299, 4354, | GRHL2 | 2.43 |
| | | | | 5422, 7907, 7656, 8209, 8281, 7516, | | |
| 8044353 | 1148 | NM_001142807 | 1738 | 4470, 4673, 6005, 7357, 6866, 6663, 6681, | ACOXL | 2.41 |
| 8138489 | 1149 | NM_018719 | 1739 | 3585, 4801, 4802, 4803, 7510, 6116, 7011, 6541, 6183, | CDCA7L | 2.40 |
| | | | | 8558, | | |
| 8099524 | 1150 | NM_001290 | 1740 | 2733, 4590, 4591, 8679, 8166, 7837, 8661, 8298, 5669, | LDB2 | 2.39 |
| | | | | 8605, 7719, 7814, 8032, 8473, 7455, 8734, | | |
| 8020384 | 1151 | NM_001142966 | 1741 | 4467, 2876, 4295, 4675, 7359, 7061, 5529, 7232, | KIAA1772 | 2.39 |
| 8103254 | 1152 | NM_003013 | 1742 | 2933, 3724, 5471, | SFRP2 | 2.38 |
| 8078435 | 1153 | NM_001039111 | 1743 | 3892, 3922, 6993, | TRIM71 | 2.38 |
| 7928429 | 1154 | NM_002658 | 1744 | 3397, 4752, 4753, 6308, 6729, 8322, 7727, 8611, 6028, | PLAU | 2.37 |
| | | | | 6300, 8374, | | |
| 8121277 | 1155 | NM_001624 | 1745 | 4575, 4051, 6064, 6887, 8412, 5596, | AIM1 | 2.36 |
| 7995797 | 1156 | NM_175617 | 1746 | 3381, 3905, 5861, 5789, | MT1E | 2.36 |
| 8015607 | 1157 | NM_139276 | 1747 | 4260, 3613, 3614, 3636, 7860, 7019, 6610, 8049, 5511, | STAT3 | 2.34 |
| | | | | 7592, 8190, 6553, | | |
| 7998921 | 1158 | NM_032805 | 1748 | 3149, 2932, 5400, | ZSCAN10 | 2.34 |
| 7983718 | 1159 | NM_001165257 | 1749 | 3014, 4909, 4908, 5333, | SCG3 | 2.33 |
| 7997336 | 1160 | NM_020927 | 1750 | 3166, 3189, 5813, | VAT1L | 2.32 |
| 7901087 | 1161 | NM_032756 | 1751 | 2922, 3690, 5860, | HPDL | 2.31 |
| 8124684 | 1162 | NM_001109809 | 1752 | 4390, 4684, 4850, 7287, 7191, 7216, 6705, 6979, 6515, | ZFP57 | 2.31 |
| | | | | 7376, 8185, 6500, 6658, | | |
| 8160260 | 1163 | NM_017637 | 1753 | 3551, 4555, 3100, 4545, 4032, 4033, 4034, 4035, 4036, | BNC2 | 2.30 |
| | | | | 4037, 2799, 3963, 6528, 6375, 7529, 7058, 6914, 7266, | | |
| | | | | 7235, 7564, 8608, 7382, 8765, | | |
| 8026424 | 1164 | NM_173483 | 1754 | 3826, 4241, 5547, | CYP4F22 | 2.30 |
| 8165217 | 1165 | NM_017617 | 1755 | 2850, 4176, 5567, | NOTCH1 | 2.30 |
| 8113278 | 1166 | NM_153234 | 1756 | 3387, 4341, 5548, | LIX1 | 2.30 |
| 7994609 | 1167 | NM_014298 | 1757 | 3509, 3569, 6447, 6560, 5343, | QPRT | 2.29 |
| 8137091 | 1168 | NM_170686 | 1758 | 3019, 4464, 4465, 8588, 6484, 7701, 5871, 8626, 7762, | ZNF398 | 2.29 |
| | | | | 7857, | | |
| 7936673 | 1169 | NM_002925 | 1759 | 3525, 3730, 3731, 8042, 7377, 6524, 6267, 6015, | RGS10 | 2.27 |
| 8162531 | 1170 | ENST00000396160 | 1760 | 7570, | MT1G | 2.26 |
| 8010354 | 1171 | NM_000152 | 1761 | 3217, 4085, 4086, 4087, 5704, 6841, | GAA | 2.25 |
| 8059301 | 1172 | NM_181460 | 1762 | 4255, 5159, 5161, 5162, 5163, 5164, 5165, 5158, 5160, | PAX3 | 2.23 |
| | | | | 7119, 5969, 5904, 7673, 5842, 7706, 6334, 5413, | | |
| 8006367 | 1173 | NM_138328 | 1763 | 3863, 3884, 5472, 7404, | RHBDL3 | 2.23 |
| 8106820 | 1174 | NM_006467 | 1764 | 4507, 4049, 6583, 8418, | POLR3G | 2.23 |
| 7914015 | 1175 | NM_052943 | 1765 | 3427, 4638, 5752, | FAM46B | 2.23 |
| 7995825 | 1176 | NM_005949 | 1766 | 3051, 4447, 5801, 6580, | MT1F | 2.22 |
| 7945296 | 1177 | NM_001080407 | 1767 | 3430, 4288, 6702, 8356, 8044, 7094, 7185, 6706, | GLB1L3 | 2.21 |
| 8097928 | 1178 | NM_144979 | 1768 | 3391, 4586, 5462, | RBM46 | 2.21 |
| 8074335 | 1179 | NM_001195226 | 1769 | 2764, 3807, 3998, 4025, 2793, 2709, 3097, 3594, 5186, | PRODH | 2.21 |
| | | | | 5187, 5737, 7271, 5953, 7991, 7069, 6983, 7547, 8254, | | |
| | | | | 6370, 7730, 6607, 5836, | | |
| 8115196 | 1180 | NM_052860 | 1770 | 2952, 4996, 4997, 4998, 5526, 6512, 6897, | ZNF300 | 2.21 |
| 8163896 | 1181 | NM_004099 | 1771 | 2946, 3496, 3495, 6038, 5639, | STOM | 2.20 |
| 7948987 | 1182 | NM_007069 | 1772 | 3359, 4422, 4423, 5706, 6689, 7225, | PLA2G16 | 2.20 |
| 7942064 | 1183 | NM_015973 | 1773 | 3055, 3933, 5477, | GAL | 2.18 |
| 8150962 | 1184 | NM_014729 | 1774 | 2740, 3709, 6856, 6017, | TOX | 2.17 |
| 8046078 | 1185 | NM_020981 | 1775 | 2763, 5191, 6587, 5756, | B3GALT1 | 2.17 |
| 7978801 | 1186 | NM_182830 | 1776 | 3421, 4330, 5167, 6351, 6182, 7688, 8193, 6799, 7207, | MDGA2 | 2.17 |
| 7912481 | 1187 | NM_006341 | 1777 | 2774, 2791, 2768, 2798, 3111, 2967, 4393, 4394, 6406, | MAD2L2 | 2.16 |
| | | | | 7367, 7044, 5373, 7566, 7290, 7330, 6832, 7175, | | |
| 8143221 | 1188 | NM_130840 | 1778 | 4271, 3924, 3925, 3923, 8501, 8715, 6101, 5610, 7586, | ATP6V0A4 | 2.16 |
| 8121949 | 1189 | NM_000426 | 1779 | 3433, 4089, 4090, 6848, 5928, 7289, 6035, | LAMA2 | 2.16 |
| 7919787 | 1190 | NM_032132 | 1780 | 3665, 3069, 3254, 3722, 5240, 8304, 6029, 6113, 5998, | HORMAD1 | 2.15 |
| | | | | 7030, 5874, 5867, 8731, 6466, 5973, | | |
| 7938035 | 1191 | AL360187 | 1781 | 2821, 2822, 2823, 4283, 4533, 4544, 4481, 3016, 3162, | TRIM22 | 2.15 |
| | | | | 4213, 4312, 4320, 4326, 5229, 7088, 6634, 6762, 8083, | | |
| | | | | 6459, 8567, 6533, 6911, 6474, 7269, 7353, | | |
| 8018652 | 1192 | NM_052916 | 1782 | 4746, 5562, 5570, 5845, | RNF157 | 2.15 |
| 8052882 | 1193 | NM_001617 | 1783 | 3312, 2549, 4252, 4541, 3221, 3555, 5136, 5137, 5138, | ADD2 | 2.15 |
| | | | | 5139, 5140, 7591, 8079, 5513, 5527, 8334, 8155, 6282, | | |
| | | | | 7652, 6883, 7881, 7694, 6520, 6309, 7614, 7098, | | |
| 8168438 | 1194 | NM_145052 | 1784 | 3674, 4272, 2966, 4297, 4940, 4941, 7089, 8335, 6323, | UPRT | 2.15 |
| | | | | 6105, 6157, | | |
| 8125500 | 1195 | NM_004159 | 1785 | 3676, 2641, 2642, 4720, 3358, 3875, 3876, 6366, 7256, | PSMB8 | 2.15 |
| | | | | 7662, 7787, 7258, 6923, 7465, 7711, 6603, 7071, 8698, | | |
| | | | | 6850, 6638, 7406, 7295, 8663, 7850, 7345, 6440, 6333, | | |
| | | | | 7938, 7517, 6750, 8634, 6788, 7146, 8416, 7360, 7022, | | |
| | | | | 6858, 8225, 6666, 7687, 7230, 6844, 6589, 8360, 7319, | | |
| | | | | 8354, 7670, | | |
| 8033801 | 1196 | NM_001130031 | 1786 | 4039, 4450, 4451, 4452, 6833, 5584, 6691, | ZNF562 | 2.14 |
| 8171338 | 1197 | NM_174901 | 1787 | 3150, 5120, 5679, 6572, 7485, 7604, | FAM9C | 2.13 |
| 8097017 | 1198 | NM_003360 | 1788 | 2689, 4419, 4420, 5810, 8336, 6908, | UGT8 | 2.13 |
| 7956522 | 1199 | AF063608 | 1789 | 2859, 7555, | KIF5A | 2.11 |
| 7981945 | 1200 | BC043194 | 1790 | 3229, | SNRPN | 2.09 |
| 8035318 | 1201 | NM_001080421 | 1791 | 4963, 8704, 7597, 8506, 5412, | UNC13A | 2.09 |
| 8000757 | 1202 | NM_003586 | 1792 | 3325, 3610, 3233, 5788, 5896, | DOC2A | 2.09 |
| 8033767 | 1203 | NM_152476 | 1793 | 2982, 4116, 5683, | ZNF560 | 2.09 |
| 7931556 | 1204 | NM_014468 | 1794 | 2743, 3330, 5776, | VENTX | 2.07 |
| 8143772 | 1205 | NM_002889 | 1795 | 3103, 4674, 8675, 7835, 7515, 5341, | RARRES2 | 2.07 |
| 8096808 | 1206 | NM_032992 | 1796 | 3120, 3873, 3874, 4445, 8500, 7859, 7100, 6201, | CCDC109B | 2.06 |
| 7995783 | 1207 | NM_005953 | 1797 | 2919, 4617, 5366, | MT2A | 2.05 |
| 8013660 | 1208 | NM_005165 | 1798 | 3335, 3853, 6651, 5330, 6714, 7379, | ALDOC | 2.03 |
| 7983143 | 1209 | NM_020759 | 1799 | 4943, 5714, | STARD9 | 2.03 |
| 8008664 | 1210 | NM_003488 | 1800 | 3357, 3981, 5585, 8407, 7660, 5628, | AKAP1 | 2.03 |

Table 11: Provided are the description of the non-membranal genes along with the sequence identifiers of the Affymetrix probes, the target sequence identified by the Affymetrix probe, and the mRNA sequences represented by the target sequences. Also shown are the log 2 fold change in expression levels of the indicated genes between the PDX1-GFP cells and the SOX17-GFP cells (PDX1-SOX17). The (+) or (−) sign means that the genes are upregulated or downregulated, respectively, in the PDX1-GFP cells as compared to the SOX17-GFP cells.

TABLE 12
Non-membranal genes which are downregulated in PDX1 cells as compared to SOX17 cells

| | Affy. | | SEQ ID | | | Log 2 |
|---|---|---|---|---|---|---|
| | target | | NO: (Of | | | fold change |
| Probe | nucl. SEQ | Rep. | Rep. Pub- | Polyn. SEQ ID NOs: | Gene | [PDX- |
| Set ID | ID NO: | Public ID | lic ID) | rep. by target | Symbol | SOX17] |
| 8065412 | 1211 | NM_001898 | 1801 | 3369, 3033, 5734, 6511, | CST1 | −7.67 |
| 8160163 | 1212 | NM_005454 | 1802 | 3880, 3883, 6763, | CER1 | −7.42 |
| 7934979 | 1213 | NM_014391 | 1803 | 2995, 3511, 5932, | ANKRD1 | −5.27 |
| 8136807 | 1214 | — | | | TRY6 | −5.22 |
| 8152617 | 1215 | NM_005328 | 1804 | 2676, 4348, 5843, 6409, | HAS2 | −5.08 |
| 7927631 | 1216 | NM_012242 | 1805 | 3471, 3796, 7543, 8463, 8242, 5959, | DKK1 | −5.03 |
| 8136790 | 1217 | — | | | PRSS2 | −4.58 |
| 7997188 | 1218 | NM_005143 | 1806 | 3893, 4016, 4018, 2580, 2622, 2977, 2992, 3453, | HP | −4.53 |
| | | | | 3617, 4365, 4366, 6921, 6310, 6000, 6001, | | |
| 7921834 | 1219 | NM_001643 | 1807 | 2901, 2758, 6253, 8161, 7460, 7113, 8544, 6696, | APOA2 | −4.50 |
| | | | | 7530, 7963, 6627, 6449, | | |
| 8106986 | 1220 | NM_014899 | 1808 | 3394, 4041, 7121, 8530, 7782, 8683, 8248, 7828, 8218, 8489, | RHOBTB3 | −4.44 |
| 8060850 | 1221 | NM_001200 | 1809 | 3900, 6567, | BMP2 | −4.43 |
| 8171449 | 1222 | NM_021804 | 1810 | 3847, 4462, 6787, 7949, 7759, 7328, 5427, | ACE2 | −4.39 |
| 8149825 | 1223 | NM_003155 | 1811 | 2725, 3793, 5771, | STC1 | −4.32 |
| 7904843 | 1224 | NM_002614 | 1812 | 3336, 5243, 4204, 7461, 7260, 6532, 6226, 7090, | PDZK1 | −4.29 |
| | | | | 6900, 7707, 7472, 6465, | | |
| 7929282 | 1225 | NM_002729 | 1813 | 2961, 4124, 7867, 5797, 8612, | HHEX | −4.18 |
| 8048319 | 1226 | NM_007127 | 1814 | 4477, 4578, 5439, | VIL1 | −4.16 |
| 8121257 | 1227 | NM_182907 | 1815 | 3253, 5303, 4361, 6228, 6168, 8184, 8624, 7700, | PRDM1 | −4.07 |
| 8085946 | 1228 | NM_005442 | 1816 | 3171, 3140, 5868, 7137, 7635, | EOMES | −4.01 |
| 7968872 | 1229 | NM_013238 | 1817 | 3842, 3846, 7013, 8073, | DNAJC15 | −3.99 |
| 8092095 | 1230 | NM_015028 | 1818 | 4193, 4852, 4853, 4854, 4855, 4856, 4857, 4858, | TNIK | −3.97 |
| | | | | 4859, 4860, 8054, 5939, 6549, 7883, 5603, 6786, | | |
| | | | | 7448, 7215, 8447, 8382, 7882, 6219, | | |
| 8058857 | 1231 | NM_000599 | 1819 | 2583, 4357, 8723, 5360, | IGFBP5 | −3.95 |
| 8121803 | 1232 | NM_001010852 | 1820 | 3116, 3869, 5483, 6248, | RLBP1L2 | −3.92 |
| 8088560 | 1233 | NM_182920 | 1821 | 4100, 3333, 8622, 8439, 8457, 6636, 7795, 8233, | ADAMTS9 | −3.90 |
| | | | | 8784, | | |
| 7971296 | 1234 | NM_033255 | 1822 | 3367, 3694, 3693, 5738, 5732, 7664, 7742, | EPSTI1 | −3.83 |
| 8163839 | 1235 | NM_001735 | 1823 | 3997, 3497, 8045, 5344, 6954, 6376, | C5 | −3.80 |
| 8096160 | 1236 | NM_001025616 | 1824 | 3856, 4013, 4014, 4012, 8287, 7220, 7970, 6456, | ARHGAP24 | −3.76 |
| | | | | 8329, 8596, 5517, 8693, 8490, | | |
| 8136801 | 1237 | NM_002770 | 1825 | 3326, 3327, 4043, 4044, 3896, 4082, 2633, 3056, | TRY6 | −3.73 |
| | | | | 4031, 3057, 4349, 5199, 5294, 5200, 4875, 7372, | | |
| | | | | 5743, 6222, 8379, | | |
| 8149071 | 1238 | NM_001147 | 1826 | 2696, 4344, 4345, 4346, 5744, 7577, | ANGPT2 | −3.73 |
| 8020795 | 1239 | NM_000371 | 1827 | 2722, 4730, 6739, 5362, | TTR | −3.71 |
| 8139307 | 1240 | NM_021223 | 1828 | 3043, 3698, 7148, 5334, 7843, 7318, 7526, | MYL7 | −3.71 |
| 8105302 | 1241 | NM_013409 | 1829 | 3343, 5282, 5281, 7924, 7913, 6382, 5408, | FST | −3.66 |
| 7965322 | 1242 | NM_000899 | 1830 | 4054, 5149, 5148, 6216, 5328, 6318, 7574, | KITLG | −3.65 |
| 7926105 | 1243 | NM_002051 | 1831 | 2915, 3695, 3696, 6780, 6049, 7381, | GATA3 | −3.65 |
| 8113358 | 1244 | NM_005668 | 1832 | 3302, 4599, 3244, 7055, 7738, 5386, | ST8SIA4 | −3.60 |
| 8057377 | 1245 | NM_173648 | 1833 | 3425, 5152, 5914, 5666, 7929, | CCDC141 | −3.57 |
| 8151931 | 1246 | NM_033512 | 1834 | 3241, 3675, 5757, | TSPYL5 | −3.56 |
| 8105013 | 1247 | NM_152403 | 1835 | 3530, 5277, 5276, 5275, 6119, 5859, 8466, 5742, | EGFLAM | −3.55 |
| | | | | 6783, 8384, 6717, 7825, 8629, 8422, 7942, 6265, | | |
| 8057056 | 1248 | NM_133378 | 1836 | 3308, 2656, 3424, 4106, 4107, 3344, 3048, 5008, | TTN | −3.52 |
| | | | | 5009, 5011, 5007, 5010, 7368, 5887, 5945, 7339, | | |
| | | | | 6830, 8194, 6950, 6315, 6321, 6481, 7366, 6697, | | |
| | | | | 6793, 7546, 6131, 5681, | | |
| 7924682 | 1249 | NM_003240 | 1837 | 3558, 4989, 4988, 8493, 7506, 6104, | LEFTY2 | −3.52 |
| 8065344 | 1250 | NM_021784 | 1838 | 2770, 4577, 4466, 5823, 7393, 7364, 6571, | FOXA2 | −3.38 |
| 8129231 | 1251 | NM_024581 | 1839 | 3503, 4208, 4209, 8021, 7766, 5900, 8495, 6262, | FAM184A | −3.38 |
| 8147030 | 1252 | NM_007029 | 1840 | 4711, 5225, 5342, | STMN2 | −3.35 |
| 7976858 | 1253 | NM_001362 | 1841 | 2990, 4161, 8265, 6088, | DIO3 | −3.33 |
| 8058765 | 1254 | NM_054034 | 1842 | 3732, 3409, 3983, 4677, 4679, 3284, 3310, 3403, | FN1 | −3.33 |
| | | | | 3404, 3405, 3406, 4727, 3110, 4551, 4571, 4572, | | |
| | | | | 4576, 3822, 3494, 2852, 3620, 3621, 3622, 3623, | | |
| | | | | 3624, 3625, 5996, 7280, 7649, 6915, 8364, 8529, | | |
| | | | | 6768, 7398, 5846, 6424, 8736, 8430, 7036, 5993, | | |
| | | | | 8153, 8487, 6324, 6771, 6223, 6205, 5539, 6620, | | |
| | | | | 6121, 6206, 8147, 5687, | | |
| 8136795 | 1255 | NM_002769 | 1843 | 4044, 4083, 5224, 7372, 8647, 5627, 7412, 7969, | PRSS1 | −3.32 |
| 7912520 | 1256 | NM_002521 | 1844 | 3029, 3909, 7941, 6388, | NPPB | −3.32 |
| 7921916 | 1257 | NM_001195303 | 1845 | 3306, 5190, 5110, 7452, 5828, 6057, | RGS5 | −3.30 |
| 8121144 | 1258 | NM_024641 | 1846 | 4179, 5290, 6156, 5952, 8006, | MANEA | −3.29 |
| 7979357 | 1259 | NM_021728 | 1847 | 3073, 3223, 3224, 6604, 6242, | OTX2 | −3.28 |
| 8047381 | 1260 | NM_003879 | 1848 | 2698, 2700, 2701, 2702, 2703, 2697, 2727, 2728, | CFLAR | −3.28 |
| | | | | 2729, 2714, 2715, 2716, 2717, 2718, 3809, 3282, | | |
| | | | | 4540, 4723, 2706, 2695, 2707, 4275, 4518, 2881, | | |
| | | | | 4319, 5254, 5255, 7718, 7181, 7213, 6901, 7210, | | |
| | | | | 7050, 6868, 7797, 7093, 6246, 6301, 6416, 6026, | | |
| | | | | 8088, 7173, 6972, 6669, 7157, 7540, 7179, 6019, | | |
| | | | | 5721, | | |
| 8058498 | 1261 | NM_003468 | 1849 | 2934, 4015, 5838, | FZD5 | −3.27 |
| 8054517 | 1262 | AK095678 | 1850 | 3119, 2978, 6378, 5623, 6529, | LOC151009 | −3.24 |
| 7961875 | 1263 | NM_152590 | 1851 | 4498, 4781, 4782, 4784, 4785, 4783, 5790, 7143, | IFLTD1 | −3.23 |
| | | | | 6544, 6853, | | |
| 7938231 | 1264 | NM_003621 | 1852 | 3011, 4355, 5626, | PPFIBP2 | −3.18 |
| 8166747 | 1265 | NM_001163335 | 1853 | 3411, 4887, 4885, 4886, 5912, 6888, 5629, | SYTL5 | −3.13 |
| 8019964 | 1266 | NM_001010000 | 1854 | 2803, 5198, 5415, 6442, 5664, 6551, 7534, 7585, | ARHGAP28 | −3.12 |
| 7957458 | 1267 | NM_006183 | 1855 | 2948, 5242, 5429, | NTS | −3.09 |
| 8050619 | 1268 | NM_000384 | 1856 | 2558, 3976, 5351, | APOB | −3.08 |
| 8065416 | 1269 | NM_001322 | 1857 | 3515, 3034, 5782, | CST2 | −3.05 |
| 7942998 | 1270 | NM_153696 | 1858 | 3060, 2843, 3280, 5177, 2766, 5178, 6173, 5921, | FOLH1B | −3.04 |
| | | | | 6307, 5425, 6469, | | |
| 8070579 | 1271 | NM_003225 | 1859 | 2552, 3668, 5847, | TFF1 | −3.04 |
| 8165663 | 1272 | AK172782 | 1860 | 3609, | GPAM | −3.04 |
| 8023598 | 1273 | NM_173557 | 1861 | 3123, 3946, 5605, | RNF152 | −3.04 |
| 8140650 | 1274 | NM_012431 | 1862 | 4218, 5112, 5113, 5625, 7193, | SEMA3E | −3.04 |
| 8111892 | 1275 | NM_000436 | 1863 | 2674, 4026, 8292, 5313, 8538, 8361, 8084, 7753, | OXCT1 | −3.03 |
| 8150076 | 1276 | NM_001394 | 1864 | 2649, 5265, 5266, 5393, | DUSP4 | −3.02 |
| 8121916 | 1277 | NM_032784 | 1865 | 3018, 3878, 6102, 6281, 8688, | RSPO3 | −3.01 |
| 8092523 | 1278 | NM_001966 | 1866 | 3206, 4927, 4926, 5368, 8762, | EHHADH | −3.00 |
| 8172670 | 1279 | NM_001013742 | 1867 | 3778, 5274, 6444, | DGKK | −2.99 |
| 8119898 | 1280 | NM_001025366 | 1868 | 3808, 4966, 4967, 4968, 4969, 4970, 4971, 4972, | VEGFA | −2.98 |
| | | | | 4973, 4974, 4975, 4976, 4977, 4978, 4979, 4980, | | |
| | | | | 4981, 8712, 5968, 6175, 7640, 5961, 5690, 7243, | | |
| | | | | 8549, 6759, 6136, 7716, 8136, 8355, 8159, 8018, | | |
| | | | | 7079, 8270, 8120, 7866, 8507, 8066, 7946, | | |
| 8077185 | 1281 | NM_000039 | 1869 | 3902, 2762, 6306, 5375, 6654, 6990, | APOA1 | −2.94 |
| 8059955 | 1282 | NM_022449 | 1870 | 3268, 4986, 4985, 7579, 8393, 6896, 6505, 7240, | RAB17 | −2.92 |
| | | | | 5533, | | |
| 8045664 | 1283 | NM_177964 | 1871 | 3039, 3538, 8714, 5464, 7286, 6455, 7123, 7806, | LYPD6B | −2.91 |
| | | | | 7494, 8341, 7067, 6613, | | |
| 8087925 | 1284 | NM_003280 | 1872 | 3054, 4613, 5365, 7708, | TNNC1 | −2.90 |
| 8074853 | 1285 | NM_080740 | 1873 | 3304, 3570, 5866, | ZNF280A | −2.90 |
| 8057506 | 1286 | NM_001463 | 1874 | 2679, 5106, 5695, | FRZB | −2.89 |
| 7951865 | 1287 | NM_000039 | 1875 | 3902, 2762, 6306, 5375, 6654, 6990, | APOA1 | −2.89 |
| 8146462 | 1288 | NM_022454 | 1876 | 3009, 4141, 5778, | SOX17 | −2.86 |
| 8129482 | 1289 | NM_001017373 | 1877 | 3098, 4120, 4122, 7473, 7107, 6192, 5825, 7041, | SAMD3 | −2.85 |
| | | | | 6688, | | |
| 8127193 | 1290 | NM_021073 | 1878 | 2611, 3202, 5997, 7150, | BMP5 | −2.85 |
| 8144643 | 1291 | NM_002052 | 1879 | 2632, 4360, 5839, 5435, | GATA4 | −2.81 |
| 8050537 | 1292 | NM_002381 | 1880 | 4246, 4011, 7511, 6871, 8601, | MATN3 | −2.80 |
| 8169995 | 1293 | NM_001170779 | 1881 | 4513, 4955, 4956, 4957, 4958, 4959, 4960, 4954, | FAM122C | −2.80 |
| | | | | 6451, 7031, 8034, 6865, 6135, 8059, 8191, 6912, | | |
| | | | | 8587, 6236, | | |
| 8103544 | 1294 | NM_016950 | 1882 | 3468, 4487, 4491, 4493, 4496, 4500, 4505, 4506, | SPOCK3 | −2.78 |
| | | | | 4522, 2959, 3354, 2879, 3351, 3951, 3952, 7390, | | |
| | | | | 8151, 7745, 8674, 7862, 8555, 8291, 8542, 7775, | | |
| | | | | 8670, 8212, 7911, 5944, 8680, 8411, 8508, 6200, | | |
| | | | | 8249, 7909, 8528, 8673, 7951, 8415, 8750, 7722, | | |
| 8091283 | 1295 | NM_182943 | 1883 | 3144, 3823, 3824, 7950, 7025, 6032, 8398, 5670, | PLOD2 | −2.76 |
| | | | | 7845, | | |
| 7908003 | 1296 | NM_030769 | 1884 | 3446, 5244, 8301, 8566, 7329, 6079, 6297, 6185, | NPL | −2.74 |
| | | | | 6208, 6540, 5453, 5873, 5974, 6575, | | |
| 8147145 | 1297 | NM_152565 | 1885 | 3548, 3146, 7959, 8150, 5593, | ATP6V0D2 | −2.73 |
| 8073585 | 1298 | NM_014509 | 1886 | 4046, 3113, 3203, 4870, 6789, 7257, 7696, 6190 | SERHL | −2.73 |
| | | | | 7885, 6148, 8055, 5575, | | |
| 8059376 | 1299 | NM_006216 | 1887 | 3235, 4656, 4657, 4658, 6475, 5410, 8093, 7735, | SERPINE2 | −2.70 |
| | | | | 6860, | | |
| 7909789 | 1300 | NM_003238 | 1888 | 2610, 5188, 5189, 7936, 5882, 8554, 5876, | TGFB2 | −2.68 |
| 8139057 | 1301 | NM_014800 | 1889 | 2968, 3692, 4045, 2861, 3438, 4258, 4520, 4479, | ELMO1 | −2.68 |
| | | | | 2801, 2880, 3542, 5280, 5279, 8359, 7086, 5936, | | |
| | | | | 6939, 6791, 6584, 7972, 7197, 7051, 6279, 5770, | | |
| | | | | 6974, 6770, 7488, 7401, 7549, 6438, 7349, | | |
| 8144656 | 1302 | BC132945 | 1890 | 4115, 2976, | C8orf49 | −2.66 |
| 8112615 | 1303 | NM_003633 | 1891 | 2731, 4418, 7756, 5853, 8481, | ENC1 | −2.65 |
| 8057620 | 1304 | NM_000393 | 1892 | 3762, 3939, 7320, 7380, 7573, | COL5A2 | −2.64 |
| 7919314 | 1305 | NM_001461 | 1893 | 3169, 4740, 4742, 4741, 5452, 5926, 6568, 7792 | FMO5 | −2.63 |
| | | | | 6924, | | |
| 7972750 | 1306 | NM_001845 | 1894 | 4195, 4166, 6498, 6735, | COL4A1 | −2.61 |
| 8097513 | 1307 | NM_002413 | 1895 | 3025, 5261, 8016, 7820, 8094, 5528, 8127, | MGST2 | −2.60 |
| 8169711 | 1308 | NM_012084 | 1896 | 2899, 3400, 5864, | GLUD2 | −2.59 |
| 8041179 | 1309 | NM_024692 | 1897 | 4055, 4666, 6910, 6677, 7982, 7631, 5684, 7192, | CLIP4 | −2.58 |
| | | | | 6576, 6387, 7420, 8380, | | |
| 8123388 | 1310 | NM_018974 | 1898 | 3851, 4738, 4736, 5385, 8261, 6041, 8180, 6272, | UNC93A | −2.57 |
| 8155192 | 1311 | NM_022343 | 1899 | 3083, 3132, 7426, 7611, 7741, 8325, 6925, | GLIPR2 | −2.54 |
| 8127065 | 1312 | NM_000846 | 1900 | 4266, 4667, 8541, | GSTA2 | −2.53 |
| 7928107 | 1313 | NM_018649 | 1901 | 2818, 3965, 5976, 7242, | H2AFY2 | −2.53 |
| 8018761 | 1314 | NM_006456 | 1902 | 2638, 4437, 5345, | ST6GALNAC2 | −2.52 |
| 7965335 | 1315 | NM_001946 | 1903 | 2898, 3562, 3563, 5485, 5764, | DUSP6 | −2.52 |
| 7965573 | 1316 | NM_021229 | 1904 | 2846, 3957, 6056, 5886, | NTN4 | −2.50 |
| 8006681 | 1317 | NM_005568 | 1905 | 2635, 5231, 5430, | LHX1 | −2.50 |
| 7962427 | 1318 | NM_031292 | 1906 | 3592, 4173, 4172, 4174, 6664, 6050, 6684, | PUS7L | −2.50 |
| 8163637 | 1319 | NM_002160 | 1907 | 2581, 5293, 6635, 7873, 8072, 6170, 6096, 6061, | TNC | −2.47 |
| | | | | 5879, 7429, 6261, 6994, | | |
| 8078286 | 1320 | NM_016152 | 1908 | 2681, 3505, 4596, 4598, 8684, 7033, 8333, 6707 | RARB | −2.47 |
| | | | | 5820, 6360, 7180, | | |
| 8014591 | 1321 | NM_000458 | 1909 | 2989, 4226, 4915, 5347, 6709, 6537, | HNF1B | −2.46 |
| 7970676 | 1322 | NM_001007538 | 1910 | 4110, 3758, 5588, | SHISA2 | −2.45 |
| 7902495 | 1323 | NM_144573 | 1911 | 3950, 4178, 4987, 5775, 6960, 6998, 8177, 6811, | NEXN | −2.45 |
| | | | | 5834, 5619, 7685, 5942, | | |
| 8059852 | 1324 | AK301707 | 1912 | 4489, 4652, 6889, | MSL3L2 | −2.43 |
| 7954969 | 1325 | NM_001114182 | 1913 | 3491, 4765, 4766, 4767, 4768, 4769, 7247, 7490, | IRAK4 | −2.42 |
| | | | | 6878, 6277, | | |
| 7920291 | 1326 | NM_080388 | 1914 | 3782, 2999, 5985, 6066, 6285, 6298, 8082, | S100A16 | −2.40 |
| 8140955 | 1327 | NM_001145306 | 1915 | 3279, 4773, 4772, 5552, 8113, 8064, 7668, 6346, | CDK6 | −2.40 |
| 8105607 | 1328 | NM_001164442 | 1916 | 4893, 7805, 7948, 7918, 8707, 6538, | FAM159B | −2.40 |
| 7921344 | 1329 | NM_012081 | 1917 | 4573, 4214, 5359, 7416, | ELL2 | −2.39 |
| 8091780 | 1330 | NM_003781 | 1918 | 3474, 2819, 3653, 4514, 2847, 2848, 3036, 3135, | B3GALNT1 | −2.39 |
| | | | | 3255, 3915, 3916, 3917, 3918, 3919, 8586, 8499, | | |
| | | | | 8324, 5811, 6661, 6367, 8420, 8708, 8676, 8027, | | |
| | | | | 7430, 8305, 8658, 7607, 8455, 6477, 8126, 8442, | | |
| | | | | 7637, | | |
| 8007493 | 1331 | NM_001661 | 1919 | 3037, 3901, 5663, | ARL4D | −2.38 |
| 8068593 | 1332 | NM_005239 | 1920 | 3390, 3760, 7144, 5963, 7355, 6033, | ETS2 | −2.36 |
| 8169145 | 1333 | NM_152423 | 1921 | 3142, 4991, 4993, 6316, 5894, 5576, | MUM1L1 | −2.36 |
| 8013606 | 1334 | NM_000638 | 1922 | 2897, 3932, 5348, | VTN | −2.35 |
| 8140668 | 1335 | NM_006080 | 1923 | 4019, 3973, 5554, 7046, 6980, | SEMA3A | −2.35 |
| 8038981 | 1336 | NM_030972 | 1924 | 3136, 4861, 4862, 4863, 4864, 5722, | ZNF611 | −2.34 |
| 7944803 | 1337 | NM_014622 | 1925 | 3486, 3642, 3641, 4463, 5983, 7513, 6905, 7593, | VWA5A | −2.33 |
| | | | | 6204, 7228, | | |
| 8099364 | 1338 | NM_053042 | 1926 | 3218, 3781, 5718, 8226, 8286, 7880, 7968, | ZNF518B | −2.32 |
| 7919193 | 1339 | NM_199040 | 1927 | 2810, 4694, 3968, 3967, 6935, 5781, | NUDT4P1 | −2.32 |
| 8101304 | 1340 | NM_152545 | 1928 | 4526, 3145, 8395, 5654, 5568, 7781, 8214, 6622, | RASGEF1B | −2.31 |
| 8143643 | 1341 | NM_022445 | 1929 | 3220, 4001, 4002, 8245, 6258, 8436, 7518, 7791, | TPK1 | −2.30 |
| | | | | 6711, | | |
| 8095986 | 1342 | NM_005139 | 1930 | 2871, 3966, 8438, 8323, 7932, 5460, 8713, 8202, | ANXA3 | −2.29 |
| | | | | 7789, | | |
| 8007921 | 1343 | NM_001002841 | 1931 | 3053, 3703, 2998, 6007, 6931, | MYL4 | −2.29 |
| 7926609 | 1344 | NM_005180 | 1932 | 3522, 5257, 6836, 7177, 8311, 6490, 6981, 7037, | BMI1 | −2.29 |
| | | | | 8773, 6757, 6570, | | |
| 8050240 | 1345 | NM_002539 | 1933 | 3273, 2760, 5350, 7567, 7441, 6418, | ODC1 | −2.28 |
| 8171161 | 1346 | NM_000047 | 1934 | 4099, 4234, 6631, 6722, | ARSE | −2.28 |
| 8096176 | 1347 | NM_006264 | 1935 | 2621, 4579, 4580, 4581, 4582, 6886, 7770, 7790, | PTPN13 | −2.27 |
| | | | | 7383, 8720, 7840, 6014, 5863, 7589, | | |
| 8039013 | 1348 | NM_203307 | 1936 | 3243, 5855, | ZNF321 | −2.26 |
| 7934156 | 1349 | NM_018055 | 1937 | 3088, 4747, 7082, 5642, | NODAL | −2.26 |
| 8138370 | 1350 | NM_020319 | 1938 | 3159, 5132, 7035, 7445, 7172, 5729, | ANKMY2 | −2.24 |
| 7924107 | 1351 | NM_014873 | 1939 | 3152, 4614, 7821, 6002, 6174, | LPGAT1 | −2.24 |
| 8089011 | 1352 | NM_000313 | 1940 | 2597, 4770, 6690, | PROS1 | −2.23 |
| 8167912 | 1353 | NM_007250 | 1941 | 3520, 4810, 4811, 7702, 8280, 6724, 6350, 6020, | KLF8 | −2.23 |
| 7947338 | 1354 | NM_000280 | 1942 | 3805, 4400, 4401, 4402, 6733, 7304, 6876, 7135, | PAX6 | −2.23 |
| | | | | 7188, 7299, 6953, 7333, 7281, 8690, 7478, 7234, | | |
| | | | | 8432, 7017, 6745, 7558, 5383, | | |
| 8155930 | 1355 | NM_001490 | 1943 | 2578, 4156, 4157, 4158, 4159, 4160, 8460, 6349, | GCNT1 | −2.23 |
| | | | | 8001, 7226, 6641, | | |
| 8106411 | 1356 | NM_130772 | 1944 | 3017, 4191, 8752, 5748, | S100Z | −2.23 |
| 8039025 | 1357 | NR_003578 | 1945 | 3075, 4190, 5550, | ZNF702P | −2.21 |
| 8132167 | 1358 | NM_194300 | 1946 | 4108, 4535, 3117, 4495, 4212, 7015, 6902, 6655, | CCDC129 | −2.21 |
| | | | | 7399, 7356, 7062, 8117, 5731, 6766, | | |
| 8049542 | 1359 | NM_001137550 | 1947 | 4662, 7654, | LRRFIP1 | −2.20 |
| 8102912 | 1360 | NM_015130 | 1948 | 4180, 4132, 6423, | TBC1D9 | −2.20 |
| 8008819 | 1361 | NM_001005404 | 1949 | 3716, 3768, 5719, | YPEL2 | −2.19 |
| 8112668 | 1362 | NM_016591 | 1950 | 2806, 4600, 5633, | GCNT4 | −2.18 |
| 7970241 | 1363 | NM_000504 | 1951 | 2567, 2573, 3210, 3246, 4308, 4309, 3935, 7894, | F10 | −2.18 |
| | | | | 6670, 7728, 6624, 7953, 7352, 6493, | | |
| 8036389 | 1364 | NM_152279 | 1952 | 3196, 4990, 5862, 6596, | ZNF585B | −2.17 |
| 8097529 | 1365 | NM_153702 | 1953 | 3416, 5196, 8425, 7800, 5802, 8102, 8387, 8257, | ELMOD2 | −2.16 |
| | | | | 8391, | | |
| 7962349 | 1366 | NM_173601 | 1954 | 3139, 4199, 4198, 7252, 5482, | GLT8D3 | −2.16 |
| 7952601 | 1367 | NM_001162422 | 1955 | 3402, 4871, 4686, 4687, 7425, 5612, 5931, | ETS1 | −2.16 |
| 8001387 | 1368 | NM_001127892 | 1956 | 4497, 4411, 4078, 6646, 5432, 7163, | SALL1 | −2.16 |
| 8040163 | 1369 | NM_001039613 | 1957 | 3912, 3107, 4261, 4549, 3207, 3482, 3588, 3934, | IAH1 | −2.16 |
| | | | | 6534, 8330, 8409, 8205, 6128, 8770, 8556, 7901, | | |
| | | | | 8340, 8747, 8755, 7721, 8381, 8091, 7817, 5835, | | |
| 8165672 | 1370 | L23320 | 1958 | 2618, | RFC1 | −2.15 |
| 7946288 | 1371 | NM_013249 | 1959 | 2781, 4224, 5492, | ZNF214 | −2.15 |
| 7953665 | 1372 | NM_199286 | 1960 | 3455, 3720, 6044, | DPPA3 | −2.15 |
| 8103311 | 1373 | NM_000508 | 1961 | 2574, 3860, 3861, 6647, 5699, 6582, | FGA | −2.15 |
| 8084206 | 1374 | NM_032047 | 1962 | 2926, 3099, 3434, 3020, 2912, 3182, 3956, 6802, | B3GNT5 | −2.13 |
| | | | | 6453, 8375, 8383, 8256, 7811, 7501, 5794, 8221, | | |
| | | | | 7151, | | |
| 8174389 | 1375 | NM_001847 | 1963 | 2623, 4165, 4167, 6197, 5733, 6413, 8100, 7288, | COL4A6 | −2.13 |
| | | | | 7442, 7535, | | |
| 7918768 | 1376 | NM_198459 | 1964 | 3586, 4103, 6796, 6943, 8236, 8046, 6164, 7594, | DENND2C | −2.13 |
| 7950885 | 1377 | NM_012193 | 1965 | 2784, 5289, | FZD4 | −2.12 |
| 8005048 | 1378 | NM_001146313 | 1966 | 3740, 4806, 4804, 4805, 7259, 6792, 7438, 6345 | MYOCD | −2.12 |
| | | | | 6178, 6713, | | |
| 8101952 | 1379 | NM_145244 | 1967 | 3382, 5197, 8631, 5514, 8733, | DDIT4L | −2.12 |
| 8111887 | 1380 | NM_001005473 | 1968 | 4020, 5772, 7331, | PLCXD3 | −2.11 |
| 8113220 | 1381 | NM_012081 | 1969 | 3398, 4214, 5359, 7416, 8279, 8525, | ELL2 | −2.10 |
| 8114287 | 1382 | NM_004598 | 1970 | 2804, 3903, 8553, 8505, 6817, 7957, 5469, 8014, | SPOCK1 | −2.10 |
| | | | | 7754 | | |
| 7955694 | 1383 | NM_002178 | 1971 | 3353, 3682, 5662, | IGFBP6 | −2.09 |
| 8073596 | 1384 | NM_014509 | 1972 | 3645, 4046, 4023, 4076, 4075, 4492, 4499, 3203 | SERHL2 | −2.09 |
| | | | | 7885, 6148, 6161, 6501, 7434, 5747, 5575, | | |
| 8083779 | 1385 | NM_005025 | 1973 | 4280, 4350, 4351, 7428, 8534, 7747, 5615, 7839, | SERPINI1 | −2.09 |
| | | | | 7903, | | |
| 8121601 | 1386 | NM_153711 | 1974 | 3931, 6155, | FAM26E | −2.08 |
| 8169174 | 1387 | NM_024539 | 1975 | 3527, 3478, 3479, 5441, 5712, 6719, | RNF128 | −2.08 |
| 8117045 | 1388 | NM_153020 | 1976 | 3092, 4733, 4735, 4734, 6592, 6373, 5606, 8008, | RBM24 | −2.08 |
| | | | | 8572, 8110, | | |
| 7965357 | 1389 | NM_003774 | 1977 | 4550, 3393, 5235, 6995, | GALNT4 | −2.08 |
| 8162652 | 1390 | NM_001333 | 1978 | 3462, 5253, 8071, 5450, | CTSL2 | −2.08 |
| 8023528 | 1391 | NM_052947 | 1979 | 3413, 4170, 6142, | ALPK2 | −2.07 |
| 8069508 | 1392 | ENST00000457997 | 1980 | 6891, 6940, | ANKRD20B | −2.06 |
| 7929388 | 1393 | NM_016341 | 1981 | 4217, 4071, 4918, 6138, 5440, 6045, 6266, | PLCE1 | −2.06 |
| 7944082 | 1394 | NM_003186 | 1982 | 2895, 3656, 3657, 3038, 7198, 5531, 6894, 5715, | TAGLN | −2.06 |
| 8057771 | 1395 | NM_003151 | 1983 | 3066, 5299, 7451, 8037, 7396, 6374, 6129, 7059 | STAT4 | −2.06 |
| | | | | 8595, 6687, 8616, | | |
| 7907702 | 1396 | NM_003101 | 1984 | 3047, 5308, 5878, 7270, | SOAT1 | −2.05 |
| 7910134 | 1397 | NM_031944 | 1985 | 2853, 2923, 6314, | MIXL1 | −2.04 |
| 8062823 | 1398 | NM_000457 | 1986 | 3706, 3707, 3708, 4669, 4387, 4383, 2677, 2664, | HNF4A | −2.04 |
| | | | | 3125, 4751, 3292, 3293, 3294, 3867, 3868, 3866, | | |
| | | | | 6660, 5774, 5653, 6081, 7077, 5571, 7633, | | |
| 8059878 | 1399 | NM_024726 | 1987 | 4003, 4483, 2974, 3095, 3137, 3231, 3536, 8451, | IQCA1 | −2.04 |
| | | | | 6443, 7562, 7057, 8660, 5402, 7302, 7122, 6806, | | |
| | | | | 5760, 7624, 7362, | | |
| 8022817 | 1400 | NM_020805 | 1988 | 4494, 3749, 6140, 6012, | KLHL14 | −2.04 |
| 8102862 | 1401 | NM_018717 | 1989 | 4377, 4379, 2704, 4531, 2925, 4284, 7322, 8019, | MAML3 | −2.03 |
| | | | | 7858, 5635, | | |
| 8096845 | 1402 | NM_001963 | 1990 | 3828, 5114, 5115, 5116, 5500, 7841, 7967, 7927, | EGF | −2.03 |
| 7924663 | 1403 | NM_020997 | 1991 | 2777, 5273, 5561, 8017, | LEFTY1 | −2.03 |
| 7970381 | 1404 | AK057820 | 1992 | 2984, 4169, 6891, 6432, 6336, 7704, 6356, 7272, | ANKRD20B | −2.03 |
| | | | | 7347, | | |
| 7922727 | 1405 | NM_001102450 | 1993 | 3879, 4223, 3904, 8112, 8121, 6042, 8709, 7965, | RGS8 | −2.03 |
| | | | | 5447, 5943, | | |
| 8157090 | 1406 | NM_005421 | 1994 | 4057, 4779, 5674, | TAL2 | −2.02 |
| 8050071 | 1407 | NM_018269 | 1995 | 3559, 5111, 5637, | ADI1 | −2.02 |
| 7944869 | 1408 | NM_017425 | 1996 | 2833, 2894, 4744, 5335, | SPA17 | −2.01 |
| 8127370 | 1409 | NM_152688 | 42 | 4263, 4412, 8227, 5521, | KHDRBS2 | −2.01 |
| 8113551 | 1410 | NM_002387 | 43 | 3412, 4147, 4148, 8175, 8246, 5792, 7893, | MCC | −2.00 |

Table 12: Provided are the description of the non-membranal genes along with the sequence identifiers of the Affymetrix probes, the target sequence identified by the Affymetrix probe, and the mRNA sequences represented by the target sequences. Also shown are the log 2 fold change in expression levels of the indicated genes between the PDX1-GFP cells and the SOX17-GFP cells (PDX1-SOX17). The (+) or (−) sign means that the genes are upregulated or downregulated, respectively, in the PDX1-GFP cells as compared to the SOX17-GFP cells.

Identification of Clusters of Genes which are Overexpressed During Differentiation into Pancreatic Cells—

The transcriptional profiles of the SOX17⁺ and PDX1⁺ progenitor cells were compared to profiles of undifferentiated ES cells and of differentiated pancreatic cells. Partek analysis enabled to define different clusters of genes according to their expression patterns (FIGS. 5B-E).

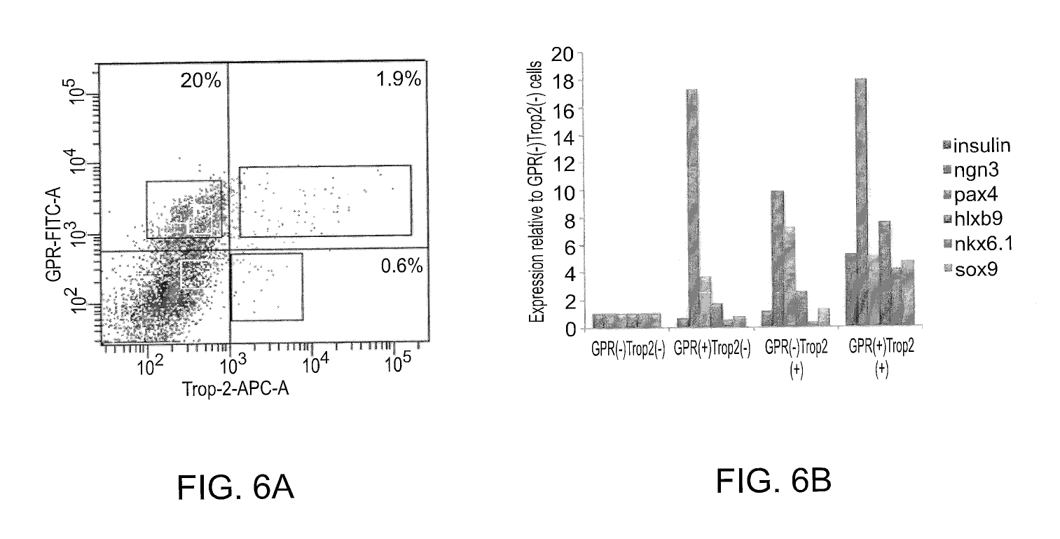

The present inventors have further studied several surface markers that were found to be enriched in the PDX1⁺ cells in the microarray analysis and were validated by qPCR analysis. These are the G protein-coupled receptor 50 (GPR50) and tumor-associated calcium signal transducer 2 (TROP-2), whose expression showed a good correlation with PDX1 expression (FIG. 6A). Cells from 25 day old EBs were dissociated and were stained with antibodies specific to TROP-2 and GPR50 and were FACS sorted. Four different populations were obtained: GPR50⁺TROP-2⁺ (1.9%), GPR50⁺TROP-2⁻ (20%), GPR50⁻TROP-2⁺ (0.6%) and a negative population which did not express either of these markers (GPR50⁻/TROP-2⁻). Quantitative-PCR analysis revealed an increase in mature pancreatic markers, especially in the GPR50⁺TROP-2⁺ population, including an induction in INSULIN, NGN3, PAX4, HLXB9, NK6 homeobox 1(NKX6.1), and SRY box 9 (SOX9).

TROP-2 and GPR50 are two exemplary markers of the large number of cell surface markers that were revealed by the Affymetrix study including markers enriched in the populations of interest as well as markers depleted in the populations of interest. As shown, these markers can be used in positive selection to enrich for the desired cells. They can also be used in negative selection to remove cells other than the cells that are desired.

The cell surface markers that were identified based on comparative analysis between pluripotent undifferentiated hESC, the GFP+ population from the cells bearing the SOX17-GFP construct and the GFP+ population from the cells bearing the PDX1-GFP construct fall into a number of categories, all of which can potentially be used for immuno-purification/immuno-isolation. Following is a short description of these marker groups:

I. Markers that could be Useful to Enrich for Endodermal Progenitor Cells:

Cell surface markers enriched in the SOX17 population relative to undifferentiated hESC;

Cell surface markers depleted in the SOX17 population relative to undifferentiated hESC;

II. Markers that could be Useful to Enrich for Pancreatic Progenitor Cells:

Cell surface markers enriched in the PDX1 population relative to undifferentiated hESC;

Cell surface markers depleted in the PDX1 population relative to undifferentiated hESC;

III. Cell Surface Markers Enriched in the PDX1 Population Relative to the SOX17 Population.

Without being bound by any theory, these markers would be useful in the event that two sorting procedures are incorporated into the process of manufacturing the therapeutic cell population—one sorting procedure at the stage of SOX17 expression and a second sorting procedure at the stage of PDX1 expression.

IV. Cell Surface Markers Depleted in the PDX1 Population Relative to the SOX17 Population.

In addition, all the cell surface markers above and additional non-cell surface markers that exhibit similar expression patterns can be used for quality control of replacement islet beta cells for transplant-based diabetes treatment during their manufacture.

### Example 4

### Identification of Stage Specific Markers Using the Partek Cluster Analysis

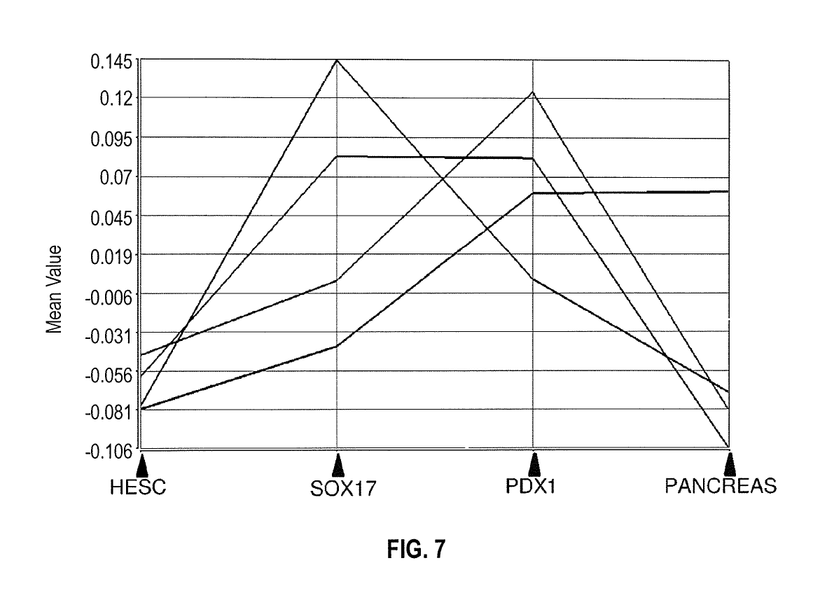

The bioinformatic analysis of cell specific markers was a central part of the entire experiment. Key steps in this part were: A. To assign those genes to a stage specific category; B. To analyze genes related to the relevant cell component. The present inventors have used the Gene Ontology data to include only genes with cell surface and membranal information. Stage specific genes were assigned to one of four categories: (i). SOX17⁺ specific; (ii). PDX1⁺ specific; (iii). SOX17⁺/PDX1⁺ specific; (iv). PDX1+/Pancreas specific. The generation of those specific lists was done by using the PARTEK partition clustering analysis (FIG. 7).

Stage and Compartment Specific Gene Identification—

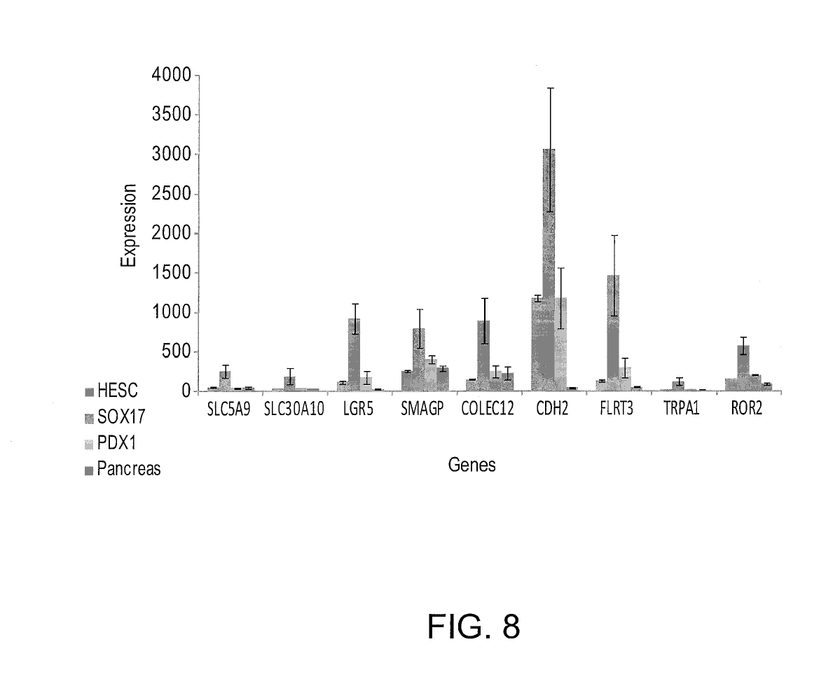

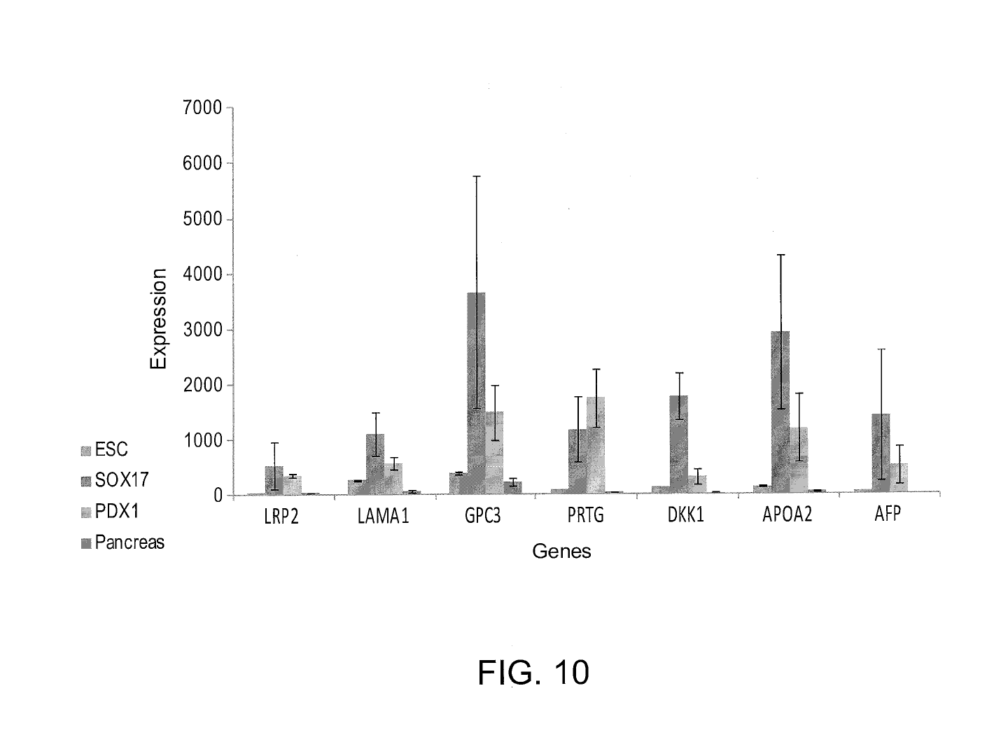

Following the PARTEK cluster analysis a further prioritization of relevant genes was needed. For this purpose a standard excel analysis was used. A threshold of Fold Change=2.0 was set (at it is an acceptable threshold in microarray studies). In addition, the present inventors have used the Student's test for the gene selection. FIGS. 8-11 present selected genes for each stage. The entire list appears in Tables 13-20 below.

TABLE 13
SOX17 specific cell surface genes

| | | | SEQ | | | SOX17/ |
|---|---|---|---|---|---|---|
| | Affy. target | | ID NO: | Polyn. SEQ | | HESC |
| Probe | nucl.SEQ | | (of Rep. | ID NOs: rep. | Gene | fold |
| set ID | ID NO: | Rep. Public ID | Public ID) | by target | Symbol | change |
| 8029280 | 1997 | NM_020406 | 2273 | 3465, 2825, 3947, 3744, | CD177 | 9.45 |
| | | | | 4962, 4250, 3064, 3545, | | |
| | | | | 3050, 4005, 6408, 6835, | | |
| | | | | 7480, | | |
| 7938090 | 1998 | NM_176875 | 2274 | 2809, 5307, 5676, | CCKBR | 5.81 |
| 8077185 | 1999 | NM_000039 | 2275 | 3902, 2762, 6306, 5375, | APOA1 | 7.79 |
| | | | | 6654, 6990, | | |
| 7951865 | 2000 | NM_000039 | 2276 | 3902, 2762, 6306, 5375, | APOA1 | 7.44 |
| | | | | 6654, 6990, | | |
| 8052072 | 2001 | NM_000145 | 2277 | 4265, 5021, 5022, 6252, | FSHR | 5.19 |
| | | | | 5713, 7612, 7221, 6397, | | |
| 8058498 | 2002 | NM_003468 | 2278 | 2934, 4015, 5838, | FZD5 | 2.38 |
| 8058765 | 2003 | NM_212482 | 2279 | 3732, 3409, 3983, 4677, | FN1 | 4.30 |
| | | | | 4679, 3284, 3310, 3403, | | |
| | | | | 3404, 3405, 3406, 4727, | | |
| | | | | 3110, 4551, 4571, 4572, | | |
| | | | | 4576, 3822, 3494, 2852, | | |
| | | | | 3620, 3621, 3622, 3623, | | |
| | | | | 3624, 3625, 5996, 7280, | | |
| | | | | 7649, 6915, 8364, 8529, | | |
| | | | | 6768, 7398, 5846, 6424, | | |
| | | | | 8736, 8430, 7036, 5993, | | |
| | | | | 8153, 8487, 6324, 6771, | | |
| | | | | 6223, 6205, 5539, 6620, | | |
| | | | | 6121, 6206, 8147, 5687, | | |
| 8060850 | 2004 | NM_001200 | 2280 | 3900, 6567, | BMP2 | 3.36 |
| 8088560 | 2005 | NM_182920 | 2281 | 4100, 3333, 8622, 8439, | ADAMTS9 | 7.20 |
| | | | | 8457, 6636, 7795, 8233, | | |
| | | | | 8784, | | |
| 8056222 | 2006 | NM_001935 | 2282 | 3604, 3605, 4611, 2562, | DPP4 | 4.32 |
| | | | | 2570, 3550, 4303, 3611, | | |
| | | | | 6918, 6975, 6558, 7617, | | |
| | | | | 8396, 7176, 6249, 7784, | | |
| | | | | 8632, 8468, | | |
| 8103311 | 2007 | NM_000508 | 2283 | 2574, 3860, 3861, 6647, | FGA | 7.36 |
| | | | | 5699, 6582, | | |
| 8021946 | 2008 | NM_130386 | 2284 | 2891, 4601, 6579, | COLEC12 | 5.08 |
| 8162283 | 2009 | NM_004560 | 2285 | 3804, 4102, 2608, 3151, | ROR2 | 3.51 |
| | | | | 3287, 5241, 6393, 7306, | | |
| | | | | 6756, 8135, | | |
| 8081298 | 2010 | NM_032787 | 2286 | 3995, 4847, 5470, 7733, | GPR128 | 4.64 |
| | | | | 8308, 8445, | | |
| 8058857 | 2011 | NM_000599 | 2287 | 2583, 4357, 8723, 5360, | IGFBP5 | 5.34 |
| 8111677 | 2012 | NM_002310 | 2288 | 2551, 4403, 4404, 5491, | LIFR | 4.97 |
| | | | | 7187, 7919, 8314, 7758, | | |
| 7950885 | 2013 | NM_012193 | 2289 | 2784, 5289, | FZD4 | 4.47 |
| 7963786 | 2014 | NM_002205 | 2290 | 3341, 3761, 5661, 6880, | ITGA5 | 4.42 |
| 8149825 | 2015 | NM_003115 | 2291 | 2725, 3793, 5771, | STC1 | 3.21 |
| 7922343 | 2016 | NM_003326 | 2292 | 3222, 4594, 5549, 8171, | TNFSF4 | 2.79 |
| | | | | 6071, | | |
| 8029280 | 2017 | NM_020406 | 2293 | 3465, 2825, 3947, 3744, | CD177 | 2.43 |
| | | | | 4962, 4250, 3064, 3545, | | |
| | | | | 3050, 4005, 6408, 6835, | | |
| | | | | 7480, | | |
| 8059067 | 2018 | NM_002181 | 2294 | 4757, 4992, 5587, | IHH | 2.06 |

| | SOX17/ | SOX17/ | | | | |
|---|---|---|---|---|---|---|
| | PDX1 | PANC | fold | SOX17 | SOX17 | SOX17 |
| Probe | fold | fold | change | vs. | vs. | vs. |
| set ID | change | change | average | HESC | PDX1 | PANC |
| 8029280 | 11.14 | 6.93 | 9.17 | 3.9E−04 | 2.4E−04 | 1.0E−03 |
| 7938090 | 8.69 | 9.10 | 7.87 | 1.6E−03 | 4.5E−04 | 3.9E−04 |
| 8077185 | 4.01 | 8.83 | 6.88 | 1.2E−02 | 1.0E−01 | 1.0E−02 |
| 7951865 | 3.85 | 8.49 | 6.59 | 1.2E−02 | 1.0E−01 | 9.6E−03 |
| 8052072 | 5.53 | 6.22 | 5.65 | 1.1E−03 | 8.9E−04 | 5.8E−04 |
| 8058498 | 4.11 | 9.65 | 5.38 | 1.7E−01 | 6.3E−02 | 2.5E−02 |
| 8058765 | 2.39 | 8.73 | 5.14 | 2.9E−04 | 3.0E−01 | 1.2E−03 |
| 8060850 | 5.86 | 5.66 | 4.96 | 1.4E−02 | 1.8E−03 | 2.1E−03 |
| 8088560 | 3.76 | 3.07 | 4.67 | 2.0E−05 | 1.8E−03 | 2.8E−04 |
| 8056222 | 2.07 | 6.97 | 4.45 | 9.4E−02 | 3.7E−01 | 3.6E−02 |
| 8103311 | 3.04 | 2.03 | 4.15 | 7.9E−02 | 4.1E−02 | 6.3E−01 |
| 8021946 | 3.50 | 3.74 | 4.10 | 1.1E−02 | 3.4E−02 | 2.7E−02 |
| 8162283 | 2.68 | 5.90 | 4.03 | 4.5E−03 | 1.1E−02 | 2.0E−03 |
| 8081298 | 4.55 | 2.52 | 3.90 | 4.5E−02 | 4.7E−02 | 1.9E−01 |
| 8058857 | 2.55 | 3.36 | 3.75 | 2.3E−02 | 1.6E−01 | 7.7E−02 |
| 8111677 | 4.00 | 1.50 | 3.49 | 4.8E−02 | 7.3E−02 | 5.8E−01 |
| 7950885 | 3.91 | 1.76 | 3.38 | 3.0E−02 | 4.2E−02 | 5.0E−01 |
| 7963786 | 3.10 | 2.50 | 3.34 | 4.3E−04 | 2.4E−03 | 7.7E−03 |
| 8149825 | 2.62 | 3.23 | 3.02 | 8.2E−02 | 1.4E−01 | 8.1E−02 |
| 7922343 | 2.70 | 2.89 | 2.79 | 7.8E−03 | 9.2E−03 | 6.5E−03 |
| 8029280 | 2.40 | 2.51 | 2.44 | 4.5E−03 | 4.2E−03 | 3.4E−03 |
| 8059067 | 2.09 | 2.13 | 2.09 | 9.2E−02 | 8.7E−02 | 8.1E−02 |

Table 13. Provided are the description of the cell surface genes along with the sequence identifiers of the Affymetrix probes, the target sequence identified by the Affymetrix probe, the polynucleotide represented by the target sequence and the Public ID (and SEQ ID NO:) of the mRNA sequence representative of the target sequence. “Polyn.” = polynucleotide; “Rep.” = representative; Also shown are the fold change and T-test p-values for change in expression levels of the indicated genes between the SOX17-GFP cells and undifferentiated hESCs (SOX17/HESC); the SOX17-GFP cells and PDX1-GFP cells (SOX17/PDX1); and the SOX17-GFP cells and the Pancreas cells (SOX17/PANC).

TABLE 14
PDX1 specific cell surface genes

| | | | SEQ | | | PDX1/ |
|---|---|---|---|---|---|---|
| | Affy. target | | ID NO: | Polyn. SEQ | | HESC |
| Probe | nucl. SEQ | Rep. | (of Rep. | ID NOs: rep. | Gene | fold |
| set ID | ID NO: | Public ID | Public ID) | by target | Symbol | change |
| 7929373 | 2019 | NM_005097 | 2295 | 2745, 4359, 8710, 8593, | LGI1 | 9.41 |
| | | | | 6230, 6132, | | |
| 8165658 | 2020 | U18810 | 2296 | 2553, 3426, 3422, 6269, | VIPR2 | 4.01 |
| 7915472 | 2021 | NM_006516 | 2297 | 4268, 4338, 5901, 6869, | SLC2A1 | 1.77 |
| | | | | 6399, 8613, 6344, 6364, | | |
| | | | | 5884, 7600, | | |
| 7947156 | 2022 | NM_001135091 | 2298 | 3463, 4618, 4620, 4619, | MUC15 | 8.35 |
| | | | | 5504, 6821, 7005, | | |
| 8135033 | 2023 | ENST00000379442 | 2299 | 4894, 7027, | MUC12 | 7.36 |
| 8095303 | 2024 | NM_015236 | 2300 | 2842, 4699, 4713, 4296, | LPHN3 | 3.77 |
| | | | | 4285, 8560, 7869, 8477, | | |
| | | | | 8293, 8434, 5644, 8571, | | |
| | | | | 8574, 8160, 8174, 5553, | | |
| | | | | 7807, 8297, 8402, 8511, | | |
| | | | | 8619, 8139, 8480, | | |
| 8033674 | 2025 | NM_024690 | 2301 | 3192, 3906, 7402, 7014, | MUC16 | 4.82 |
| | | | | 7634, 7481, | | |
| 7918936 | 2026 | NM_024626 | 2302 | 2837, 3971, 6218, 6213, | VTCN1 | 6.12 |
| | | | | 7307, 8522, 5804, 6025, | | |
| 8151684 | 2027 | AL136588 | 2303 | 5826, | MMP16 | 2.44 |
| 8145611 | 2028 | NM_017412 | 2304 | 2808, 5230, 8548, 5392, | FZD3 | 2.33 |
| 8056184 | 2029 | NM_000888 | 2305 | 3091, 2830, 8224, 8635, | ITGB6 | 5.53 |
| | | | | 5643, 7596, 6710, | | |
| 8114415 | 2030 | NM_001496 | 2306 | 3167, 3584, 7363, 5508, | GFRA3 | 3.92 |
| 8170476 | 2031 | NM_004224 | 2307 | 2669, 4964, 5346, | GPR50 | 2.51 |
| 8088919 | 2032 | NM_133631 | 2308 | 2713, 3930, 3948, 4146, | ROBO1 | 1.93 |
| | | | | 3949, 4391, 4683, 3115, | | |
| | | | | 4548, 4716, 4797, 4798, | | |
| | | | | 5105, 6728, 8746, 5966, | | |
| | | | | 7836, 8600, 7464, 8561, | | |
| | | | | 6404, 8251, 6433, 7621, | | |
| 8083968 | 2033 | NM_014932 | 2309 | 4961, 2776, 3071, 4307, | NLGN1 | 2.49 |
| | | | | 3297, 6467, 7268, 7261, | | |
| | | | | 7427, 6874, 7249, 6559, | | |
| | | | | 6760, 8521, 5935, | | |
| 8035304 | 2034 | NM_004335 | 2310 | 3090, 2805, 5403, 7124, | BST2 | 4.49 |
| 8135033 | 2035 | ENST00000379442 | 2311 | 4894, 7027, | MUC12 | 4.59 |
| 8160823 | 2036 | NM_147164 | 2312 | 2568, 5285, 5284, 6097, | CNTFR | 4.25 |
| | | | | 6693, 6678, | | |
| 8034783 | 2037 | NM_001008701 | 2313 | 2841, 4650, 4651, 5915, | LPHN1 | 1.72 |
| | | | | 5875, | | |
| 8146863 | 2038 | NM_001128205 | 2314 | 3240, 4424, 4425, 4426, | SULF1 | 3.46 |
| | | | | 4427, 6483, 8782, 7482, | | |
| | | | | 6383, 5417, | | |
| 8047788 | 2039 | NM_003812 | 2315 | 4109, 4113, 4530, 2862, | ADAM23 | 1.50 |
| | | | | 2734, 3877, 7431, 7068, | | |
| | | | | 6740, 7531, 5501, | | |
| 8118890 | 2040 | NM_152753 | 2316 | 3214, 3300, 5524, 6429, | SCUBE3 | 3.05 |
| 7928429 | 2041 | NM_002658 | 2317 | 3397, 4752, 4753, 6308, | PLAU | 2.31 |
| | | | | 6729, 8322, 7727, 8611, | | |
| | | | | 6028, 6300, 8374, | | |
| 7952526 | 2042 | NM_016952 | 2318 | 3857, 4137, 6357, 5459, | CDON | 2.51 |
| 8094301 | 2043 | NM_004787 | 2319 | 2748, 2765, 3985, 4678, | SLIT2 | 2.35 |
| | | | | 3436, 2744, 2767, 8366, | | |
| | | | | 7786, 6676, 7740, 5522, | | |
| | | | | 8033, 7772, 7956, 8641, | | |
| | | | | 8725, | | |
| 8135915 | 2044 | NM_013332 | 2320 | 2771, 4188, 4189, 8677, | C7orf68 | 2.08 |
| | | | | 7435, 5442, 6618, 7939, | | |
| 7926545 | 2045 | NM_032812 | 2321 | 3459, 3535, 6338, 7091, | PLXDC2 | 2.13 |
| | | | | 7374, | | |

| | PDX1/ | PDX1/ | | | | |
|---|---|---|---|---|---|---|
| | SOX17 | PANC | | PDX1 | PDX1 | PDX1 |
| Probe | fold | fold | FC | vs. | vs. | vs. |
| set ID | change | change | average | HESC | SOX17 | PANC |
| 7929373 | 4.10 | 10.86 | 8.12 | 2.7E−02 | 1.0E−01 | 2.2E−02 |
| 8165658 | 4.29 | 13.53 | 7.27 | 2.1E−01 | 2.9E−01 | 5.1E−02 |
| 7915472 | 2.04 | 16.02 | 6.61 | 5.1E−02 | 2.6E−01 | 5.2E−04 |
| 7947156 | 7.47 | 2.59 | 6.14 | 4.0E−02 | 4.6E−02 | 4.1E−01 |
| 8135033 | 4.01 | 2.56 | 4.64 | 3.5E−02 | 9.5E−02 | 2.2E−01 |
| 8095303 | 2.02 | 8.14 | 4.64 | 4.0E−02 | 2.5E−01 | 1.6E−02 |
| 8033674 | 3.99 | 4.97 | 4.60 | 9.6E−02 | 1.4E−01 | 9.1E−02 |
| 7918936 | 5.23 | 2.07 | 4.47 | 2.0E−02 | 4.0E−02 | 3.9E−01 |
| 8151684 | 2.18 | 8.56 | 4.40 | 2.5E−02 | 1.2E−01 | 6.4E−04 |
| 8145611 | 2.96 | 7.13 | 4.14 | 3.9E−03 | 2.4E−02 | 1.5E−02 |
| 8056184 | 4.77 | 1.82 | 4.04 | 1.4E−01 | 1.8E−01 | 6.1E−01 |
| 8114415 | 4.05 | 3.49 | 3.82 | 1.5E−02 | 1.8E−02 | 2.2E−02 |
| 8170476 | 1.70 | 7.24 | 3.81 | 2.2E−03 | 1.1E−01 | 9.7E−04 |
| 8088919 | 3.79 | 4.74 | 3.49 | 3.6E−02 | 9.4E−03 | 1.3E−02 |
| 8083968 | 3.63 | 4.30 | 3.48 | 1.5E−02 | 7.4E−03 | 3.8E−03 |
| 8035304 | 2.36 | 3.52 | 3.46 | 5.8E−02 | 3.7E−01 | 1.1E−01 |
| 8135033 | 2.70 | 2.87 | 3.39 | 4.1E−02 | 1.5E−01 | 1.1E−01 |
| 8160823 | 2.81 | 2.87 | 3.31 | 2.3E−02 | 1.4E−01 | 1.8E−01 |
| 8034783 | 1.83 | 6.32 | 3.29 | 1.0E−03 | 1.2E−01 | 3.9E−05 |
| 8146863 | 4.15 | 1.86 | 3.16 | 1.1E−01 | 1.2E−01 | 3.9E−01 |
| 8047788 | 1.55 | 6.27 | 3.11 | 2.7E−02 | 1.1E−01 | 1.4E−03 |
| 8118890 | 2.37 | 3.23 | 2.88 | 1.0E−01 | 2.0E−01 | 9.0E−02 |
| 7928429 | 3.69 | 2.63 | 2.88 | 3.7E−04 | 4.8E−03 | 2.0E−02 |
| 7952526 | 2.00 | 4.00 | 2.84 | 5.4E−02 | 1.1E−01 | 7.5E−02 |
| 8094301 | 3.57 | 2.55 | 2.82 | 4.4E−01 | 3.2E−01 | 4.1E−01 |
| 8135915 | 2.50 | 2.60 | 2.40 | 2.1E−02 | 3.1E−02 | 8.3E−02 |
| 7926545 | 2.18 | 2.09 | 2.13 | 1.7E−01 | 2.1E−01 | 2.2E−01 |

Table 14. Provided are the description of the cell surface genes along with the sequence identifiers of the Affymetrix probes, the target sequence identified by the Affymetrix probe, the polynucleotide represented by the target sequence and the Public ID (and SEQ ID NO:) of the mRNA sequence representative of the target sequence. “Polyn.” = polynucleotide; “Rep.” = representative; Also shown are the fold change and T-test p-values for change in expression levels of the indicated genes between the PDX1-GFP cells and undifferentiated hESCs (PDX1/HESC); the PDX1-GFP cells and SOX17-GFP cells (PDX1/SOX17); and the PDX1-GFP cells and the Pancreas cells (PDX1/PANC).

TABLE 15
SOX17 and PDX1 specific cell surface

| | | | | Polyn. | | PDX1/ | SOX17/ | SOX17/ |
|---|---|---|---|---|---|---|---|---|
| | Affy. target | | SEQ ID | SEQ ID | | PANC | PANC | HESC |
| Probe | nucl.SEQ | Rep. | NO: (of Rep | NOs: rep. | Gene | fold | fold | fold |
| set ID | ID NO: | Public ID | Public ID) | by target | Symbol | change | change | change |
| 8056611 | 2046 | NM_004525 | 2322 | 2684, | LRP2 | 11.62 | 8.04 | 5.82 |
| | | | | 4119, | | | | |
| | | | | 5456, | | | | |
| | | | | 8262, | | | | |
| 8022176 | 2047 | NM_005559 | 2323 | 4685, | LAMA1 | 9.23 | 16.26 | 3.68 |
| | | | | 5278, | | | | |
| | | | | 8564, | | | | |
| | | | | 7980, | | | | |
| | | | | 8761, | | | | |
| | | | | 6083, | | | | |
| | | | | 7657, | | | | |
| 8175234 | 2048 | NM_004484 | 2324 | 3157, | GPC3 | 6.29 | 12.26 | 6.24 |
| | | | | 4899, | | | | |
| | | | | 4900, | | | | |
| | | | | 4901, | | | | |
| | | | | 4902, | | | | |
| | | | | 7303, | | | | |
| | | | | 7576, | | | | |
| | | | | 6111, | | | | |
| 7929373 | 2049 | NM_005097 | 2325 | 2745, | LGI1 | 10.86 | 2.65 | 2.29 |
| | | | | 4359, | | | | |
| | | | | 8710, | | | | |
| | | | | 8593, | | | | |
| | | | | 6230, | | | | |
| | | | | 6132, | | | | |
| 8058765 | 2050 | NM_212482 | 2326 | 3732, | FN1 | 3.65 | 8.73 | 4.30 |
| | | | | 3409, | | | | |
| | | | | 3983, | | | | |
| | | | | 4677, | | | | |
| | | | | 4679, | | | | |
| | | | | 3284, | | | | |
| | | | | 3310, | | | | |
| | | | | 3403, | | | | |
| | | | | 3404, | | | | |
| | | | | 3405, | | | | |
| | | | | 3406, | | | | |
| | | | | 4727, | | | | |
| | | | | 3110, | | | | |
| | | | | 4551, | | | | |
| | | | | 4571, | | | | |
| | | | | 4572, | | | | |
| | | | | 4576, | | | | |
| | | | | 3822, | | | | |
| | | | | 3494, | | | | |
| | | | | 2852, | | | | |
| | | | | 3620, | | | | |
| | | | | 3621, | | | | |
| | | | | 3622, | | | | |
| | | | | 3623, | | | | |
| | | | | 3624, | | | | |
| | | | | 3625, | | | | |
| | | | | 5996, | | | | |
| | | | | 7280, | | | | |
| | | | | 7649, | | | | |
| | | | | 6915, | | | | |
| | | | | 8364, | | | | |
| | | | | 8529, | | | | |
| | | | | 6768, | | | | |
| | | | | 7398, | | | | |
| | | | | 5846, | | | | |
| | | | | 6424, | | | | |
| | | | | 8736, | | | | |
| | | | | 8430, | | | | |
| | | | | 7036, | | | | |
| | | | | 5993, | | | | |
| | | | | 8153, | | | | |
| | | | | 8487, | | | | |
| | | | | 6324, | | | | |
| | | | | 6771, | | | | |
| | | | | 6223, | | | | |
| | | | | 6205, | | | | |
| | | | | 5539, | | | | |
| | | | | 6620, | | | | |
| | | | | 6121, | | | | |
| | | | | 6206, | | | | |
| | | | | 8147, | | | | |
| | | | | 5687, | | | | |
| 8095303 | 2051 | NM_015236 | 2327 | 2842, | LPHN3 | 8.14 | 4.02 | 1.86 |
| | | | | 4699, | | | | |
| | | | | 4713, | | | | |
| | | | | 4296, | | | | |
| | | | | 4285, | | | | |
| | | | | 8560, | | | | |
| | | | | 7869, | | | | |
| | | | | 8477, | | | | |
| | | | | 8293, | | | | |
| | | | | 8434, | | | | |
| | | | | 5644, | | | | |
| | | | | 8571, | | | | |
| | | | | 8574, | | | | |
| | | | | 8160, | | | | |
| | | | | 8174, | | | | |
| | | | | 5553, | | | | |
| | | | | 7807, | | | | |
| | | | | 8297, | | | | |
| | | | | 8402, | | | | |
| | | | | 8511, | | | | |
| | | | | 8619, | | | | |
| | | | | 8139, | | | | |
| | | | | 8480, | | | | |
| 8097910 | 2052 | NM_005141 | 2328 | 3895, | FGB | 3.34 | 4.95 | 5.54 |
| | | | | 5121, | | | | |
| | | | | 5122, | | | | |
| | | | | 8372, | | | | |
| | | | | 7847, | | | | |
| | | | | 5858, | | | | |
| | | | | 8448, | | | | |
| | | | | 6585, | | | | |
| | | | | 8337, | | | | |
| 8095110 | 2053 | NM_000222 | 2329 | 3646, | KIT | 4.19 | 8.30 | 3.08 |
| | | | | 4153, | | | | |
| | | | | 4152, | | | | |
| | | | | 5648, | | | | |
| | | | | 8162, | | | | |
| 8056222 | 2054 | NM_001935 | 2330 | 3604, | DPP4 | 3.37 | 6.97 | 4.32 |
| | | | | 3605, | | | | |
| | | | | 4611, | | | | |
| | | | | 2562, | | | | |
| | | | | 2570, | | | | |
| | | | | 3550, | | | | |
| | | | | 4303, | | | | |
| | | | | 3611, | | | | |
| | | | | 6918, | | | | |
| | | | | 6975, | | | | |
| | | | | 6558, | | | | |
| | | | | 7617, | | | | |
| | | | | 8396, | | | | |
| | | | | 7176, | | | | |
| | | | | 6249, | | | | |
| | | | | 7784, | | | | |
| | | | | 8632, | | | | |
| | | | | 8468, | | | | |
| 8167835 | 2055 | NM_001039705 | 2331 | 2908, | TRO | 3.40 | 4.69 | 2.20 |
| | | | | 2909, | | | | |
| | | | | 2910, | | | | |
| | | | | 2911, | | | | |
| | | | | 3329, | | | | |
| | | | | 4523, | | | | |
| | | | | 4561, | | | | |
| | | | | 2927, | | | | |
| | | | | 3265, | | | | |
| | | | | 3266, | | | | |
| | | | | 3938, | | | | |
| | | | | 6828, | | | | |
| | | | | 7971, | | | | |
| | | | | 8198, | | | | |
| | | | | 6749, | | | | |
| | | | | 7539, | | | | |
| | | | | 6497, | | | | |
| | | | | 5600, | | | | |
| | | | | 6368, | | | | |
| | | | | 6521, | | | | |
| | | | | 6543, | | | | |
| | | | | 7262, | | | | |
| | | | | 6752, | | | | |
| | | | | 7979, | | | | |
| | | | | 7021, | | | | |
| | | | | 6782, | | | | |
| | | | | 7644, | | | | |
| | | | | 6999, | | | | |
| | | | | 7279, | | | | |
| | | | | 6597, | | | | |
| | | | | 5857, | | | | |
| | | | | 7072, | | | | |
| | | | | 7725, | | | | |
| | | | | 5327, | | | | |
| | | | | 8749, | | | | |
| | | | | 7136, | | | | |
| 7944869 | 2056 | NM_017425 | 2332 | 2833, | SPA17 | 2.63 | 3.76 | 3.08 |
| | | | | 2894, | | | | |
| | | | | 4744, | | | | |
| | | | | 5335, | | | | |
| 8081001 | 2057 | NM_002942 | 2333 | 4181, | ROBO2 | 3.08 | 2.63 | 2.60 |
| | | | | 5144, | | | | |
| | | | | 5143, | | | | |
| | | | | 7229, | | | | |
| | | | | 7140, | | | | |
| | | | | 7162, | | | | |
| | | | | 7040, | | | | |
| | | | | 8742, | | | | |
| | | | | 7889, | | | | |
| 7976783 | 2058 | NM_003836 | 2334 | 4273, | DLK1 | 3.61 | 2.14 | 1.89 |
| | | | | 4994, | | | | |
| | | | | 6774, | | | | |
| | | | | 5796, | | | | |
| | | | | 6319, | | | | |

| | PDX1/ | | | | | |
|---|---|---|---|---|---|---|
| | HESC | | PDX11 | PDX1 | SOX17 | SOX17 |
| Probe | fold | FC | vs. | vs. | vs. | vs. |
| set ID | change | average | HESC | PANC | HESC | PANC |
| 8056611 | 8.40 | 8.47 | 4.0E−05 | 1.1E−04 | 1.2E−01 | 7.9E−02 |
| 8022176 | 2.09 | 7.82 | 3.1E−02 | 2.6E−03 | 2.8E−02 | 3.6E−03 |
| 8175234 | 3.20 | 7.00 | 7.0E−02 | 3.7E−02 | 5.5E−02 | 3.2E−02 |
| 7929373 | 9.41 | 6.30 | 2.7E−02 | 2.2E−02 | 9.0E−03 | 3.6E−03 |
| 8058765 | 1.80 | 4.62 | 4.7E−01 | 1.7E−01 | 2.9E−04 | 1.2E−03 |
| 8095303 | 3.77 | 4.45 | 4.0E−02 | 1.6E−02 | 1.5E−01 | 3.7E−02 |
| 8097910 | 3.74 | 4.39 | 2.4E−01 | 3.3E−01 | 2.3E−01 | 2.9E−01 |
| 8095110 | 1.56 | 4.28 | 4.9E−03 | 2.4E−04 | 5.9E−02 | 8.4E−03 |
| 8056222 | 2.09 | 4.19 | 2.2E−01 | 1.6E−01 | 1.5E−01 | 1.1E−01 |
| 8167835 | 1.60 | 2.97 | 5.1E−04 | 6.3E−05 | 7.6E−02 | 1.0E−02 |
| 7944869 | 2.15 | 2.90 | 2.3E−01 | 1.4E−01 | 3.2E−02 | 1.6E−02 |
| 8081001 | 3.05 | 2.84 | 2.5E−02 | 2.4E−02 | 5.4E−02 | 5.3E−02 |
| 7976783 | 3.19 | 2.71 | 6.3E−04 | 2.0E−02 | 1.2E−03 | 8.4E−02 |

Table 15. Provided are the description of the cell surface genes along with the sequence identifiers of the Affymetrix probes, the target sequence identified by the Affymetrix probe, the polynucleotide represented by the target sequence and the Public ID (and SEQ ID NO:) of the mRNA sequence representative of the target sequence. “Polyn.” = polynucleotide; “ Rep.” = representative; Also shown are the fold change and T-test p-values for change in expression levels of the indicated genes between the PDX1-GFP cells and the Pancreas cells (PDX1/PANC); SOX17-GFP cells and the Pancreas cells (SOX17/PANC); SOX17-GFP cells and undifferentiated hESCs (PDX1/HESC); the PDX1-GFP cells and undifferentiated hESCs (PDX1/HESC).

TABLE 16
PDX1 and Pancreas specific cell surface

| | | | | | | PDX1/ | PDX1/ |
|---|---|---|---|---|---|---|---|
| | Affy. target | | | Polyn. SEQ | | HESC | SOX17 |
| Probe | nucl. SEQ | Rep. | SEQ | ID NOs: rep. | Gene | fold | fold |
| Set ID | ID NO: | Public ID | ID NO: | by target | Symbol | change | change |
| 8115147 | 2059 | NM_001025159 | 2335 | 2996, 5297, | CD74 | 2.79 | 2.22 |
| | | | | 5295, 5296, | | | |
| | | | | 7571, 5325, | | | |
| | | | | 8303, 8172, | | | |
| | | | | 8179, 8156, | | | |
| | | | | 6067, 8367, | | | |
| | | | | 8576, 7868, | | | |
| 7947156 | 2060 | NM_001135091 | 2336 | 3463, 4618, | MUC15 | 8.35 | 7.47 |
| | | | | 4620, 4619, | | | |
| | | | | 5504, 6821, | | | |
| | | | | 7005, | | | |
| 7996081 | 2061 | NM_201524 | 2337 | 3334, 4786, | GPR56 | 4.74 | 3.21 |
| | | | | 4787, 4788, | | | |
| | | | | 4789, 4790, | | | |
| | | | | 4791, 4792, | | | |
| | | | | 4793, 6472, | | | |
| | | | | 7052, 6947, | | | |
| | | | | 6626, 7618, | | | |
| 7916584 | 2062 | NM_002353 | 2338 | 2675, 4337, | TACSTD2 | 2.77 | 2.65 |
| | | | | 6094, | (TROP- | | |
| | | | | | 2) | | |
| 7918936 | 2063 | NM_024626 | 2339 | 2837, 3971, | VTCN1 | 6.12 | 5.23 |
| | | | | 6218, 6213, | | | |
| | | | | 7307, 8522, | | | |
| | | | | 5804, 6025, | | | |
| 8056184 | 2064 | NM_000888 | 2340 | 3091, 2830, | ITGB6 | 5.53 | 4.77 |
| | | | | 8224, 8635, | | | |
| | | | | 5643, 7596, | | | |
| | | | | 6710, | | | |
| 8156134 | 2065 | NM_006180 | 2341 | 3803, 3756, | NTRK2 | 3.42 | 3.52 |
| | | | | 4629, 4630, | | | |
| | | | | 3840, 3841, | | | |
| | | | | 7335, 5675, | | | |
| | | | | 5488, 5700, | | | |
| | | | | 5977, 6773, | | | |
| | | | | 7194, 6916, | | | |
| | | | | 6615, | | | |
| 8095744 | 2066 | NM_001657 | 2342 | 2941, 3130, | AREG | 3.27 | 2.86 |
| | | | | 6586, 5463, | | | |
| | | | | 6936, | | | |
| 8081686 | 2067 | NM_033254 | 2343 | 2900, 3458, | BOC | 4.26 | 2.87 |
| | | | | 4375, 3408, | | | |
| | | | | 2980, 3094, | | | |
| | | | | 3128, 3691, | | | |
| | | | | 4325, 3512, | | | |
| | | | | 7851, 7097, | | | |
| | | | | 8462, 8015, | | | |
| | | | | 8182, 8307, | | | |
| | | | | 8026, 7628, | | | |
| | | | | 7853, 5922, | | | |
| | | | | 7736, 5497, | | | |
| | | | | 8579, | | | |
| 8046695 | 2068 | NM_000885 | 2344 | 2653, 3850, | ITGA4 | 2.24 | 2.71 |
| | | | | 4838, 4839, | | | |
| | | | | 4840, 4841, | | | |
| | | | | 4842, 4843, | | | |
| | | | | 4844, 7580, | | | |
| | | | | 7623, 8157, | | | |
| | | | | 8345, 8766, | | | |
| | | | | 7537, 7987, | | | |
| | | | | 8342, 8492, | | | |
| 7961151 | 2069 | NM_007360 | 2345 | 4264, 2991, | KLRK1 | 2.51 | 2.29 |
| | | | | 3980, 5238, | | | |
| | | | | 5378, 6831, | | | |
| | | | | 5634, | | | |

| | PANC/ | PANC/ | | | | | |
|---|---|---|---|---|---|---|---|
| | SOX17 | HESC | | PDX1 | PDX1 | PANC | PANC |
| Probe | fold | fold | FC | vs. | vs. | vs. | vs. |
| Set ID | change | change | average | HESC | SOX17 | HESC | SOX17 |
| 8115147 | 12.88 | 16.18 | 8.52 | 2.8E−02 | 2.8E−02 | 2.6E−03 | 4.9E−03 |
| 7947156 | 2.89 | 3.22 | 5.48 | 4.6E−02 | 4.6E−02 | 1.9E−01 | 2.3E−01 |
| 7996081 | 5.30 | 7.82 | 5.27 | 1.5E−01 | 1.5E−01 | 8.2E−04 | 5.7E−03 |
| 7916584 | 6.30 | 6.58 | 4.57 | 1.9E−02 | 1.9E−02 | 7.8E−02 | 9.1E−02 |
| 7918936 | 2.52 | 2.95 | 4.21 | 4.0E−02 | 4.0E−02 | 1.4E−01 | 2.3E−01 |
| 8056184 | 2.62 | 3.04 | 3.99 | 1.8E−01 | 1.8E−01 | 1.1E−01 | 1.7E−01 |
| 8156134 | 4.32 | 4.20 | 3.87 | 1.5E−01 | 1.5E−01 | 2.6E−04 | 4.0E−03 |
| 8095744 | 3.81 | 4.36 | 3.57 | 1.7E−01 | 1.7E−01 | 4.9E−02 | 7.2E−02 |
| 8081686 | 2.32 | 3.44 | 3.22 | 1.0E−01 | 1.0E−01 | 4.1E−02 | 1.8E−01 |
| 8046695 | 3.61 | 3.00 | 2.89 | 2.9E−02 | 2.9E−02 | 2.2E−02 | 2.1E−02 |
| 7961151 | 3.05 | 3.34 | 2.80 | 7.9E−02 | 7.9E−02 | 6.9E−02 | 8.2E−02 |

Table 16. Provided are the description of the cell surface genes along with the sequence identifiers of the Affymetrix probes, the target sequence identified by the Affymetrix probe, the polynucleotide represented by the target sequence and the Public ID (and SEQ ID NO:) of the mRNA sequence representative of the target sequence. “Polyn.” = polynucleotide; “Rep.” = representative; Also shown are the fold change and T-test p-values for change in expression levels of the indicated genes between the PDX1-GFP cells and undifferentiated hESCs (PDX1/HESC); the PDX1-GFP cells and SOX17-GFP cells (PDX1/SOX17); the Pancreas cells and SOX17 cells (PANC/SOX17); and the Pancreas cells and the hESCs (PANC/HESC);

TABLE 17
SOX17 specific putative cell surface genes

| | | | | | | SOX17/ |
|---|---|---|---|---|---|---|
| | Affy. target | | SEQ ID | Polyn. SEQ | | HESC |
| Probe | nucl. SEQ | Rep. | NO: (of Rep. | ID NOs: rep. | Gene | fold |
| set ID | ID NO: | Public ID | Public ID) | by target | Symbol | change |
| 8065412 | 2070 | NM_001898 | 2346 | 3369, 3033, 5734, | CST1 | 8.15 |
| | | | | 6511, | | |
| 7927631 | 2071 | NM_012242 | 2347 | 3471, 3796, 7543, | DKK1 | 12.95 |
| | | | | 8463, 8242, 5959, | | |
| 8152617 | 2072 | NM_005328 | 2348 | 2676, 4348, 5843, | HAS2 | 4.17 |
| | | | | 6409, | | |
| 7921834 | 2073 | NM_001643 | 2349 | 2901, 2758, 6253, | APOA2 | 17.82 |
| | | | | 8161, 7460, 7113, | | |
| | | | | 8544, 6696, 7530, | | |
| | | | | 7963, 6627, 6449, | | |
| 8022674 | 2074 | NM_001792 | 2350 | 3134, 4668, 5546, | CDH2 | 2.39 |
| 7957140 | 2075 | NM_003667 | 2351 | 2732, 3193, 6795, | LGR5 | 7.91 |
| | | | | 5509, | | |
| 8095646 | 2076 | NM_001134 | 2352 | 3041, 2756, 7886, | AFP | 8.81 |
| | | | | 7273, 8047, 8272, | | |
| 8065071 | 2077 | NM_198391 | 2353 | 2812, 5172, 5173, | FLRT3 | 9.61 |
| | | | | 5918, 7056, 6380, | | |
| 8050619 | 2078 | NM_000384 | 2354 | 2558, 3976, 5351, | APOB | 17.42 |
| 8172670 | 2079 | NM_001013742 | 2355 | 3778, 5274, 6444, | DGKK | 9.55 |
| 7997188 | 2080 | NM_005143 | 2356 | 3893, 4016, 4018, | HP | 7.70 |
| | | | | 2580, 2622, 2977, | | |
| | | | | 2992, 3453, 3617, | | |
| | | | | 4365, 4366, 6921, | | |
| | | | | 6310, 6000, 6001, | | |
| 8166747 | 2081 | NM_138780 | 2357 | 3411, 4887, 4885, | SYTL5 | 13.15 |
| | | | | 4886, 5912, 6888, | | |
| | | | | 5629, | | |
| 7977786 | 2082 | NM_003982 | 2358 | 2789, 5292, 5291, | SLC7A7 | 6.69 |
| | | | | 7158, 7255, 5870, | | |
| | | | | 8255, 6471, 7642, | | |
| 8020795 | 2083 | NM_000371 | 2359 | 2722, 4730, 6739, | TTR | 14.97 |
| | | | | 5362, | | |
| 8057506 | 2084 | NM_001463 | 2360 | 2679, 5106, 5695, | FRZB | 3.96 |
| 8121916 | 2085 | NM_032784 | 2361 | 3018, 3878, 6102, | RSPO3 | 9.02 |
| | | | | 6281, 8688, | | |
| 8155930 | 2086 | NM_001490 | 2362 | 2578, 4156, 4157, | GCNT1 | 3.13 |
| | | | | 4158, 4159, 4160, | | |
| | | | | 8460, 6349, 8001, | | |
| | | | | 7226, 6641, | | |
| 8151341 | 2087 | NM_007332 | 2363 | 4060, 8269, 5433, | TRPA1 | 6.01 |
| 8143534 | 2088 | NM_000420 | 2364 | 2886, 2987, 6934, | KEL | 5.85 |
| | | | | 8187, 8288, 6818, | | |
| | | | | 6144, 8559, | | |
| 7976858 | 2089 | NM_001362 | 2365 | 2990, 4161, 8265, | DIO3 | 4.61 |
| | | | | 6088, | | |
| 8171472 | 2090 | NM_020665 | 2366 | 2960, 4942, 6430, | TMEM27 | 4.68 |
| 8029536 | 2091 | NM_001645 | 2367 | 3322, 3714, 5431, | APOC1 | 3.42 |
| 8059279 | 2092 | NM_004438 | 2368 | 3926, 3577, 6427, | EPHA4 | 3.81 |
| | | | | 7497, 5543, | | |
| 8103415 | 2093 | NM_001128424 | 2369 | 3395, 4433, 4434, | C4orf18 | 7.34 |
| | | | | 4435, 7152, 7195, | | |
| | | | | 5766, 5649, | | |
| 8155192 | 2094 | NM_022343 | 2370 | 3083, 3132, 7426, | GLIPR2 | 3.24 |
| | | | | 7611, 7741, 8325, | | |
| | | | | 6925, | | |
| 8097449 | 2095 | NM_032961 | 2371 | 2914, 2936, 2935, | PCDH10 | 2.82 |
| | | | | 7099, 5512, 8691, | | |
| 7970241 | 2096 | NM_000504 | 2372 | 2567, 2573, 3210, | F10 | 6.85 |
| | | | | 3246, 4308, 4309, | | |
| | | | | 3935, 7894, 6670, | | |
| | | | | 7728, 6624, 7953, | | |
| | | | | 7352, 6493, | | |
| 8127193 | 2097 | NM_021073 | 2373 | 2611, 3202, 5997, | BMP5 | 7.19 |
| | | | | 7150, | | |
| 7919314 | 2098 | NM_001461 | 2374 | 3169, 4740, 4742, | FMO5 | 6.01 |
| | | | | 4741, 5452, 5926, | | |
| | | | | 6568, 7792, 6924, | | |
| 7968872 | 2099 | NM_013238 | 2375 | 3842, 3846, 7013, | DNAJC15 | 3.99 |
| | | | | 8073, | | |
| 7942332 | 2100 | NM_016730 | 2376 | 2548, 4931, 4932, | FOLR1 | 5.10 |
| | | | | 4933, 4934, 5730, | | |
| | | | | 7111, 7703, 7300, | | |
| 8147030 | 2101 | NM_007029 | 2377 | 4711, 5225, 5342, | STMN2 | 4.91 |
| 7901316 | 2102 | NM_001135181 | 2378 | 4682, 4626, 4627, | SLC5A9 | 4.12 |
| | | | | 6649, 6403, 7403, | | |
| | | | | 7829, 6781, 6361, | | |
| 8167287 | 2103 | NM_022825 | 2379 | 2854, 2855, 2856, | PORCN | 4.41 |
| | | | | 2857, 4728, 2616, | | |
| | | | | 4468, 2979, 2983, | | |
| | | | | 3366, 3574, 3575, | | |
| | | | | 3573, 3576, 5975, | | |
| | | | | 8479, 5883, 6961, | | |
| | | | | 5689, 8317, 8547, | | |
| | | | | 6074, 7887, 5982, | | |
| | | | | 7832, 6053, 6535, | | |
| 8105013 | 2104 | NM_152403 | 2380 | 3530, 5277, 5276, | EGFLAM | 4.32 |
| | | | | 5275, 6119, 5859, | | |
| | | | | 8466, 5742, 6783, | | |
| | | | | 8384, 6717, 7825, | | |
| | | | | 8629, 8422, 7942, | | |
| | | | | 6265, | | |
| 8059955 | 2105 | NM_022449 | 2381 | 3268, 4986, 4985, | RAB17 | 2.97 |
| | | | | 7579, 8393, 6896, | | |
| | | | | 6505, 7240, 5533, | | |
| 8065416 | 2106 | NM_001322 | 2382 | 3515, 3034, 5782, | CST2 | 4.29 |
| 8073633 | 2107 | NM_025225 | 2383 | 3547, 2988, 7337, | PNPLA3 | 2.32 |
| | | | | 5314, 8107, 8426, | | |
| 8175288 | 2108 | NM_019556 | 2384 | 2903, 2824, 6997, | MOSPD1 | 2.05 |
| | | | | 8283, 6003, 6290, | | |
| | | | | 7788, 7752, 5881, | | |
| 8139057 | 2109 | NM_014800 | 2385 | 2968, 3692, 4045, | ELMO1 | 5.57 |
| | | | | 2861, 3438, 4258, | | |
| | | | | 4520, 4479, 2801, | | |
| | | | | 2880, 3542, 5280, | | |
| | | | | 5279, 8359, 7086, | | |
| | | | | 5936, 6939, 6791, | | |
| | | | | 6584, 7972, 7197, | | |
| | | | | 7051, 6279, 5770, | | |
| | | | | 6974, 6770, 7488, | | |
| | | | | 7401, 7549, 6438, | | |
| | | | | 7349, | | |
| 8022666 | 2110 | NM_031422 | 2386 | 3460, 4937, 5865, | CHST9 | 3.68 |
| 7924342 | 2111 | NM_018713 | 2387 | 3493, 3718, 6268, | SLC30A10 | 4.77 |
| | | | | 6130, 8273, 8216, | | |
| 7993588 | 2112 | NM_024847 | 2388 | 3156, 4846, 4845, | TMC7 | 3.45 |
| | | | | 5727, 7486, | | |
| 8144656 | 2113 | BC132945 | 2389 | 4115, 2976, | C8orf49 | 4.28 |
| 8111234 | 2114 | NM_004061 | 2390 | 3260, 4595, 7174, | CDH12 | 4.25 |
| | | | | 8759, 8207, 8652, | | |
| | | | | 8503, 7723, 8276, | | |
| 8113358 | 2115 | NM_005668 | 2391 | 3302, 4599, 3244, | ST8SIA4 | 3.69 |
| | | | | 7055, 7738, 5386, | | |
| 8090823 | 2116 | NM_005630 | 2392 | 2724, 4396, 7489, | SLCO2A1 | 5.26 |
| | | | | 6355, 8401, 8414, | | |
| | | | | 8074, 5640, | | |
| 8121144 | 2117 | NM_024641 | 2393 | 4179, 5290, 6156, | MANEA | 4.38 |
| | | | | 5952, 8006, | | |
| 7964602 | 2118 | NM_153377 | 2394 | 3457, 3537, 4639, | LRIG3 | 3.70 |
| | | | | 5656, 6683, 7048, | | |
| 8111998 | 2119 | NM_021072 | 2395 | 3252, 5301, 5807, | HCN1 | 3.60 |
| 8111387 | 2120 | NM_030955 | 2396 | 2863, 3711, 8350, | ADAMTS12 | 3.27 |
| | | | | 8347, 7769, 6108, | | |
| | | | | 8583, | | |
| 7948058 | 2121 | NM_004476 | 2397 | 3060, 3615, 2779, | FOLH1 | 3.84 |
| | | | | 2843, 3871, 2598, | | |
| | | | | 4703, 4705, 4140, | | |
| | | | | 4138, 2773, 3026, | | |
| | | | | 3280, 5177, 5176, | | |
| | | | | 2766, 5178, 3821, | | |
| | | | | 6173, 5921, 6307, | | |
| | | | | 5425, 6469, 6968, | | |
| 8098041 | 2122 | NM_018342 | 2398 | 2802, 3163, 3170, | TMEM144 | 4.83 |
| | | | | 3261, 3319, 3506, | | |
| | | | | 3267, 4584, 8697, | | |
| | | | | 8424, 5686, 8259, | | |
| | | | | 8744, 8370, 7977, | | |
| | | | | 8192, 7923, 7849, | | |
| | | | | 8039, 7954, 8413, | | |
| | | | | 7830, | | |
| 8013606 | 2123 | NM_000638 | 2399 | 2897, 3932, 5348, | VTN | 5.44 |
| 7913237 | 2124 | NM_018584 | 2400 | 3291, 4040, 7514, | CAMK2N1 | 5.38 |
| | | | | 8167, | | |
| 7972297 | 2125 | NM_005845 | 2401 | 3298, 4237, 4238, | ABCC4 | 3.94 |
| | | | | 6825, 6506, 6826, | | |
| | | | | 6736, 7298, | | |
| 8094520 | 2126 | NM_032456 | 2402 | 2723, 5014, 5015, | PCDH7 | 2.10 |
| | | | | 2937, 5262, 5768, | | |
| | | | | 5971, | | |
| 7911273 | 2127 | NM_001004696 | 2403 | 3717, 6107, | OR2T4 | 3.58 |
| 8123388 | 2128 | NM_018974 | 2404 | 3851, 4738, 4736, | UNC93A | 2.39 |
| | | | | 5385, 8261, 6041, | | |
| | | | | 8180, 6272, | | |
| 7902235 | 2129 | NM_020794 | 2405 | 2985, 3272, 3712, | LRRC7 | 4.47 |
| | | | | 4096, 4697, 4027, | | |
| | | | | 5312, 5716, 5818, | | |
| | | | | 7008, 6195, 6139, | | |
| 8145736 | 2130 | NM_013958 | 2406 | 2651, 4050, 4820, | NRG1 | 2.76 |
| | | | | 4824, 4825, 4826, | | |
| | | | | 4827, 4828, 4829, | | |
| | | | | 4830, 4823, 4821, | | |
| | | | | 4822, 4831, 4832, | | |
| | | | | 4833, 4834, 4835, | | |
| | | | | 5786, 8022, 8533, | | |
| | | | | 5741, 6039, 6077, | | |
| | | | | 7952, 8077, 7456, | | |
| | | | | 5800, 5979, 7905, | | |
| | | | | 8268, 8671, | | |
| 8174389 | 2131 | NM_033641 | 2407 | 2623, 4165, 4167, | COL4A6 | 2.45 |
| | | | | 6197, 5733, 6413, | | |
| | | | | 8100, 7288, 7442, | | |
| | | | | 7535, | | |
| 7947512 | 2132 | NM_015430 | 2408 | 3786, 3701, 3702, | PAMR1 | 4.04 |
| | | | | 6790, 5537, 6920, | | |
| 8154135 | 2133 | NM_004170 | 2409 | 3070, 5025, 5401, | SLC1A1 | 2.42 |
| | | | | 7218, 8331, | | |
| 8089011 | 2134 | NM_000313 | 2410 | 2597, 4770, 6690, | PROS1 | 3.68 |
| 8118209 | 2135 | NM_019101 | 2411 | 2785, 3005, 2797, | APOM | 3.46 |
| | | | | 3131, 7568, 7419, | | |
| | | | | 7154, 6913, 7443, | | |
| | | | | 7676, 7525, 6531, | | |
| | | | | 7361, 7365, 7639, | | |
| | | | | 7323, 7713, 7039, | | |
| | | | | 6734, 7139, 6637, | | |
| | | | | 6503, 6767, 6861, | | |
| | | | | 6765, | | |
| 8045664 | 2136 | NM_177964 | 2412 | 3039, 3538, 8714, | LYPD6B | 3.68 |
| | | | | 5464, 7286, 6455, | | |
| | | | | 7123, 7806, 7494, | | |
| | | | | 8341, 7067, 6613, | | |
| 8004691 | 2137 | NM_203411 | 2413 | 3456, 3564, 5821, | TMEM88 | 2.64 |
| 7921702 | 2138 | NM_080878 | 2414 | 3464, 3488, 6169, | ITLN2 | 2.45 |
| | | | | 7935, 8767, | | |
| 8132250 | 2139 | NM_133468 | 2415 | 3324, 4608, 8606, | BMPER | 2.95 |
| | | | | 5589, 7995, 6712, | | |
| | | | | 6435, 7169, 8472, | | |
| 8132347 | 2140 | NM_181791 | 2416 | 3313, 3318, 6342, | GPR141 | 2.93 |
| | | | | 5701, 6692, | | |
| 8119898 | 2141 | NM_001025366 | 2417 | 3808, 4966, 4967, | VEGFA | 3.81 |
| | | | | 4968, 4969, 4970, | | |
| | | | | 4971, 4972, 4973, | | |
| | | | | 4974, 4975, 4976, | | |
| | | | | 4977, 4978, 4979, | | |
| | | | | 4980, 4981, 8712, | | |
| | | | | 5968, 6175, 7640, | | |
| | | | | 5961, 5690, 7243, | | |
| | | | | 8549, 6759, 6136, | | |
| | | | | 7716, 8136, 8355, | | |
| | | | | 8159, 8018, 7079, | | |
| | | | | 8270, 8120, 7866, | | |
| | | | | 8507, 8066, 7946, | | |

| | SOX17/ | SOX17/ | | | | |
|---|---|---|---|---|---|---|
| | PDX1 | PANC | | SOX17 | SOX17 | SOX17 |
| Probe | fold | fold | FC | vs. | vs. | vs. |
| set ID | change | change | average | HESC | PDX1 | PANC |
| 8065412 | 10.57 | 155.94 | 58.22 | 0.0006 | 0.1703 | 0.0001 |
| 7927631 | 7.52 | 73.19 | 31.22 | 0.0004 | 0.0534 | 0.0002 |
| 8152617 | 5.70 | 73.73 | 27.86 | 0.0191 | 0.0616 | 0.0009 |
| 7921834 | 4.25 | 45.73 | 22.60 | 0.0032 | 0.3017 | 0.0010 |
| 8022674 | 2.80 | 61.48 | 22.22 | 0.0457 | 0.1237 | 0.0002 |
| 7957140 | 8.46 | 32.45 | 16.28 | 0.0013 | 0.0713 | 0.0001 |
| 8095646 | 2.45 | 30.64 | 13.97 | 0.1268 | 0.6168 | 0.0390 |
| 8065071 | 5.74 | 24.40 | 13.25 | 0.0051 | 0.0774 | 0.0017 |
| 8050619 | 3.68 | 15.61 | 12.24 | 0.0141 | 0.3073 | 0.0164 |
| 8172670 | 8.75 | 14.66 | 10.99 | 0.0227 | 0.0287 | 0.0133 |
| 7997188 | 14.45 | 4.02 | 8.72 | 0.0000 | 0.0003 | 0.0848 |
| 8166747 | 7.21 | 4.94 | 8.44 | 0.0348 | 0.0865 | 0.1644 |
| 7977786 | 6.71 | 5.99 | 6.46 | 0.0520 | 0.0694 | 0.0685 |
| 8020795 | 2.62 | 1.70 | 6.43 | 0.0295 | 0.5580 | 0.7566 |
| 8057506 | 2.12 | 13.08 | 6.39 | 0.0323 | 0.2374 | 0.0095 |
| 8121916 | 2.83 | 6.85 | 6.24 | 0.0361 | 0.4235 | 0.0693 |
| 8155930 | 2.82 | 12.65 | 6.20 | 0.0322 | 0.0469 | 0.0024 |
| 8151341 | 5.25 | 6.96 | 6.07 | 0.0138 | 0.0181 | 0.0118 |
| 8143534 | 3.84 | 7.89 | 5.86 | 0.0101 | 0.0277 | 0.0058 |
| 7976858 | 5.01 | 7.09 | 5.57 | 0.0164 | 0.0135 | 0.0069 |
| 8171472 | 4.58 | 7.38 | 5.55 | 0.0062 | 0.0088 | 0.0031 |
| 8029536 | 3.05 | 9.63 | 5.37 | 0.1049 | 0.1256 | 0.0172 |
| 8059279 | 2.77 | 9.52 | 5.36 | 0.0070 | 0.0234 | 0.0083 |
| 8103415 | 5.08 | 3.39 | 5.27 | 0.0084 | 0.1540 | 0.0567 |
| 8155192 | 6.10 | 6.17 | 5.17 | 9.69E−02 | 2.99E−02 | 3.47E−02 |
| 8097449 | 2.41 | 9.33 | 4.85 | 2.75E−02 | 2.95E−01 | 2.51E−03 |
| 7970241 | 3.59 | 3.89 | 4.77 | 1.83E−02 | 9.73E−02 | 5.48E−02 |
| 8127193 | 2.33 | 4.68 | 4.73 | 1.45E−02 | 3.43E−01 | 4.07E−02 |
| 7919314 | 6.14 | 1.82 | 4.66 | 2.69E−02 | 2.24E−02 | 4.69E−01 |
| 7968872 | 4.94 | 4.96 | 4.63 | 1.82E−02 | 3.52E−02 | 1.17E−02 |
| 7942332 | 2.93 | 5.60 | 4.54 | 1.56E−02 | 7.53E−02 | 1.31E−02 |
| 8147030 | 2.31 | 6.26 | 4.49 | 4.21E−03 | 2.20E−01 | 1.10E−02 |
| 7901316 | 4.66 | 4.70 | 4.49 | 3.29E−02 | 2.49E−02 | 2.98E−02 |
| 8167287 | 3.25 | 5.77 | 4.47 | 5.07E−03 | 1.25E−02 | 3.33E−03 |
| 8105013 | 4.61 | 4.31 | 4.41 | 2.36E−02 | 2.67E−02 | 6.88E−02 |
| 8059955 | 5.62 | 4.64 | 4.41 | 9.80E−02 | 2.73E−02 | 5.52E−02 |
| 8065416 | 3.10 | 5.27 | 4.22 | 1.11E−03 | 8.10E−02 | 6.18E−04 |
| 8073633 | 2.76 | 7.56 | 4.21 | 1.20E−01 | 8.06E−02 | 1.41E−02 |
| 8175288 | 3.50 | 6.98 | 4.18 | 6.67E−02 | 1.46E−02 | 2.67E−03 |
| 8139057 | 4.60 | 2.36 | 4.18 | 1.38E−02 | 1.85E−02 | 2.21E−01 |
| 8022666 | 2.03 | 6.76 | 4.16 | 9.17E−02 | 2.98E−01 | 4.17E−02 |
| 7924342 | 3.28 | 4.38 | 4.15 | 3.27E−02 | 7.14E−02 | 4.01E−02 |
| 7993588 | 2.13 | 6.80 | 4.13 | 8.15E−02 | 2.29E−01 | 2.94E−02 |
| 8144656 | 4.29 | 3.78 | 4.12 | 1.72E−02 | 1.62E−02 | 2.23E−02 |
| 8111234 | 3.48 | 4.28 | 4.01 | 4.39E−02 | 6.74E−02 | 4.30E−02 |
| 8113358 | 5.57 | 2.70 | 3.99 | 4.46E−02 | 2.36E−02 | 1.69E−01 |
| 8090823 | 3.69 | 2.94 | 3.96 | 5.59E−02 | 1.74E−01 | 1.69E−01 |
| 8121144 | 3.33 | 4.07 | 3.93 | 4.95E−03 | 4.71E−02 | 4.99E−03 |
| 7964602 | 4.41 | 3.36 | 3.82 | 5.09E−02 | 4.70E−02 | 7.91E−02 |
| 8111998 | 3.20 | 4.59 | 3.80 | 3.66E−02 | 5.55E−02 | 2.73E−02 |
| 8111387 | 2.68 | 5.03 | 3.66 | 3.37E−02 | 6.13E−02 | 3.57E−02 |
| 7948058 | 3.34 | 3.70 | 3.63 | 5.45E−02 | 8.44E−02 | 5.93E−02 |
| 8098041 | 2.59 | 3.36 | 3.59 | 4.76E−02 | 1.51E−01 | 8.13E−02 |
| 8013606 | 2.52 | 2.78 | 3.58 | 3.57E−02 | 2.92E−01 | 1.39E−01 |
| 7913237 | 2.04 | 2.93 | 3.45 | 2.99E−03 | 1.68E−01 | 1.56E−02 |
| 7972297 | 2.02 | 4.33 | 3.43 | 3.04E−02 | 1.61E−01 | 2.42E−02 |
| 8094520 | 2.44 | 5.60 | 3.38 | 6.57E−03 | 3.94E−02 | 3.10E−04 |
| 7911273 | 3.35 | 3.17 | 3.37 | 1.66E−02 | 2.69E−02 | 2.30E−02 |
| 8123388 | 3.04 | 4.59 | 3.34 | 3.61E−02 | 2.35E−02 | 5.64E−03 |
| 7902235 | 2.42 | 2.94 | 3.27 | 4.41E−03 | 1.18E−01 | 1.57E−02 |
| 8145736 | 2.44 | 4.60 | 3.26 | 3.01E−03 | 3.23E−02 | 2.10E−03 |
| 8174389 | 2.49 | 4.84 | 3.26 | 8.73E−02 | 8.17E−02 | 1.62E−02 |
| 7947512 | 2.34 | 3.37 | 3.25 | 1.98E−02 | 1.39E−01 | 4.14E−02 |
| 8154135 | 3.71 | 3.59 | 3.24 | 2.52E−01 | 1.18E−01 | 1.28E−01 |
| 8089011 | 3.53 | 2.45 | 3.22 | 5.10E−02 | 5.29E−02 | 1.40E−01 |
| 8118209 | 2.22 | 3.77 | 3.15 | 2.08E−01 | 3.99E−01 | 1.84E−01 |
| 8045664 | 2.06 | 3.60 | 3.12 | 3.42E−02 | 2.45E−01 | 3.90E−02 |
| 8004691 | 2.18 | 4.46 | 3.09 | 1.78E−01 | 3.55E−01 | 1.06E−01 |
| 7921702 | 2.26 | 4.52 | 3.08 | 2.29E−02 | 5.63E−02 | 3.65E−03 |
| 8132250 | 2.80 | 3.43 | 3.06 | 1.67E−02 | 1.55E−02 | 6.52E−02 |
| 8132347 | 2.87 | 2.84 | 2.88 | 1.22E−01 | 1.27E−01 | 1.25E−01 |
| 8119898 | 2.14 | 2.02 | 2.66 | 8.92E−03 | 1.74E−01 | 8.31E−02 |

Table 17. Provided are the description of the putative cell surface genes along with the sequence identifiers of the Affymetrix probes, the target sequence identified by the Affymetrix probe, the polynucleotide represented by the target sequence and the Public ID (and SEQ ID NO:) of the mRNA sequence representative of the target sequence. “Polyn.” = polynucleotide; “Rep.” = representative; Also shown are the fold change and T-test p-values for change in expression levels of the indicated genes between the SOX17-GFP cells and undifferentiated hESCs (SOX17/HESC); the SOX17-GFP cells and PDX1-GFP cells (SOX17/PDX1); and the SOX17-GFP cells and the Pancreas cells (SOX17/PANC).

TABLE 18
PDX1 specific putative cell surface genes

| | Affy. target | | SEQ ID NO: | | | |
|---|---|---|---|---|---|---|
| Probe | nucl. SEQ | Rep. | (of Rep. | Polyn. SEQ ID NOs: | Gene | PDX1/HESC |
| set ID | ID NO: | Public ID | Public ID) | rep. by target | Symbol | fold change |
| 7989073 | 2142 | NM_173814 | 2418 | 3710, 4778, 6379, | PRTG | 17.99 |
| 8135774 | 2143 | NM_002851 | 2419 | 2599, 3943, 7457, | PTPRZ1 | 1.82 |
| | | | | 8030, 7815, 8011, | | |
| | | | | 8237, 7646, | | |
| 8109926 | 2144 | NM_014211 | 2420 | 3898, 4597, 7818, | GABRP | 10.28 |
| | | | | 8036, 8604, 8494, | | |
| | | | | 8777, 8597, 5487, | | |
| 7963970 | 2145 | NM_006928 | 2421 | 3105, 5245, 6941, | SILV | 3.32 |
| | | | | 6287, | | |
| 8020384 | 2146 | AK293321 | 2422 | 4467, 2876, 4295, | KIAA1772 | 12.92 |
| | | | | 4675, 7359, 7061, | | |
| | | | | 5529, 7232, | | |
| 8169061 | 2147 | NM_000533 | 2423 | 3671, 3836, 3741, | PLP1 | 3.49 |
| | | | | 3742, 2596, 2595, | | |
| | | | | 2588, 4267, 4502, | | |
| | | | | 4503, 2878, 4324, | | |
| | | | | 4329, 4438, 3554, | | |
| | | | | 3553, 8469, 6590, | | |
| | | | | 7115, 7231, 7458, | | |
| | | | | 6933, 7196, 7209, | | |
| | | | | 8108, 8781, 8623, | | |
| | | | | 8141, 8486, 7855, | | |
| | | | | 5832, 8385, 5923, | | |
| | | | | 8694, 6335, 7709, | | |
| | | | | 6348, 8343, 7178, | | |
| | | | | 7629, | | |
| 7953873 | 2148 | BX647938 | 2424 | 3414, 3204, 8476, | OVOS | 3.13 |
| | | | | 8450, | | |
| 8112971 | 2149 | NM_001884 | 2425 | 2661, 3452, 4443, | HAPLN1 | 4.27 |
| | | | | 8313, 8478, 8578, | | |
| | | | | 8217, 5490, 8326, | | |
| | | | | 8275, | | |
| 8128284 | 2150 | NM_004440 | 2426 | 2636, 4616, 6203, | EPHA7 | 2.00 |
| | | | | 6224, | | |
| 7953532 | 2151 | NM_001975 | 2427 | 3363, 2973, 5374, | ENO2 | 2.71 |
| 8108697 | 2152 | NM_015669 | 2428 | 2872, 2931, 5379, | PCDHB5 | 1.92 |
| 7953873 | 2153 | BX647938 | 2429 | 3414, 3204, 8476, | OVOS | 2.96 |
| | | | | 8450, | | |
| 7957338 | 2154 | NM_005639 | 2430 | 3480, 4632, 4633, | SYT1 | 2.87 |
| | | | | 4634, 7165, 5424, | | |
| | | | | 6857, | | |
| 7972259 | 2155 | NM_001922 | 2431 | 2627, 4441, 4442, | DCT | 6.21 |
| | | | | 6478, 8000, 7156, | | |
| | | | | 7726, 6808, 8056, | | |
| 8113356 | 2156 | AK172782 | 2432 | 3609, | GPAM | 5.84 |
| 7972239 | 2157 | NM_032229 | 2433 | 3864, 3532, 6395, | SLITRK6 | 5.14 |
| 8021245 | 2158 | NM_005215 | 2434 | 4508, 4919, 7276, | DCC | 5.58 |
| | | | | 5767, 6546, | | |
| 7968678 | 2159 | NM_207361 | 2435 | 3444, 4093, 5476, | FREM2 | 5.40 |
| | | | | 7899, | | |
| 8018114 | 2160 | NM_001144952 | 2436 | 2814, 4748, 7774, | SDK2 | 5.29 |
| | | | | 5765, 6851, 6326, | | |
| 8128001 | 2161 | NM_000735 | 2437 | 2949, 5306, 5947, | CGA | 5.24 |
| 8004545 | 2162 | NM_001678 | 2438 | 2604, 3681, 5449, | ATP1B2 | 5.83 |
| 8140686 | 2163 | NM_152754 | 2439 | 3466, 3557, 8721, | SEMA3D | 5.79 |
| | | | | 5583, | | |
| 8108753 | 2164 | NM_018935 | 2440 | 3191, 2929, 5380, | PCDHB15 | 4.40 |
| 7996837 | 2165 | NM_004360 | 2441 | 4149, 4182, 2614, | CDH1 | 2.09 |
| | | | | 4278, 4556, 2794, | | |
| | | | | 2795, 4415, 4294, | | |
| | | | | 4315, 4347, 5506, | | |
| | | | | 7453, 5404, 7133, | | |
| 7929822 | 2166 | NM_003393 | 2442 | 3013, 4006, 6207, | WNT8B | 4.75 |
| 8168517 | 2167 | NM_005296 | 2443 | 3834, 4756, 7854, | LPAR4 | 1.95 |
| | | | | 5927, | | |
| 7994026 | 2168 | NM_130464 | 2444 | 3833, 4524, 5020, | NPIPL3 | 5.42 |
| | | | | 4635, 3908, 7496, | | |
| | | | | 7414, 7388, 6046, | | |
| | | | | 6948, 7475, 6819, | | |
| | | | | 8569, 5650, 6371, | | |
| | | | | 7765, 6554, 6829, | | |
| | | | | 5907, 7686, 6199, | | |
| | | | | 8130, 8706, 8211, | | |
| | | | | 7864, | | |
| 8046895 | 2169 | NM_177454 | 2445 | 4274, 4070, 5467, | FAM171B | 3.56 |
| | | | | 5763, | | |
| 8143144 | 2170 | NM_002825 | 2446 | 2905, 3560, 6250, | PTN | 2.21 |
| 7929779 | 2171 | NM_000392 | 2447 | 2682, 4397, 7717, | ABCC2 | 5.16 |
| | | | | 5949, 5908, | | |
| 8100870 | 2172 | NM_014243 | 2448 | 4101, 5286, 8348, | ADAMTS3 | 3.79 |
| | | | | 5586, | | |
| 8141843 | 2173 | NM_006989 | 2449 | 5151, 5150, 7699, | RASA4 | 5.77 |
| | | | | 5542, 6353, | | |
| 8030448 | 2174 | NM_001136052 | 2450 | 3049, 5236, 5237, | CPT1C | 3.10 |
| | | | | 7371, 6022, 7446, | | |
| | | | | 5780, 5691, | | |
| 8078014 | 2175 | NM_003043 | 2451 | 3914, 4382, 4385, | SLC6A6 | 1.83 |
| | | | | 4527, 2650, 2637, | | |
| | | | | 3349, 4300, 4293, | | |
| | | | | 4603, 3999, 4602, | | |
| | | | | 6095, 6731, 7138, | | |
| | | | | 7680, 7533, 7619, | | |
| | | | | 7898, 7128, 6509, | | |
| | | | | 7006, | | |
| 8108688 | 2176 | NM_018937 | 2452 | 2924, 2930, 5388, | PCDHB3 | 3.94 |
| 8006336 | 2177 | NM_052888 | 2453 | 3521, 3984, 4680, | LRRC37B | 4.03 |
| | | | | 4511, 4567, 4302, | | |
| | | | | 3733, 5844, 6194, | | |
| | | | | 7424, | | |
| 7959025 | 2178 | NM_001109903 | 2454 | 3352, 4242, 4243, | RNFT2 | 2.20 |
| | | | | 5418, 5592, 7222, | | |
| | | | | 7129, | | |
| 8051785 | 2179 | NM_133329 | 2455 | 3647, 5227, 5228, | KCNG3 | 1.99 |
| | | | | 5783, 7561, | | |
| 8083166 | 2180 | NM_003304 | 2456 | 2670, 3955, 8655, | TRPC1 | 2.51 |
| | | | | 8076, 5551, 6798, | | |
| | | | | 7437, | | |
| 8049128 | 2181 | NM_031313 | 2457 | 2557, 4233, 5696, | ALPPL2 | 3.77 |
| 8148962 | 2182 | NM_001005504 | 2458 | 4378, 4092, 3719, | OR4F21 | 3.52 |
| | | | | 3723, 3734, 5799, | | |
| | | | | 7532, 5680, 6665, | | |
| | | | | 7961, | | |
| 8006433 | 2183 | NM_002982 | 2459 | 2971, 3759, 5331, | CCL2 | 4.78 |
| 7956522 | 2184 | AF063608 | 2460 | 2859, 7555, | KIF5A | 1.94 |
| 8033818 | 2185 | NM_058164 | 2461 | 2754, 4134, 5475, | OLFM2 | 3.68 |
| 8031076 | 2186 | NM_031896 | 2462 | 3827, 5226, 7212, | CACNG7 | 2.65 |
| | | | | 5339, | | |
| 7967386 | 2187 | NM_022782 | 2463 | 3124, 3475, 5829, | MPHOSPH9 | 2.00 |
| | | | | 5803, 7370, | | |
| 8143110 | 2188 | NM_012450 | 2464 | 3802, 3305, 7278, | SLC13A4 | 2.72 |
| | | | | 8106, 8640, 7462, | | |
| | | | | 8737, 6233, 7314, | | |
| | | | | 7467, | | |
| 8129573 | 2189 | NM_015529 | 2465 | 2831, 4077, 8607, | MOXD1 | 3.57 |
| | | | | 6186, | | |
| 8128837 | 2190 | NM_001123364 | 2466 | 4362, | C6orf186 | 3.86 |
| 7955469 | 2191 | NM_001039960 | 2467 | 4335, 3940, 3941, | SLC4A8 | 1.83 |
| | | | | 6177, 6955, 5856, | | |
| | | | | 6574, | | |
| 8063636 | 2192 | NM_001001433 | 2468 | 4565, 5270, 5269, | STX16 | 1.57 |
| | | | | 5271, 5272, 6016, | | |
| | | | | 6278, 6873, 8633, | | |
| | | | | 8620, 8642, 7876, | | |
| | | | | 6090, 8358, 7184, | | |
| | | | | 6337, 8702, 6864, | | |
| | | | | 5934, 6359, 5902, | | |
| | | | | 6322, | | |

| Probe | PDX1/SOX17 | PDX1/PANC | FC | PDX1/ | PDX1/ | PDX1/ |
|---|---|---|---|---|---|---|
| set ID | fold change | fold change | average | HESC | SOX17 | PANC |
| 7989073 | 1.67 | 44.84 | 21.50 | 2.18E−03 | 4.65E−01 | 1.27E−03 |
| 8135774 | 7.84 | 43.05 | 17.57 | 2.84E−02 | 9.92E−04 | 8.98E−05 |
| 8109926 | 14.17 | 13.85 | 12.77 | 6.22E−02 | 4.32E−02 | 4.56E−02 |
| 7963970 | 8.34 | 26.38 | 12.68 | 2.55E−03 | 1.25E−03 | 8.28E−05 |
| 8020384 | 3.53 | 11.66 | 9.37 | 1.98E−05 | 1.24E−02 | 8.94E−04 |
| 8169061 | 6.21 | 13.94 | 7.88 | 4.42E−04 | 1.55E−03 | 3.02E−04 |
| 7953873 | 4.15 | 13.46 | 6.91 | 1.90E−02 | 2.03E−02 | 2.06E−03 |
| 8112971 | 5.91 | 10.17 | 6.78 | 1.95E−02 | 9.53E−03 | 3.52E−03 |
| 8128284 | 2.17 | 15.05 | 6.41 | 2.20E−01 | 1.86E−01 | 4.99E−03 |
| 7953532 | 3.64 | 11.81 | 6.05 | 3.42E−02 | 8.95E−02 | 2.06E−03 |
| 8108697 | 11.87 | 3.96 | 5.92 | 3.08E−01 | 1.83E−02 | 1.25E−01 |
| 7953873 | 4.05 | 10.68 | 5.90 | 1.67E−02 | 1.84E−02 | 2.26E−03 |
| 7957338 | 4.38 | 10.29 | 5.85 | 1.17E−02 | 5.02E−03 | 5.53E−04 |
| 7972259 | 6.19 | 4.67 | 5.69 | 1.32E−01 | 1.33E−01 | 1.99E−01 |
| 8113356 | 5.67 | 5.49 | 5.67 | 1.15E−01 | 1.24E−01 | 1.27E−01 |
| 7972239 | 5.03 | 5.97 | 5.38 | 1.07E−01 | 1.10E−01 | 8.58E−02 |
| 8021245 | 4.92 | 5.31 | 5.27 | 1.01E−01 | 1.39E−01 | 1.08E−01 |
| 7968678 | 1.62 | 8.14 | 5.06 | 1.60E−02 | 4.62E−01 | 2.29E−02 |
| 8018114 | 3.87 | 6.00 | 5.05 | 7.13E−03 | 2.36E−02 | 6.24E−03 |
| 8128001 | 4.37 | 5.35 | 4.99 | 2.50E−02 | 3.39E−02 | 2.05E−02 |
| 8004545 | 4.82 | 4.24 | 4.96 | 2.18E−03 | 8.00E−03 | 8.78E−03 |
| 8140686 | 2.31 | 6.55 | 4.88 | 5.75E−02 | 4.03E−01 | 7.48E−02 |
| 8108753 | 5.61 | 3.46 | 4.49 | 1.30E−02 | 6.15E−03 | 3.65E−02 |
| 7996837 | 7.44 | 3.84 | 4.45 | 5.03E−04 | 5.81E−04 | 3.27E−01 |
| 7929822 | 3.80 | 4.79 | 4.45 | 1.08E−01 | 1.55E−01 | 1.06E−01 |
| 8168517 | 3.31 | 7.09 | 4.12 | 1.71E−01 | 5.48E−02 | 8.05E−03 |
| 7994026 | 2.63 | 3.75 | 3.93 | 2.06E−02 | 2.06E−01 | 3.92E−02 |
| 8046895 | 1.84 | 6.16 | 3.85 | 2.89E−02 | 2.67E−01 | 7.34E−03 |
| 8143144 | 3.53 | 5.72 | 3.82 | 1.00E−01 | 8.57E−02 | 1.75E−02 |
| 7929779 | 1.91 | 4.16 | 3.74 | 1.77E−01 | 6.36E−01 | 2.27E−01 |
| 8100870 | 3.04 | 4.35 | 3.73 | 3.65E−02 | 6.50E−02 | 3.23E−02 |
| 8141843 | 1.96 | 3.11 | 3.61 | 1.73E−02 | 1.93E−01 | 4.40E−02 |
| 8030448 | 1.73 | 5.83 | 3.55 | 9.71E−04 | 5.50E−02 | 2.61E−04 |
| 8078014 | 2.25 | 6.42 | 3.50 | 2.76E−02 | 3.80E−02 | 2.83E−03 |
| 8108688 | 3.21 | 3.27 | 3.47 | 3.06E−02 | 5.11E−02 | 6.64E−02 |
| 8006336 | 2.00 | 4.37 | 3.46 | 2.70E−02 | 2.13E−01 | 3.59E−02 |
| 7959025 | 2.09 | 5.91 | 3.40 | 6.84E−03 | 1.43E−02 | 1.87E−03 |
| 8051785 | 4.61 | 3.32 | 3.31 | 2.11E−01 | 3.59E−02 | 6.45E−02 |
| 8083166 | 1.92 | 5.44 | 3.29 | 1.48E−04 | 2.85E−02 | 1.01E−03 |
| 8049128 | 3.01 | 2.98 | 3.25 | 2.85E−01 | 3.75E−01 | 3.66E−01 |
| 8148962 | 2.75 | 3.48 | 3.25 | 1.01E−01 | 1.78E−01 | 1.06E−01 |
| 8006433 | 2.60 | 2.30 | 3.22 | 7.29E−02 | 2.46E−01 | 2.62E−01 |
| 7956522 | 3.39 | 4.30 | 3.21 | 6.96E−02 | 1.39E−02 | 6.49E−03 |
| 8033818 | 2.33 | 3.58 | 3.20 | 6.17E−03 | 1.08E−01 | 9.26E−03 |
| 8031076 | 2.97 | 3.68 | 3.10 | 1.49E−02 | 1.44E−01 | 5.39E−03 |
| 7967386 | 2.05 | 5.23 | 3.09 | 3.11E−02 | 1.48E−01 | 9.67E−04 |
| 8143110 | 1.89 | 4.63 | 3.08 | 2.25E−02 | 1.00E−01 | 6.15E−03 |
| 8129573 | 2.63 | 2.94 | 3.05 | 1.08E−01 | 1.97E−01 | 1.57E−01 |
| 8128837 | 3.24 | 1.97 | 3.02 | 9.06E−02 | 1.28E−01 | 3.35E−01 |
| 7955469 | 2.07 | 5.16 | 3.02 | 1.77E−04 | 4.25E−03 | 4.18E−05 |
| 8063636 | 1.54 | 1.76 | 1.62 | 2.08E−01 | 3.28E−01 | 1.30E−01 |

Table 18. Provided are the description of the putative cell surface genes along with the sequence identifiers of the Affymetrix probes, the target sequence identified by the Affymetrix probe, the polynucleotide represented by the target sequence and the Public ID (and SEQ ID NO:) of the mRNA sequence representative of the target sequence. “Polyn.” = polynucleotide; “Rep.” = representative; Also shown are the fold change and T-test p-values for change in expression levels of the indicated genes between the PDX1-GFP cells and undifferentiated hESCs (PDX1/HESC); the PDX1-GFP cells and the Pancreas cells (PDX1/PANC); and the PDX1-GFP cells and SOX17-GFP cells (PDX1/SOX17).

TABLE 19
SOX17 and PDX1 specific putative cell surface genes

| | Affy. target | | SEQ ID NO: | | | | |
|---|---|---|---|---|---|---|---|
| Probe | nucl. SEQ | Rep. | (of Rep. | Polyn. SEQ ID NOs: | Gene | PDX1/PANC | SOX17/PANC |
| set ID | ID NO: | Public ID | Public ID) | rep. by target | Symbol | fold change | fold change |
| 7989073 | 2193 | NM_173814 | 2469 | 3710, 4778, | PRTG | 44.84 | 26.86 |
| | | | | 6379, | | | |
| 7927631 | 2194 | NM_012242 | 2470 | 3471, 3796, | DKK1 | 9.73 | 73.19 |
| | | | | 7543, 8463, | | | |
| | | | | 8242, 5959, | | | |
| 7921834 | 2195 | NM_001643 | 2471 | 2901, 2758, | APOA2 | 10.76 | 45.73 |
| | | | | 6253, 8161, | | | |
| | | | | 7460, 7113, | | | |
| | | | | 8544, 6696, | | | |
| | | | | 7530, 7963, | | | |
| | | | | 6627, 6449, | | | |
| 8095646 | 2196 | NM_001134 | 2472 | 3041, 2756, | AFP | 12.50 | 30.64 |
| | | | | 7886, 7273, | | | |
| | | | | 8047, 8272, | | | |
| 8050619 | 2197 | NM_000384 | 2473 | 2558, 3976, | APOB | 4.24 | 15.61 |
| | | | | 5351, | | | |
| 8065071 | 2198 | NM_198391 | 2474 | 2812, 5172, | FLRT3 | 4.25 | 24.40 |
| | | | | 5173, 5918, | | | |
| | | | | 7056, 6380, | | | |
| 8020384 | 2199 | AK293321 | 2475 | 4467, 2876, | KIAA1772 | 11.66 | 3.30 |
| | | | | 4295, 4675, | | | |
| | | | | 7359, 7061, | | | |
| | | | | 5529, 7232, | | | |
| 8113800 | 2200 | NM_001999 | 2476 | 2620, 3843, | FBN2 | 9.22 | 12.60 |
| | | | | 5455, 7802, | | | |
| | | | | 7852, 8234, | | | |
| 8057506 | 2201 | NM_001463 | 2477 | 2679, 5106, | FRZB | 6.16 | 13.08 |
| | | | | 5695, | | | |
| 8038683 | 2202 | NM_002774 | 2478 | 2673, 3799, | KLK6 | 4.93 | 6.26 |
| | | | | 3800, 3801, | | | |
| | | | | 7560, 5608, | | | |
| | | | | 6746, 7343, | | | |
| | | | | 6725, 6526, | | | |
| 7968678 | 2203 | NM_207361 | 2479 | 3444, 4093, | FREM2 | 8.14 | 5.01 |
| | | | | 5476, 7899, | | | |
| 8121916 | 2204 | NM_032784 | 2480 | 3018, 3878, | RSPO3 | 2.42 | 6.85 |
| | | | | 6102, 6281, | | | |
| | | | | 8688, | | | |
| 7951865 | 2205 | NM_000039 | 2481 | 3902, 2762, | APOA1 | 2.20 | 8.83 |
| | | | | 6306, 5375, | | | |
| | | | | 6654, 6990, | | | |
| 7951865 | 2206 | NM_000039 | 2482 | 3902, 2762, | APOA1 | 2.21 | 8.49 |
| | | | | 6306, 5375, | | | |
| | | | | 6654, 6990, | | | |
| 8140686 | 2207 | NM_152754 | 2483 | 3466, 3557, | SEMA3D | 6.55 | 2.84 |
| | | | | 8721, 5583, | | | |
| 8143534 | 2208 | NM_000420 | 2484 | 2886, 2987, | KEL | 2.06 | 7.89 |
| | | | | 6934, 8187, | | | |
| | | | | 8288, 6818, | | | |
| | | | | 6144, 8559, | | | |
| 8127193 | 2209 | NM_021073 | 2485 | 2611, 3202, | BMP5 | 2.01 | 4.68 |
| | | | | 5997, 7150, | | | |
| 8167973 | 2210 | NM_138737 | 2486 | 3469, 2884, | HEPH | 6.78 | 6.77 |
| | | | | 4726, 4486, | | | |
| | | | | 4512, 3021, | | | |
| | | | | 2834, 2951, | | | |
| | | | | 4313, 3058, | | | |
| | | | | 4952, 4953, | | | |
| | | | | 5726, 7553, | | | |
| | | | | 7348, 6504, | | | |
| | | | | 7890, 6899, | | | |
| | | | | 5938, 6468, | | | |
| | | | | 6569, | | | |
| 8147030 | 2211 | NM_007029 | 2487 | 4711, 5225, | STMN2 | 2.71 | 6.26 |
| | | | | 5342, | | | |
| 7955142 | 2212 | NM_000725 | 2488 | 3227, 5283, | CACNB3 | 5.25 | 4.99 |
| | | | | 5827, | | | |
| 8022666 | 2213 | NM_031422 | 2489 | 3460, 4937, | CHST9 | 3.33 | 6.76 |
| | | | | 5865, | | | |
| 7993588 | 2214 | NM_024847 | 2490 | 3156, 4846, | TMC7 | 3.19 | 6.80 |
| | | | | 4845, 5727, | | | |
| | | | | 7486, | | | |
| 8046895 | 2215 | NM_177454 | 2491 | 4274, 4070, | FAM171B | 6.16 | 3.35 |
| | | | | 5467, 5763, | | | |
| 8008132 | 2216 | NM_005175 | 2492 | 3118, 3700, | ATP5G1 | 6.24 | 3.00 |
| | | | | 3699, 7206, | | | |
| | | | | 8638, 7714, | | | |
| | | | | 8758, 6255, | | | |
| | | | | 7875, 7750, | | | |
| | | | | 8115, | | | |
| 7942332 | 2217 | NM_016730 | 2493 | 2548, 4931, | FOLR1 | 1.91 | 5.60 |
| | | | | 4932, 4933, | | | |
| | | | | 4934, 5730, | | | |
| | | | | 7111, 7703, | | | |
| | | | | 7300, | | | |
| 7929779 | 2218 | NM_000392 | 2494 | 2682, 4397, | ABCC2 | 4.16 | 2.18 |
| | | | | 7717, 5949, | | | |
| | | | | 5908, | | | |
| 8113433 | 2219 | NM_001962 | 2495 | 2657, 4446, | EFNA5 | 5.21 | 5.44 |
| | | | | 8787, 5710, | | | |
| | | | | 8776, 8760, | | | |
| 8030448 | 2220 | NM_001136052 | 2496 | 3049, 5236, | CPT1C | 5.83 | 3.37 |
| | | | | 5237, 7371, | | | |
| | | | | 6022, 7446, | | | |
| | | | | 5780, 5691, | | | |
| 8167656 | 2221 | NM_001005333 | 2497 | 3365, 3727, | MAGED1 | 3.13 | 5.78 |
| | | | | 3728, 3726, | | | |
| | | | | 8338, 8682, | | | |
| | | | | 7309, 7060, | | | |
| | | | | 6807, 7682, | | | |
| | | | | 8515, 8260, | | | |
| | | | | 6701, 5806, | | | |
| | | | | 7744, | | | |
| 8141843 | 2222 | NM_006989 | 2498 | 5151, 5150, | RASA4 | 3.11 | 1.59 |
| | | | | 7699, 5542, | | | |
| | | | | 6353, | | | |
| 8073015 | 2223 | NM_006855 | 2499 | 2873, 2817, | KDELR3 | 2.26 | 3.38 |
| | | | | 2816, 5309, | | | |
| | | | | 7392, 7239, | | | |
| 8018761 | 2224 | NM_006456 | 2500 | 2638, 4437, | ST6GALNAC2 | 1.54 | 2.34 |
| | | | | 5345, | | | |
| 8097692 | 2225 | NM_001957 | 2501 | 2600, 4920, | EDNRA | 2.63 | 2.16 |
| | | | | 4921, 4922, | | | |
| | | | | 8628, 5591, | | | |
| | | | | 5960, 8235, | | | |
| | | | | 5962, 8220, | | | |
| | | | | 6628, 7962, | | | |
| 8006336 | 2226 | NM_052888 | 2502 | 3521, 3984, | LRRC37B | 4.37 | 2.19 |
| | | | | 4680, 4511, | | | |
| | | | | 4567, 4302, | | | |
| | | | | 3733, 5844, | | | |
| | | | | 6194, 7424, | | | |
| 7972297 | 2227 | NM_005845 | 2503 | 3298, 4237, | ABCC4 | 2.14 | 4.33 |
| | | | | 4238, 6825, | | | |
| | | | | 6506, 6826, | | | |
| | | | | 6736, 7298, | | | |
| 8167603 | 2228 | NM_001127899 | 2504 | 2659, 4413, | CLCN5 | 2.79 | 4.87 |
| | | | | 4414, 4062, | | | |
| | | | | 7768, 5769, | | | |
| | | | | 6329, 6401, | | | |
| | | | | 6907, 7160, | | | |
| 8050160 | 2229 | NM_138799 | 2505 | 4760, 3543, | MBOAT2 | 4.88 | 3.47 |
| | | | | 5812, 8164, | | | |
| | | | | 8465, 8453, | | | |
| | | | | 7310, 8461, | | | |
| | | | | 8368, 6454, | | | |
| | | | | 7808, | | | |
| 7917052 | 2230 | NM_152697 | 2506 | 3153, 4453, | SLC44A5 | 2.71 | 3.64 |
| | | | | 4454, 7183, | | | |
| | | | | 6087, 6160, | | | |
| 7980146 | 2231 | NM_006432 | 2507 | 3361, 3263, | NPC2 | 2.91 | 3.66 |
| | | | | 7305, 5357, | | | |
| 8131096 | 2232 | NM_001128636 | 2508 | 4884, | ELFN1 | 3.03 | 2.52 |
| 8016832 | 2233 | NM_012329 | 2509 | 3499, 3725, | MMD | 4.53 | 3.01 |
| | | | | 5396, | | | |
| 8068353 | 2234 | NM_006933 | 2510 | 3065, 4367, | SLC5A3 | 3.32 | 3.03 |
| | | | | 4168, 7291, | | | |
| | | | | 8103, 8705, | | | |
| | | | | 8188, 8498, | | | |
| | | | | 6457, | | | |
| 8150906 | 2235 | NM_017813 | 2511 | 4236, 5520, | IMPAD1 | 2.34 | 4.01 |
| 8096771 | 2236 | NM_021227 | 2512 | 3331, 3544, | OSTC | 3.36 | 3.77 |
| | | | | 8513, 8662, | | | |
| | | | | 6166, | | | |
| 8022711 | 2237 | NM_024422 | 2513 | 3516, 4406, | DSC2 | 3.66 | 4.14 |
| | | | | 4407, 5438, | | | |
| | | | | 5505, 6352, | | | |
| | | | | 7116, | | | |
| 8157270 | 2238 | NM_001859 | 2514 | 2699, 4628, | SLC31A1 | 4.11 | 4.15 |
| | | | | 6193, 6305, | | | |
| 7947165 | 2239 | NM_178498 | 2515 | 3423, 4239, | SLC5A12 | 4.13 | 3.22 |
| | | | | 7203, | | | |
| 8135488 | 2240 | NM_001099660 | 2516 | 3450, 4205, | LRRN3 | 2.69 | 1.97 |
| | | | | 4202, 4200, | | | |
| | | | | 6645, 7164, | | | |
| | | | | 5798, | | | |
| 7994026 | 2241 | NM_130464 | 2517 | 3833, 4524, | NPIPL3 | 3.51 | 2.04 |
| | | | | 5020, 4635, | | | |
| | | | | 3908, 7496, | | | |
| | | | | 7414, 7388, | | | |
| | | | | 6046, 6948, | | | |
| | | | | 7475, 6819, | | | |
| | | | | 8569, 5650, | | | |
| | | | | 6371, 7765, | | | |
| | | | | 6554, 6829, | | | |
| | | | | 5907, 7686, | | | |
| | | | | 6199, 8130, | | | |
| | | | | 8706, 8211, | | | |
| | | | | 7864, | | | |
| 8008802 | 2242 | NM_182569 | 2518 | 3086, 4924, | GDPD1 | 3.19 | 2.86 |
| | | | | 4925, 4923, | | | |
| | | | | 5759, | | | |
| 7994026 | 2243 | NM_130464 | 2519 | 3833, 4524, | NPIPL3 | 3.44 | 2.08 |
| | | | | 5020, 4635, | | | |
| | | | | 3908, 7496, | | | |
| | | | | 7414, 7388, | | | |
| | | | | 6046, 6948, | | | |
| | | | | 7475, 6819, | | | |
| | | | | 8569, 5650, | | | |
| | | | | 6371, 7765, | | | |
| | | | | 6554, 6829, | | | |
| | | | | 5907, 7686, | | | |
| | | | | 6199, 8130, | | | |
| | | | | 8706, 8211, | | | |
| | | | | 7864, | | | |
| 7994026 | 2244 | NM_130464 | 2520 | 3833, 4524, | NPIPL3 | 3.48 | 2.07 |
| | | | | 5020, 4635, | | | |
| | | | | 3908, 7496, | | | |
| | | | | 7414, 7388, | | | |
| | | | | 6046, 6948, | | | |
| | | | | 7475, 6819, | | | |
| | | | | 8569, 5650, | | | |
| | | | | 6371, 7765, | | | |
| | | | | 6554, 6829, | | | |
| | | | | 5907, 7686, | | | |
| | | | | 6199, 8130, | | | |
| | | | | 8706, 8211, | | | |
| | | | | 7864, | | | |
| 8160546 | 2245 | NM_152570 | 2521 | 4386, 3147, | LINGO2 | 2.80 | 2.77 |
| | | | | 5841, 7024, | | | |
| 8045664 | 2246 | NM_177964 | 2522 | 3039, 3538, | LYPD6B | 1.75 | 3.60 |
| | | | | 8714, 5464, | | | |
| | | | | 7286, 6455, | | | |
| | | | | 7123, 7806, | | | |
| | | | | 7494, 8341, | | | |
| | | | | 7067, 6613, | | | |

| | | | | PDX1 | PDX1 | SOX17 | SOX17 |
|---|---|---|---|---|---|---|---|
| Probe | SOX17/HESC | PDX1/HESC | FC | vs. | vs. | vs. | vs. |
| set ID | fold change | fold change | average | HESC | PANC | HESC | PANC |
| 7989073 | 10.78 | 17.99 | 25.12 | 2.18E−03 | 1.27E−03 | 9.95E−03 | 4.28E−03 |
| 7927631 | 12.95 | 1.72 | 24.40 | 4.85E−01 | 3.70E−02 | 4.00E−04 | 1.77E−04 |
| 7921834 | 17.82 | 4.19 | 19.63 | 2.79E−01 | 1.06E−01 | 3.17E−03 | 9.95E−04 |
| 8095646 | 8.81 | 3.59 | 13.89 | 3.53E−01 | 1.07E−01 | 1.27E−01 | 3.90E−02 |
| 8050619 | 17.42 | 4.73 | 10.50 | 1.55E−01 | 1.80E−01 | 1.41E−02 | 1.64E−02 |
| 8065071 | 9.61 | 1.67 | 9.98 | 4.67E−01 | 9.17E−02 | 5.11E−03 | 1.73E−03 |
| 8020384 | 3.66 | 12.92 | 7.89 | 1.98E−05 | 8.94E−04 | 1.07E−02 | 3.58E−02 |
| 8113800 | 3.03 | 2.22 | 6.77 | 5.94E−02 | 2.49E−03 | 1.98E−02 | 1.36E−03 |
| 8057506 | 3.96 | 1.87 | 6.27 | 1.38E−01 | 1.99E−02 | 3.23E−02 | 9.55E−03 |
| 8038683 | 6.87 | 5.41 | 5.87 | 5.13E−02 | 5.92E−02 | 1.34E−01 | 1.49E−01 |
| 7968678 | 3.33 | 5.40 | 5.47 | 1.60E−02 | 2.29E−02 | 4.98E−02 | 5.29E−02 |
| 8121916 | 9.02 | 3.19 | 5.37 | 2.83E−01 | 4.23E−01 | 3.61E−02 | 6.93E−02 |
| 7951865 | 7.79 | 1.94 | 5.19 | 2.33E−01 | 1.74E−01 | 1.18E−02 | 1.00E−02 |
| 7951865 | 7.44 | 1.94 | 5.02 | 2.29E−01 | 1.67E−01 | 1.16E−02 | 9.63E−03 |
| 8140686 | 2.50 | 5.79 | 4.42 | 5.75E−02 | 7.48E−02 | 2.06E−01 | 2.31E−01 |
| 8143534 | 5.85 | 1.53 | 4.33 | 1.95E−02 | 2.85E−03 | 1.01E−02 | 5.80E−03 |
| 8127193 | 7.19 | 3.09 | 4.24 | 1.49E−01 | 3.51E−01 | 1.45E−02 | 4.07E−02 |
| 8167973 | 1.56 | 1.56 | 4.16 | 1.17E−01 | 7.23E−03 | 4.00E−01 | 2.71E−02 |
| 8147030 | 4.91 | 2.12 | 4.00 | 2.20E−01 | 1.74E−01 | 4.21E−03 | 1.10E−02 |
| 7955142 | 2.69 | 2.83 | 3.94 | 4.21E−02 | 9.05E−03 | 7.93E−03 | 1.19E−03 |
| 8022666 | 3.68 | 1.82 | 3.90 | 2.88E−02 | 1.92E−02 | 9.17E−02 | 4.17E−02 |
| 7993588 | 3.45 | 1.62 | 3.76 | 2.02E−02 | 1.03E−02 | 8.15E−02 | 2.94E−02 |
| 8046895 | 1.94 | 3.56 | 3.75 | 2.89E−02 | 7.34E−03 | 1.36E−01 | 2.25E−02 |
| 8008132 | 1.73 | 3.61 | 3.65 | 6.59E−02 | 2.56E−02 | 5.24E−01 | 2.42E−01 |
| 7942332 | 5.10 | 1.74 | 3.59 | 4.99E−02 | 3.32E−02 | 1.56E−02 | 1.31E−02 |
| 7929779 | 2.70 | 5.16 | 3.55 | 1.77E−01 | 2.27E−01 | 2.68E−01 | 3.68E−01 |
| 8113433 | 1.78 | 1.70 | 3.53 | 2.74E−01 | 3.02E−02 | 3.25E−02 | 6.71E−03 |
| 8030448 | 1.79 | 3.10 | 3.52 | 9.71E−04 | 2.61E−04 | 2.27E−02 | 2.18E−03 |
| 8167656 | 3.33 | 1.80 | 3.51 | 1.86E−03 | 6.73E−03 | 2.72E−02 | 1.27E−02 |
| 8141843 | 2.95 | 5.77 | 3.35 | 1.73E−02 | 4.40E−02 | 2.15E−02 | 8.07E−02 |
| 8073015 | 4.63 | 3.09 | 3.34 | 5.00E−02 | 1.47E−01 | 2.55E−02 | 6.57E−02 |
| 8018761 | 5.70 | 3.73 | 3.33 | 1.28E−01 | 5.74E−01 | 1.89E−02 | 1.49E−01 |
| 8097692 | 3.77 | 4.59 | 3.29 | 6.31E−02 | 2.87E−01 | 1.87E−03 | 2.30E−01 |
| 8006336 | 2.02 | 4.03 | 3.15 | 2.70E−02 | 3.59E−02 | 8.81E−02 | 1.18E−01 |
| 7972297 | 3.94 | 1.95 | 3.09 | 2.00E−03 | 7.17E−04 | 3.04E−02 | 2.42E−02 |
| 8167603 | 2.90 | 1.66 | 3.06 | 4.81E−04 | 5.85E−05 | 4.81E−02 | 1.41E−02 |
| 8050160 | 1.60 | 2.26 | 3.05 | 6.44E−02 | 9.59E−03 | 1.64E−01 | 1.42E−02 |
| 7917052 | 3.22 | 2.40 | 2.99 | 3.64E−03 | 2.74E−02 | 9.92E−02 | 9.89E−02 |
| 7980146 | 3.01 | 2.39 | 2.99 | 2.93E−02 | 5.07E−02 | 1.71E−02 | 3.13E−02 |
| 8131096 | 2.91 | 3.50 | 2.99 | 7.80E−03 | 1.14E−02 | 8.80E−02 | 1.23E−01 |
| 8016832 | 1.61 | 2.41 | 2.89 | 6.75E−03 | 1.87E−01 | 1.50E−01 | 3.20E−01 |
| 8068353 | 2.47 | 2.71 | 2.88 | 6.65E−02 | 1.10E−01 | 4.94E−04 | 6.50E−02 |
| 8150906 | 3.26 | 1.91 | 2.88 | 3.17E−01 | 2.27E−01 | 1.13E−01 | 8.71E−02 |
| 8096771 | 2.27 | 2.03 | 2.86 | 4.86E−02 | 2.51E−02 | 1.02E−01 | 4.36E−02 |
| 8022711 | 1.85 | 1.64 | 2.82 | 2.46E−01 | 3.49E−02 | 1.35E−01 | 2.09E−02 |
| 8157270 | 1.52 | 1.50 | 2.82 | 2.50E−01 | 1.64E−02 | 3.33E−01 | 2.82E−02 |
| 7947165 | 1.68 | 2.16 | 2.80 | 1.62E−02 | 1.45E−03 | 4.66E−02 | 2.41E−03 |
| 8135488 | 2.75 | 3.76 | 2.79 | 1.53E−03 | 3.07E−02 | 1.35E−04 | 5.92E−02 |
| 7994026 | 2.06 | 3.55 | 2.79 | 2.47E−02 | 2.52E−02 | 1.04E−01 | 1.07E−01 |
| 8008802 | 2.40 | 2.68 | 2.78 | 6.34E−05 | 3.96E−04 | 2.05E−02 | 1.40E−02 |
| 7994026 | 2.10 | 3.49 | 2.78 | 2.59E−02 | 2.62E−02 | 9.45E−02 | 9.69E−02 |
| 7994026 | 2.07 | 3.48 | 2.78 | 2.55E−02 | 2.52E−02 | 9.97E−02 | 9.87E−02 |
| 8160546 | 2.73 | 2.76 | 2.76 | 1.09E−02 | 1.44E−02 | 3.38E−02 | 3.79E−02 |
| 8045664 | 3.68 | 1.79 | 2.71 | 1.63E−01 | 1.89E−01 | 3.42E−02 | 3.90E−02 |

Table 19. Provided are the description of the putative cell surface genes along with the sequence identifiers of the Affymetrix probes, the target sequence identified by the Affymetrix probe, the polynucleotide represented by the target sequence and the Public ID (and SEQ ID NO:) of the mRNA sequence representative of the target sequence. “Polyn.” = polynucleotide; “Rep.” = representative; Also shown are the fold change and T-test p-values for change in expression levels of the indicated genes between the PDX1-GFP cells and the Pancreas cells (PDX1/PANC); SOX17-GFP cells and the Pancreas cells (SOX17/PANC); SOX17-GFP cells and undifferentiated hESCs (PDX1/HESC); the PDX1-GFP cells and undifferentiated hESCs (PDX1/HESC).

TABLE 20
PDX1 and Pancreas specific putative cell surface genes

| | Affy. target | | SEQ ID NO: | | | | |
|---|---|---|---|---|---|---|---|
| Probe | nucl. SEQ | Rep. | (of Rep. | Polyn. SEQ ID NOs: | Gene | PDX1/ | PDX1/ |
| set ID | ID NO: | Public ID | Public ID) | rep. by target | Symbol | HESC | SOX17 |
| 7903414 | 2247 | NM_000699 | 2523 | 3814, 2634, | AMY2A | 3.79 | 2.83 |
| | | | | 2921, 3063, | | | |
| | | | | 3766, 3767, | | | |
| | | | | 3777, 3765, | | | |
| | | | | 3769, 6181, | | | |
| | | | | 8186, 6513, | | | |
| | | | | 8562, 7109, | | | |
| | | | | 6188, 7492, | | | |
| | | | | 7387, 8726, | | | |
| | | | | 6179, 8240, | | | |
| | | | | 6758, 6648, | | | |
| | | | | 5740, 6977, | | | |
| | | | | 6257, 5819, | | | |
| 8101659 | 2248 | NM_001128310 | 2524 | 3607, 4539, | SPARCL1 | 3.14 | 2.98 |
| | | | | 4554, 4700, | | | |
| | | | | 4490, 4558, | | | |
| | | | | 4476, 3089, | | | |
| | | | | 2943, 4431, | | | |
| | | | | 4430, 6384, | | | |
| | | | | 8285, 5454, | | | |
| | | | | 8543, 7693, | | | |
| | | | | 7326, 8142, | | | |
| | | | | 7471, | | | |
| 7961514 | 2249 | NM_000900 | 2525 | 3815, 5155, | MGP | 2.07 | 2.10 |
| | | | | 5156, 5372, | | | |
| 7960947 | 2250 | NM_000014 | 2526 | 3209, 3849, | A2M | 7.83 | 4.71 |
| | | | | 5717, 8772, | | | |
| | | | | 8168, 6820, | | | |
| | | | | 8158, 6446, | | | |
| | | | | 6742, | | | |
| 7965410 | 2251 | NM_001920 | 2527 | 2902, 3628, | DCN | 2.02 | 4.12 |
| | | | | 3629, 3630, | | | |
| | | | | 3631, 3632, | | | |
| | | | | 3633, 7344, | | | |
| | | | | 7248, 6470, | | | |
| | | | | 7199, 5678, | | | |
| | | | | 5337, 5322, | | | |
| | | | | 6594, | | | |
| 8023497 | 2252 | NM_005603 | 2528 | 2736, 5024, | ATP8B1 | 2.34 | 2.80 |
| | | | | 5707, | | | |
| 8096415 | 2253 | NM_007351 | 2529 | 3528, 3568, | MMRN1 | 7.09 | 5.79 |
| | | | | 6962, 7912, | | | |
| | | | | 6838, 5465, | | | |
| 7954090 | 2254 | NM_001423 | 2530 | 2678, 4610, | EMP1 | 2.26 | 2.14 |
| | | | | 6653, 7265, | | | |
| | | | | 5426, | | | |
| 7913216 | 2255 | NM_000300 | 2531 | 2906, 4866, | PLA2G2A | 2.85 | 5.25 |
| | | | | 4867, 4868, | | | |
| | | | | 4869, 6945, | | | |
| | | | | 6892, 8598, | | | |
| | | | | 8048, 8615, | | | |
| | | | | 6396, 7663, | | | |
| 7954293 | 2256 | NM_000921 | 2532 | 3510, 5300, | PDE3A | 2.94 | 2.38 |
| | | | | 5941, | | | |
| 8098611 | 2257 | NM_003265 | 2533 | 3835, 3032, | TLR3 | 2.25 | 2.60 |
| | | | | 8206, 5688, | | | |
| 8051583 | 2258 | NM_000104 | 2534 | 2630, 4421, | CYP1B1 | 3.41 | 2.85 |
| | | | | 6727, 8527, | | | |
| | | | | 5409, | | | |
| 8066925 | 2259 | NM_000961 | 2535 | 3882, 3795, | PTGIS | 4.05 | 5.49 |
| | | | | 5363, 8250, | | | |
| | | | | 8695, | | | |
| 8058063 | 2260 | NM_144629 | 2536 | 3913, 4395, | RFTN2 | 4.30 | 3.80 |
| | | | | 5599, 7926, | | | |
| 8145977 | 2261 | NM_021623 | 2537 | 4259, 4207, | PLEKHA2 | 2.02 | 2.85 |
| | | | | 8302, 7202, | | | |
| | | | | 6843, 7732, | | | |
| | | | | 8390, | | | |
| 7975390 | 2262 | NM_001034852 | 2538 | 4277, 5268, | SMOC1 | 3.45 | 3.29 |
| | | | | 5267, 7459, | | | |
| | | | | 5893, | | | |
| 8163896 | 2263 | NM_004099 | 2539 | 2946, 3496, | STOM | 2.23 | 2.77 |
| | | | | 3495, 6038, | | | |
| | | | | 5639, | | | |
| 8068024 | 2264 | NM_021219 | 2540 | 2864, 3085, | JAM2 | 3.47 | 3.81 |
| | | | | 8520, 7625, | | | |
| | | | | 6556, 8774, | | | |
| | | | | 7964, 7436, | | | |
| | | | | 6761, 5705, | | | |
| 8077270 | 2265 | NM_006614 | 2541 | 2694, 3806, | CHL1 | 2.67 | 2.03 |
| | | | | 3927, 4681, | | | |
| | | | | 3440, 3238, | | | |
| | | | | 6369, 6718, | | | |
| | | | | 6522, 7572, | | | |
| | | | | 7141, 5444, | | | |
| | | | | 7590, 6487, | | | |
| | | | | 6827, 7208, | | | |
| 7982366 | 2266 | NM_001144757 | 2542 | 3813, 4731, | SCG5 | 3.05 | 3.07 |
| | | | | 4732, 8086, | | | |
| | | | | 6330, 8119, | | | |
| | | | | 8148, 5657, | | | |
| 8100541 | 2267 | NM_001553 | 2543 | 3500, 2759, | IGFBP7 | 2.65 | 2.26 |
| | | | | 5754, 7872, | | | |
| | | | | 7990, | | | |
| 8104746 | 2268 | NM_000908 | 2544 | 4247, 5263, | NPR3 | 2.87 | 2.48 |
| | | | | 6508, 8718, | | | |
| | | | | 5468, 7324, | | | |
| 7914127 | 2269 | NM_002038 | 2545 | 3350, 3961, | IFI6 | 2.10 | 2.58 |
| | | | | 3962, 3960, | | | |
| | | | | 6118, 6098, | | | |
| | | | | 6254, | | | |
| 7909390 | 2270 | NM_175710 | 2546 | 3897, 4185, | CR1L | 2.16 | 2.05 |
| | | | | 8470, 6548, | | | |
| | | | | 5851, | | | |
| 8129558 | 2271 | NM_030908 | 2547 | 4048, 4376, | OR2A4 | 2.17 | 2.16 |
| | | | | 2917, 5157, | | | |
| | | | | 3729, 5808, | | | |
| | | | | 7931, | | | |
| 8143633 | 2272 | NM_001005328 | 8793 | 4048, 4376, | OR2A7 | 2.15 | 2.14 |
| | | | | 2917, 5157, | | | |
| | | | | 3729, 5808, | | | |
| | | | | 7931, | | | |

| | | | | PDX1 | PDX1 | PANC | PANC |
|---|---|---|---|---|---|---|---|
| Probe | PANC/ | PANC/ | FC | vs. | vs. | vs. | vs. |
| set ID | SOX17 | HESC | average | HESC | SOX17 | HESC | SOX17 |
| 7903414 | 63.39 | 84.98 | 38.75 | 6.94E−03 | 6.94E−03 | 3.93E−02 | 4.82E−02 |
| 8101659 | 59.17 | 62.31 | 31.90 | 7.73E−02 | 7.73E−02 | 7.74E−04 | 8.61E−04 |
| 7961514 | 51.99 | 51.42 | 26.89 | 2.49E−01 | 2.49E−01 | 1.55E−03 | 1.73E−03 |
| 7960947 | 35.60 | 59.17 | 26.83 | 2.35E−01 | 2.35E−01 | 2.79E−04 | 4.65E−04 |
| 7965410 | 42.02 | 20.66 | 17.21 | 1.69E−01 | 1.69E−01 | 8.61E−03 | 4.37E−03 |
| 8023497 | 10.73 | 8.96 | 6.21 | 4.46E−03 | 4.46E−03 | 2.60E−02 | 2.05E−02 |
| 8096415 | 4.78 | 5.86 | 5.88 | 1.60E−01 | 1.60E−01 | 2.49E−02 | 3.48E−02 |
| 7954090 | 9.03 | 9.56 | 5.75 | 2.69E−01 | 2.69E−01 | 1.32E−02 | 1.54E−02 |
| 7913216 | 8.17 | 4.44 | 5.18 | 1.13E−02 | 1.13E−02 | 7.39E−02 | 4.19E−02 |
| 7954293 | 5.02 | 6.18 | 4.13 | 2.66E−01 | 2.66E−01 | 5.56E−04 | 2.20E−02 |
| 8098611 | 6.01 | 5.20 | 4.02 | 2.63E−02 | 2.63E−02 | 1.79E−04 | 6.80E−04 |
| 8051583 | 4.07 | 4.87 | 3.80 | 3.83E−02 | 3.83E−02 | 4.62E−02 | 7.35E−02 |
| 8066925 | 3.11 | 2.30 | 3.74 | 9.29E−04 | 9.29E−04 | 6.62E−02 | 2.86E−02 |
| 8058063 | 2.58 | 2.92 | 3.40 | 1.45E−01 | 1.45E−01 | 7.03E−02 | 1.21E−01 |
| 8145977 | 5.08 | 3.60 | 3.39 | 4.17E−02 | 4.17E−02 | 3.87E−02 | 4.15E−02 |
| 7975390 | 3.18 | 3.34 | 3.31 | 1.41E−01 | 1.41E−01 | 4.59E−03 | 3.14E−02 |
| 8163896 | 4.38 | 3.53 | 3.23 | 3.49E−02 | 3.49E−02 | 1.08E−02 | 2.35E−02 |
| 8068024 | 2.75 | 2.50 | 3.14 | 1.36E−01 | 1.36E−01 | 9.78E−02 | 8.38E−02 |
| 8077270 | 3.09 | 4.08 | 2.97 | 2.29E−01 | 2.29E−01 | 3.29E−03 | 2.04E−02 |
| 7982366 | 2.83 | 2.81 | 2.94 | 1.24E−02 | 1.24E−02 | 2.50E−01 | 2.63E−01 |
| 8100541 | 2.94 | 3.45 | 2.82 | 4.45E−01 | 4.45E−01 | 5.51E−03 | 1.56E−01 |
| 8104746 | 2.05 | 2.37 | 2.44 | 2.26E−01 | 2.26E−01 | 3.10E−01 | 4.14E−01 |
| 7914127 | 2.75 | 2.25 | 2.42 | 1.47E−02 | 1.47E−02 | 5.76E−02 | 3.14E−02 |
| 7909390 | 2.06 | 2.17 | 2.11 | 2.28E−01 | 2.28E−01 | 3.57E−02 | 5.29E−02 |
| 8129558 | 2.03 | 2.04 | 2.10 | 2.92E−03 | 2.92E−03 | 1.24E−01 | 1.32E−01 |
| 8143633 | 2.02 | 2.03 | 2.09 | 6.13E−03 | 6.13E−03 | 1.43E−01 | 1.56E−01 |

Table 20. Provided are the description of the putative cell surface genes along with the sequence identifiers of the Affymetrix probes, the target sequence identified by the Affymetrix probe, the polynucleotide represented by the target sequence and the Public ID (and SEQ ID NO:) of the mRNA sequence representative of the target sequence. “Polyn.” = polynucleotide; “Rep.” = representative; Also shown are the fold change and T-test p-values for change in expression levels of the indicated genes between the PDX1-GFP cells and undifferentiated hESCs (PDX1/HESC); the PDX1-GFP cells and SOX17-GFP cells (PDX1/SOX17); the Pancreas cells and SOX17 cells (PANC/SOX17); and the Pancreas cells and the hESCs (PANC/HESC);

### Example 5

### TROP-2 FACS Sorting of Cells Derived from Embryoid Bodies Results in an Isolated Popupation of PDX1-Positive Cells (at Least 84% PDX1 Positive)

Experimental Results

Separation of TROP-2 Expressing Cells by FACS ARIA and Staining with PDX1—

Embryoid bodies were prepared from H9.2 hESCs and grown for 28 days in EBs medium (DMEM, 20% serum replacement, 1 mM Glutamax and 1% nonessential amino acid—all from GIBCO Invitrogen) with a change of medium every 3 days. The EBs were separated to single cells by treating for 20-30 minutes with TrypLE Select (Invitrogen) in 37° C. The cells were washed once with EBs medium, once more in FACS buffer (0.5% BSA, 10 mM EDTA, 25 mM Hepes in PBS). 10⁷ cells were diluted in 950 μl FACS buffer with 50 μl of anti-human TROP-2-APC (R&D Systems Inc., Minneapolis, Minn.) and incubated for 30 minutes at room temperature in the dark with shaking every 5 minutes. The cells were washed twice with PBS and centrifuged for 3 minutes at 1500 rpm. The cells were filtered with a 40 μm strainer (BD) and resuspended in FACS buffer. The FACS ARIA (BD) sorter was used to separate TROP-2 positive cells from the whole EB population. 1.3% of the cells were positive for allophycocyanin (APC) and were collected into DMEM with 10% FBS. The cells were spun down by Cytospin onto glass slides and fixed with cold acetone.

In order to stain the cells with PDX1 antibody, the cells were fixed again with 4% paraformaldehyde for 15 minutes and washed twice with PBS. The cells were blocked for one hour by adding 5% Normal Goat Serum (NGS), 1% BSA (bovine serum albumin), 0.5% Triton in PBS (phosphate buffered saline). The cells were washed twice with PBS and stained with goat polyclonal PDX1 (ABcam, Cambridge, UK) at a dilution of 1:1000 in PBS with 1% Triton, 0.5% BSA and 1% FBS over night in 4° C. The cells were washed twice with PBS and stained with anti-goat Cy3 secondary antibody 1:100 (Jackson Laboratories, West Grove, Pa.). Cells were rinsed twice with PBS and the nuclei were stained with DAPI 1:1000 (Sigma) and then the cells were mounted in fluorescent mounting media (Dako). The slides were analyzed using a confocal microscope (LSM 700, Zeiss).

Experimental Results

As shown in FIGS. 14A-C (representative confocal microscopy images) 84% of the cells which were isolated using an anti-human TROP-2 antibody were positive for PDX1 expression. These results demonstrate the isolation, for the first time, of a population of cells comprising at least 84% PDX1-positive cells which are not genetically modified.

### Example 6

### Endoderm Cells Expressing BST2, FLRT3, COLEC12, GPR49/LGR5 OR LIF-R can be Separated by FACs from Cell Populations Derived from Young Embryoid Bodies

Seven days old EBs contain cells of mesoderm, ectoderm and endoderm lineages but not fully differentiated tissues. These cells were tested for the expression of several surface markers which were found to be increased in the SOX17 expressing definitive endoderm cells by Affymetrix expression analysis.

Experimental Results

Sorting with Endoderm Markers Using FACS with Single Markers on Young EBs

H9.2 ESCs were grown for 7 days in EBs medium (DMEM, 20% serum replacement, 1 mM Glutamax and 1% nonessential amino acid—all from GIBCO Invitrogen) with a change of medium every 3 days. The EBs were separated to single cells by treating for 20-30 minutes with TrypLE Select (Invitrogen) in 37° C. The cells were washed once with EBs medium and once more in FACS buffer (0.5% BSA, 10 mM EDTA, 25 mM Hepes in PBS). The following antibodies were tested:

1. Rabbit anti BST-2 (Santa Cruz) 1:200
2. Rabbit anti FLRT-3 (Santa Cruz) 1:50
3. Goat anti CL-P1/COLEC12 (R&D) 1:40
4. Rabbit anti GPR-49/LGR5 (Novus) 1:100
5. Anti human LIF-R-PE (R&D) 1:10
6. Control non treated cells

The cells were incubated for 30 minutes at room temperature with the antibodies in FACS buffer (0.5% BSA, 10 mM EDTA, 25 mM Hepes in PBS) and washed twice with PBS, followed by secondary antibodies anti rabbit Cy3 (for BST2, FLRT-3 and GPR-49) or anti goat Cy3 (COLEC12) for 15 minutes at room temperature. The cells were washed twice more with PBS and resuspended in FACS buffer. The expression levels of these markers in the EBS was compared to those in undifferentiated hESCs cells by FACS calibrator (BD) using Flow-Jo analysis.

Experimental Results

The results are presented in FIGS. 12A-E and are summarized in Table 21 hereinbelow.

TABLE 21
Expression analysis of antibodies in undifferentiated embryonic stem cells
and in 7-day old embryoid bodies

| Antibody | ESCs | 7 days EBs |
|---|---|---|
| BST-2 | 2.25% | 43.3% |
| FLRT-3* | 1.56% | 1.71% |
| COLEC12 | 0.26% | 70.19% |
| GPR-49/LGR5 | 1.34% | 38.2% |
| LIF-R | 2.28% | 16.13% |

*The result with FLRT3 may change after optimization of the antibody which is not usually used in FACS analysis.

The results presented in FIGS. 12A-E and in Table 21 above show that in young embryoid bodies there is increase in cells expressing the endodermal specific markers identified in the SOX17 expressing cells as compared to undifferentiated hESC. These results point to the potential usefulness of these markers for characterization and isolation of populations enriched in definitive endoderm.

### Example 7

### Negative Selection with Underexpressed Markers can be Used to Enrich for Definitive Endoderm

### Experimental Methods

Induction of Differentiation of Undifferentiated hESCs into Definite Endodermal Cells Using the IDE1/IDE2 Protocol

Undifferentiated H9.2 cells were treated with IDE1 or IDE2 as follows: The cells were washed once with PBS followed by incubation for 24 hours in RPMI with 5 μM IDE1/IDE2. The next day the medium was changed to RPMI with 0.2% FBS and 5 μM IDE1/IDE2 was added for 2 days. The cells were separated to single cells by treating the cells for 20-30 minutes with TrypLE Select (Invitrogen) in 37° C. The cells were washed once with EBs medium and once more in FACS buffer (0.5% BSA, 10 mM EDTA, 25 mM Hepes in PBS).

BST2 Expression on Adherent Cells

The cells which were induced by IDE1/IDE2 as described above were stained 1:100-1:200 with anti rabbit BST-2 (Santa Cruz) for 30 minutes in room temperature followed by secondary antibody Cy3 anti rabbit for 15 minutes at room temperature. Finally the cells were resuspended in FACS buffer and were tested by FACS calibrator.

BST2 Positive/KDR Negative and CXCR4 Positive/KDR Negative

The cells which were induced by IDE1/IDE2 as described above were stained 1:100-1:200 with anti rabbit BST-2 (Santa-Cruz) or 1:10 anti-human CXCR4-PerCP and 1:10 anti-human VEGF R2/KDR-FITC (both from R&D) for 30 min in room temperature followed by secondary antibody Cy3 anti rabbit for 15 min at room temperature (for the samples with BST2). Finally the cells were resuspended in FACS buffer and were tested by FACS calibrator.

Experimental Results

Cells which are Induced to Differentiate into Definite Endodermal Cells Express BST2—

The results of the FACS analysis showed that 11.9% of the cells in IDE1/IDE2 treated population express BST-2, and that only a subset of these, 10.7%, are BST2⁺/KDR⁻ (FIGS. 13A-C). These results demonstrate that combined use of the BST-2 marker with the KDR marker, which is underexpressed in definitive endoderm, may enable greater purification of an enriched definite endodermal cell population

Cells which are Induced to Differentiate into Definite Endodermal Cells Exhibit a BST2+/KDR− or CXCR4+/KDR− Expression Pattern—

The results of the FACS analysis showed that 1.05% of the cells treated with IDE1/2 express CXCR4 but not KDR (i.e., they exhibit the CXCR4+/KDR− expression signature) (FIG. 13D). As above, the combined use of positive and negative selection may yield a more highly enriched definitive endoderm population.

Altogether, these results show that the markers identified in the present study, individually and in combinations that include both overexpressed and underexpressed markers, can be used to isolate definite endodermal cells from a non-genetically modified population of cells that are differentiated from pluripotent stem cells.

### ANALYSIS AND DISCUSSION

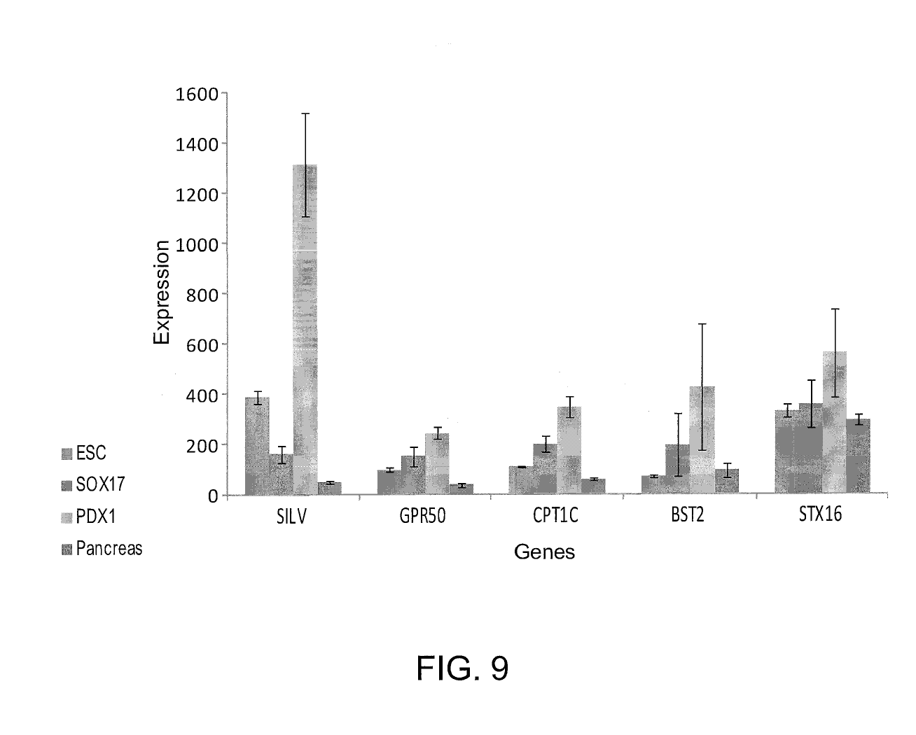

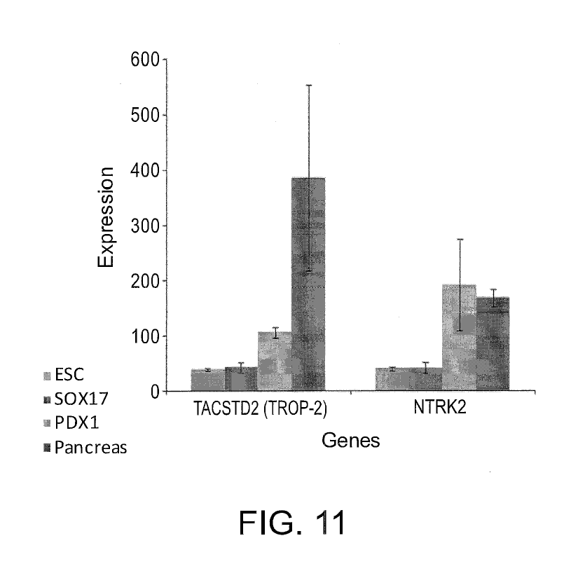

Differentiation of cells towards pancreatic beta—cells is still a challenging task. Based on genetic labeling approach the present inventors have succeeded in isolating from HESCs an enriched population of endoderm cells and pancreatic progenitor cells. These populations were then used in microarray analysis to identify candidate markers for defining (alone or in combination with other markers) specific precursor cell types characteristic of particular stages of the differentiation process. From the present analysis, a PDX1 specific gene GPR50 (FIG. 9, Table 14) and PDX1/Pancreas specific gene TROP2 (FIG. 11, Table 16) were selected for further study as promising genes. Particular emphasis has been placed on cell surface markers that by replacing genetic modification provide a more robust and stable approach for defining and isolating cell precursor populations. The information gained through these studies will permit more efficient generation of populations enriched with functional beta cells derived from stem cells. These protocols will rely on more efficient production of pancreatic progenitor cells and on their enrichment to enable improved treatment of diabetes.

hESCs represent one source of pluripotent cells capable of differentiating into almost any cell type (2). As a result, the therapeutic potential of these and other renewable stem cells for treating various diseases has generated much excitement. The present inventors disclose a method for selecting differentiating cells that will develop into functional insulin-producing beta cells. Such enriched populations may eventually be used in replacement therapy (e.g., cell transplantation) for the treatment of insulin dependent diabetes.

Selection and enrichment processes are needed because presently available protocols for differentiation of hESCs to definitive endoderm and pancreatic progenitors cells (5, 19, 21-24), do not yield pure populations. The importance of isolating the progenitors lies in the facts that, in contrast to mature beta cells, the progenitor cells have a proliferative capacity, and in contrast to hESCs they are non-tumorigenic. Thus purification or enrichment of progenitors followed by their continued culture would yield a more suitable transplant population.

The present inventors generated hESC clones harboring SOX17 and PDX1 BAC reporter constructs and identified a subpopulation of GFP⁺ cells.

SOX17 was originally identified as a stage-specific transcription activator during mouse spermatogenesis (26). Members of this gene family encode transcription factors that regulate the specification of cell types and tissue differentiation. Consistent with the general role of SOX genes in lineage specification, SOX17 is expressed specifically in the endoderm during gastrulation and plays a key role in endoderm formation.

PDX1 is expressed broadly in the pancreas during the first several days of pancreatic development, as the organ grows and branches. PDX1 regulates the insulin gene and from E15.5 (mouse embryonic days) onwards its expression becomes mainly restricted to β-cells. The transitions of PDX1 expression coincide with the overall conversion of progenitors to mature endocrine and exocrine cells (27).

The hESC clones harboring the SOX17 or PDX1⁺ BAC reporter and expressing GFP were isolated by FACS sorting and were analyzed by qPCR and microarray to identify cell surface and other markers expressed at these particular stages of the differentiation process.

The present inventors chose to use BACs, which are composed of relatively large stretches of genomic DNA, in order to render the transgenes less susceptible to mosaic or position effect variation. The larger size of BACs might also provide a more complete set of regulatory sequences (28).

Using this approach, the present inventors have succeeded in isolating enriched populations of endoderm and, for the first time pancreatic progenitor cells derived from human ESCs. Cell sorting based on SOX17 expression in reporter lines revealed markers which were previously correlated with definitive endoderm, thus validating the approach. Similarly, cells sorted base on PDX1 expression, revealed pancreatic progenitor markers. Affymetrix analysis then identified stage specific cell surface markers that enable cell type enrichment without the need for genetically modified hESCs. These markers provide a more robust, stable and clinically relevant approach for defining and isolating cell precursor populations.

To exemplify the above approach, use was made of two surface markers which are overexpressed in the PDX1⁺ cells, GPR50 and TROP-2. The latter was also found to be overexpressed in the pancreas array. Little is known about GPR50. The X-linked orphan receptor GPR50 shares 45% homology with the melatonin receptors, yet its ligand and physiological function remain mostly unknown. It is an orphan GPCR which has no affinity for melatonin, but as a dimer with MT1, it inhibits melatonin signaling (29, 30). Other reports have shown that GPR50 is also an important regulator of energy metabolism (31) The role of TROP-2 is not well understood and the physiological ligand is still unknown. TROP-2, the human trophoblast cell-surface glycoprotein, has been shown to play a role in regulating the growth of variety epithelial cancers including pancreatic tumors (32-35). Cell sorting of unmodified hESCs using these two markers enabled isolation of a cell population with a high relative expression of pancreatic progenitor transcription factors. These results exemplify the potential usefulness of the newly identified markers each alone, in combination with one another or in combination with other markers, in enabling isolation of enriched endoderm and pancreas progenitor populations. Other uses of these markers include quality control of candidate replacement therapy cell populations.

The availability of stage specific reporter constructs for key stages of pancreatic beta cell differentiation enables systematic assessment of the effect of signaling factors, small molecules or other compounds on growth, differentiation and survival of these progenitor cells. These constructs also provide valuable tools for efficient isolation of cell populations enriched for endoderm progenitor or pancreatic progenitor cells and their subsequent characterization by strategies such as microarray expression profiling. The discovery, through such an analysis, of new stage specific cell markers, including cell surface markers, opens the possibility for purification or enrichment of critical cell populations undergoing beta cell differentiation without the need for genetic modification. They also provide information about a “molecular signature” that can be used for “quality control” in assessing potential islet replacement cells developed from non-pancreatic sources.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

### Additional References are Cited in Text

1. White S A, J A Shaw and D E Sutherland. (2009). Pancreas transplantation. Lancet 373:1808-1817.
2. Itskovitz-Eldor J, M Schuldiner, D Karsenti, A Eden, O Yanuka, M Amit, H Soreq and N Benvenisty. (2000). Differentiation of human embryonic stem cells into embryoid bodies compromising the three embryonic germ layers. Mol Med 6:88-95.
3. Schuldiner M, O Yanuka, J Itskovitz-Eldor, D A Melton and N Benvenisty. (2000). Effects of eight growth factors on the differentiation of cells derived from human embryonic stem cells. Proc Natl Acad Sci USA 97:11307-11312.
4. Thomson J A, J Itskovitz-Eldor, S S Shapiro, M A Waknitz, J J Swiergiel, V S Marshall and J M Jones. (1998). Embryonic stem cell lines derived from human blastocysts. Science 282:1145-1147.
5. D'Amour K A, A G Bang, S Eliazer, O G Kelly, A D Agulnick, N G Smart, M A Moorman, E Kroon, M K Carpenter, E E Baetge, K A D'Amour, A D Agulnick, S Eliazer, O G Kelly, E Kroon and E E Baetge. (2006). Production of pancreatic hormone-expressing endocrine cells from human embryonic stem cells. Efficient differentiation of human embryonic stem cells to definitive endoderm. Nat Biotechnol 24:1392-1401.
6. Jiang J, M Au, K Lu, A Eshpeter, G Korbutt, G Fisk and A S Majumdar. (2007). Generation of insulin-producing islet-like clusters from human embryonic stem cells. Stem Cells 25:1940-1953.
7. Jiang W, Y Shi, D Zhao, S Chen, J Yong, J Zhang, T Qing, X Sun, P Zhang, M Ding, D Li and H Deng. (2007). In vitro derivation of functional insulin-producing cells from human embryonic stem cells. Cell Res 17:333-344.
8. Kroon E, L A Martinson, K Kadoya, A G Bang, O G Kelly, S Eliazer, H Young, M Richardson, N G Smart, J Cunningham, A D Agulnick, K A D'Amour, M K Carpenter and E E Baetge. (2008). Pancreatic endoderm derived from human embryonic stem cells generates glucose-responsive insulin-secreting cells in vivo. Nat Biotechnol 26:443-452.
9. Segev H, B Fishman, A Ziskind, M Shulman and J Itskovitz-Eldor. (2004). Differentiation of human embryonic stem cells into insulin-producing clusters. Stem Cells 22:265-274.
10. Maehr R, S Chen, M Snitow, T Ludwig, L Yagasaki, R Goland, R L Leibel and D A Melton. (2009). Generation of pluripotent stem cells from patients with type 1 diabetes. Proc Natl Acad Sci USA 106:15768-15773.
11. Tateishi K, J He, O Taranova, G Liang, A C D'Alessio and Y Zhang. (2008). Generation of insulin-secreting islet-like clusters from human skin fibroblasts. J Biol Chem 283:31601-31607.
12. Wells J M and D A Melton. (1999). Vertebrate endoderm development. Annu Rev Cell Dev Biol 15:393-410.
13. Amit M and J Itskovitz-Eldor. (2002). Derivation and spontaneous differentiation of human embryonic stem cells. J Anat 200:225-232.
14. Biancotti J C, K Narwani, N Buehler, B Mandefro, T Golan-Lev, O Yanuka, A Clark, D Hill, N Benvenisty and N Lavon. (2010). Human embryonic stem cells as models for aneuploid chromosomal syndromes. Stem Cells 28:1530-1540.
15. Lavon N, K Narwani, T Golan-Lev, N Buehler, D Hill and N Benvenisty. (2008). Derivation of euploid human embryonic stem cells from aneuploid embryos. Stem Cells 26:1874-1882.
16. Amit M, C Shariki, V Margulets and J Itskovitz-Eldor. (2004). Feeder layer- and serum-free culture of human embryonic stem cells. Biol Reprod 70:837-845. Epub 2003 November 2019.
17. Copeland N G, N A Jenkins and D L Court. (2001). Recombineering: a powerful new tool for mouse functional genomics. Nat Rev Genet 2:769-779.
18. Zwaka T P and J A Thomson. (2003). Homologous recombination in human embryonic stem cells. Nat Biotechnol 21:319-321.
19. D'Amour K A, A D Agulnick, S Eliazer, O G Kelly, E Kroon and E E Baetge. (2005). Efficient differentiation of human embryonic stem cells to definitive endoderm. Nat Biotechnol 23:1534-1541. Epub 2005 October 1528.
20. Yasunaga M, S Tada, S Torikai-Nishikawa, Y Nakano, M Okada, L M Jakt, S Nishikawa, T Chiba, T Era and S Nishikawa. (2005). Induction and monitoring of definitive and visceral endoderm differentiation of mouse ES cells. Nat Biotechnol 23:1542-1550. Epub 2005 November 1527.
21. Chen S, M Borowiak, J L Fox, R Maehr, K Osafune, L Davidow, K Lam, L F Peng, S L Schreiber, L L Rubin and D Melton. (2009). A small molecule that directs differentiation of human ESCs into the pancreatic lineage. Nat Chem Biol 5:258-265.
22. Frandsen U, A D Porneki, C Floridon, B M Abdallah and M Kassem. (2007). Activin B mediated induction of Pdx1 in human embryonic stem cell derived embryoid bodies. Biochem Biophys Res Commun 362:568-574.
23. Shi Y, L Hou, F Tang, W Jiang, P Wang, M Ding and H Deng. (2005). Inducing embryonic stem cells to differentiate into pancreatic beta cells by a novel three-step approach with activin A and all-trans retinoic acid. Stem Cells 23:656-662.
24. Shim J H, S E Kim, D H Woo, S K Kim, C H Oh, R McKay and J H Kim. (2007). Directed differentiation of human embryonic stem cells towards a pancreatic cell fate. Diabetologia 50:1228-1238.
25. Lavon N, O Yanuka and N Benvenisty. (2004). Differentiation and isolation of hepatic-like cells from human embryonic stem cells. Differentiation 72:230-238.
26. Kanai Y, M Kanai-Azuma, T Noce, T C Saido, T Shiroishi, Y Hayashi and K Yazaki. (1996). Identification of two SOX17 messenger RNA isoforms, with and without the high mobility group box region, and their differential expression in mouse spermatogenesis. J Cell Biol 133:667-681.
27. Gu G, J Dubauskaite and D A Melton. (2002). Direct evidence for the pancreatic lineage: NGN3+ cells are islet progenitors and are distinct from duct progenitors. Development 129:2447-2457.
28. Shizuya H and H Kouros-Mehr. (2001). The development and applications of the bacterial artificial chromosome cloning system. Keio J Med 50:26-30.
29. Chaste P, N Clement, O Mercati, J L Guillaume, R Delorme, H G Botros, C Pagan, S Perivier, I Scheid, G Nygren, H Anckarsater, M Rastam, O Stahlberg, C Gillberg, E Serrano, N Lemiere, J M Launay, M C Mouren-Simeoni, M Leboyer, C Gillberg, R Jockers and T Bourgeron. (2010). Identification of pathway-biased and deleterious melatonin receptor mutants in autism spectrum disorders and in the general population. PLoS One 5:e11495.
30. Levoye A, J Dam, M A Ayoub, J L Guillaume, C Couturier, P Delagrange and R Jockers. (2006). The orphan GPR50 receptor specifically inhibits MT1 melatonin receptor function through heterodimerization. Embo J 25:3012-3023.
31. Ivanova E A, D A Bechtold, S M Dupre, J Brennand, P Barrett, S M Luckman and A S Loudon. (2008). Altered metabolism in the melatonin-related receptor (GPR50) knockout mouse. Am J Physiol Endocrinol Metab 294:E176-182.
32. Fong D, P Moser, C Krammel, J M Gostner, R Margreiter, M Mitterer, G Gastl and G Spizzo. (2008). High expression of TROP2 correlates with poor prognosis in pancreatic cancer. Br J Cancer 99:1290-1295.
33. Fong D, G Spizzo, J M Gostner, G Gastl, P Moser, C Krammel, S Gerhard, M Rasse and K Laimer. (2008). TROP2: a novel prognostic marker in squamous cell carcinoma of the oral cavity. Mod Pathol 21:186-191.
34. Ohmachi T, F Tanaka, K Mimori, H Inoue, K Yanaga and M Mori. (2006). Clinical significance of TROP2 expression in colorectal cancer. Clin Cancer Res 12:3057-3063.
35. Chang C H, P Gupta, R Michel, M Loo, Y Wang, T M Cardillo and D M Goldenberg. (2010). Ranpirnase (frog RNase) targeted with a humanized, internalizing, anti-Trop-2 antibody has potent cytotoxicity against diverse epithelial cancer cells. Mol Cancer Ther 9:2276-2286.

What is claimed is:

1. A method of isolating a population of cells enriched in pancreatic progenitor cells, comprising:
(a) detecting in a population of cells comprising pancreatic progenitor cells, cells that express at least one marker selected from the group consisting of TROP-2, BST2, GPR50, ROBO1, NTRK2, ITGA4, LGI1, VIPR2, SLC2A1, MUC15, MUC12, LPHN3, MUC16, VTCN1, MMP16, FZD3, ITGB6, GFRA3, NLGN1, CNTFR, LPHN1, SULF1, ADAM23, SCUBE3, PLAU, CDON, SLIT2, C7orf68, PLXDC2, CD74, GPR56, AREG, BOC, and KLRK1; and
(b) isolating the cells expressing said at least one marker detected according to step (a) to thereby obtain an isolated population of cells enriched in pancreatic progenitor cells, thereby isolating the population of cells enriched in pancreatic progenitor cells.

2. The method of claim 1, further comprising detecting an additional marker selected from the group consisting of KDR, PTPRN, PRTG, PTPRZ1, GABRP, SILV, KIAA1772, PLP1, OVOS, HAPLN1, EPHA7, ENO2, PCDHB5, SYT1, DCT, GPAM, SLITRK6, DCC, FREM2, SDK2, CGA, ATP1B2, SEMA3D, PCDHB15, CDH1, WNT8B, LPAR4, NPIPL3, FAM171B, PTN, ABCC2, ADAMTS3, RASA4, CPT1C, SLC6A6, PCDHB3, LRRC37B, RNFT2, KCNG3, TRPC1, ALPPL2, OR4F21, CCL2, KIF5A, OLFM2, CACNG7, MPHOSPH9, SLC13A4, MOXD1, C6orf186, SLC4A8, STX16, AMY2A, SPARCL1, MGP, A2M, DCN, ATP8B1, MMRN1, EMP1, PLA2G2A, PDE3A, TLR3, CYP1B1, PTGIS, RFTN2, PLEKHA2, SMOC1, STOM, JAM2, CHL1, SCG5, IGFBP7, NPR3, IFI6, CR1L, OR2A4, OR2A7, GRID2, LPAR3, SEMA6A, ATP1A3, CAMKV, SCNN1G, SYT6, SLC18A2, ABCG2, HLA-DRA, HTR2C, EDNRB, PCDH11X, SLC17A7, SCNN1A, CD9, CXCL16, FXYD5, GABRQ, CACNA2D2, CLDN4, DPN, MMP24, GPR176, GPR64, GPR160, PCDH11Y, NKAIN4, SCN8A, THBS4, CR2, HLA-DQA1, HTR7, KCNS3, SLC7A3, HLA-DPB2, CACNA1B and GPR143.

3. The method of claim 1, wherein said population of cells enriched in pancreatic progenitor cells expresses a transcription factor selected from the group consisting of PDX1, ngn3, pax4, hlxb9, nkx6.1, Hnf6, and sox9.

4. The method of claim 1, wherein step (b) is effected by an immunological isolation technique selected from the group consisting of fluorescent activated cell sorter (FACS), Magnetic-activated cell sorting (MACS) or immunopanning.

5. The method of claim 1, wherein presence of at least a predetermined percentage of the pancreatic progenitor cells in said cell population indicates the suitability of the pancreatic progenitor cells for transplantation in a subject.

6. A method of isolating endocrine progenitors or insulin producing cells, comprising culturing the population of cells enriched in pancreatic progenitor cells isolated by the method of claim 1, under conditions suitable for maturation of the pancreatic progenitor cells into endocrine progenitors or beta cells, thereby isolating insulin producing cells.

7. An isolated population of cells enriched in pancreatic progenitor cells obtained according to the method of claim 1.

8. The isolated population of cells enriched in pancreatic progenitor cells of claim 7, wherein said pancreatic progenitor cells are genetically unmodified.

9. An isolated population of pancreatic progenitor cells, comprising at least about 50% of cells having a TROP-2+ and/or TROP-2+/GPR50+ expression pattern.

10. The isolated population of pancreatic progenitor cells of claim 9, wherein said cells are further characterized by the expression of at least one transcription factor selected from the group consisting of PDX1, ngn3, pax4, hlxb9, nkx6.1, Hnf6, and sox9.

11. The isolated population of pancreatic progenitor cells of claim 9, wherein said cells are obtained according to the method of claim 1.

12. The isolated population of pancreatic progenitor cells of claim 9, wherein said pancreatic progenitor cells are genetically unmodified.

13. A method of isolating a population of cells enriched in definite endodermal cells, comprising:
(a) detecting in a population of cells comprising definite endodermal cells, cells that express at least one marker selected from the group consisting of LIFR, LGR5, FLRT3, BST2, COLEC12, FSHR, ROR2, ITGA5, CD177, CCKBR, APOA1, FZD5, FN1, BMP2, ADAMTS9, DPP4, FGA, GPR128, IGFBP5, FZD4, STC1, TNFSF4, IHH, LRP2, LAMA1, GPC3, LPHN3, FGB, KIT, TRO, SPA17, ROBO2, and DLK1; and
(b) isolating the cells expressing said at least one marker detected according to step (a) to thereby obtain an isolated population of cells enriched in definite endodermal cells, thereby isolating a population of cells enriched in definite endodermal cells.

14. The method of claim 13, wherein said definite endodermal cells are further characterized by a SOX17+ or SOX17+/SOX7+ expression signature.

15. The method of claim 13, wherein said definite endodermal cells are further characterized by the expression of at least one marker selected from the group consisting of SOX17, SOX7, GSC, CER, FOXA2, CD34 and CXCR4; and a NANOG− expression signature.

16. The method of claim 13, wherein step (b) is by an immunological isolation assay technique selected from the group consisting of fluorescent activated cell sorter (FACS), Magnetic-activated cell sorting (MACS) or immunopanning.

17. An isolated population of cells enriched in definite endodermal cells obtained according to the method of claim 13.

18. The isolated population of cells enriched in definite endodermal cells of claim 17, comprising at least about 50% of cells having a SOX17+ or SOX17+/SOX7+ expression pattern.

19. The isolated population of cells enriched in definite endodermal cells of claim 17, wherein said definite endodermal cells are characterized by the expression of at least one marker selected from the group consisting of SOX17, SOX7, GSC, CER, FOXA2, CD34 and CXCR4; and a NANOG− expression signature.
